SEQUENCE LISTING (147 sequences)

SEQ ID NO: 1 - DNA, 21 bp, Artificial Sequence
  Synthetic oligonucleotide
  misc_feature (1)..(19): (1,2,3,4,5,6,7,8,9,10,11,12,13,14,15,16,17, 18,19)=RNA
  misc_feature (20)..(21): (20,21)=DNA
acuacugagugacaguagatt

SEQ ID NO: 2 - DNA, 21 bp, Artificial Sequence
  Synthetic oligonucleotide
  misc_feature (1)..(19): (1,2,3,4,5,6,7,8,9,10,11,12,13,14,15,16,17, 18,19)=RNA
  misc_feature (20)..(21): (20,21)=DNA
ucuacugucacucaguagutt

SEQ ID NO: 3 - DNA, 21 bp, Artificial Sequence
  Synthetic oligonucleotide
  misc_feature (1)..(19): (1,2,3,4,5,6,7,8,9,10,11,12,13,14,15,16,17, 18,19)=RNA
  misc_feature (20)..(21): (20,21)=DNA
ggugacauuugugaaacuutt

SEQ ID NO: 4 - DNA, 21 bp, Artificial Sequence
  Synthetic oligonucleotide
  misc_feature (1)..(19): (1,2,3,4,5,6,7,8,9,10,11,12,13,14,15,16,17, 18,19)=RNA
  misc_feature (20)..(21): (20,21)=DNA
aaguuucacaaaugucacctt

SEQ ID NO: 5 - DNA, 21 bp, Artificial Sequence
  Synthetic oligonucleotide
  misc_feature (1)..(19): (1,2,3,4,5,6,7,8,9,10,11,12,13,14,15,16,17, 18,19)=RNA
  misc_feature (20)..(21): (20,21)=DNA
agacgaggccuaagcaacatt

SEQ ID NO: 6 - DNA, 21 bp, Artificial Sequence
  Synthetic oligonucleotide
  misc_feature (1)..(19): (1,2,3,4,5,6,7,8,9,10,11,12,13,14,15,16,17, 18,19)=RNA
  misc_feature (20)..(21): (20,21)=DNA
uguugcuuaggccucgucutt

SEQ ID NO: 7 - DNA, 21 bp, Artificial Sequence
  Synthetic oligonucleotide
  misc_feature (1)..(19): (1,2,3,4,5,6,7,8,9,10,11,12,13,14,15,16,17, 18,19)=RNA
  misc_feature (20)..(21): (20,21)=DNA
uuguacacuuggucaacautt

SEQ ID NO: 8 - DNA, 21 bp, Artificial Sequence
  Synthetic oligonucleotide
  misc_feature (1)..(19): (1,2,3,4,5,6,7,8,9,10,11,12,13,14,15,16,17, 18,19)=RNA
  misc_feature (20)..(21): (20,21)=DNA
auguugaccaaguguacaatt

SEQ ID NO: 9 - DNA, 21 bp, Artificial Sequence
  Synthetic oligonucleotide
  misc_feature (1)..(19): (1,2,3,4,5,6,7,8,9,10,11,12,13,14,15,16,17, 18,19)=RNA
  misc_feature (20)..(21): (20,21)=DNA
cacuggaguucgagacgagtt

SEQ ID NO: 10 - DNA, 21 bp, Artificial Sequence
  Synthetic oligonucleotide
  misc_feature (1)..(19): (1,2,3,4,5,6,7,8,9,10,11,12,13,14,15,16,17, 18,19)=RNA
  misc_feature (20)..(21): (20,21)=DNA
cucgucucgaacuccagugtt

SEQ ID NO: 11 - RNA, 18 nt, Artificial Sequence
  Synthetic oligonucleotide
  modified_base (1)..(18): (1,2,3,4,5,6,7,8,9,10,11,12,13,14,15,16,17, 18) = phosphorothioate (PS) backbone modification
  modified_base (1)..(18): (1,2,3,4,5,6,7,8,9,10,11,12,13,14,15,16,17, 18) = 2′-O-methoxyethyl (2MOE)
  modified_base (1)..(16): (1,5,6,7,10,13,16) = 5-methyl uracil
  modified_base (2)..(15): (2,4,8,15) = 5-methyl cytosine
ucacuuucauaaugcugg

SEQ ID NO: 12 - DNA, 21 bp, Artificial Sequence
  Synthetic oligonucleotide
  misc_feature (1)..(19): (1,2,3,4,5,6,7,8,9,10,11,12,13,14,15,16,17, 18,19)=RNA
  misc_feature (20)..(21): (20,21)=DNA
uacuuuuguguaguacaaatt

SEQ ID NO: 13 - RNA, 20 nt, Artificial Sequence
  Synthetic oligonucleotide
  modified_base (1)..(20): (1,2,3,13,14,15,16,17,18, 19,20) = phosphorothioate (PS) backbone modification
  modified_base (1)..(19): (1,3,5,7,9,11,13,15,17,19) = 2′-O-methyl
  modified_base (1)..(1): (1) = 5′-phosophate
  modified_base (2)..(20): (2,4,6,8,10,12,14,16,18,20) = 2′-fluoro
uguugcuuaggccucgucuc

SEQ ID NO: 14 - RNA, 33 nt, Artificial Sequence
  Synthetic oligonucleotide
  modified_base (1)..(33): (1,2,3,13,14,15,16,17,18,19,20,21,22,23,24,25, 26,27,28,29,30,31,3 2,33) = phosphorothioate (PS) backbone modification
  modified_base (1)..(15): (1,3,5,7,9,11,13,15) = 2′-fluoro
  modified_base (2)..(14): (2,4,6,8,10,12,14) = 2′-O-methyl
  modified_base (15)..(16): (15,16) = L1, spacer-18 (S18 linker)
  modified_base (16)..(33): (16,17,18,19,20,21,22,23,24,25,26,27,28,29,30, 31,32,33) = 2′-O-methoxyethyl (2MOE)
  modified_base (16)..(31): (16,20,21,22,25,28,31) = 5-methyl uracil
  modified_base (17)..(30): (17,19,23,30) = 5-methyl cytosine
gaggccuaagcaacaucacuuucauaaugcugg

SEQ ID NO: 15 - RNA, 33 nt, Artificial Sequence
  Synthetic oligonucleotide
  modified_base (1)..(33): (1,2,3,13,14,15,16,17,18,19,20,21,22,23,24,25, 26,27,28,29,30,31,3 2,33) = phosphorothioate (PS) backbone modification
  modified_base (1)..(15): (1,3,5,7,9,11,13,15) = 2′-fluoro
  modified_base (2)..(14): (2,4,6,8,10,12,14) = 2′-O-methyl
  modified_base (15)..(16): (15,16) = L1, spacer-18 (S18 linker)
  modified_base (16)..(33): (16,17,18,19,20,21,22,23,24,25,26,27,28,29,30, 31,32,33) = 2′-O-methoxyethyl (2MOE)
  modified_base (18)..(33): (18,21,24,27,28,29,33) = 5-methyl uracil
  modified_base (19)..(32): (19,26,30,32) = 5-methyl cytosine
gaggccuaagcaacaggucguaauacuuucacu

SEQ ID NO: 16 - RNA, 30 nt, Artificial Sequence
  Synthetic oligonucleotide
  modified_base (1)..(30): (1,2,3,13,14,15,16,17,18,28,29, 30) = phosphorothioate (PS) backbone modification
  modified_base (1)..(30): (1,3,5,7,9,11,13,15,16,18,20,22,24,26,28, 30) = 2′-fluoro
  modified_base (2)..(29): (2,4,6,8,10,12,14,17,19,21,23,25,27, 29) = 2′-O-methyl
  modified_base (15)..(16): (15,16) = L1, spacer-S18 (S18 linker)
gaggccuaagcaacagaggccuaagcaaca

SEQ ID NO: 17 - RNA, 20 nt, Artificial Sequence
  Synthetic oligonucleotide
  modified_base (1)..(20): (1,2,3,13,14,15,16,17,18,19, 20) = phosphorothioate (PS) backbone modification
  modified_base (1)..(19): (1,3,5,7,9,11,13,15,17,19) = 2′-O-methyl
  modified_base (1)..(1): (1) = 5′-(E)-vinylphosphonate
  modified_base (2)..(20): (2,4,6,8,10,12,14,16,18,20) = 2′-fluoro
uguugcuuaggccucgucuc

SEQ ID NO: 18 - RNA, 36 nt, Artificial Sequence
  Synthetic oligonucleotide
  modified_base (1)..(36): (1,2,3,16,17,18,19,20,21,22,23,24,25,26,27,28, 29,30,31,32,33,34,3 5,36) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(36): (19,20,21,22,23,24,25,26,27,28,29,30,31,32,33, 34,35,36) = 2′-O-methoxyethyl (2MOE)
  modified_base (19)..(34): (19,23,24,25,28,31,34) = 5-methyl uracil
  modified_base (20)..(33): (20,22,26,33) = 5-methyl cytosine
gacgaggccuaagcaacaucacuuucauaaugcugg

SEQ ID NO: 19 - DNA, 39 bp, Artificial Sequence
  Synthetic oligonucleotide
  misc_feature (1)..(39): (1,2,3,4,5,6,7,8,9,10,11,12,13,14,15,16,17, 18,19,22,23,24,25,26,2 7,28,29,30,31,32,33,34,35,36,37,38,39)=RNA
  misc_feature (20)..(21): (20,21)=DNA
  modified_base (21)..(22): (21,22) = L1, spacer-S18 (S18 linker)
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine
uuguacacuuggucaacauttucacuuucauaaugcugg

SEQ ID NO: 20 - DNA, 39 bp, Artificial Sequence
  Synthetic oligonucleotide
  misc_feature (1)..(39): (1,2,3,4,5,6,7,8,9,10,11,12,13,14,15,16,17,18, 19,22,23,24,25,26,2 7,28,29,30,31,32,33,34,35,36,37,38,39)=RNA
  misc_feature (20)..(21): (20,21)=DNA
  modified_base (21)..(22): (21,22) = L1, spacer-S18 (S18 linker)
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine
auguugaccaaguguacaattucacuuucauaaugcugg

SEQ ID NO: 21 - DNA, 39 bp, Artificial Sequence
  Synthetic oligonucleotide
  misc_feature (1)..(39): (1,2,3,4,5,6,7,8,9,10,11,12,13,14,15,16,17,18, 19,22,23,24,25,26,2 7,28,29,30,31,32,33,34,35,36,37,38,39)=RNA
  misc_feature (20)..(21): (20,21)=DNA
  modified_base (21)..(22): (21,22) = L1, spacer-S18 (S18 linker)
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine
cacuggaguucgagacgagttucacuuucauaaugcugg

SEQ ID NO: 22 - DNA, 39 bp, Artificial Sequence
  Synthetic oligonucleotide
  misc_feature (1)..(39): (1,2,3,4,5,6,7,8,9,10,11,12,13,14,15,16,17,18, 19,22,23,24,25,26,2 7,28,29,30,31,32,33,34,35,36,37,38,39)=RNA
  misc_feature (20)..(21): (20,21)=DNA
  modified_base (21)..(22): (21,22) = L1, spacer-S18 (S18 linker)
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine
cucgucucgaacuccagugttucacuuucauaaugcugg

SEQ ID NO: 23 - DNA, 39 bp, Artificial Sequence; identical to SEQ ID NO: 21
  Synthetic oligonucleotide
  misc_feature (1)..(39): (1,2,3,4,5,6,7,8,9,10,11,12,13,14,15,16,17,18, 19,22,23,24,25,26,27,28,29,30,31,32,33,34,35,36,37,38,39)=RNA
  misc_feature (20)..(21): (20,21)=DNA
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine

SEQ ID NO: 24 - DNA, 39 bp, Artificial Sequence; identical to SEQ ID NO: 21
  Synthetic oligonucleotide
  misc_feature (1)..(39): (1,2,3,4,5,6,7,8,9,10,11,12,13,14,15,16,17,18, 19,22,23,24,25,26,2 7,28,29,30,31,32,33,34,35,36,37,38,39)=RNA
  misc_feature (20)..(21): (20,21)=DNA
  modified_base (21)..(22): (21,22) = L9, spacer-9 (S9 linker)
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine

SEQ ID NO: 25 - DNA, 39 bp, Artificial Sequence; identical to SEQ ID NO: 21
  Synthetic oligonucleotide
  misc_feature (1)..(39): (1,2,3,4,5,6,7,8,9,10,11,12,13,14,15,16,17,18, 19,22,23,24,25,26,27,28,29,30,31,32,33,34,35,36,37,38,39)=RNA
  misc_feature (20)..(21): (20,21)=DNA
  modified_base (21)..(22): (21,22) = L4, spacer-C6 (C6 linker)
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine

SEQ ID NO: 26 - DNA, 39 bp, Artificial Sequence; identical to SEQ ID NO: 22
  Synthetic oligonucleotide
  misc_feature (1)..(39): (1,2,3,4,5,6,7,8,9,10,11,12,13,14,15,16,17,18, 19,22,23,24,25,26,27,28,29,30,31,32,33,34,35,36,37,38,39)=RNA
  misc_feature (20)..(21): (20,21)=DNA
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine

SEQ ID NO: 27 - DNA, 39 bp, Artificial Sequence
  Synthetic oligonucleotide
  misc_feature (1)..(39): (1,2,3,4,5,6,7,8,9,10,11,12,13,14,15,16,17,18, 19,22,23,24,25,26,27,28,29,30,31,32,33,34,35,36,37,38,39)=RNA
  modified_base (1)..(18): (1,2,3,4,5,6,7,8,9,10,11,12,13,14,15,16,17, 18) = phosphorothioate (PS) backbone modification
  modified_base (1)..(18): (1,2,3,4,5,6,7,8,9,10,11,12,13,14,15,16,17, 18) = 2′-O-methoxyethyl (2MOE)
  modified_base (1)..(16): (1,5,6,7,10,13,16) = 5-methyl uracil
  modified_base (2)..(15): (2,4,8,15) = 5-methyl cytosine
  modified_base (18)..(19): (18,19) = L1, spacer-S18 (S18 linker)
  misc_feature (20)..(21): (20,21)=DNA
ucacuuucauaaugcuggcucgucucgaacuccagugtt

SEQ ID NO: 28 - DNA, 21 bp, Artificial Sequence
  Synthetic oligonucleotide
  misc_feature (1)..(19): (1,2,3,4,5,6,7,8,9,10,11,12,13,14,15,16,17,18, 19)=RNA
  misc_feature (20)..(21): (20,21)=DNA
uuuguacuacacaaaaguatt

SEQ ID NO: 29 - DNA, 39 bp, Artificial Sequence; identical to SEQ ID NO: 27
  Synthetic oligonucleotide
  misc_feature (1)..(37): (1,2,3,4,5,6,7,8,9,10,11,12,13,14,15,16,17,18, 19,20,21,22,23,24,25,26,27,28,29,30,31,32,33,34,35,36,37)=RNA
  modified_base (1)..(18): (1,2,3,4,5,6,7,8,9,10,11,12,13,14,15,16,17, 18) = phosphorothioate (PS) backbone modification
  modified_base (1)..(18): (1,2,3,4,5,6,7,8,9,10,11,12,13,14,15,16,17, 18) = 2′-O-methoxyethyl (2MOE)
  modified_base (1)..(16): (1,5,6,7,10,13,16) = 5-methyl uracil
  modified_base (2)..(15): (2,4,8,15) = 5-methyl cytosine
  modified_base (18)..(19): (18,19) = L9, spacer-9 (S9 linker)
  misc_feature (38)..(39): (38,39)=DNA

SEQ ID NO: 30 - DNA, 39 bp, Artificial Sequence; identical to SEQ ID NO: 27
  Synthetic oligonucleotide
  misc_feature (1)..(37): (1,2,3,4,5,6,7,8,9,10,11,12,13,14,15,16,17,18, 19,20,21,22,23,24,25,26,27,28,29,30,31,32,33,34,35,36,37)=RNA
  modified_base (1)..(18): (1,2,3,4,5,6,7,8,9,10,11,12,13,14,15,16,17, 18) = phosphorothioate (PS) backbone modification
  modified_base (1)..(18): (1,2,3,4,5,6,7,8,9,10,11,12,13,14,15,16,17, 18) = 2′-O-methoxyethyl (2MOE)
  modified_base (1)..(16): (1,5,6,7,10,13,16) = 5-methyl uracil
  modified_base (2)..(15): (2,4,8,15) = 5-methyl cytosine
  modified_base (18)..(19): (18,19) = L4, spacer-C6 (C6 linker)
  misc_feature (38)..(39): (38,39)=DNA

SEQ ID NO: 31 - DNA, 39 bp, Artificial Sequence; identical to SEQ ID NO: 22
  Synthetic oligonucleotide
  misc_feature (1)..(39): (1,2,3,4,5,6,7,8,9,10,11,12,13,14,15,16,17,18, 19,22,23,24,25,26,27,28,29,30,31,32,33,34,35,36,37,38,39)=RNA
  misc_feature (20)..(21): (20,21)=DNA
  modified_base (21)..(22): (21,22) = L9, spacer-9 (S9 linker)
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine

SEQ ID NO: 32 - DNA, 39 bp, Artificial Sequence; identical to SEQ ID NO: 22
  Synthetic oligonucleotide
  misc_feature (1)..(39): (1,2,3,4,5,6,7,8,9,10,11,12,13,14,15,16,17,18, 19,22,23,24,25,26,27,28,29,30,31,32,33,34,35,36,37,38,39)=RNA
  misc_feature (20)..(21): (20,21)=DNA
  modified_base (21)..(22): (21,22) = L4, spacer-C6 (C6 linker)
  modified_base (22)..(39): (22,23,24,25,26,27,28,29,30,31,32,33,34,35,36, 37,38,39) =  phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine

SEQ ID NO: 33 - DNA, 41 bp, Artificial Sequence
  Synthetic oligonucleotide
  misc_feature (1)..(41): (1,2,3,4,5,6,7,8,9,10,11,12,13,14,15,16,17,18, 19,20,21,24,25,26,2 7,28,29,30,31,32,33,34,35,36,37,38,39,40,41)=RNA
  misc_feature (22)..(23): (22,23)=DNA
  modified_base (23)..(24): (23,24) = L1, spacer-S18 (S18 linker)
  modified_base (24)..(41): (24,25,26,27,28,29,30,31,32,33,34,35,36,37,38, 39,40,41) =  phosphorothioate (PS) backbone modification
  modified_base (24)..(41): (24,25,26,27,28,29,30,31,32,33,34,35,36,37,38, 39,40,41) = 2′-O-methoxyethyl (2MOE)
  modified_base (24)..(39): (24,28,29,30,33,36,39) = 5-methyl uracil
  modified_base (25)..(38): (25,27,31,38) = 5-methyl cytosine
cacuggaguucgagacgaggcttucacuuucauaaugcugg

SEQ ID NO: 34 - DNA, 23 bp, Artificial Sequence
  Synthetic oligonucleotide
  misc_feature (1)..(21): (1,2,3,4,5,6,7,8,9,10,11,12,13,14,15,16,17,18, 19,20,21)=RNA
  misc_feature (22)..(23): (22,23)=DNA
gccucgucucgaacuccagugtt

SEQ ID NO: 35 - DNA, 23 bp, Artificial Sequence
  Synthetic oligonucleotide
  misc_feature (1)..(21): (1,2,3,4,5,6,7,8,9,10,11,12,13,14,15,16,17,18, 19,20,21)=RNA
  misc_feature (22)..(23): (22,23)=DNA
cacuggaguucgagacgaggctt

SEQ ID NO: 36 - DNA, 41 bp, Artificial Sequence
  Synthetic oligonucleotide
  misc_feature (1)..(39): (1,2,3,4,5,6,7,8,9,10,11,12,13,14,15,16,17,18, 19,20,21,24,25,26,27,28,29,30,31,32,33,34,35,36,37,38,39,40, 41)=RNA
  misc_feature (22)..(23): (22,23)=DNA
  modified_base (23)..(24): (23,24) = L1, spacer-S18 (S18 linker)
  modified_base (24)..(41): (24,25,26,27,28,29,30,31,32,33,34,35,36,37,38, 39,40,41) = phosphorothioate (PS) backbone modification
  modified_base (24)..(41): (24,25,26,27,28,29,30,31,32,33,34,35,36,37,38, 39,40,41) = 2′-O-methoxyethyl (2MOE)
  modified_base (24)..(39): (24,28,29,30,33,36,39) = 5-methyl uracil
  modified_base (25)..(38): (25,27,31,38) = 5-methyl cytosine
gccucgucucgaacuccagugttucacuuucauaaugcugg

SEQ ID NO: 37 - DNA, 39 bp, Artificial Sequence
  Synthetic oligonucleotide
  misc_feature (1)..(39): (1,2,3,4,5,6,7,8,9,10,11,12,13,14,15,16,17,18, 19,22,23,24,25,26,27,28,29,30,31,32,33,34,35,36,37,38,39)=RNA
  misc_feature (20)..(21): (20,21)=DNA
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine
agacgaggccuaagcaacattucacuuucauaaugcugg

SEQ ID NO: 38 - DNA, 39 bp, Artificial Sequence
  Synthetic oligonucleotide
  misc_feature (1)..(37): (1,2,3,4,5,6,7,8,9,10,11,12,13,14,15,16,17,18, 19,20,21,22,23,24,25,26,27,28,29,30,31,32,33,34,35,36,37)=RNA
  modified_base (1)..(18): (1,2,3,4,5,6,7,8,9,10,11,12,13,14,15,16,17, 18) = phosphorothioate (PS) backbone modification
  modified_base (1)..(18): (1,2,3,4,5,6,7,8,9,10,11,12,13,14,15,16,17, 18) = 2′-O-methoxyethyl (2MOE)
  modified_base (1)..(16): (1,5,6,7,10,13,16) = 5-methyl uracil
  modified_base (2)..(15): (2,4,8,15) = 5-methyl cytosine
  modified_base (18)..(19): (18,19) = L1, spacer-S18 (S18 linker)
  misc_feature (38)..(39): (38,39)=DNA
ucacuuucauaaugcuggagacgaggccuaagcaacatt

SEQ ID NO: 39 - DNA, 39 bp, Artificial Sequence; identical to SEQ ID NO: 38
  Synthetic oligonucleotide
  misc_feature (1)..(37): (1,2,3,4,5,6,7,8,9,10,11,12,13,14,15,16,17,18, 19,20,21,22,23,24,25,26,27,28,29,30,31,32,33,34,35,36,37)=RNA
  modified_base (1)..(18): (1,2,3,4,5,6,7,8,9,10,11,12,13,14,15,16,17, 18) = phosphorothioate (PS) backbone modification
  modified_base (1)..(18): (1,2,3,4,5,6,7,8,9,10,11,12,13,14,15,16,17, 18) = 2′-O-methoxyethyl (2MOE)
  modified_base (1)..(16): (1,5,6,7,10,13,16) = 5-methyl uracil
  modified_base (2)..(15): (2,4,8,15) = 5-methyl cytosine
  modified_base (18)..(19): (18,19) = L9, spacer-9 (S9 linker)
  misc_feature (38)..(39): (38,39)=DNA

SEQ ID NO: 40 - DNA, 39 bp, Artificial Sequence; identical to SEQ ID NO: 38
  Synthetic oligonucleotide
  misc_feature (1)..(37): (1,2,3,4,5,6,7,8,9,10,11,12,13,14,15,16,17,18, 19,20,21,22,23,24,25,26,27,28,29,30,31,32,33,34,35,36,37)=RNA
  modified_base (1)..(18): (1,2,3,4,5,6,7,8,9,10,11,12,13,14,15,16,17, 18) = phosphorothioate (PS) backbone modification
  modified_base (1)..(18): (1,2,3,4,5,6,7,8,9,10,11,12,13,14,15,16,17, 18) = 2′-O-methoxyethyl (2MOE)
  modified_base (1)..(16): (1,5,6,7,10,13,16) = 5-methyl uracil
  modified_base (2)..(15): (2,4,8,15) = 5-methyl cytosine
  modified_base (18)..(19): (18,19) = L4, spacer-C6 (C6 linker)
  misc_feature (38)..(39): (38,39)=DNA

SEQ ID NO: 41 - DNA, 39 bp, Artificial Sequence; identical to SEQ ID NO: 37
  Synthetic oligonucleotide
  misc_feature (1)..(39): (1,2,3,4,5,6,7,8,9,10,11,12,13,14,15,16,17,18, 19,22,23,24,25,26,27,28,29,30,31,32,33,34,35,36,37,38,39)=RNA
  misc_feature (20)..(21): (20,21)=DNA
  modified_base (21)..(22): (21,22) = L1, spacer-S18 (S18 linker)
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine

SEQ ID NO: 42 - DNA, 39 bp, Artificial Sequence; identical to SEQ ID NO: 37
  Synthetic oligonucleotide
  misc_feature (1)..(39): (1,2,3,4,5,6,7,8,9,10,11,12,13,14,15,16,17,18, 19,22,23,24,25,26,27,28,29,30,31,32,33,34,35,36,37,38,39)=RNA
  misc_feature (20)..(21): (20,21)=DNA
  modified_base (21)..(22): (21,22) = L9, spacer-9 (S9 linker)
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine

SEQ ID NO: 43 - DNA, 39 bp, Artificial Sequence; identical to SEQ ID NO: 37
  Synthetic oligonucleotide
  misc_feature (1)..(39): (1,2,3,4,5,6,7,8,9,10,11,12,13,14,15,16,17,18, 19,22,23,24,25,26,27,28,29,30,31,32,33,34,35,36,37,38,39)=RNA
  misc_feature (20)..(21): (20,21)=DNA
  modified_base (21)..(22): (21,22) = L4, spacer-C6 (C6 linker)
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine

SEQ ID NO: 44 - DNA, 39 bp, Artificial Sequence
  Synthetic oligonucleotide
  misc_feature (1)..(39): (1,2,3,4,5,6,7,8,9,10,11,12,13,14,15,16,17,18, 19,22,23,24,25,26,27,28,29,30,31,32,33,34,35,36,37,38,39)=RNA
  misc_feature (20)..(21): (20,21)=DNA
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine
uguugcuuaggccucgucuttucacuuucauaaugcugg

SEQ ID NO: 45 - DNA, 39 bp, Artificial Sequence; identical to SEQ ID NO: 44
  Synthetic oligonucleotide
  misc_feature (1)..(39): (1,2,3,4,5,6,7,8,9,10,11,12,13,14,15,16,17,18, 19,22,23,24,25,26,27,28,29,30,31,32,33,34,35,36,37,38,39)=RNA
  misc_feature (20)..(21): (20,21)=DNA
  modified_base (21)..(22): (21,22) = L1, spacer-S18 (S18 linker)
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine

SEQ ID NO: 46 - DNA, 39 bp, Artificial Sequence; identical to SEQ ID NO: 44
  Synthetic oligonucleotide
  misc_feature (1)..(39): (1,2,3,4,5,6,7,8,9,10,11,12,13,14,15,16,17,18, 19,22,23,24,25,26,27,28,29,30,31,32,33,34,35,36,37,38,39)=RNA
  misc_feature (20)..(21): (20,21)=DNA
  modified_base (21)..(22): (21,22) = L9, spacer-9 (S9 linker)
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine

SEQ ID NO: 47 - DNA, 39 bp, Artificial Sequence; identical to SEQ ID NO: 44
  Synthetic oligonucleotide
  misc_feature (1)..(39): (1,2,3,4,5,6,7,8,9,10,11,12,13,14,15,16,17,18, 19,22,23,24,25,26,27,28,29,30,31,32,33,34,35,36,37,38,39)=RNA
  misc_feature (20)..(21): (20,21)=DNA
  modified_base (21)..(22): (21,22) = L4, spacer-C6 (C6 linker)
  modified_base (22)..(39): (22,23,24,25,26,27,28,29,30,31,32,33,34,35,36, 37,38,39) = phosphorothioate (PS) backbone modification
  modified_base (22)..(39): (22,23,24,25,26,27,28,29,30,31,32,33,34,35,36, 37,38,39) = 2′-O-methoxyethyl (2MOE)
  modified_base (22)..(37): (22,26,27,28,31,34,37) = 5-methyl uracil
  modified_base (23)..(36): (23,25,29,36) = 5-methyl cytosine

SEQ ID NO: 48 - DNA, 41 bp, Artificial Sequence
  Synthetic oligonucleotide
  misc_feature (1)..(41): (1,2,3,4,5,6,7,8,9,10,11,12,13,14,15,16,17,18, 19,20,21,24,25,26,27,28,29,30,31,32,33,34,35,36,37,38,39,40, 41)=RNA
  misc_feature (22)..(23): (22,23)=DNA
  modified_base (23)..(24): (23,24) = L1, spacer-S18 (S18 linker)
  modified_base (24)..(41): (24,25,26,27,28,29,30,31,32,33,34,35,36,37,38, 39,40,41) = phosphorothioate (PS) backbone modification
  modified_base (24)..(41): (24,25,26,27,28,29,30,31,32,33,34,35,36,37,38, 39,40,41) = 2′-O-methoxyethyl (2MOE)
  modified_base (24)..(39): (24,28,29,30,33,36,39) = 5-methyl uracil
  modified_base (25)..(38): (25,27,31,38) = 5-methyl cytosine
cgagacgaggccuaagcaacattucacuuucauaaugcugg

SEQ ID NO: 49 - DNA, 23 bp, Artificial Sequence
  Synthetic oligonucleotide
  misc_feature (1)..(21): (1,2,3,4,5,6,7,8,9,10,11,12,13,14,15,16,17,18, 19,20,21)=RNA
  misc_feature (22)..(23): (22,23)=DNA
uguugcuuaggccucgucucgtt

SEQ ID NO: 50 - DNA, 23 bp, Artificial Sequence
  Synthetic oligonucleotide
  misc_feature (1)..(21): (1,2,3,4,5,6,7,8,9,10,11,12,13,14,15,16,17,18, 19,20,21)=RNA
  misc_feature (22)..(23): (22,23)=DNA
cgagacgaggccuaagcaacatt

SEQ ID NO: 51 - DNA, 41 bp, Artificial Sequence
  Synthetic oligonucleotide
  misc_feature (1)..(41): (1,2,3,4,5,6,7,8,9,10,11,12,13,14,15,16,17,18, 19,20,21,24,25,26,27,28,29,30,31,32,33,34,35,36,37,38,39,40, 41)=RNA
  misc_feature (22)..(23): (22,23)=DNA
  modified_base (23)..(24): (23,24) = L1, spacer-S18 (S18 linker)
  modified_base (24)..(41): (24,25,26,27,28,29,30,31,32,33,34,35,36,37,38, 39,40,41) = phosphorothioate (PS) backbone modification
  modified_base (24)..(41): (24,25,26,27,28,29,30,31,32,33,34,35,36,37,38, 39,40,41) = 2′-O-methoxyethyl (2MOE)
  modified_base (24)..(39): (24,28,29,30,33,36,39) = 5-methyl uracil
  modified_base (25)..(38): (25,27,31,38) = 5-methyl cytosine
uguugcuuaggccucgucucgttucacuuucauaaugcugg

SEQ ID NO: 52 - RNA, 35 nt, Artificial Sequence
  Synthetic oligonucleotide
  modified_base (18)..(19): (18,19) = L1, spacer-S18 (S18 linker)
gacgaggccuaagcaacacgucucgaacuccagug

SEQ ID NO: 53 - RNA, 20 nt, Artificial Sequence
  Synthetic oligonucleotide
uguugcuuaggccucgucuc

SEQ ID NO: 54 - RNA, 53 nt, Artificial Sequence
  Synthetic oligonucleotide
  modified_base (18)..(19): (18,19) = L1, spacer-S18 (S18 linker)
  modified_base (19)..(36): (19,20,21,22,23,24,25,26,27,28,29,30,31,32,33, 34,35,36) = phosphorothioate (PS) backbone modification
  modified_base (19)..(36): (19,20,21,22,23,24,25,26,27,28,29,30,31,32,33, 34,35,36) = 2′-O-methoxyethyl (2MOE)
  modified_base (19)..(34): (19,23,24,25,28,31,34) = 5-methyl uracil
  modified_base (20)..(33): (20,22,26,33) = 5-methyl cytosine
  modified_base (36)..(37): (36,37) = L1, spacer-S18 (S18 linker)
gacgaggccuaagcaacaucacuuucauaaugcuggcgucucgaacuccagug

SEQ ID NO: 55 - RNA, 53 nt, Artificial Sequence
  Synthetic oligonucleotide
  modified_base (18)..(19): (18,19) = L1, spacer-S18 (S18 linker)
  modified_base (35)..(36): (35,36) = L1, spacer-S18 (S18 linker)
  modified_base (36)..(53): (36,37,38,39,40,41,42,43,44,45,46,47,48,49,50, 51,52,53) = phosphorothioate (PS) backbone modification
  modified_base (36)..(53): (36,37,38,39,40,41,42,43,44,45,46,47,48,49,50, 51,52,53) = 2′-O-methoxyethyl (2MOE)
  modified_base (36)..(51): (36,40,41,42,45,48,51) = 5-methyl uracil
  modified_base (37)..(50): (37,39,43,50) = 5-methyl cytosine
gacgaggccuaagcaacacgucucgaacuccagugucacuuucauaaugcugg

SEQ ID NO: 56 - RNA, 37 nt, Artificial Sequence
  Synthetic oligonucleotide
  modified_base (18)..(19): (18,19) = L1, spacer-S18 (S18 linker)
gacgaggccuaagcaacacucgucucgaacuccagug

SEQ ID NO: 57 - RNA, 55 nt, Artificial Sequence
  Synthetic oligonucleotide
  modified_base (18)..(19): (18,19) = L1, spacer-S18 (S18 linker)
  modified_base (19)..(36): (19,20,21,22,23,24,25,26,27,28,29,30,31,32,33, 34,35,36) = phosphorothioate (PS) backbone modification
  modified_base (19)..(36): (19,20,21,22,23,24,25,26,27,28,29,30,31,32,33, 34,35,36) = 2′-O-methoxyethyl (2MOE)
  modified_base (19)..(34): (19,23,24,25,28,31,34) = 5-methyl uracil
  modified_base (20)..(33): (20,22,26,33) = 5-methyl cytosine
  modified_base (36)..(37): (36,37) = L1, spacer-S18 (S18 linker)
gacgaggccuaagcaacaucacuuucauaaugcuggcucgucucgaacuccagug

SEQ ID NO: 58 - RNA, 55 nt, Artificial Sequence
  Synthetic oligonucleotide
  modified_base (18)..(19): (18,19) = L1, spacer-S18 (S18 linker)
  modified_base (37)..(38): (37,38) = L1, spacer-S18 (S18 linker)
  modified_base (38)..(55): (38,39,40,41,42,43,44,45,46,47,48,49,50,51,52, 53,54,55) = phosphorothioate (PS) backbone modification
  modified_base (38)..(55): (38,39,40,41,42,43,44,45,46,47,48,49,50,51,52, 53,54,55) = 2′-O-methoxyethyl (2MOE)
  modified_base (38)..(53): (38,42,43,44,47,50,53) = 5-methyl uracil
  modified_base (39)..(52): (39,41,45,52) = 5-methyl cytosine
gacgaggccuaagcaacacucgucucgaacuccagugucacuuucauaaugcugg

SEQ ID NO: 59 - RNA, 18 nt, Artificial Sequence
  Synthetic oligonucleotide
gacgaggccuaagcaaca

SEQ ID NO: 60 - RNA, 18 nt, Artificial Sequence
  Synthetic oligonucleotide
  modified_base (1)..(18): (1,2,3,16,17,18) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
gacgaggccuaagcaaca

SEQ ID NO: 61 - RNA, 36 nt, Artificial Sequence; identical to SEQ ID NO: 18
  Synthetic oligonucleotide
  modified_base (1)..(36): (1,2,3,16,17,18,19,20,21,22,23,24,25,26,27,28, 29,30,31,32,33,34,35,36) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(36): (19,20,21,22,23,24,25,26,27,28,29,30,31,32,33, 34,35,36) = 2′-O-methoxyethyl (2MOE)
  modified_base (19)..(34): (19,23,24,25,28,31,34) = 5-methyl uracil
  modified_base (20)..(33): (20,22,26,33) = 5-methyl cytosine

SEQ ID NO: 62 - RNA, 19 nt, Artificial Sequence
  Synthetic oligonucleotide
ccaacucauucuccaaguc

SEQ ID NO: 63 - RNA, 21 nt, Artificial Sequence
  Synthetic oligonucleotide
uacuuggagaaugaguuggca

SEQ ID NO: 64 - DNA, 21 bp, Artificial Sequence
  Synthetic oligonucleotide
  misc_feature (1)..(19): (1,2,3,4,5,6,7,8,9,10,11,12,13,14,15,16,17,18, 19)=RNA
  misc_feature (20)..(21): (20,21)=DNA
ccuauaugugguagaguautt

SEQ ID NO: 65 - DNA, 21 bp, Artificial Sequence
  Synthetic oligonucleotide
  misc_feature (1)..(19): (1,2,3,4,5,6,7,8,9,10,11,12,13,14,15,16,17,18, 19)=RNA
  misc_feature (20)..(21): (20,21)=DNA
auacucuaccacauauaggtt

SEQ ID NO: 66 - DNA, 21 bp, Artificial Sequence
  Synthetic oligonucleotide
  misc_feature (1)..(19): (1,2,3,4,5,6,7,8,9,10,11,12,13,14,15,16,17,18, 19)=RNA
  misc_feature (20)..(21): (20,21)=DNA
gccgaagucaucuggacaatt

SEQ ID NO: 67 - DNA, 21 bp, Artificial Sequence
  Synthetic oligonucleotide
  misc_feature (1)..(19): (1,2,3,4,5,6,7,8,9,10,11,12,13,14,15,16,17,18, 19)=RNA
  misc_feature (20)..(21): (20,21)=DNA
uuguccagaugacuucggctt

SEQ ID NO: 68 - RNA, 20 nt, Artificial Sequence
  Synthetic oligonucleotide
  modified_base (1)..(20): (1,2,3,4,5,6,7,8,9,10,11,12,13,14,15,16,17,18, 19,20) = phosphorothioate (PS) backbone modification
  modified_base (1)..(20): (1,2,3,4,5,16,17,18,19,20) = 2′-O-methoxyethyl (2MOE)
  modified_base (1)..(20): (1,19,20) = 5-methyl cytosine
  modified_base (3)..(5): (3,5) = 5-methyl uracil
cauauagguccuugggaacc

SEQ ID NO: 69 - RNA, 20 nt, Artificial Sequence
  Synthetic oligonucleotide
  modified_base (1)..(20): (1,2,3,4,5,6,7,8,9,10,11,12,13,14,15,16,17,18, 19,20) = phosphorothioate (PS) backbone modification
  modified_base (1)..(20): (1,2,3,4,5,16,17,18,19,20) = 2′-O-methoxyethyl (2MOE)
  modified_base (1)..(5): (1,5) = 5-methyl cytosine
  modified_base (3)..(17): (3,17) = 5-methyl uracil
cauacucuaccacauauagg

SEQ ID NO: 70 - RNA, 20 nt, Artificial Sequence
  Synthetic oligonucleotide
  modified_base (1)..(20): (1,2,3,4,5,6,7,8,9,10,11,12,13,14,15,16,17,18, 19,20) = phosphorothioate (PS) backbone modification
  modified_base (1)..(20): (1,2,3,4,5,16,17,18,19,20) = 2′-O-methoxyethyl (2MOE)
  modified_base (1)..(5): (1,5) = 5-methyl cytosine
  modified_base (3)..(17): (3,17) = 5-methyl uracil
cauacucuaccacauauagg

SEQ ID NO: 71 - DNA, 40 bp, Artificial Sequence
  Synthetic oligonucleotide
  misc_feature (1)..(38): (1,2,3,4,5,6,7,8,9,10,11,12,13,14,15,16,17,18, 1920,21,22,23,24,25,26,27,28,29,30,31,32,33,34,35,36,37,38)=RNA
  modified_base (19)..(20): (19,20) = L1, spacer-S18 (S18 linker)
  misc_feature (39)..(40): (39,40)=DNA
ccaacucauucuccaagucccuauaugugguagaguautt

SEQ ID NO: 72 - RNA, 39 nt, Artificial Sequence
  Synthetic oligonucleotide
  modified_base (19)..(20): (19,20) = L1, spacer-S18 (S18 linker)
  modified_base (20)..(39): (20,21,22,23,24,25,26,27,28,29,30,31,32,33,34, 35,36,37,38,39) = phosphorothioate (PS) backbone modification
  modified_base (20)..(39): (20,21,22,23,24,35,36,37,38,39) = 2′-O-methoxyethyl (2MOE)
  modified_base (20)..(39): (20,38,39) = 5-methyl cytosine
  modified_base (22)..(24): (22,24) = 5-methyl uracil
ccaacucauucuccaaguccauauagguccuugggaacc

SEQ ID NO: 73 - RNA, 39 nt, Artificial Sequence
  Synthetic oligonucleotide
  modified_base (19)..(20): (19,20) = L1, spacer-S18 (S18 linker)
  modified_base (20)..(39): (20,21,22,23,24,25,26,27,28,29,30,31,32,33,34, 35,36,37,38,39) = phosphorothioate (PS) backbone modification
  modified_base (20)..(39): (20,21,22,23,24,35,36,37,38,39) = 2′-O-methoxyethyl (2MOE)
  modified_base (20)..(24): (20,24) = 5-methyl cytosine
  modified_base (22)..(36): (22,36) = 5-methyl uracil
ccaacucauucuccaaguccauacucuaccacauauagg

SEQ ID NO: 74 - RNA, 39 nt, Artificial Sequence
  Synthetic oligonucleotide
  modified_base (19)..(20): (19,20) = L1, spacer-S18 (S18 linker)
  modified_base (20)..(39): (20,21,22,23,24,25,26,27,28,29,30,31,32,33,34, 35,36,37,38,39) = phosphorothioate (PS) backbone modification
  modified_base (20)..(39): (20,21,22,23,24,35,36,37,38,39) = 2′-O-methoxyethyl (2MOE)
  modified_base (20)..(39): (20,36,39) = 5-methyl cytosine
  modified_base (21)..(35): (21,22,24,35) = 5-methyl uracil
ccaacucauucuccaaguccuuguccagaugacuucggc

SEQ ID NO: 75 - RNA, 39 nt, Artificial Sequence
  Synthetic oligonucleotide
  modified_base (19)..(20): (19,20) = L1, spacer-S18 (S18 linker)
  modified_base (20)..(39): (20,21,22,23,24,25,26,27,28,29,30,31,32,33,34, 35,36,37,38,39) = phosphorothioate (PS) backbone modification
  modified_base (20)..(39): (20,21,22,23,24,35,36,37,38,39) = 2′-O-methoxyethyl (2MOE)
  modified_base (20)..(39): (20,21,39) = 5-methyl cytosine
  modified_base (35)..(37): (35,37) = 5-methyl uracil
ccaacucauucuccaagucccaaggguuccuggauauac

SEQ ID NO: 76 - RNA, 39 nt, Artificial Sequence
  Synthetic oligonucleotide
  modified_base (19)..(20): (19,20) = L1, spacer-S18 (S18 linker)
  modified_base (20)..(39): (20,21,22,23,24,25,26,27,28,29,30,31,32,33,34, 35,36,37,38,39) = phosphorothioate (PS) backbone modification
  modified_base (20)..(39): (20,21,22,23,24,35,36,37,38,39) = 2′-O-methoxyethyl (2MOE)
  modified_base (23)..(37): (23,37) = 5-methyl uracil
  modified_base (35)..(39): (35,39) = 5-methyl cytosine
ccaacucauucuccaagucggauauacaccaucucauac

SEQ ID NO: 77 - RNA, 39 nt, Artificial Sequence
  Synthetic oligonucleotide
  modified_base (19)..(20): (19,20) = L1, spacer-S18 (S18 linker)
  modified_base (20)..(39): (20,21,22,23,24,25,26,27,28,29,30,31,32,33,34, 35,36,37,38,39) = phosphorothioate (PS) backbone modification
  modified_base (20)..(39): (20,21,22,23,24,35,36,37,38,39) = 2′-O-methoxyethyl (2MOE)
  modified_base (20)..(39): (20,23,39) = 5-methyl cytosine
  modified_base (24)..(38): (24,35,37,38) = 5-methyl uracil
ccaacucauucuccaaguccggcuucaguagaccuguuc

SEQ ID NO: 78 - RNA, 19 nt, Artificial Sequence
  Synthetic oligonucleotide
cacuggaguucgagacgag

SEQ ID NO: 79 - RNA, 34 nt, Artificial Sequence
  Synthetic oligonucleotide
  modified_base (1)..(34): (1,2,3,16,17,18,19,20,21,22,23,24,25,26,27,28, 29,30,31,32,33,34) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(34): (19,20,21,22,23,24,25,26,27,28,29,30,31,32,33, 34) = 2′-O-methoxyethyl (2MOE)
  modified_base (20)..(31): (20,24,31) = 5-methyl cytosine
  modified_base (21)..(32): (21,22,23,26,29,32) = 5-methyl uracil
gacgaggccuaagcaacaacuuucauaaugcugg

SEQ ID NO: 80 - RNA, 33 nt, Artificial Sequence
  Synthetic oligonucleotide
  modified_base (1)..(33): (1,2,3,16,17,18,19,20,21,22,23,24,25,26,27,28, 29,30,31,32,33) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(33): (19,20,21,22,23,24,25,26,27,28,29,30,31,32, 33) = 2′-O-methoxyethyl (2MOE)
  modified_base (19)..(30): (19,20,21,24,27,30) = 5-methyl uracil
  modified_base (22)..(33): (22,29,33) = 5-methyl cytosine
gacgaggccuaagcaacauuucauaaugcuggc

SEQ ID NO: 81 - RNA, 32 nt, Artificial Sequence
  Synthetic oligonucleotide
  modified_base (1)..(32): (1,2,3,16,17,18,19,20,21,22,23,24,25,26,27,28, 29,30,31,32) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(32): (19,20,21,22,23,24,25,26,27,28,29,30,31, 32) = 2′-O-methoxyethyl (2MOE)
  modified_base (19)..(29): (19,20,23,26,29) = 5-methyl uracil
  modified_base (21)..(32): (21,28,32) = 5-methyl cytosine
gacgaggccuaagcaacauucauaaugcuggc

SEQ ID NO: 82 - RNA, 31 nt, Artificial Sequence
  Synthetic oligonucleotide
  modified_base (1)..(31): (1,2,3,16,17,18,19,20,21,22,23,24,25,26,27,28, 29,30,31) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(31): (19,20,21,22,23,24,25,26,27,28,29,30, 31) = 2′-O-methoxyethyl (2MOE)
  modified_base (19)..(28): (19,22,25,28) = 5-methyl uracil
  modified_base (20)..(31): (20,27,31) = 5-methyl cytosine
gacgaggccuaagcaacaucauaaugcuggc

SEQ ID NO: 83 - RNA, 30 nt, Artificial Sequence
  Synthetic oligonucleotide
  modified_base (1)..(30): (1,2,3,16,17,18,19,20,21,22,23,24,25,26,27,28, 29,30) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(30): (19,20,21,22,23,24,25,26,27,28,29, 30) = 2′-O-methoxyethyl (2MOE)
  modified_base (20)..(26): (20,23,26) = 5-methyl uracil
  modified_base (25)..(29): (25,29) = 5-methyl cytosine
gacgaggccuaagcaacaauaaugcuggca

SEQ ID NO: 84 - RNA, 29 nt, Artificial Sequence
  Synthetic oligonucleotide
  modified_base (1)..(29): (1,2,3,16,17,18,19,20,21,22,23,24,25,26,27,28, 29) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(29): (19,20,21,22,23,24,25,26,27,28, 29) = 2′-O-methoxyethyl (2MOE)
  modified_base (21)..(24): (21,24) = 5-methyl uracil
  modified_base (23)..(27): (23,27) = 5-methyl cytosine
gacgaggccuaagcaacaaaugcuggcag

SEQ ID NO: 85 - RNA, 29 nt, Artificial Sequence
  Synthetic oligonucleotide
  modified_base (1)..(29): (1,2,3,16,17,18,19,20,21,22,23,24,25,26,27,28, 29) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(29): (19,20,21,22,23,24,25,26,27,28, 29) = 2′-O-methoxyethyl (2MOE)
  modified_base (21)..(24): (21,24) = 5-methyl uracil
  modified_base (23)..(27): (23,27) = 5-methyl cytosine
gacgaggccuaagcaacaaaugcuggcag

SEQ ID NO: 86 - RNA, 27 nt, Artificial Sequence
  Synthetic oligonucleotide
  modified_base (1)..(27): (1,2,3,16,17,18,19,20,21,22,23,24,25,26, 27) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(27): (19,20,21,22,23,24,25,26, 27) = 2′-O-methoxyethyl (2MOE)
  modified_base (19)..(22): (19,22) = 5-methyl uracil
  modified_base (21)..(25): (21,25) = 5-methyl cytosine
gacgaggccuaagcaacaugcuggcag

SEQ ID NO: 87 - RNA, 26 nt, Artificial Sequence
  Synthetic oligonucleotide
  modified_base (1)..(26): (1,2,3,16,17,18,19,20,21,22,23,24,25, 26) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(26): (19,20,21,22,23,24,25,26) = 2′-O-methoxyethyl (2MOE)
  modified_base (20)..(24): (20,24) = 5-methyl cytosine
  modified_base (21)..(21): (21) = 5-methyl uracil
gacgaggccuaagcaacagcuggcag

SEQ ID NO: 88 - RNA, 25 nt, Artificial Sequence
  Synthetic oligonucleotide
  modified_base (1)..(25): (1,2,3,16,17,18,19,20,21,22,23,24, 25) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(25): (19,20,21,22,23,24,25) = 2′-O-methoxyethyl (2MOE)
  modified_base (20)..(24): (20,24) = 5-methyl cytosine
  modified_base (21)..(21): (21) = 5-methyl uracil
gacgaggccuaagcaacagcuggca

SEQ ID NO: 89 - RNA, 24 nt, Artificial Sequence
  Synthetic oligonucleotide
  modified_base (1)..(24): (1,2,3,16,17,18,19,20,21,22,23, 24) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(24): (19,20,21,22,23,24) = 2′-O-methoxyethyl (2MOE)
  modified_base (20)..(24): (20,24) = 5-methyl cytosine
  modified_base (21)..(21): (21) = 5-methyl uracil
gacgaggccuaagcaacagcuggc

SEQ ID NO: 90 - RNA, 36 nt, Artificial Sequence
  Synthetic oligonucleotide
  modified_base (1)..(36): (1,2,3,16,17,18,19,20,21,22,23,24,25,26,27,28, 29,30,31,32,33,34,35,36) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(36): (19,20,21,22,23,24,25,26,27,28,29,30,31,32,33, 34,35,36) = 2′-O-methoxyethyl (2MOE)
  modified_base (19)..(33): (19,24,28,31,33) = 5-methyl uracil
  modified_base (23)..(29): (23,29) = 5-methyl cytosine
gacgaggccuaagcaacauagacuagaucauaugag

SEQ ID NO: 91 - RNA, 34 nt, Artificial Sequence
  Synthetic oligonucleotide
  modified_base (1)..(34): (1,2,3,16,17,18,19,20,21,22,23,24,25,26,27,28, 29,30,31,32,33,34) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(34): (19,20,21,22,23,24,25,26,27,28,29,30,31,32, 33,34) = 2′-O-methoxyethyl (2MOE)
  modified_base (19)..(33): (19,24,28,31,33) = 5-methyl uracil
  modified_base (23)..(29): (23,29) = 5-methyl cytosine
gacgaggccuaagcaacauagacuagaucauaug

SEQ ID NO: 92 - RNA, 33 nt, Artificial Sequence
  Synthetic oligonucleotide
  modified_base (1)..(33): (1,2,3,16,17,18,19,20,21,22,23,24,25,26,27,28, 29,30,31,32,33) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(33): (19,20,21,22,23,24,25,26,27,28,29,30,31,32, 33) = 2′-O-methoxyethyl (2MOE)
  modified_base (19)..(33): (19,24,28,31,33) = 5-methyl uracil
  modified_base (23)..(29): (23,29) = 5-methyl cytosine
gacgaggccuaagcaacauagacuagaucauau

SEQ ID NO: 93 - RNA, 32 nt, Artificial Sequence
  Synthetic oligonucleotide
  modified_base (1)..(32): (1,2,3,16,17,18,19,20,21,22,23,24,25,26,27,28, 29,30,31,32) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(32): (19,20,21,22,23,24,25,26,27,28,29,30,31, 32) = 2′-O-methoxyethyl (2MOE)
  modified_base (19)..(31): (19,24,28,31) = 5-methyl uracil
  modified_base (23)..(29): (23,29) = 5-methyl cytosine
gacgaggccuaagcaacauagacuagaucaua

SEQ ID NO: 94 - RNA, 31 nt, Artificial Sequence
  Synthetic oligonucleotide
  modified_base (1)..(31): (1,2,3,16,17,18,19,20,21,22,23,24,25,26,27,28, 29,30,31) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(31): (19,20,21,22,23,24,25,26,27,28,29,30, 31) = 2′-O-methoxyethyl (2MOE)
  modified_base (19)..(31): (19,24,28,31) = 5-methyl uracil
  modified_base (23)..(29): (23,29) = 5-methyl cytosine
gacgaggccuaagcaacauagacuagaucau

SEQ ID NO: 95 - RNA, 30 nt, Artificial Sequence
  Synthetic oligonucleotide
  modified_base (1)..(30): (1,2,3,16,17,18,19,20,21,22,23,24,25,26,27,28, 29,30) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(30): (19,20,21,22,23,24,25,26,27,28,29, 30) = 2′-O-methoxyethyl (2MOE)
  modified_base (19)..(28): (19,24,28) = 5-methyl uracil
  modified_base (23)..(29): (23,29) = 5-methyl cytosine
gacgaggccuaagcaacauagacuagauca

SEQ ID NO: 96 - RNA, 29 nt, Artificial Sequence
  Synthetic oligonucleotide
  modified_base (1)..(29): (1,2,3,16,17,18,19,20,21,22,23,24,25,26,27,28, 29) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(29): (19,20,21,22,23,24,25,26,27,28, 29) = 2′-O-methoxyethyl (2MOE)
  modified_base (19)..(28): (19,24,28) = 5-methyl uracil
  modified_base (23)..(29): (23,29) = 5-methyl cytosine
gacgaggccuaagcaacauagacuagauc

SEQ ID NO: 97 - RNA, 28 nt, Artificial Sequence
  Synthetic oligonucleotide
  modified_base (1)..(28): (1,2,3,16,17,18,19,20,21,22,23,24,25,26,27, 28) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(28): (19,20,21,22,23,24,25,26,27, 28) = 2′-O-methoxyethyl (2MOE)
  modified_base (19)..(28): (19,24,28) = 5-methyl uracil
  modified_base (23)..(23): (23) = 5-methyl cytosine
gacgaggccuaagcaacauagacuagau

SEQ ID NO: 98 - RNA, 27 nt, Artificial Sequence
  Synthetic oligonucleotide
  modified_base (1)..(27): (1,2,3,16,17,18,19,20,21,22,23,24,25,26, 27) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(27): (19,20,21,22,23,24,25,26, 27) = 2′-O-methoxyethyl (2MOE)
  modified_base (19)..(24): (19,24) = 5-methyl uracil
  modified_base (23)..(23): (23) = 5-methyl cytosine
gacgaggccuaagcaacauagacuaga

SEQ ID NO: 99 - RNA, 26 nt, Artificial Sequence
  Synthetic oligonucleotide
  modified_base (1)..(26): (1,2,3,16,17,18,19,20,21,22,23,24,25, 26) = phosphorothioate (PS) backbone modification
  modified_base (1)..(17): (1,3,5,7,13,15,17) = 2′-O-methyl
  modified_base (2)..(18): (2,4,6,8,9,10,11,12,14,16,18) = 2′-fluoro
  modified_base (18)..(19): (18,19) = L1, spacer-S18 (S18 linker)
  modified_base (19)..(26): (19,20,21,22,23,24,25,26) = 2′-O-methoxyethyl (2MOE)
  modified_base (19)..(24): (19,24) = 5-methyl uracil
  modified_base (23)..(23): (23) = 5-methyl cytosine
gacgaggccuaagcaacauagacuag

SEQ ID NO: 100 - DNA, 40 bp, Artificial Sequence
  Synthetic oligonucleotide
  misc_feature (1)..(38): (1,2,3,4,5,6,7,8,9,10,11,12,13,14,15,16,17,18, 19,20,21,22,23,24,2 5,26,27,28,29,30,31,32,33,34,35,36,37,38)=RNA
  modified_base (19)..(20): (19,20) = L1, spacer-S18 (S18 linker)
  misc_feature (39)..(40): (39,40)=DNA
ccaacucauucuccaagucgccgaagucaucuggacaatt

SEQ ID NO: 101 - DNA, 25 bp, Artificial Sequence
  Synthetic primer
tcatactggctattatatgggtttt

SEQ ID NO: 102 - DNA, 21 bp, Artificial Sequence
  Synthetic primer
tgctctatgccagcatttctc

SEQ ID NO: 103 - DNA, 26 bp, Artificial Sequence
  Synthetic primer
gctattatatggaaatgctggcatag

SEQ ID NO: 104 - DNA, 24 bp, Artificial Sequence
  Synthetic primer
ttccagatctgtctgatcgtttct

SEQ ID NO: 105 - DNA, 20 bp, Artificial Sequence
  Synthetic primer
cccccaccacctcccatatg

SEQ ID NO: 106 - DNA, 25 bp, Artificial Sequence
  Synthetic primer
cccttctcacagctcataaaattac

SEQ ID NO: 107 - DNA, 20 bp, Artificial Sequence
  Synthetic primer
ggaagaccatgtggacctgt

SEQ ID NO: 108 - DNA, 20 bp, Artificial Sequence
  Synthetic primer
ggattagggcttcctcttgg

SEQ ID NO: 109 - DNA, 20 bp, Artificial Sequence
  Synthetic primer
tgtgaccagcacactgagaa

SEQ ID NO: 110 - DNA, 20 bp, Artificial Sequence
  Synthetic primer
tggaggatgtgccagaggta

SEQ ID NO: 111 - DNA, 22 bp, Artificial Sequence
  Synthetic primer
tgctcacccaccaacaatttag

SEQ ID NO: 112 - DNA, 22 bp, Artificial Sequence
  Synthetic primer
tctgctctgactttagcacctg

SEQ ID NO: 113 - DNA, 21 bp, Artificial Sequence
  Synthetic primer
atcaccatcttccaggagcga

SEQ ID NO: 114 - DNA, 21 bp, Artificial Sequence
  Synthetic primer
ttctccatggtggtgaagacg

SEQ ID NO: 115 - DNA, 20 bp, Artificial Sequence
  Synthetic primer
gctctggaattgtaccgcag

SEQ ID NO: 116 - DNA, 20 bp, Artificial Sequence
  Synthetic primer
ctgcagcaaatcgcttggga

SEQ ID NO: 117 - DNA, 19 bp, Artificial Sequence
  Synthetic primer
tgaaggtcggtgtgaacgg

SEQ ID NO: 118 - DNA, 21 bp, Artificial Sequence
  Synthetic primer
ttgaggtcaatgaaggggtcg

SEQ ID NO: 119 - DNA, 21 bp, Artificial Sequence
  Synthetic primer
ataacaccaccactcttactc

SEQ ID NO: 120 - DNA, 21 bp, Artificial Sequence
  Synthetic primer
gtagccgtgatgccattgtca

SEQ ID NO: 121 - DNA, 21 bp, Artificial Sequence
  Synthetic primer
agcctgaagaacgagatcagc

SEQ ID NO: 122 - DNA, 24 bp, Artificial Sequence
  Synthetic primer
aagcattaaaggactgactgaagg

SEQ ID NO: 123 - DNA, 21 bp, Artificial Sequence
  Synthetic primer
caagtctccaacatgcctctc

SEQ ID NO: 124 - DNA, 159 bp, Homo sapiens
agtcgcactctgtcactcaggctggagtgcagtggcgtgatcttggctcactgcaacctccgcctcccgagttcaagtgattctcctggctcagcctcccaagcagctgtcattacaggcctgcaccaccacacccggctgatttttgtatttttagga

SEQ ID NO: 125 - DNA, 82 bp, Homo sapiens
aatactggaggcccggtgtggtggctcacacctgtaatcccagcactttgggaggccgaggcggtcggattacgaggtcagg

SEQ ID NO: 126 - DNA, 815 bp, Homo sapiens
ctggccaacatggtgaaaccccatctttactaaaaatacaaaaattagccgggtgtggtggtgggcgcctgtaatcccagctactcggggggctgaggcagaattgcttgaacctgggaggcagaggttgcagtgagctgagatcacgccactgcattccagcctgggtgacagagcaatactctgtcgcaaaaaaaaaaaagaatactggaggctgggcgaggtggctcacacctgtaatcccagcattttgggatgccagaggcgggcggaatatcttgagctcaggagttcgagaccagcctacacaatatgctccaaacgccgcctctacaaaacatacagaaactagccgggtgtggtggcgtgcccctgtggtcctagctacttgggaggttgaggcgggaggatcgcttgagctcgggaggtcgaggctgcaatgagccgagatggtgccactgcactctgacgacagagcgagactccgtctcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacactggagttcgagacgaggcctaagcaacatgccgaaaccccgtctctactaaatacaaaaaatagctgagcttggtggcgcacgcctatagtcctagctactggggaggctg

SEQ ID NO: 127 - DNA, 108 bp, Homo sapiens
ctgcagtgagccgagatcgcgccgctgcactccagcctgagcgacagggcgaggctctgtctcaaaacaaacaaacaaaaaaaaaaggaaaggaaatataacacagtg

SEQ ID NO: 128 - RNA, 15 nt, Artificial Sequence
  Synthetic oligonucleotide
  modified_base (1)..(15): (1,2,3,13,14,15) = phosphorothioate (PS) backbone modification
  modified_base (1)..(15): (1,3,5,7,9,11,13,15) = 2′-fluoro
  modified_base (2)..(14): (2,4,6,8,10,12,14) = 2′-O-methyl
  modified_base (15)..(15): (15) = L5, DIO linker
gaggccuaagcaaca

SEQ ID NO: 129 - RNA, 22 nt, Artificial Sequence
  Synthetic oligonucleotide
  modified_base (1)..(22): (1,2,3,15,16,17,18,19,20,21, 22) = phosphorothioate (PS) backbone modification
  modified_base (1)..(21): (1,3,5,7,9,11,13,15,17,19,21) = 2′-O-methyl
  modified_base (2)..(22): (2,4,6,8,10,12,14,16,18,20,22) = 2′-fluoro
cacuggaguucgagacgaggcc

SEQ ID NO: 130 - RNA, 38 nt, Artificial Sequence
  Synthetic oligonucleotide
  modified_base (1)..(38): (1,2,3,21,22,23,24,25,26,27,28,29,30,31,32,33, 34,35,36,37,38) = phosphorothioate (PS) backbone modification
  modified_base (1)..(19): (1,3,5,7,13,15,17,19) = 2′-O-methyl
  modified_base (2)..(20): (2,4,6,8,9,10,11,12,14,16,18,20) = 2′-fluoro
  modified_base (20)..(21): (20,21) = L1, spacer-S18 (S18 linker)
  modified_base (21)..(38): (21,22,23,24,25,26,27,28,29,30,31,32,33,34,35, 36,37,38) = 2′-O-methoxyethyl (2MOE)
  modified_base (21)..(36): (21,25,26,27,30,33,36) = 5-methyl uracil
  modified_base (22)..(35): (22,24,28,35) = 5-methyl cytosine
ccucgucucgaacuccagugucacuuucauaaugcugg

SEQ ID NO: 131 - RNA, 36 nt, Artificial Sequence
  Synthetic oligonucleotide
  modified_base (1)..(36): (1,2,3,21,22,23,24,25,26,27,28,29,30,31,32,33, 34,35,36) = phosphorothioate (PS) backbone modification
  modified_base (1)..(19): (1,3,5,7,13,15,17,19) = 2′-O-methyl
  modified_base (2)..(20): (2,4,6,8,9,10,11,12,14,16,18,20) = 2′-fluoro
  modified_base (20)..(21): (20,21) = L1, spacer-S18 (S18 linker)
  modified_base (21)..(36): (21,22,23,24,25,26,27,28,29,30,31,32,33,34, 35,36) = 2′-O-methoxyethyl (2MOE)
  modified_base (22)..(33): (22,26,33) = 5-methyl cytosine
  modified_base (23)..(34): (23,24,25,28,31,34) = 5-methyl uracil
ccucgucucgaacuccagugacuuucauaaugcugg

SEQ ID NO: 132 - RNA, 35 nt, Artificial Sequence
  Synthetic oligonucleotide
  modified_base (1)..(35): (1,2,3,21,22,23,24,25,26,27,28,29,30,31,32,33, 34,35) = phosphorothioate (PS) backbone modification
  modified_base (1)..(19): (1,3,5,7,13,15,17,19) = 2′-O-methyl
  modified_base (2)..(20): (2,4,6,8,9,10,11,12,14,16,18,20) = 2′-fluoro
  modified_base (20)..(21): (20,21) = L1, spacer-S18 (S18 linker)
  modified_base (21)..(35): (21,22,23,24,25,26,27,28,29,30,31,32,33,34, 35) = 2′-O-methoxyethyl (2MOE)
  modified_base (21)..(32): (21,22,23,26,29,32) = 5-methyl uracil
  modified_base (24)..(35): (24,31,35) = 5-methyl cytosine
ccucgucucgaacuccaguguuucauaaugcuggc

SEQ ID NO: 133 - RNA, 34 nt, Artificial Sequence
  Synthetic oligonucleotide
  modified_base (1)..(34): (1,2,3,21,22,23,24,25,26,27,28,29,30,31,32,33, 34) = phosphorothioate (PS) backbone modification
  modified_base (1)..(19): (1,3,5,7,13,15,17,19) = 2′-O-methyl
  modified_base (2)..(20): (2,4,6,8,9,10,11,12,14,16,18,20) = 2′-fluoro
  modified_base (20)..(21): (20,21) = L1, spacer-S18 (S18 linker)
  modified_base (21)..(34): (21,22,23,24,25,26,27,28,29,30,31,32,33, 34) = 2′-O-methoxyethyl (2MOE)
  modified_base (21)..(31): (21,22,25,28,31) = 5-methyl uracil
  modified_base (23)..(34): (23,30,34) = 5-methyl cytosine
ccucgucucgaacuccaguguucauaaugcuggc

SEQ ID NO: 134 - RNA, 33 nt, Artificial Sequence
  Synthetic oligonucleotide
  modified_base (1)..(33): (1,2,3,21,22,23,24,25,26,27,28,29,30,31,32, 33) = phosphorothioate (PS) backbone modification
  modified_base (1)..(19): (1,3,5,7,13,15,17,19) = 2′-O-methyl
  modified_base (2)..(20): (2,4,6,8,9,10,11,12,14,16,18,20) = 2′-fluoro
  modified_base (20)..(21): (20,21) = L1, spacer-S18 (S18 linker)
  modified_base (21)..(33): (21,22,23,24,25,26,27,28,29,30,31,32, 33) = 2′-O-methoxyethyl (2MOE)
  modified_base (21)..(30): (21,24,27,30) = 5-methyl uracil
  modified_base (22)..(33): (22,29,33) = 5-methyl cytosine
ccucgucucgaacuccagugucauaaugcuggc

SEQ ID NO: 135 - RNA, 32 nt, Artificial Sequence
  Synthetic oligonucleotide
  modified_base (1)..(32): (1,2,3,21,22,23,24,25,26,27,28,29,30,31, 32) = phosphorothioate (PS) backbone modification
  modified_base (1)..(19): (1,3,5,7,13,15,17,19) = 2′-O-methyl
  modified_base (2)..(20): (2,4,6,8,9,10,11,12,14,16,18,20) = 2′-fluoro
  modified_base (20)..(21): (20,21) = L1, spacer-S18 (S18 linker)
  modified_base (21)..(32): (21,22,23,24,25,26,27,28,29,30,31, 32) = 2′-O-methoxyethyl (2MOE)
  modified_base (21)..(32): (21,28,32) = 5-methyl cytosine
  modified_base (23)..(29): (23,26,29) = 5-methyl uracil
ccucgucucgaacuccagugcauaaugcuggc

SEQ ID NO: 136 - RNA, 29 nt, Artificial Sequence
  Synthetic oligonucleotide
  modified_base (1)..(29): (1,2,3,21,22,23,24,25,26,27,28, 29) = phosphorothioate (PS) backbone modification
  modified_base (1)..(19): (1,3,5,7,13,15,17,19) = 2′-O-methyl
  modified_base (2)..(20): (2,4,6,8,9,10,11,12,14,16,18,20) = 2′-fluoro
  modified_base (20)..(21): (20,21) = L1, spacer-18 (S18 linker)
  modified_base (21)..(29): (21,22,23,24,25,26,27,28, 29) = 2′-O-methoxyethyl (2MOE)
  modified_base (21)..(24): (21,24) = 5-methyl uracil
  modified_base (23)..(27): (23,27) = 5-methyl cytosine
ccucgucucgaacuccagugugcuggcag

SEQ ID NO: 137 - RNA, 28 nt, Artificial Sequence
  Synthetic oligonucleotide
  modified_base (1)..(28): (1,2,3,21,22,23,24,25,26,27, 28) = phosphorothioate (PS) backbone  modification
  modified_base (1)..(19): (1,3,5,7,13,15,17,19) = 2′-O-methyl
  modified_base (2)..(20): (2,4,6,8,9,10,11,12,14,16,18,20) = 2′-fluoro
  modified_base (20)..(21): (20,21) = L1, spacer-S18 (S18 linker)
  modified_base (21)..(28): (21,22,23,24,25,26,27,28) = 2′-O-methoxyethyl (2MOE)
  modified_base (22)..(26): (22,26) = 5-methyl cytosine
  modified_base (23)..(23): (23) = 5-methyl uracil
ccucgucucgaacuccaguggcuggcag

SEQ ID NO: 138 - RNA, 19 nt, Artificial Sequence
  Synthetic oligonucleotide
  modified_base (1)..(19): (1,2,3,16,17,18,19) = phosphorothioate (PS) backbone modification
  modified_base (1)..(19): (1,3,5,7,9,10,11,13,15,17,19) = 2′-fluoro
  modified_base (2)..(18): (2,4,6,8,12,14,16,18) = 2′-O-methyl
gguggaaaugaagaaagua

SEQ ID NO: 139 - RNA, 21 nt, Artificial Sequence
  Synthetic oligonucleotide
  modified_base (1)..(21): (1,2,3,19,20,21) = phosphorothioate (PS) backbone modification
  modified_base (1)..(21): (1,3,5,7,9,11,13,15,17,19,21) = 2′-O-methyl
  modified_base (2)..(20): (2,4,6,8,10,12,14,16,18,20) = 2′-fluoro
uacuuucuucauuuccaccuu

SEQ ID NO: 140 - RNA, 37 nt, Artificial Sequence
  Synthetic oligonucleotide
  modified_base (1)..(37): (1,2,3,16,17,18,19,20,21,34,35,36, 37) = phosphorothioate (PS) backbone modification
  modified_base (1)..(36): (1,3,5,7,13,15,17,20,22,24,26,30,32,34, 36) = 2′-O-methyl
  modified_base (2)..(37): (2,4,6,8,9,10,11,12,14,16,18,19,21,23,25,27, 28,29,31,33,35,37) = 2′-fluoro
  modified_base (18)..(19): (18,19) = L1, spacer-S18 (S18 linker)
gacgaggccuaagcaacagguggaaaugaagaaagua

SEQ ID NO: 141 - RNA, 21 nt, Artificial Sequence
  Synthetic oligonucleotide
  modified_base (1)..(21): (1,2,3,19,20,21) = phosphorothioate (PS) backbone modification
  modified_base (1)..(21): (1,3,5,7,9,11,13,15,17,19,21) = 2′-O-methyl
  modified_base (1)..(1): (1) = 5′-(E)-vinylphosphonate
  modified_base (2)..(20): (2,4,6,8,10,12,14,16,18,20) = 2′-fluoro
uacuuucuucauuuccaccuu

SEQ ID NO: 142 - RNA, 24 nt, Artificial Sequence
  Synthetic oligonucleotide
  modified_base (1)..(24): (1,2,3,4,5,6,7,8,9,10,11,12,13,14,15,16,17,18, 19,20,21,22,23,24) = phosphorothioate (PS) backbone modification
  modified_base (1)..(24): (1,2,3,4,5,6,7,8,9,10,11,12,13,14,15,16,17,18, 19,20,21,22,23,24) = 2′-O-methoxyethyl (2MOE)
  modified_base (2)..(22): (2,3,5,11,18,19,20,22) = 5-methyl uracil
  modified_base (6)..(16): (6,8,9,10,12,13,14,16) = 5-methyl cytosine
guuaucgcccucccacauuugugg

SEQ ID NO: 143 - RNA, 42 nt, Artificial Sequence
  Synthetic oligonucleotide
  modified_base (1)..(42): (1,2,3,4,5,6,7,8,9,10,11,12,13,14,15,16,17,18, 19,20,21,22,23,24,25,26,27,28,29,30,31,32,33,34,35,36,37,38,39,40, 41,42) = phosphorothioate (PS) backbone modification
  modified_base (1)..(42): (1,2,3,4,5,6,7,8,9,10,11,12,13,14,15,16,17,18, 19,20,21,22,23,24,25,26,27,28,29,30,31,32,33,34,35,36,37,38,39,40, 41,42) = 2′-O-methoxyethyl (2MOE)
  modified_base (1)..(40): (1,5,6,7,10,13,16,20,21,23,29,36,37,38, 40) = 5-methyl uracil
  modified_base (2)..(34): (2,4,8,15,24,26,27,28,30,31,32, 34) = 5-methyl cytosine
  modified_base (18)..(19): (18,19) = L1, spacer-S18 (S18 linker)
ucacuuucauaaugcuggguuaucgcccucccacauuugugg

SEQ ID NO: 144 - RNA, 42 nt, Artificial Sequence
  Synthetic oligonucleotide
  modified_base (1)..(42): (1,2,3,4,5,6,7,8,9,10,11,12,13,14,15,16,17,18, 19,20,21,22,23,24,25,26,27,28,29,30,31,32,33,34,35,36,37,38,39,40, 41,42) = phosphorothioate (PS) backbone modification
  modified_base (1)..(42): (1,2,3,4,5,6,7,8,9,10,11,12,13,14,15,16,17,18, 19,20,21,22,23,24,25,26,27,28,29,30,31,32,33,34,35,36,37,38,39,40, 41,42) = 2′-O-methoxyethyl (2MOE)
  modified_base (2)..(40): (2,3,5,11,18,19,20,22,25,29,30,31,34,37, 40) = 5-methyl uracil
  modified_base (6)..(39): (6,8,9,10,12,13,14,16,26,28,32, 39) = 5-methyl cytosine
  modified_base (24)..(25): (24,25) = L1, spacer-S18 (S18 linker)
guuaucgcccucccacauuuguggucacuuucauaaugcugg

SEQ ID NO: 145 - RNA, 18 nt, Artificial Sequence
  Synthetic oligonucleotide
gacgaggccuaagcaaca

SEQ ID NO: 146 - RNA, 15 nt, Artificial Sequence
  Synthetic oligonucleotide
  modified_base (1)..(15): (1,2,3,13,14,15) = phosphorothioate (PS) backbone modification
  modified_base (1)..(15): (1,3,5,7,9,11,13,15) = 2′-fluoro
  modified_base (2)..(14): (2,4,6,8,10,12,14) = 2′-O-methyl
gaggccuaagcaaca

SEQ ID NO: 147 - RNA, 20 nt, Artificial Sequence
  Synthetic oligonucleotide
  modified_base (1)..(20): (1,2,3,13,14,15,16,17,18,19, 20) = phosphorothioate (PS) backbone modification
  modified_base (1)..(19): (1,3,5,7,9,11,13,15,17,19) = 2′-O-methyl
  modified_base (2)..(20): (2,4,6,8,10,12,14,16,18,20) = 2′-fluoro
uguugcuuaggccucgucuc

(19) United States
(12) Patent Application Publication
(10) Pub. No.: US 2024/0318174 A1
(43) Pub. Date: Sep. 26, 2024

(54) Multi-Valent Oligonucleotide Agent and Methods of Use Thereof

(71) Applicant: Ractigen Therapeutics, Jiangsu (CN)

(72) Inventors: Longcheng Li, Jiangsu (CN); Moorim Kang, Jiangsu (CN)

(21) Appl. No.: 18/275,895

(22) PCT Filed: Jan. 28, 2022
(86) PCT No.: PCT/CN2022/074779

§ 371 Date: Aug. 4, 2023

(30) Foreign Application Priority Data
Feb. 8, 2021 (WO) PCT/CN2021/075958

(51) Int. Cl. C12N 15/113

(52) U.S. Cl. CPC C12N 15/113; C12N 2310/14; C12N 2310/315; C12N 2310/321; C12N 2310/322; C12N 2310/3341; C12N 2310/335; C12N 2310/3519; C12N 2320/33

(57) ABSTRACT

Provided herein are multi-valent oligonucleotide agents comprising two or more functional oligonucleotide units independently selected from single-stranded antisense oligonucleotides (ASOs, such as gapmers, mixmers and steric block ASOs) and duplex (double-stranded) RNAs (dsRNAs, such as siRNA and saRNA), and methods for the preparation thereof. Also provided herein are products, comprising the multi-valent oligonucleotide agents, and methods of using the multi-valent oligonucleotide agents or products in treatment of diseases (such as spinal muscular atrophy (SMA) and cancers).

### RELATED APPLICATIONS

This application is a National Stage Application under 35 U.S.C. 371 of co-pending PCT application PCT/CN2022/074779 designating the United States and filed Jan. 28, 2022; which claims the benefit of PCT application number PCT/CN2021/075958 and filed Feb. 8, 2021, each of which are hereby incorporated by reference in their entireties.

### FIELD OF THE INVENTION

The present disclosure relates to multi-valent oligonucleotide agent comprising two or more functional oligonucleotides which may be selected from single-stranded antisense oligonucleotides (ASOs, such as gapmers and mixmers) and duplex (double-stranded) RNAs (dsRNAs, such as siRNA and saRNA). In some aspects, the functional oligonucleotides in the multi-valent oligonucleotide agent are identical or different, have different targets or the same target, and/or connected directly or via linkers. In some aspects, the multi-valent oligonucleotide agents are multi-targeting oligonucleotide agents and/or have improved activities. The present disclosure also relates to products comprising the multi-valent oligonucleotide agents, such as compositions and medicaments, and methods of using the multi-valent oligonucleotide agents or products in treatment of diseases.

### SEQUENCE LISTING

The instant application contains a Sequence Listing which has been submitted electronically in computer readable format and is hereby incorporated by reference in its entirety. Said ASCII copy, created on Aug. 4, 2023, is named “23-UBNAG2-034_Replacement Sequence Listing_20230802_PatentIN_ST25.txt” and is 95.6 kb in size.

### 1. BACKGROUND OF THE INVENTION

Oligonucleotides are an emerging class of therapeutics currently under development for treatment of a wide variety of diseases via a myriad of different mechanisms of action (MOA). Major categories of oligonucleotide therapeutics include single-stranded antisense oligonucleotides (ASOs) and duplex (double-stranded) RNAs (dsRNAs).

Single-stranded ASOs in the form of “gapmers” can be used to suppress gene expression by degrading complementary mRNA via RNase H hydrolysis. Characteristic “gapmer” ASOs have a central DNA region required for RNase H activity and two ribonucleotide wings to increase target binding affinity. Another category of ASOs called “mixmers” function as steric blockers, which are typically composed entirely of ribonucleotide analogs and bind pre-mRNA in the nucleus to alter splicing.

Double-stranded RNAs can be classified into two categories including small interfering RNA (siRNA) and small activating RNA (saRNA); both of which require Argonaute (AGO) proteins for function. Small interfering RNAs bind to target mRNAs in the cytoplasm of cells and down-regulate gene expression via a post-transcriptional mechanism of gene silencing called RNA interference (RNAi). Small activating RNAs have a reciprocal function and upregulate gene expression by targeting regulatory sequences (i.e., gene promoters) in the cell nucleus via a transcriptional mechanism of gene activation termed RNAa (RNA activation).

Many single gene disorders are caused by a loss in gene function leading to insufficiencies in gene expression levels and/or translated protein activity. “Mixmer” ASOs have found successes for the treatment of some such disorders amendable through correction of abnormal splicing errors. Several ASO drugs have been approved by the U.S. Food and Drug Administration (FDA) including an exon inclusion ASO called SPINRAZA® (nusinersen) for the treatment of spinal muscular atrophy (SMA) and an exon exclusion ASO marketed as AMONDYS 45 (casimersen) for the treatment of Duchene muscular dystrophy (DMD). Unfortunately, drug efficacy is limited by the physical amount of target transcript expressed in patient cells. As such, not all patients respond equally to therapy. Epigenetic modifiers (e.g., HDAC inhibitors) have been tested in SMA models as means to further increase gene dosage by non-specifically boosting global gene transcription in order to provide Spinraza with a larger pool of target transcript [Pagliarini, 2020 #2082]. However, aberrant activation of the transcriptome is accompanied by its own adverse effects and offsets the gene-specific precision of targeted oligonucleotide therapies needed for single gene disorders. Inventors' prior work demonstrated an alternative approach by using saRNA to specifically activate target gene transcription in combination with Spinraza ASO. Treatment with saRNA selectively enhanced transcriptional output of target gene SMN2, while Spinraza modified SMN2 pre-mRNA splicing to produce full-length SMN2 transcript (SMN2FL) and its cognate protein at levels beyond either treatment alone.

RNA duplexes (e.g., saRNAs) are typically of larger mass and structurally more rigid than ASOs disparately affecting their intrinsic biophysical properties [Crooke, 2017 #23; Shen, 2018 #40]. As such, dsRNAs have their own distinctive biodistribution and tissue diffusion patterns compared to single-stranded oligonucleotides. In order for ASO and saRNA co-treatments to function cooperatively on a single gene target, both drug molecules would need enrichment in the same intended target tissue/cells in vivo. Technologies that can direct both drug modalities to the intended target tissue and provide similar distribution properties would enhance therapeutic efficacy of such combination treatments.

Thus, there is a need for improved oligonucleotide-based therapeutics to address these challenges.

### 2. SUMMARY OF THE INVENTION

Double-stranded RNAs (dsRNAs) targeting gene regulatory sequences, including promoters, have been shown to upregulate target gene transcription in a sequence-specific manner through a mechanism known as RNA activation (RNAa) (Li, L. C., et al. Small dsRNAs induce transcriptional activation in human cells. PNAS (2006)). The inventors have demonstrated in prior works that gene dosage can be further enhanced for a specific splicing variant through cotreatment with ASO splicing modulators beyond levels capable than either treatment alone. However, duplex RNAs (e.g., saRNAS) and single-stranded ASOs have different biophysical properties and distinctive patterns for tissue biodistribution, diffusion, and cellular uptake. Typically, ASOs diffuse throughout organ tissues based on the route of administration. Cellular uptake and ASO activity are readily measurable without the need of an adjuvant delivery vehicle, carrier system, or targeting conjugate. On the other hand, duplex RNAs (e.g., saRNAs, siRNAs, and miRNAs) do not readily penetrate into solid tissues, nor transverse cell membranes. In vivo activity typically requires a delivery vehicle, targeting conjugate, and/or novel chemistries. In order for saRNA and ASO cotreatments to have combined therapeutics effects in vivo, both drug modalities need access to same tissues and/or similar biodistribution requirements.

To overcome innate differences in tissue distribution properties, inventors have developed a novel chemical construct covalently linking ASO splicing modulators (e.g., Spinraza) to saRNAs within a single molecule. Biophysical properties are shared between the different drug modalities providing uniform biodistribution and broad activity within different tissues. This chemical strategy was further expanded and testing with different oligonucleotide drug combinations including saRNA-saRNA, saRNA-siRNA, saRNA-saRNA-ASO, ASO-ASO, etc. Collectively, these novel constructs comprise a class of pleiotropic oligonucleotides herein refer to as multi-valent oligonucleotides (MVO).

Embodiments of the present disclosure are based in part on the surprising discovery that two or more functional oligonucleotides, when covalently linked, can upregulate and/or downregulate the expression of one or more genes of interest, e.g., for the purpose of treating a disease or condition associated with the one or more genes of interest.

In some aspects, provided herein is a multi-valent oligonucleotide (MVO) agent, comprising two or more functional oligonucleotides that are covalently linked, wherein the two or more functional oligonucleotides are independently selected from: a) a double stranded RNA (dsRNA); and b) an antisense oligonucleotide (ASO).

In some embodiments, the MVO agent increases the expression of a SMN2 gene or protein. In some embodiments of the MVO agent, the dsRNA(s) increases the expression of the SMN2 gene or protein; and/or the ASO(s) increases the production of functional SMN protein by modulating SMN2 mRNA splicing or stability (SMN2 mRNA modulators).

In some embodiments, the MVO agent increases the expression of CDKN1A/p21 gene or protein and/or decreases the expression CD274/PDL-1. In some embodiments of the MVO agent, the dsRNA(s) are independently selected from: a saRNA that increases the expression of the CDKN1A/p21 gene or protein; and a siRNA that decreases the expression CD274/PDL-1.

In some aspects, provided herein is a product, comprising the multi-valent oligonucleotide agent. In some embodiments, the product is selected from a pharmaceutical composition further comprising at least one pharmaceutically acceptable carrier or a kit.

In some aspects, provided herein is a method for disease treatment, comprising administering sufficient amount of one or more of the multi-valent oligonucleotide agent or a product disclosed herein to a subject in need of such treatment. For example, the method is for treating or delaying the onset or progression of SMN-deficiency-related conditions or p21/PDL-1 associated disease in a subject. Also provided herein is use of one or more of the multi-valent oligonucleotide agent or a product disclosed herein in the preparation of a product for disease treatment. Also provided herein is one or more of the multi-valent oligonucleotide agent or a product disclosed herein for treatment of diseases.

In some aspects, provided herein is a method for the preparation of the multi-valent oligonucleotide agent disclosed herein, comprising: providing said two or more funcational oligonucleotides and covalently linking the same; or synthesizing the full length oligonucleotide agent.

Also provided herein is an isolated or synthesized oligonucleotide comprising: a nucleotide sequence of a saRNA sense strand that is at least 90% identical to the nucleotide sequence of SEQ ID NO: 62. In some embodiment, the oligonucleotide further comprises an antisense strand that has partial complementarity with the above sense saRNA strand. In some embodiments, the oligonucleotide further comprises an antisense strand that is at least 90% identical to the nucleotide sequence of SEQ ID NO: 63. In some embodiment, the isolated or synthesized oligonucleotide comprising: a nucleotide sequence of a saRNA sense strand of SEQ ID NO: 62 and a saRNA antisense strand of SEQ ID NO: 63. Also provided herein is a pharmaceutical composition or kit comprising the isolated or synthesized oligonucleotide disclosed herein. Also provided herein is a method for disease treatment, comprising administering sufficient amount of one or more of the isolated or synthesized oligonucleotide or a pharmaceutical composition or kit disclosed herein to a subject in need of such treatment. Also provided herein is use of one or more of the isolated or synthesized oligonucleotide or a pharmaceutical composition or kit disclosed herein in the preparation of a product for disease treatment. Also provided herein is one or more of the isolated or synthesized oligonucleotide or a pharmaceutical composition or kit disclosed herein for treatment of diseases.

The following are some aspects and embodiments of the present disclosure:

1. A multi-valent oligonucleotide agent comprising two or more functional oligonucleotides that are covalently linked, wherein the two or more functional oligonucleotides are independently selected from:
a) a double stranded RNA (dsRNA); and
b) an antisense oligonucleotide (ASO).

2. The multi-valent oligonucleotide agent of item 1, wherein the number of the functional oligonucleotides comprised in the multi-valent oligonucleotide agent is ranged from 2 to X, wherein X is an integer ranged from 3 to 10.

3. The multi-valent oligonucleotide agent of item 2, wherein the number of dsRNA comprised in the agent is from 0 to X, with the rest functional oligonucleotides being ASO(s).

4. The multi-valent oligonucleotide agent of item 1, wherein the dsRNA(s) are independently selected from a small interfering RNA (siRNA) and a small activating RNA (saRNA); and/or
the ASO(s) are independently selected from a gapmer and a mixmer.

5. The multi-valent oligonucleotide agent of item 1, wherein the two or more functional oligonucleotides independently modulate the expression of one or more genes, modulate the expression of one or more proteins (such as by binding to a mRNA sequence), or modulate non-coding regulatory nucleic acid sequences.

6. The multi-valent oligonucleotide agent of item 5, wherein the non-coding regulatory nucleic acid sequence is a promoter sequence, enhancer, silencer, and/or transcription factor.

7. The multi-valent oligonucleotide agent of item 1, wherein each dsRNA comprises a sense strand that is at least 10 contiguous nucleotides and an antisense strand that is at least 10 contiguous nucleotides.

8. The multi-valent oligonucleotide agent of item 1, wherein each dsRNA comprises a sense strand that is of 10-60 nucleotides in length and/or each dsRNA comprises an antisense strand that is of 10-60 nucleotides in length.

9. The multi-valent oligonucleotide agent of item 1, wherein each ASO has a nucleotide sequence that is at least 5 contiguous nucleotides in length.

10. The multi-valent oligonucleotide agent of item 1, wherein each ASO has a nucleotide sequence that is 5-30 nucleotides in length.

11. The multi-valent oligonucleotide agent of item 1, wherein the two or more functional oligonucleotides have a total length ranging from 12 to 200 nucleotides.

12. The multi-valent oligonucleotide agent of any one of items 1-10, wherein any two adjacent functional oligonucleotides of the two or more functional oligonucleotides are covalently linked by a linking component or with no linking component.

13. The multi-valent oligonucleotide agent of item 12, wherein the linking component is selected from the following linkers or derivatives thereof:

| Name | Formula | Structure |
|---|---|---|
| Spacer-9 | C₃₆H₄₉N₂O₇P | 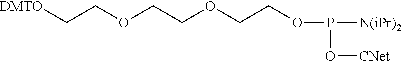 |
| Spacer-18 | C₄₂H₆₁N₂O₁₀P | 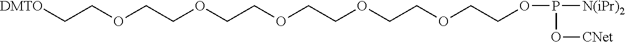 |
| Spacer-C3 | C₃₃H₄₃N₂O₅P | 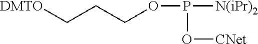 |
| Spacer-C6 | C₃₆H₄₉N₂O₅P | 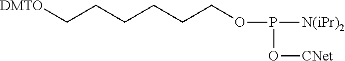 |

14. The multi-valent oligonucleotide agent of item 12, wherein the two adjacent functional oligonucleotides are covalently linked by:

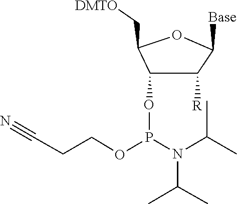

wherein R represents —H or —OH or —OMe, or -MOE, or —F, or other 2′ chemical modifications.

15. The multi-valent oligonucleotide agent of item 12, wherein the two adjacent functional oligonucleotides are covalently linked by a phosphodiester bond or a phosphorothioate bond.

16. The multi-valent oligonucleotide agent of item 12, wherein the two adjacent functional oligonucleotides are covalently linked by one or more nucleotides.

17. The multi-valent oligonucleotide agent of any one of items 1-10, wherein one or more of the functional oligonucleotides comprise at least one chemically modified nucleotide.

18. The multi-valent oligonucleotide agent of item 17, wherein the chemical modification of the at least one chemically modified nucleotide is a 2′ sugar modification selected from one or more of: 2′-fluoro-2′-deoxynucleoside (2′-F) modification, 2′-O-methyl (2′-O—Me), modification, and 2′-O-(2-methoxyethyl) (2′-O-MOE) modification.

19. The multi-valent oligonucleotide agent of item 17, wherein the chemical modification of the at least one chemically modified nucleotide is a Phosphorothioate (PS) backbone modification.

20. The multi-valent oligonucleotide agent of item 17, wherein the chemical modification of the at least one chemically modified nucleotide is an addition of a 5′-phosophate moiety at the 5′ end of the nucleotide sequence.

21. The multi-valent oligonucleotide agent of item 17, wherein the chemical modification of the at least one chemically modified nucleotide is an addition of an (E)-vinylphosphonate moiety at the 5′ end of the nucleotide sequence.

22. The multi-valent oligonucleotide agent of item 17, wherein the chemical modification of the at least one chemically modified nucleotide is an addition of a 5-methyl cytosine moiety at the 5′ end of the nucleotide sequence.

23. The multi-valent oligonucleotide agent of any one of items 1-22, wherein each of the ASO in the agent is covalently linked to the adjacent targeting oligonucleotide in a 3′ to 5′ orientation or in a 5′ to 3′ orientation.

24. The multi-valent oligonucleotide agent of any one of items 1-22, wherein each of the dsRNA in the agent is covalently linked to an adjacent ASO at its 3′ end of the sense or antisense strand; or at its 5′ end of the sense or antisense strand.

25. The multi-valent oligonucleotide agent of items 1-24, wherein the sequences of the two or more functional oligonucleotides are identical or different; and/or the functions of the two or more functional oligonucleotides are identical or different.

26. The multi-valent oligonucleotide agent of item 1-25, wherein the multi-valent oligonucleotide agent comprises:
a) a first double stranded RNA (dsRNA) and a first antisense oligonucleotide (ASO);
b) a first double stranded RNA (dsRNA) and a second dsRNA;
c) a first antisense oligonucleotide (ASO) and a second ASO;
d) a first double stranded RNA (dsRNA), a second dsRNA, and a third dsRNA;
e) a first double stranded RNA (dsRNA), a second dsRNA, and a first antisense oligonucleotide (ASO);
f) a first double stranded RNA (dsRNA), a first antisense oligonucleotide (ASO) and a second ASO; or
g) a first antisense oligonucleotide (ASO), a second ASO, and a third ASO, wherein in any one of a)˜ g), the functional oligonucleotides are arranged in any order and covalently linked with or without linking component(s).

27. The multi-valent oligonucleotide agent of item 23, wherein if presented, the first dsRNA, the second dsRNA and the third dsRNAs are independently selected from a siRNA and a saRNA.

28. The multi-valent oligonucleotide agent of item 23, wherein if presented, the first ASO, the second ASO and the third ASO are independently selected from a gapmer and a mixmer.

29. The multi-valent oligonucleotide agent of item 23, wherein the multi-valent oligonucleotide agent comprises functional oligonucleotides selected from:
a) siRNA-siRNA; b) siRNA-saRNA; c) saRNA-saRNA; d) siRNA-gapmer;
e) siRNA-mixmer; f) saRNA-gapmer; g) saRNA-mixmer; h) gapmer-gapmer;
i) gapmer-mixmer; j) mixmer-mixmer;
k) siRNA-siRNA-siRNA; 1) siRNA-siRNA-saRNA; m) siRNA-saRNA-saRNA;
n) saRNA-saRNA-saRNA; o) siRNA-siRNA-gapmer; p) siRNA-siRNA-mixmer;
q) siRNA-saRNA-gapmer; r) siRNA-saRNA-mixmer; s) saRNA-saRNA-gapmer;
t) saRNA-saRNA-mixmer; u) siRNA-gapmer-gapmer; v) saRNA-gapmer-gapmer;
w) siRNA-gapmer-mixmer; x) saRNA-gapmer-mixmer; y) siRNA-mixmer-mixmer;
z) saRNA-mixmer-mixmer; aa) gapmer-gapmer-gapmer; ab) gapmer-gapmer-mixmer;
ac) gapmer-mixmer-mixmer; and, ad) mixmer-mixmer-mixmer, wherein in any one of a)˜ ad), the functional oligonucleotides are arranged in any order and covalently linked with or without linking component(s).

30. The multi-valent oligonucleotide agent of any one of items 26-29, wherein:
a) the ASO is covalently linked to a 3′ end of the sense or antisense strand of the first dsRNA; or
b) the ASO is covalently linked to a 5′ end of the sense or antisense strand of the first dsRNA.

31. The multi-valent oligonucleotide agent of item 27, wherein the 5′ end of the ASO is conjugated to a linking component; or the 3′ end of the ASO is conjugated to a linking component.

32. The multi-valent oligonucleotide agent of any one of items 1-31, comprising one or more additional targeting oligonucleotide(s).

33. The multi-valent oligonucleotide agent of item 32, wherein the additionally targeting oligonucleotide(s) are independently selected from: a double stranded RNA (dsRNA) and an antisense oligonucleotide (ASO).

34. The multi-valent oligonucleotide agent of item 33, wherein the additionally double stranded RNA (dsRNA) is selected from a small interfering RNA (siRNA) and a small activating RNA (saRNA); and/or the targeting oligonucleotide(s) are independently selected from a gapmer and a mixmer.

35. The multi-valent oligonucleotide agent of any one of items 1-34, wherein one or more of the ASO targets 5′-UTR.

36. The multi-valent oligonucleotide agent of any one of items 1-35, wherein one or more of functional oligonucleotides increase the expression of a SMN2 gene or protein.

37. The multi-valent oligonucleotide agent of item 36, wherein the dsRNA(s) increases the expression of the SMN2 gene or protein; and/or the ASO(s) increases the production of functional SMN protein by modulating SMN2 mRNA splicing or stability (SMN2 mRNA modulators).

38. The multi-valent oligonucleotide agent of item 36, wherein the dsRNA(s) comprises a saRNA having a nucleotide sequence of a sense strand that is at least 90% identical to the nucleotide sequence selected from:
a) DS06-0004 (SEQ ID NO: 5);
b) DS06-0031 (SEQ ID NO: 7);
c) DS06-0067 (SEQ ID NO: 9);
d) DS06-4A3 (SEQ ID NO: 146);
e) R6-04-S1 (SEQ ID NO: 59); and
f) R6-04(20)-S1V1v(CM-4) (SEQ ID NO: 60).

39. The multi-valent oligonucleotide agent of item 36, wherein the dsRNA(s) comprises a saRNA having a nucleotide sequence of an antisense strand that is at least 90% identical to the nucleotide sequence selected from:
a) DS06-0004 (SEQ ID NO: 6);
b) DS06-0031 (SEQ ID NO: 8);
c) DS06-0067 (SEQ ID NO: 10);
d) DS06-4A3 (SEQ ID NO: 147);
e) R6-04-S1 (SEQ ID NO: 53); and
f) R6-04M1-27A-S1L1V3(CM-26) (SEQ ID NO: 17). 40. The multi-valent oligonucleotide agent of item 36, wherein the dsRNA(s) comprises a saRNA having a pair of nucleotide sequences of a sense strand and an antisense strand that is at least 90% identical to the nucleotide sequence pairs selected from:
a) DS06-0004: SEQ ID NO: 5 and SEQ ID NO: 6;
b) DS06-0031: SEQ ID NO: 7 and SEQ ID NO: 8;
c) DS06-0067: SEQ ID NO: 9 and SEQ ID NO: 10;
d) DS06-4A3: SEQ ID NO: 146 and SEQ ID NO: 147;
e) R6-04-S1: SEQ ID NO: 59 and SEQ ID NO: 53; and
f) R6-04(20)-S1V1v(CM-4): SEQ ID NO: 60 and SEQ ID NO: 17.

41. The multi-valent oligonucleotide agent of item 36, wherein the dsRNA comprises a siRNA having a nucleotide sequence of a sense strand that is at least 90% identical to the nucleotide sequence of DS06-332i (SEQ ID NO: 3) or siSOD1-388-ESC (SEQ ID NO: 138).

42. The multi-valent oligonucleotide agent of item 36, wherein the dsRNA comprises a siRNA having a nucleotide sequence of an antisense strand that is at least 90% identical to the nucleotide sequence of DS06-332i (SEQ ID NO: 4) or siSOD1-388-ESC (SEQ ID NO: 139).

43. The multi-valent oligonucleotide agent of item 36, wherein the dsRNA comprises a siRNA having a pair of nucleotide sequences of a sense strand and an antisense strand that is at least 90% identical to the nucleotide sequence pairs selected from: DS06-332i: SEQ ID NO: 3 and SEQ ID NO: 4; and siSOD1-388-ESC: SEQ ID NO: 138 and SEQ ID NO: 139.

44. The multi-valent oligonucleotide agent of item 36, wherein the ASO has a nucleotide sequence that is at least 90% identical to the nucleotide sequence of ASO10-27 (SEQ ID NO: 11) or 5′UTR ASO (SEQ ID NO: 142).

45. The multi-valent oligonucleotide agent of any one of items 36-44, wherein the multi-valent oligonucleotide agent has a nucleotide sequence that is at least 90% identical to the nucleotide sequences selected from:
a) DA06-4A-27A (SEQ ID NO: 14) and an antisense saRNA strand having a nucleotide sequence of SEQ ID NO: 13 that has partial complementarity with the sense saRNA strand of SEQ ID NO: 14;
b) DA06-4A-27B (SEQ ID NO: 15) and an antisense saRNA strand having a nucleotide sequence of SEQ ID NO: 13 that has partial complementarity with the sense saRNA strand of SEQ ID NO: 15;
c) R6-04M1-27A-S1L1V3 (SEQ ID NO: 18) and an antisense saRNA strand having a nucleotide sequence of SEQ ID NO: 13 that has partial complementarity with the sense saRNA strand of SEQ ID NO: 18;
d) DA06-31A-27A (SEQ ID NO: 19) and an antisense saRNA strand having a nucleotide sequence of SEQ ID NO: 8 that has partial complementarity with the sense saRNA strand of SEQ ID NO: 19;
e) DA06-31B-27A (SEQ ID NO: 20) and a sense saRNA strand having a nucleotide sequence of SEQ ID NO: 7 that has partial complementarity with the antisense saRNA strand of SEQ ID NO: 20;
f) DA06-67A-27A (SEQ ID NO: 21) and an antisense saRNA strand having a nucleotide sequence of SEQ ID NO: 10 that has partial complementarity with the sense saRNA strand of SEQ ID NO: 21;
g) DA06-67B-27A (SEQ ID NO: 22) and a sense saRNA strand having a nucleotide sequence of SEQ ID NO: 9 that has partial complementarity with the antisense saRNA strand of SEQ ID NO: 22;
h) DA6-67A3′L0-27A (SEQ ID NO: 23) and an antisense saRNA strand having a nucleotide sequence of SEQ ID NO: 10 that has partial complementarity with the sense saRNA strand of SEQ ID NO: 23;
j) DA6-67A3′L9-27A (SEQ ID NO: 24) and an antisense saRNA strand having a nucleotide sequence of SEQ ID NO: 10 that has partial complementarity with the sense saRNA strand of SEQ ID NO: 24;
k) DA6-67A3′L4-27A (SEQ ID NO: 25) and an antisense saRNA strand having a nucleotide sequence of SEQ ID NO: 10 that has partial complementarity with the sense saRNA strand of SEQ ID NO: 25;
l) DA6-67B3′L0-27A (SEQ ID NO: 26) and a sense saRNA strand having a nucleotide sequence of SEQ ID NO: 9 that has partial complementarity with the antisense saRNA strand of SEQ ID NO: 26;
m) DA6-67B5′L1-27A (SEQ ID NO: 27) and a sense saRNA strand having a nucleotide sequence of SEQ ID NO: 9 that has partial complementarity with the antisense saRNA strand of SEQ ID NO: 27;
0) DA6-67B5′L9-27A (SEQ ID NO: 29) and a sense saRNA strand having a nucleotide sequence of SEQ ID NO: 9 that has partial complementarity with the antisense saRNA strand of SEQ ID NO: 29;
p) DA6-67B5′L4-27A (SEQ ID NO: 30) and a sense saRNA strand having a nucleotide sequence of SEQ ID NO: 9 that has partial complementarity with the antisense saRNA strand of SEQ ID NO: 30;
q) DA6-67B3′L9-27A (SEQ ID NO: 31) and a sense saRNA strand having a nucleotide sequence of SEQ ID NO: 9 that has partial complementarity with the antisense saRNA strand of SEQ ID NO: 31;
r) DA6-67B3′L4-27A (SEQ ID NO: 32) and a sense saRNA strand having a nucleotide sequence of SEQ ID NO: 9 that has partial complementarity with the antisense saRNA strand of SEQ ID NO: 32;
s) DA06-67A21L1-27A (SEQ ID NO: 33) and an antisense saRNA strand having a nucleotide sequence of SEQ ID NO: 34 that has partial complementarity with the sense saRNA strand of SEQ ID NO: 33;
t) DA06-67B21L1-27A (SEQ ID NO: 36) and sense saRNA strand of SEQ ID NO: 35 that has partial complementarity with the antisense saRNA strand of SEQ ID NO: 36;
u) DA6-04A3′L0-27A (SEQ ID NO: 37) and an antisense saRNA strand having a nucleotide sequence of SEQ ID NO: 6 that has partial complementarity with the sense saRNA strand of SEQ ID NO: 37;
v) DA6-04A5′L1-27A (SEQ ID NO: 38) and an antisense saRNA strand having a nucleotide sequence of SEQ ID NO: 6 that has partial complementarity with the sense saRNA strand of SEQ ID NO: 38;
w) DA6-04A5′L9-27A (SEQ ID NO: 39) and an antisense saRNA strand having a nucleotide sequence of SEQ ID NO: 6 that has partial complementarity with the sense saRNA strand of SEQ ID NO: 39;
x) DA6-04A5′L4-27A (SEQ ID NO: 40) and an antisense saRNA strand having a nucleotide sequence of SEQ ID NO: 6 that has partial complementarity with the sense saRNA strand of SEQ ID NO: 40;
y) DA6-04A3′L1-27A (SEQ ID NO: 41) and an antisense saRNA strand having a nucleotide sequence of SEQ ID NO: 6 that has partial complementarity with the sense saRNA strand of SEQ ID NO: 41;
z) DA6-04A3′L9-27A (SEQ ID NO: 42) and an antisense saRNA strand having a nucleotide sequence of SEQ ID NO: 6 that has partial complementarity with the sense saRNA strand of SEQ ID NO: 42;
aa) DA6-04A3′LA-27A (SEQ ID NO: 43) and an antisense saRNA strand having a nucleotide sequence of SEQ ID NO: 6 that has partial complementarity with the sense saRNA strand of SEQ ID NO: 43;
bb) DA6-04B3′L0-27A (SEQ ID NO: 44) and a sense saRNA strand having a nucleotide sequence of SEQ ID NO: 5 that has partial complementarity with the antisense saRNA strand of SEQ ID NO: 44;
cc) DA6-04B3′L1-27A (SEQ ID NO: 45) and a sense saRNA strand having a nucleotide sequence of SEQ ID NO: 5 that has partial complementarity with the antisense saRNA strand of SEQ ID NO: 45;
dd) DA6-04B3′L9-27A (SEQ ID NO: 46) and a sense saRNA strand having a nucleotide sequence of SEQ ID NO: 5 that has partial complementarity with the antisense saRNA strand of SEQ ID NO: 46;
ee) DA6-04B3′L4-27A (SEQ ID NO: 47) and a sense saRNA strand having a nucleotide sequence of SEQ ID NO: 5 that has partial complementarity with the antisense saRNA strand of SEQ ID NO: 47;
ff) DA06-04A21L1-27A (SEQ ID NO: 48) and an antisense saRNA strand having a nucleotide sequence of SEQ ID NO: 49 that has partial complementarity with the sense saRNA strand of SEQ ID NO: 48;
gg) DA06-04B21L1-27A (SEQ ID NO: 51) and a sense saRNA strand of SEQ ID NO: 50 that has partial complementarity with the antisense saRNA strand of SEQ ID NO: 51;
hh) R6-04M1-16nt-S1L1V3v (SEQ ID NO: 79) and an antisense saRNA strand of SEQ ID NO: 17 that has partial complementarity with the sense saRNA strand of SEQ ID NO: 79;
ii) R6-04M1-15nt-S1L1V3v (SEQ ID NO: 80) and an antisense saRNA strand of SEQ ID NO: 17 that has partial complementarity with the sense saRNA strand of SEQ ID NO: 80;
jj) R6-04M1-14nt-S1L1V3v (SEQ ID NO: 81) and an antisense saRNA strand of SEQ ID NO: 17 that has partial complementarity with the sense saRNA strand of SEQ ID NO: 81;
kk) R6-04M1-13nt-S1L1V3v (SEQ ID NO: 82) and an antisense saRNA strand of SEQ ID NO: 17 that has partial complementarity with the sense saRNA strand of SEQ ID NO: 82;
ll) R6-04M1-(12nt-B)-S1L1V3v (SEQ ID NO: 83) and an antisense saRNA strand of SEQ ID NO: 17 that has partial complementarity with the sense saRNA strand of SEQ ID NO: 83;
mm) R6-04M1-11nt-S1L1V3v (SEQ ID NO: 84) and an antisense saRNA strand of SEQ ID NO: 17 that has partial complementarity with the antisense saRNA strand of SEQ ID NO: 84;
nn) R6-04M1-10nt-S1L1V3v (SEQ ID NO: 85) and an antisense saRNA strand of SEQ ID NO: 17 that has partial complementarity with the antisense saRNA strand of SEQ ID NO: 85;
oo) R6-04M1-9nt-S1L1V3v (SEQ ID NO: 86) and an antisense saRNA strand of SEQ ID NO: 17 that has partial complementarity with the sense saRNA strand of SEQ ID NO: 86;
pp) R6-04M1-8nt-S1L1V3v (SEQ ID NO: 87) and an antisense saRNA strand of SEQ ID NO: 17 that has partial complementarity with the sense saRNA strand of SEQ ID NO: 87;
qq) R6-04M1-7nt-S1L1V3v (SEQ ID NO: 88) and an antisense saRNA strand of SEQ ID NO: 17 that has partial complementarity with the sense saRNA strand of SEQ ID NO: 88;
rr) R6-04M1-6nt-S1L1V3v (SEQ ID NO: 89) and an antisense saRNA strand of SEQ ID NO: 17 that has partial complementarity with the sense saRNA strand of SEQ ID NO: 89;
ss) DS06-4A-S2L5V (SEQ ID NO: 128) and an antisense saRNA strand having a nucleotide sequence of SEQ ID NO: 17 that has partial complementarity with the sense saRNA strand of SEQ ID NO: 128;
ss′) DS06-4A-S2L1v (SEQ ID NO: 16) and an antisense saRNA strand having a nucleotide sequence of SEQ ID NO: 17 that has partial complementarity with the sense saRNA strand of SEQ ID NO: 16;
tt) DA6-27A-5′UTR (SEQ ID NO: 143);
uu) DA6-5′UTR-27A (SEQ ID NO: 144);
vv) R6-67M3-27A-S1L1V3 (SEQ ID NO: 129) and an antisense saRNA strand having a nucleotide sequence of SEQ ID NO: 130 that has partial complementarity with the sense saRNA strand of SEQ ID NO: 129;
ww) R6-67M3-16nt-S1L1V3 (SEQ ID NO: 129) and an antisense saRNA strand having a nucleotide sequence of SEQ ID NO: 131 that has partial complementarity with the sense saRNA strand of SEQ ID NO: 129;
xx) R6-67M3-15nt-S1L1V3 (SEQ ID NO: 129) and an antisense saRNA strand having a nucleotide sequence of SEQ ID NO: 132 that has partial complementarity with the sense saRNA strand of SEQ ID NO: 129;
yy) R6-67M3-14nt-S1L1V3 (SEQ ID NO: 129) and an antisense saRNA strand having a nucleotide sequence of SEQ ID NO: 133 that has partial complementarity with the sense saRNA strand of SEQ ID NO: 129;
zz) R6-67M3-13nt-S1L1V3 (SEQ ID NO: 129) and an antisense saRNA strand having a nucleotide sequence of SEQ ID NO: 134 that has partial complementarity with the sense saRNA strand of SEQ ID NO: 129;
aaa) R6-67M3-12nt-S1L1V3 (SEQ ID NO: 129) and an antisense saRNA strand having a nucleotide sequence of SEQ ID NO: 135 that has partial complementarity with the sense saRNA strand of SEQ ID NO: 129;
bbb) R6-67M3-9nt-S1L1V3 (SEQ ID NO: 129) and an antisense saRNA strand having a nucleotide sequence of SEQ ID NO: 136 that has partial complementarity with the sense saRNA strand of SEQ ID NO: 129;
ccc) R6-67M3-8nt-S1L1V3 (SEQ ID NO: 129) and an antisense saRNA strand having a nucleotide sequence of SEQ ID NO: 137 that has partial complementarity with the sense saRNA strand of SEQ ID NO: 129;
wherein linkers selected from the group consisting of L1, L4 and L9 is present or absent, wherein L1 represents spacer-18; L4 represents spacer-C6; and L9 represents spacer-9. 46. The multi-valent oligonucleotide agent of any one of items 1-43, wherein the multi-valent oligonucleotide agent has a nucleotide sequence that is at least 90% identical to the nucleotide sequences selected from:
a) R6-04S1&67S1R-L1V2 (SEQ ID NO: 52) and an antisense saRNA strand having a nucleotide sequence of SEQ ID NO: 53 that has partial complementarity with the strand of SEQ ID NO: 52 and an antisense saRNA strand having a nucleotide sequence of SEQ ID NO: 78 that has partial complementarity with the strand of SEQ ID NO: 52;
b) R6-04S1&67S5-L1V2 (SEQ ID NO: 56) and an antisense saRNA strand having a nucleotide sequence of SEQ ID NO: 53 that has partial complementarity with the strand of SEQ ID NO: 56 and an antisense saRNA strand having a nucleotide sequence of SEQ ID NO: 78 that has partial complementarity with the strand of SEQ ID NO: 56; and
c) R6-04M1&R17-388E-L1V2 (SEQ ID NO: 140) and an antisense saRNA strand of SEQ ID NO: 17 that has partial complementarity with the strand of SEQ ID NO: 140 and an antisense siRNA strand of SEQ ID NO: 141 that has partial complementarity with the strand of SEQ ID NO: 140.

47. The multi-valent oligonucleotide agent of any one of items 36-44, wherein the multi-valent oligonucleotide agent has a nucleotide sequence that is at least 90% identical to the nucleotide sequences selected from:
a) R6-04S1&27A&67S1R-L1V2 (SEQ ID NO: 54) and an antisense saRNA strand having a nucleotide sequence of SEQ ID NO: 53 that has partial complementarity with the strand of SEQ ID NO: 54 and an antisense saRNA strand having a nucleotide sequence of SEQ ID NO: 78 that has partial complementarity with the strand of SEQ ID NO: 54;
b) R6-04S1&67S1R&27A-L1V2 (SEQ ID NO: 55) and an antisense saRNA strand having a nucleotide sequence of SEQ ID NO: 53 that has partial complementarity with the strand of SEQ ID NO: 55 and an antisense saRNA strand having a nucleotide sequence of SEQ ID NO: 78 that has partial complementarity with the strand of SEQ ID NO: 55;
c) R6-04S1&27A&67S5-L1V2 (SEQ ID NO: 57) and an antisense saRNA strand having a nucleotide sequence of SEQ ID NO: 53 that has partial complementarity with the strand of SEQ ID NO: 57 and an antisense saRNA strand having a nucleotide sequence of SEQ ID NO: 78 that has partial complementarity with the strand of SEQ ID NO: 57; and
d) R6-04S1&67S5&27A-L1V2 (SEQ ID NO: 58) and an antisense saRNA strand having a nucleotide sequence of SEQ ID NO: 53 that has partial complementarity with the strand of SEQ ID NO: 58 and an antisense saRNA strand having a nucleotide sequence of SEQ ID NO: 78 that has partial complementarity with the strand of SEQ ID NO: 58.

48. The multi-valent oligonucleotide agent of any one of items 36-47, wherein the multi-valent oligonucleotide agent is selected from or has at least 90% sequence identity to those shown in any one of Tables 7-20, and wherein the linking components and/or linkage bonds and/or orientation of those multi-valent oligonucleotide agents are changeable.

49. The multi-valent oligonucleotide agent of item 48, wherein the linking components and/or linkage bonds and/or orientation of the multi-valent oligonucleotide agent are selected from those defined in any one of items 12-25.

50. The multi-valent oligonucleotide agent of any one of items 1-35, wherein one or more of the functional oligonucleotides increase the expression of CDKN1A/p21 gene or protein and/or decreases the expression CD274/PDL-1.

51. The multi-valent oligonucleotide agent of item 50, wherein the dsRNA(s) are independently selected from: a saRNA that increases the expression of the CDKN1A/p21 gene or protein; and a siRNA that decreases the expression CD274/PDL-1; and/or wherein the ASO(s) are independently selected from an ASO that increases the expression of CDKN1A/p21 gene or protein and/or decreases the expression CD274/PDL-1.

52. The multi-valent oligonucleotide agent of item 50, wherein the dsRNA is a saRNA having a nucleotide sequence of a sense strand that is at least 90% identical to the nucleotide sequence of RAG1-40 (SEQ ID NO: 62).

53. The multi-valent oligonucleotide agent of item 50, wherein the dsRNA is a saRNA having a nucleotide sequence of a antisense strand that is at least 90% identical to the nucleotide sequence of RAG1-40 (SEQ ID NO: 63).

54. The multi-valent oligonucleotide agent of item 50, wherein the dsRNA is a saRNA having a nucleotide sequence of a sense strand that is at least 90% identical to the nucleotide sequence of RAG1-40 (SEQ ID NO: 62) and a nucleotide sequence of an antisense strand that is at least 90% identical to the nucleotide sequence of RAG1-40 (SEQ ID NO: 63).

55. The multi-valent oligonucleotide agent of item 50, wherein the dsRNA is a siRNA having a nucleotide sequence of a sense strand that is at least 90% identical to the nucleotide sequence selected from:
a) siPDL1-2 (SEQ ID NO: 64); and
b) siPDL1-3 (SEQ ID NO: 66).

56. The multi-valent oligonucleotide agent of item 50, wherein the dsRNA is a siRNA having a nucleotide sequence of an antisense strand that is at least 90% identical to the nucleotide sequence selected from:
a) siPDL1-2 (SEQ ID NO: 65); and
b) siPDL1-3 (SEQ ID NO: 67).

57. The multi-valent oligonucleotide agent of item 50, wherein the dsRNA is a siRNA selected from:
a) a siRNA having a nucleotide sequence of a sense strand that is at least 90% identical to the nucleotide sequence siPDL1-2 (SEQ ID NO: 64) and a nucleotide sequence of an antisense strand that is at least 90% identical to the nucleotide sequence of siPDL1-2 (SEQ ID NO: 65); and
b) a siRNA having a nucleotide sequence of a sense strand that is at least 90% identical to the nucleotide sequence siPDL1-2 (SEQ ID NO: 66) and a nucleotide sequence of an antisense strand that is at least 90% identical to the nucleotide sequence of siPDL1-2 (SEQ ID NO: 67). 58. The multi-valent oligonucleotide agent of item 50, wherein the ASO has a nucleotide sequence that is at least 90% identical to the nucleotide sequence selected from:
a) aPDL1-1 (SEQ ID NO: 68);
b) aPDL1-2 (SEQ ID NO: 69); and
c) aPDL1-3 (SEQ ID NO: 70).

59. The multi-valent oligonucleotide agent of item 50, wherein the multi-valent oligonucleotide agent has a nucleotide sequence that is at least 90% identical to the nucleotide sequences selected from:
a) saP21-40/siPDL1-2 (SEQ ID NO: 71) and an antisense saRNA strand having a nucleotide sequence of SEQ ID NO: 63 that has partial complementarity with the strand of SEQ ID NO: 71 and an antisense siRNA strand having a nucleotide sequence of SEQ ID NO: 65 that has partial complementarity with the strand of SEQ ID NO: 71;
b) saP21-40/siPDL1-3 (SEQ ID NO: 100) and an antisense saRNA strand having a nucleotide sequence of SEQ ID NO: 63 that has partial complementarity with the sense saRNA strand of SEQ ID NO: 100 and an antisense saRNA strand having a nucleotide sequence of SEQ ID NO: 65 that has partial complementarity with the strand of SEQ ID NO: 100;
c) saP21-40/aPDL1-1 (SEQ ID NO: 72) and an antisense saRNA strand having a nucleotide sequence of SEQ ID NO: 63 that has partial complementarity with the sense saRNA strand of SEQ ID NO: 72;
d) saP21-40/aPDL1-2 (SEQ ID NO: 73) and an antisense saRNA strand having a nucleotide sequence of SEQ ID NO: 63 that has partial complementarity with the sense saRNA strand of SEQ ID NO: 73;
e) saP21-40/aPDL1-3 (SEQ ID NO: 74) and an antisense saRNA strand having a nucleotide sequence of SEQ ID NO: 63 that has partial complementarity with the sense saRNA strand of SEQ ID NO: 74;
f) saP21-40/aPDL1-1R (SEQ ID NO: 75) and an antisense saRNA strand having a nucleotide sequence of SEQ ID NO: 63 that has partial complementarity with the sense saRNA strand of SEQ ID NO: 75;
g) saP21-40/aPDL1-2R (SEQ ID NO: 76) and an antisense saRNA strand having a nucleotide sequence of SEQ ID NO: 63 that has partial complementarity with the sense saRNA strand of SEQ ID NO: 76; and
h) saP21-40/aPDL1-3R (SEQ ID NO: 77) and an antisense saRNA strand having a nucleotide sequence of SEQ ID NO: 63 that has partial complementarity with the sense saRNA strand of SEQ ID NO: 77.

60. The multi-valent oligonucleotide agent of item 50, wherein the multi-valent oligonucleotide agent is selected from or has at least 90% sequence identity to those shown in Table 16, and wherein the linking components and/or linkage bonds and/or orientation of those multi-valent oligonucleotide agents are changeable.

61. The multi-valent oligonucleotide agent of item 50, wherein the linking components and/or linkage bonds and/or orientation of the multi-valent oligonucleotide agent are selected from those defined in any one of items 12-25.

62. A product comprising the multi-valent oligonucleotide agent of any one of items 1-61.

63. The product of item 62, wherein the product is selected from a pharmaceutical composition further comprising at least one pharmaceutically acceptable carrier or a kit.

64. The product of item 63, wherein the at least one pharmaceutically acceptable carrier is selected from an aqueous carrier, liposome, polymeric polymer, polypeptide and nanoparticle.

65. The product of item 63, wherein the kit is used for quality control, drug candidate screening, drug delivery to a tissue or into a cell, or improving pharmacological properties of a molecule.

66. A method for disease treatment comprising administering sufficient amount of one or more of the multi-valent oligonucleotide agent of any one of items 1-61 or a product of items 62-65 to a subject in need of such treatment.

67. The method of item 66, wherein the method further comprises providing to the subject one or more additional medicament or therapy, for example,
the additional medicament is one or more selected from the group consisting of Nusinersen, Risdiplam, Branaplam, Zolgensma, Fomivirsen, Mipomersen, Eteplirsen, Inotersen, Golodirsen, Volanesorsen, Defibrotide, Patisiran, Givosiran, Lumasiran, Inclisiran or Pegaptanib; and/or
the additional therapy is one or more selected from physical therapy, diet control and surgery.

68. The method of item 66, wherein the method is for treating or delaying the onset or progression of SMN-deficiency-related conditions in a subject.

69. The method of item 66, wherein the subject is suffering from or in the risk of having spinal muscular atrophy (SMA); and/or the subject has decreased or abnormal SMN full length protein expression.

70. The method of any one of items 68-69, wherein at least one of the two or more targeting oligonucleotide increases the expression of an SMN2 gene or protein and/or increases the production of functional SMN protein by modulating SMN2 mRNA splicing or stability (SMN2 mRNA modulators).

71. The method of item 68, further comprising providing to the subject one or more additional medicament or therapy for treating or delaying the onset or progression of SMN.

72. The method of item 71, wherein the additional medicament is one or more selected from the group consisting of Nusinersen, Risdiplam, Branaplam, Zolgensma.

73. The method of item 71, wherein the additional therapy is one or more selected from physical therapy, diet control and surgery.

74. The method of item 66, wherein the multi-valent oligonucleotide agent increasing the expression of a CDKN1A/p21 gene or protein, and decreasing the expression CD274/PDL-1.

75. The method of item 66, wherein the disease is a p21/PDL-1 associated disease.

76. The method of items 66, wherein the patient is suffering from cancer or in high risk of having cancer.

77. The method of item 76, wherein the cancer is a solid tumor or a non-solid tumor.

78. The method of item 76, wherein the cancer is selected from the group consisting of fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma/colorectal cancer, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

79. The method of any one of items 74-78 further comprising administering one or more additional agents or therapies used for treating cancer.

80. The method of item 79, wherein the additional agent or therapy is selected from radiation, chemotherapy, surgery, immunotherapy, genetherapy.

81. The method of item 80, wherein the chemotherapy is of cisplatin, carboplatin, paclitaxel, docetaxel, gemcitabine, vinorelbine, vinblastine, irinotecan, etoposide, or pemetrexed, or combinations thereof, or a pharmaceutically acceptable salt thereof.

82. The method of item 79, wherein the additional agent is selected from an immune modulating antibody and an antibody drug conjugate.

83. The method of item 82, wherein the immune modulating antibody is selected from an anti-PD-1 antibody, an anti-PD-L1 antibody, an anti-CD40 antibody, an anti-CTLA-4 antibody, or an anti-OX40 antibody, or any combination thereof; and/or the additional antibody drug conjugate targets c-Met kinase, LRRC15, EGFR, or CS1, or any combination thereof.

84. A method for the preparation of the multi-valent oligonucleotide agent of any one of items 1-61 comprising:
providing said two or more functional oligonucleotides and covalently linking the same; or
synthesizing the full length oligonucleotide agent.

85. The method of item 84, wherein the two or more functional oligonucleotides are covalently linked by a linking component or with no linking component.

86. The method of item 85, wherein the linking component is selected from the following linkers or derivatives thereof:

| Name | Formula | Structure |
|---|---|---|
| Spacer-9 | C₃₆H₄₉N₂O₇P | 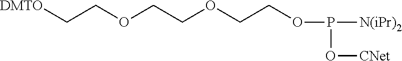 |
| Spacer-18 | C₄₂H₆₁N₂O₁₀P | 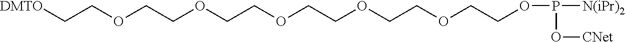 |
| Spacer-C3 | C₃₃H₄₃N₂O₅P | 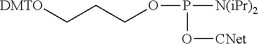 |
| Spacer-C6 | C₃₆H₄₉N₂O₅P | 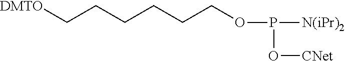 |

87. The method of item 86, wherein the two adjacent functional oligonucleotides are covalently linked by:

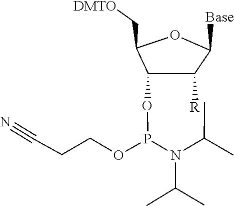

wherein R represents —H or —OH or —OMe, or -MOE, or —F, or other 2′ chemical modifications.

88. The method of item 86, wherein the two adjacent functional oligonucleotides are covalently linked by a phosphodiester bond or a phosphorothioate bond.

89. The method of item 86, wherein the two adjacent functional oligonucleotides are covalently linked by one or more nucleotides.

90. The method of item 85, wherein the method further comprising providing one or more chemically modified nucleotides in the functional oligonucleotides.

91. The method of item 90, wherein the chemical modification of the at least one chemically modified nucleotide is a 2′ sugar modification selected from one or more of: 2′-fluoro-2′-deoxynucleoside (2′-F) modification, 2′-O-methyl (2′-O—Me), modification, and 2′-O-(2-methoxyethyl) (2′-O-MOE) modification.

92. The method of item 90, wherein the chemical modification of the at least one chemically modified nucleotide is a Phosphorothioate (PS) backbone modification.

93. The method of item 90, wherein the chemical modification of the at least one chemically modified nucleotide is an addition of a 5′-phosophate moiety at the 5′ end of the nucleotide sequence.

94. The method of item 90 wherein the chemical modification of the at least one chemically modified nucleotide is an addition of an (E)-vinylphosphonate moiety at the 5′ end of the nucleotide sequence.

95. The method of item 90, wherein the chemical modification of the at least one chemically modified nucleotide is an addition of a 5-methyl cytosine moiety at the 5′ end of the nucleotide sequence.

96. The method of item 84, wherein each of the ASO in the agent is covalently linked to the adjacent targeting oligonucleotide in a 3′ to 5′ orientation or in a 5′ to 3′ orientation.

97. The method of item 84, wherein each of the dsRNA in the agent is covalently linked to an adjacent ASO at its 3′ end of the sense or antisense strand; or at its 5′ end of the sense or antisense strand.

98. An isolated or synthesized oligonucleotide comprising: a nucleotide sequence of a saRNA sense strand that is at least 90% identical to the nucleotide sequence of SEQ ID NO: 62.

99. The isolated or synthesized oligonucleotide of item 98, wherein the oligonucleotide further comprises an antisense strand that has partial complementarity with the sense saRNA strand of item 98.

100. The isolated or synthesized oligonucleotide of item 99, wherein the oligonucleotide further comprises an antisense strand that is at least 90% identical to the nucleotide sequence of SEQ ID NO: 63.

101. The isolated or synthesized oligonucleotide of item 98, wherein the isolated or synthesized oligonucleotide comprising: a nucleotide sequence of a saRNA sense strand of SEQ ID NO: 62 and a saRNA antisense strand of SEQ ID NO: 63.

102. A pharmaceutical composition or kit comprising the isolated or synthesized oligonucleotide of any one or items 98-101.

103. A method for disease treatment comprising administering sufficient amount of one or more of the isolated or synthesized oligonucleotide of any one of items 98-101 or a pharmaceutical composition or kit of items 102 to a subject in need of such treatment.

### 3. BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the effect of DAO structure on the expression of full-length (SMN2FL) and exon 7 skipped (SMN2Δ7) SMN2 mRNA in GM03813 cells. GM03813 cells were treated with the indicated concentration of saRNA, ASO, combo treatment (saRNA+ASO) and DAO (DA06-4A-27A and DA06-4A-27B) for 72 hours. Mock samples, as a control treatment, were transfected in the absence of an oligonucleotide. dsCon2 was transfected as an unrelated oligonucleotide control. DS06-332i is an siRNA for SMN2 and was transfected as a control treatment. mRNA levels of SMN2FL and SMNA7 were determined by RT-qPCR using two pairs of primers in separate PCR reactions.

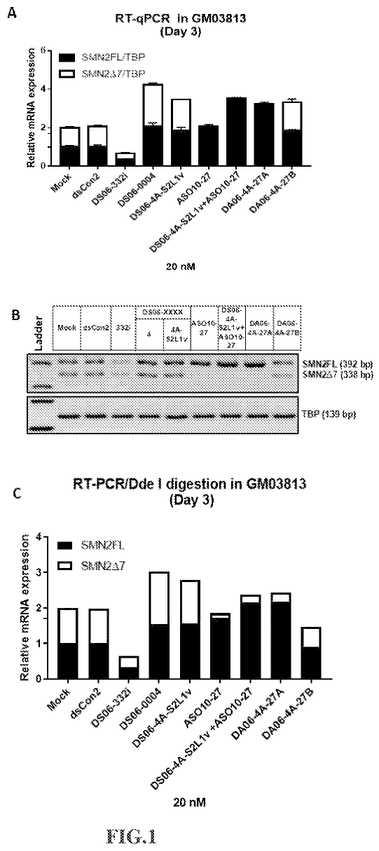

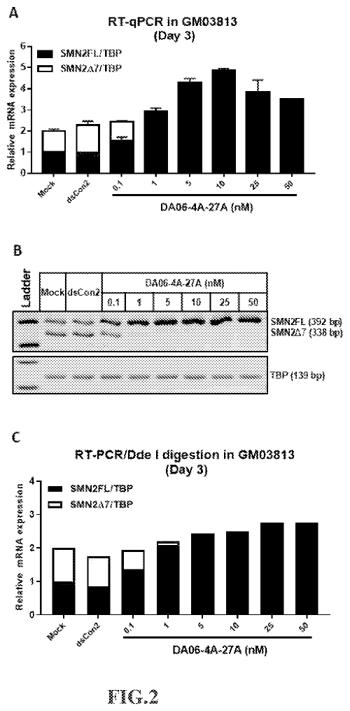

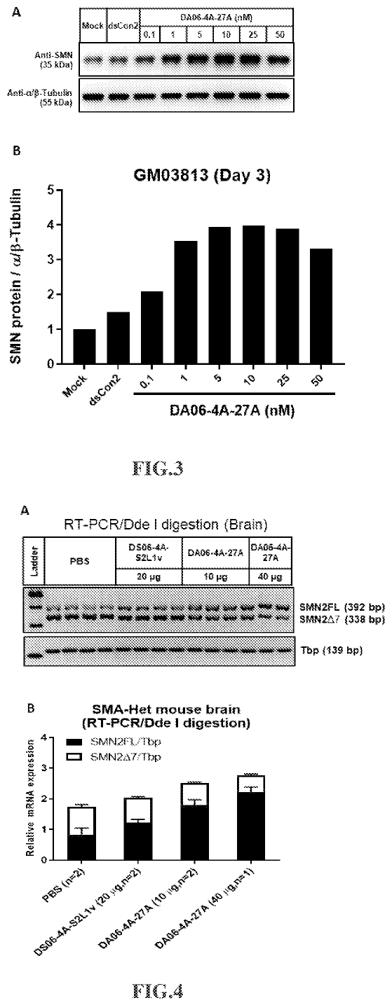

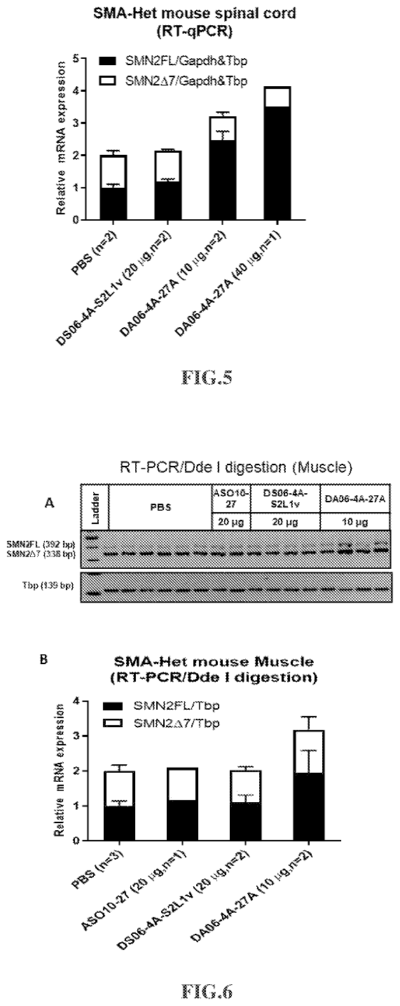

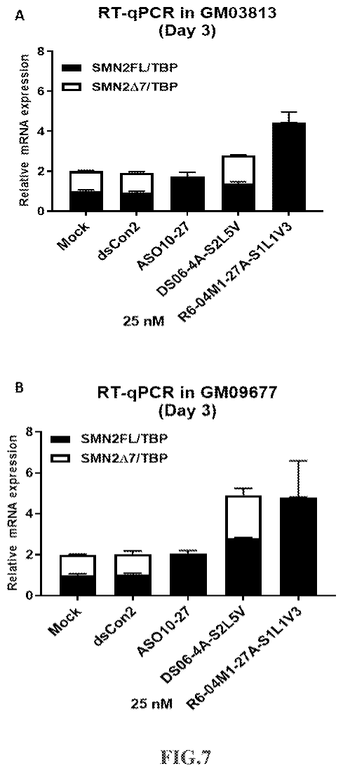

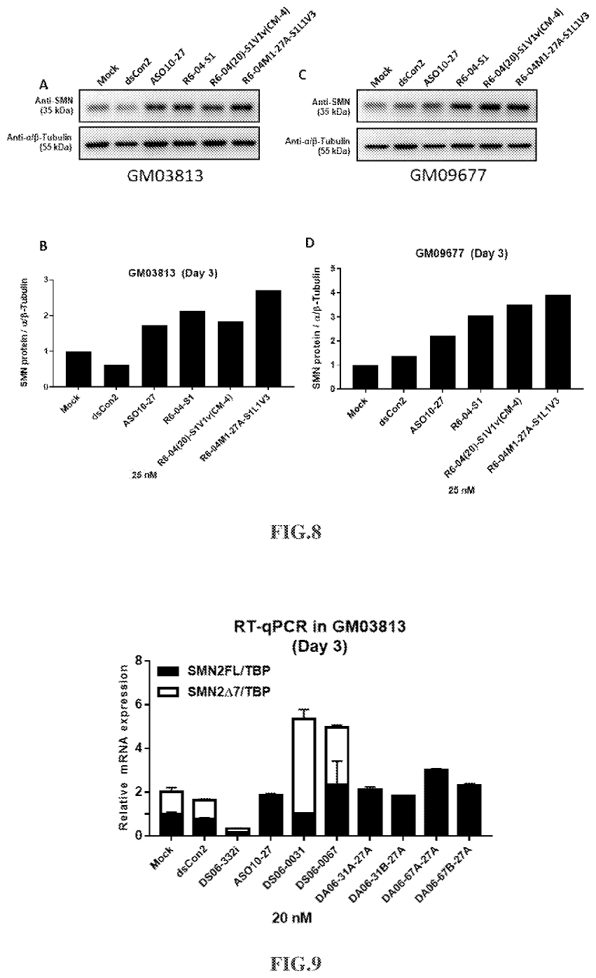

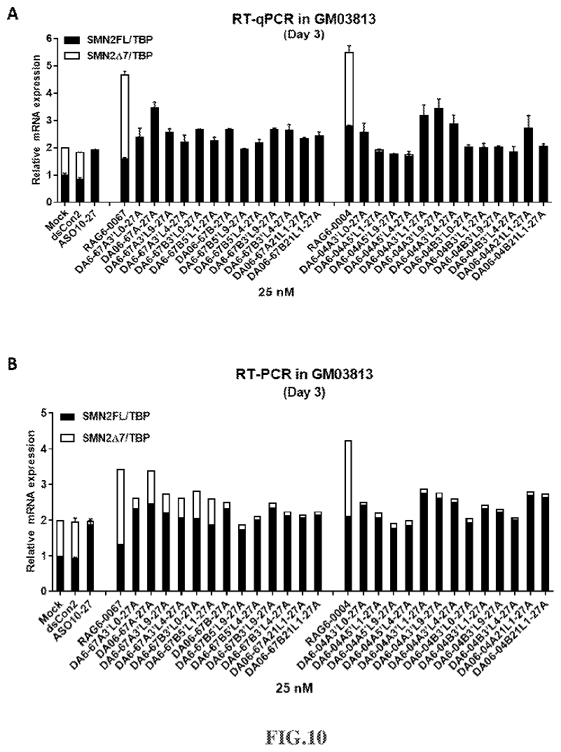

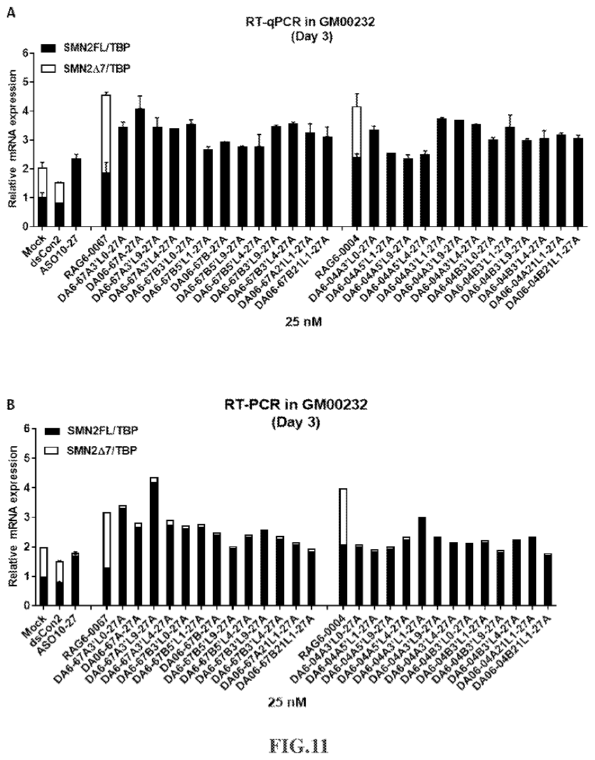

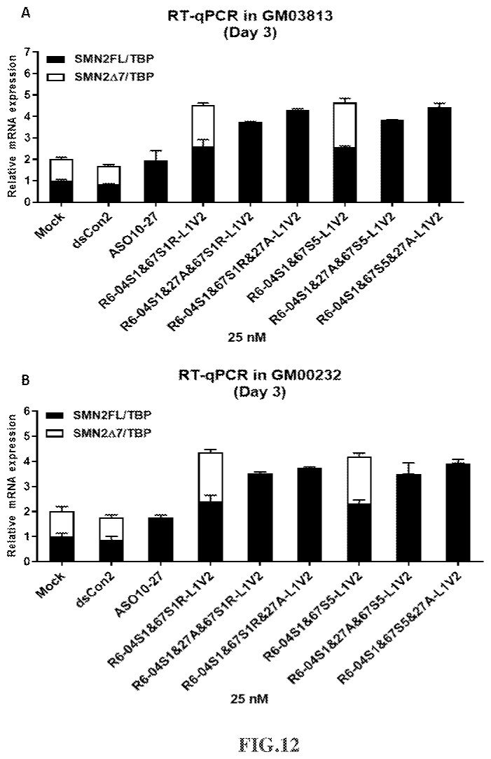

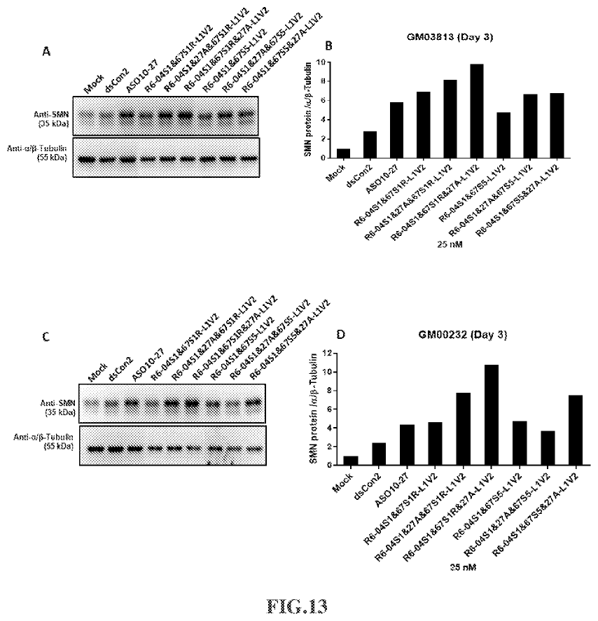

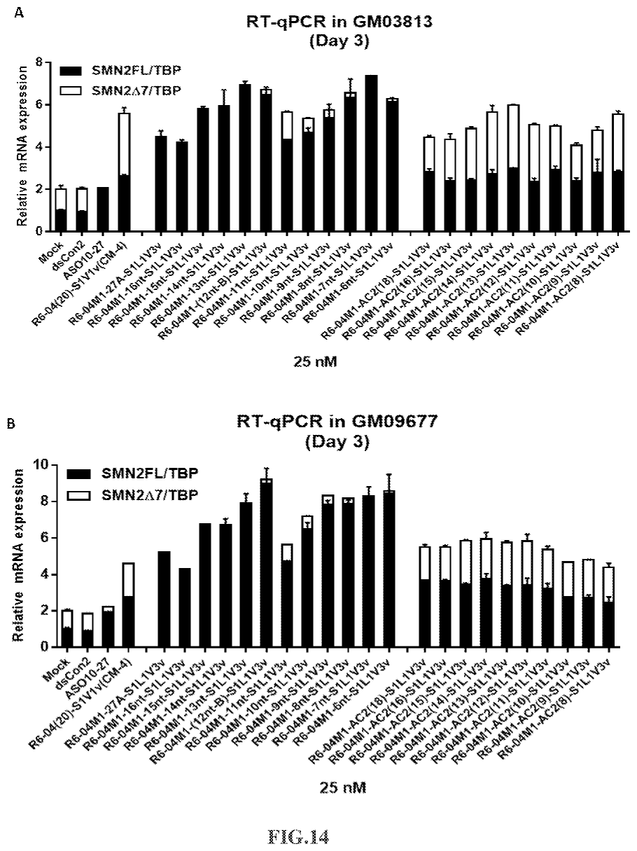

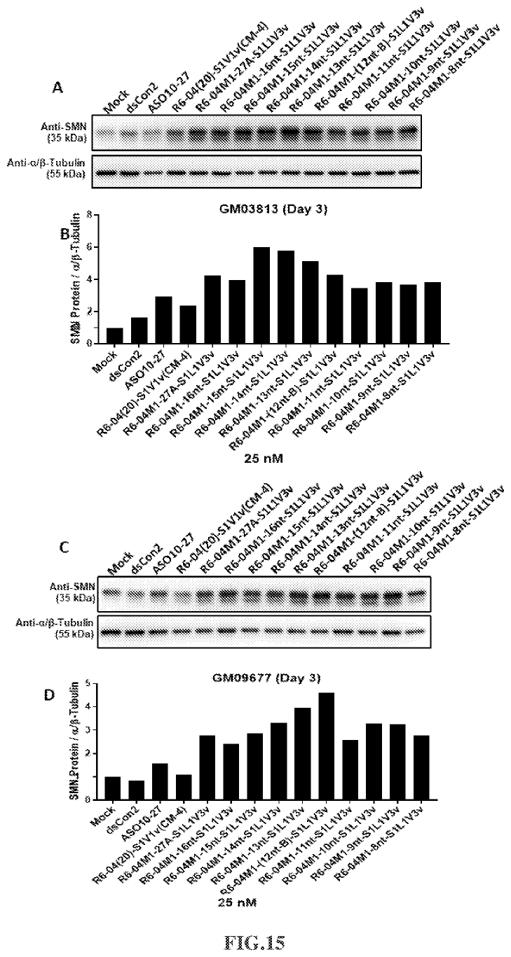

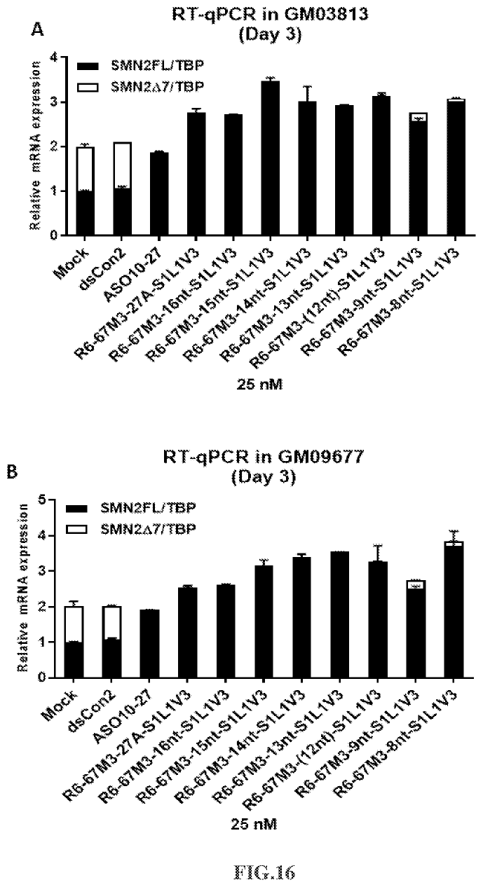

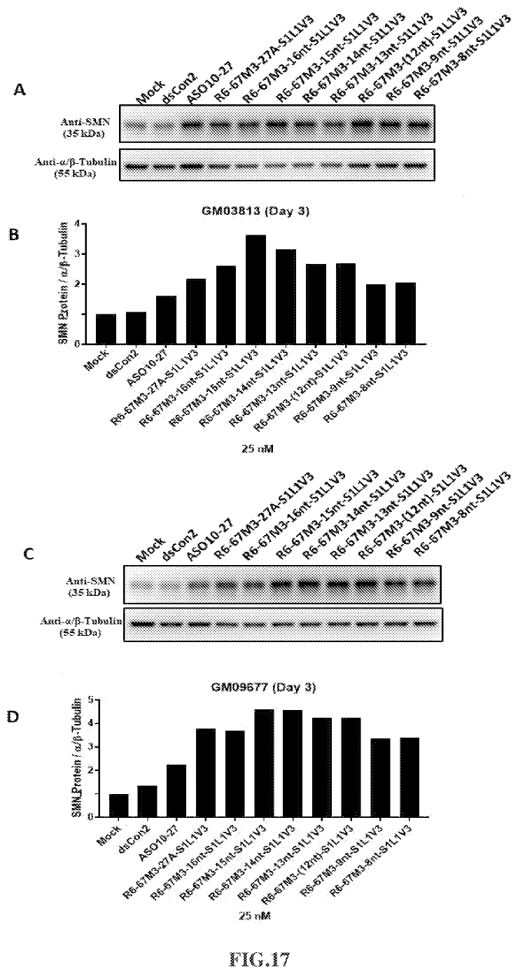

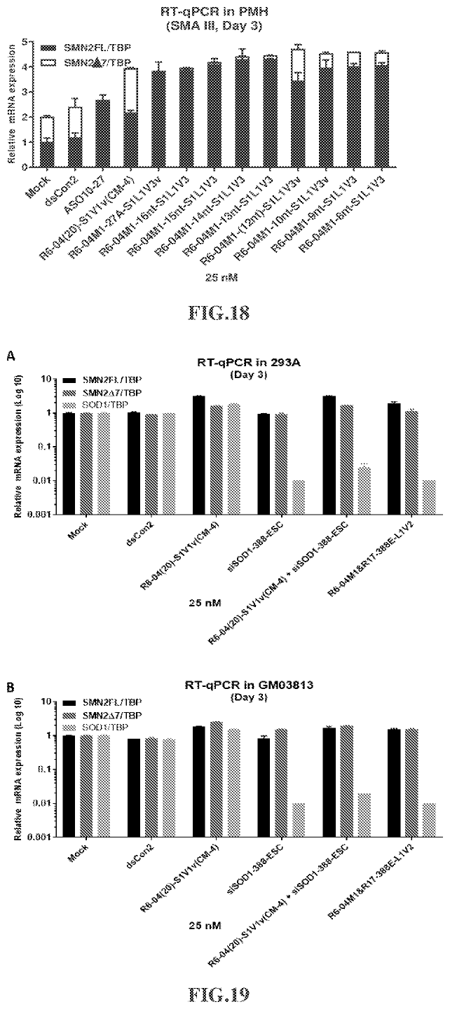

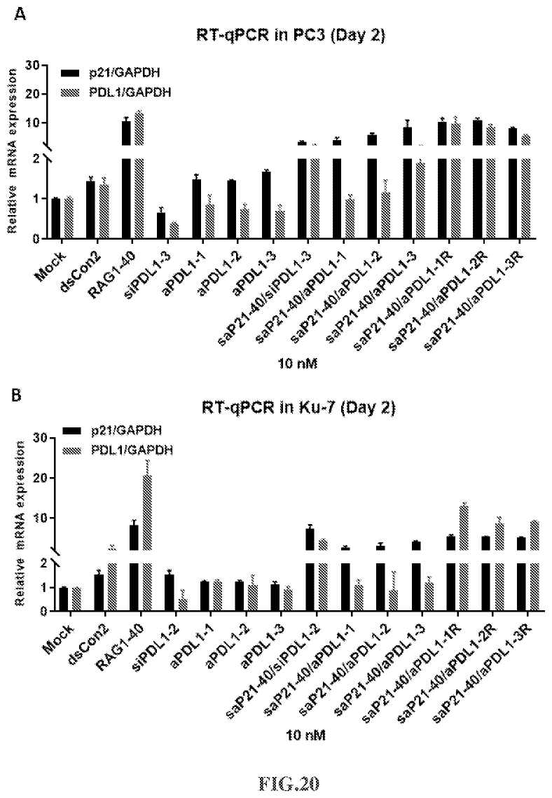

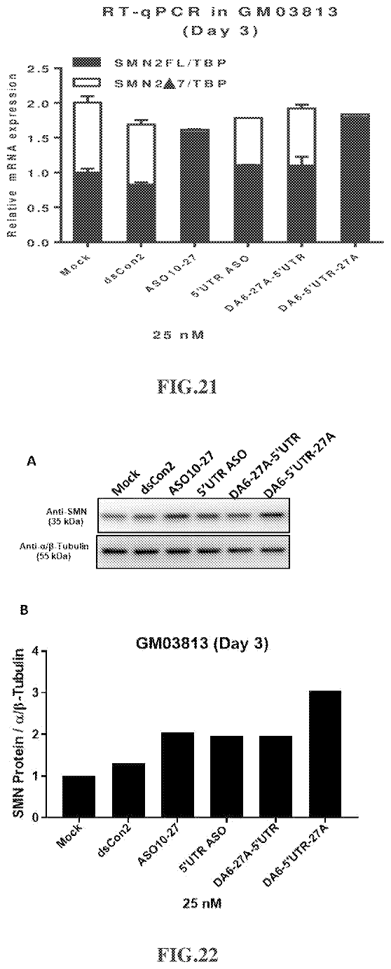

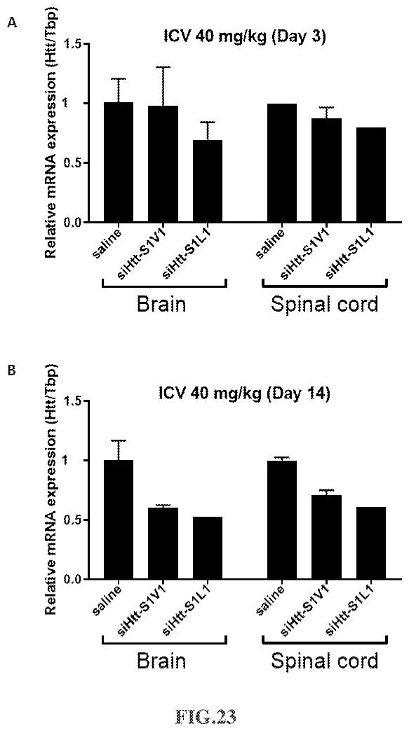

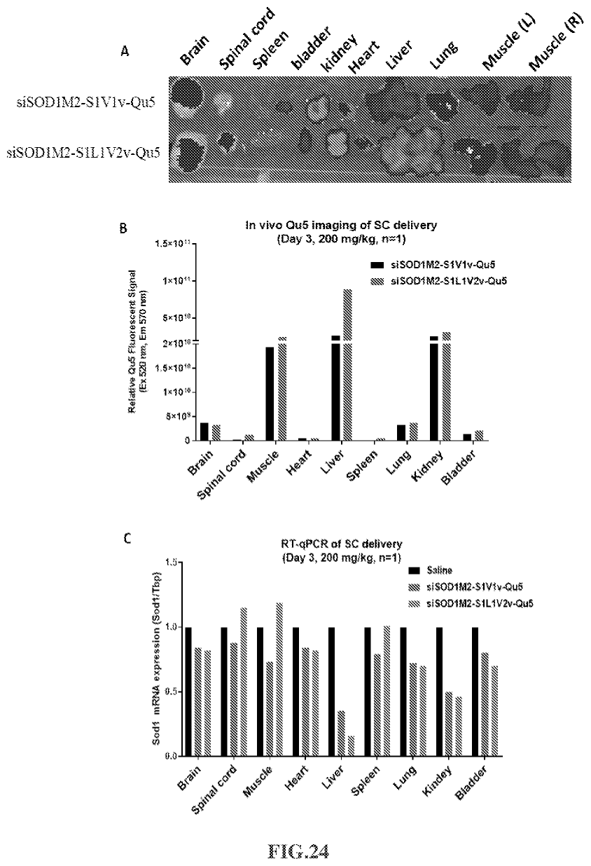

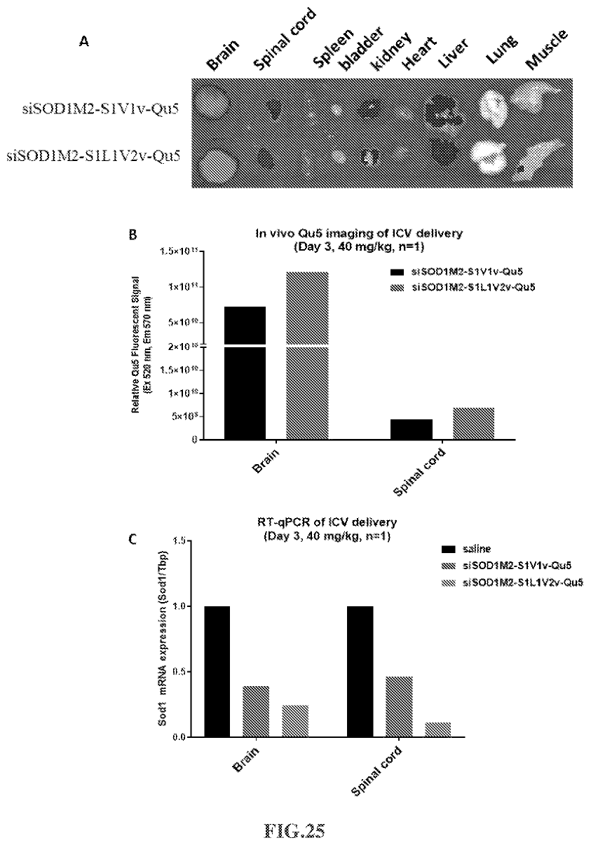

In particular, patient-derived SMA fibroblasts (GM03813 cells) were treated with 20 nM of the indicated oligonucleotides for 72 hours. Exemplary saRNA duplex DS06-0004 is an established activator of human SMN2 gene transcription. DS06-4A-S2LIA is a divalent saRNA structure based on DS06-0004 sequence in which two chemically-modified duplexes of the same composition are covalently linked together. ASO10-27 is a “mixmer” splicing modulator that increases cellular levels of SMN2FL transcript via inclusion of exon 7. Combination of both DS06-4A-S2L1v and ASO10-27 (DS06-4A-S2L1v+ASO10-27) were treated at 20 nM each. DAO constructs were synthesized by covalently linking ASO10-27 sequence to a saRNA variant of DS06-0004 in conventional (DA-06-4A27A) or reverse (DA-06-4A27B) sequence orientation. Mock treatments were transfected in absence of oligonucleotide. dsCon2 served as a non-specific control duplex, while an SMN2-specific siRNA (DS06-332i) served as a transfection control by monitoring target gene knockdown. In FIG. 1A, both full length (SMN2FL) and 47 (SMN2Δ7) splicing variants of SMN2 were quantified by RT-qPCR using isoform-specific primer sets. TBP was amplified as an internal reference. Shown are changes in SMN2FL and SMN2A7 expression levels relative to Mock treatment after normalization to TBP reference levels. In FIG. 1B, SMN2FL and SMN2Δ7 levels were visualized on an agarose gel by semi-quantitative RT-PCR using an alternative primer set spanning exon 7. Digestion with DdeI enzyme was performed to distinguish amplicons originating from SMN2 or SMN1 sequence. Indicated is SMN2 product size after digestion. Scanning optical densitometry was subsequently performed on the above agarose gel images to quantify band intensity. Shown in FIG. 1C are SMN2FL and SMN2Δ7 band intensity levels relative to Mock treatment normalization to TBP reference levels.

FIG. 2 shows the dose dependent study of DAO (DA06-4A-27A) on the expression of SMN2FL and SMN2Δ7 mRNA and SMN protein in GM03813 cells. Mock and dsCon2 are described as in FIG. 1. mRNA levels of SMN2FL and SMNA7 were determined by RT-qPCR using two pairs of primers in separate PCR reactions. Proteins were harvested from the treated cells and immunoblotted by western blotting assay using an antibody against human SMN protein. An antibody against α/β-Tubulin was also blotted to serve as a control for protein loading.

In particular, dose-dependent effect of DAO (DA06-4A-27A) on SMN2FL and SMN2Δ7 transcript levels in vitro was shown. GM03813 cells were treated at the indicated concentrations of DA06-4A-27A for 72 hours. Mock treatments were transfected in absence of oligonucleotide. dsCon2 served as a non-specific control duplex. In FIG. 2A, both full length (SMN2FL) and Δ7 (SMN2Δ7) splicing variants of SMN2 were quantified by RT-qPCR using isoform-specific primer sets. TBP was amplified as an internal reference. Shown are changes in SMN2FL and SMN2Δ7 expression levels relative to Mock treatment after normalization to TBP reference levels. In FIG. 2B, SMN2FL and SMN2Δ7 levels were visualized on an agarose gel by semi-quantitative RT-PCR using an alternative primer set spanning exon 7. Digestion with DdeI enzyme was performed to distinguish amplicons originating from SMN2 or SMN1 sequence. Indicated is SMN2 product size after digestion. Scanning optical densitometry was subsequently performed on the above agarose gel images to quantify band intensity. Shown in FIG. 2C are SMN2FL and SMN2Δ7 band intensity levels relative to Mock treatment normalization to TBP reference levels.

FIG. 3 shows the dose-dependent effect of DAO (DA06-4A-27A) on the expression of SMN protein in GM03813 cells. Mock and dsCon2 are described as FIG. 1. mRNA levels of SMN2FL and SMNΔ7 were determined by RT-qPCR using two pairs of primers in separate PCR reactions. Proteins were harvested from the treated cells and immunoblotted by western blotting assay using an antibody against human SMN protein. An antibody against α/β-Tubulin was also blotted to serve as a control for protein loading.

In particular, the dose-dependent effect of DAO (DA06-4A-27A) on total SMN protein levels in vitro was shown. GM03813 cells were treated at the indicated concentrations of DA06-4A-27A for 72 hours. Mock treatments were transfected in absence of oligonucleotide. dsCon2 served as a non-specific control duplex. Whole cell protein extracts were harvested for immunoblot analysis. In FIG. 3A, total SMN protein levels were detected using an indiscriminate monoclonal antibody that recognized both SMN1 and SMN2 gene product. Immunodetection of α/β-Tubulin served as a protein loading control. Scanning optical densitometry was used to quantify protein band intensity from the above immunoblot. Shown in FIG. 3B are changes in total SMN protein levels relative to Mock treatment after normalizing to α/β-Tubulin.

FIG. 4 shows the efficacy of DAO (DA06-4A-27A) on the expression of SMN2FL and SMN2Δ7 mRNA in SMA-Het mouse brain. RNA was collected from SMA-Het mouse brain by RNAVzol with Qiagen RNeasy column. PBS means no treatment control.

In particular, demonstrated in this figure was DAO (DA06-4A-27A) activity in the brain of SMA-heterozygous (Het) mice (Smn1^{+/-}, SMN2^{+/-}). DA06-4A-27A was administered into pup mice via ICV injection at the indicated doses on postnatal day 1 (PND1). PBS treatments contained Fast Green as a procedural control to visually confirm biodistribution throughout mouse brain and spinal cord. Mice were sacrificed 72 hours later and whole tissue samples were collected for RNA isolation. The n-value indicates animal number in each treatment group. FIG. 4A shows SMN2FL and SMN2Δ7 mRNA levels visualized on an agarose gel via semi-quantitative RT-PCR using the exon7 spanning primer set digested with DdeI enzyme. Amplicon size of SMN2FL (392 bp) and SMN2Δ7 (338 bp) after digestion are indicated with comparison to a 100 bp DNA ladder. Tbp gene was amplified as a loading control. Scanning optical densitometry was subsequently performed on the agarose gel image to quantify band intensity. Shown in FIG. 4B are mean values+SD of SMN2FL and SMN2Δ7 band intensity levels relative to PBS treatments after normalization to Tbp reference levels.

FIG. 5 shows the efficacy of DAO (DA06-4A-27A) on the expression of SMN2FL and SMN2Δ7 mRNA in SMA-Het mouse spinal cord. RNA was collected from SMA-Het mouse spinal cord by RNAVzol with Qiagen RNeasy column. PBS means no treatment control.

In particular, the figure shows the in vivo activity of DAO (DA06-4A-27A) in the spinal cord of SMA-Het mice. Pub mice were treated on PND1 as described in FIG. 4. Mice were sacrificed 72 hours later and spinal cord samples were collected for RNA isolation. The n-value indicates animal number in each treatment group. SMN2FL and SMN2Δ7 splicing variants were quantified by RT-qPCR using isoform-specific primer sets. Both Gapdh & Tbp were amplified as internal reference controls. Shown are changes in SMN2FL and SMN2Δ7 expression levels relative to Mock treatment after normalization to mean values of both reference gene levels.

FIG. 6 shows the efficacy of DAO (DA06-4A-27A) on the expression of SMN2FL and SMN2Δ7 mRNA in SMA-Het mouse muscle. RNA was collected from SMA-Het mouse muscle by RNAVzol with Qiagen RNeasy column. PBS means no treatment control.

In particular, the figure demonstrates DAO (DA06-4A-27A) activity in muscle of SMA-Het mice. Pub mice were treated on PND1 as described in FIG. 4. Mice were sacrificed 72 hours later and muscle tissue samples were collected for RNA isolation. The n-value indicates animal number in each treatment group. FIG. 6A shows SMN2FL and SMN2Δ7 mRNA levels visualized on an agarose gel via semi-quantitative RT-PCR using the exon7 spanning primer set digested with DdeI enzyme. Amplicon size of SMN2FL (392 bp) and SMN2Δ7 (338 bp) after digestion are indicated with comparison to a 100 bp DNA ladder. Tbp gene was amplified as a loading control. Scanning optical densitometry was subsequently performed on the agarose gel image to quantify band intensity. Shown in FIG. 6B are mean values+SD of SMN2FL and SMN2Δ7 band intensity levels relative to PBS treatments after normalization to Tbp reference levels.

FIG. 7 shows the effect of different DAO structure on the expression of SMN2FL and SMN2Δ7 mRNA in GM03813 and GM09677 cells. Mock and dsCon2 are described as FIG. 1. mRNA levels of SMN2FL and SMNΔ7 were determined by RT-qPCR using two pairs of primers in separate PCR reactions. The values (y-axis) are SMN2 band intensity relative to Mock treatment after normalizing to the band intensity of TBP.

In particular, the figure demonstrates DAO (R6-04M1-27A-S1L1V3) activity with optimized medicinal chemistry in patient-derived SMA fibroblasts. GM03813 and GM09677 cells were treated with 25 nM of the indicated oligonucleotides for 72 hours. DS06-4A-S2L5V is a divalent saRNA variant based on DS06-4A-S2LIV sequence/scaffold harboring optimized medicinal chemistry. R6-04M1-27A-S1L1V3 is an updated DAO construct based on DA-06-4A27A with similarly enhanced chemistry. Mock treatments were transfected in absence of oligonucleotide. dsCon2 served as a non-specific control. SMN2FL and SMN2Δ7 splicing variants were quantified by RT-qPCR using isoform-specific primer sets. TBP was amplified as an internal reference. Shown are changes in SMN2FL and SMN2Δ7 expression levels relative to Mock treatment after normalization to TBP reference levels in GM03813 (FIG. 7A) and GM09677 (FIG. 7B) cells, respectively.

FIG. 8 shows the effect of DAO structure on the expression of SMN2 protein in GM03813 and GM09677 cells.

In particular, the figure confirms DAO (R6-04M1-27A-S1L1V3) activity on total SMN protein levels in patient-derived SMA fibroblasts. GM03813 and GM09677 cells were treated with 25 nM of the indicated oligonucleotides for 72 hours. R6-04(20)-S1V1v(CM-4) is an exemplary saRNA with optimized medicinal chemistry, while R6-04-S1 is its non-chemically-modified variant duplex. Mock treatments were transfected in absence of oligonucleotide. dsCon2 served as a non-specific control duplex. Whole cell protein extracts were harvested for immunoblot analysis. Total SMN protein levels were detected using an indiscriminate monoclonal antibody that recognized both SMN1 and SMN2 gene product in GM03813 (FIG. 8A) and GM09677 (FIG. 8C) cells. Immunodetection of α/β-Tubulin served as a protein loading control. Scanning optical densitometry was used to quantify protein band intensity from the above immunoblots. Shown in FIG. 8B and FIG. 8D are changes in total SMN protein levels relative to Mock treatment after normalization to α/β-Tubulin in GM03813 and GM09677 cells, respectively.

FIG. 9 shows the effect of different DAO structure on the expression of SMN2FL and SMN2Δ7 mRNA in GM03813 cells. Mock and dsCon2 are described as FIG. 1. mRNA levels of SMN2FL and SMN47 were determined by RT-qPCR using two pairs of primers in separate PCR reactions. The new DAO structures were used other saRNAs (DS06-0031 and DS06-0067) connected with ASO on different location.

In particular, the figure shows the impact of DAO structures on the expression of SMN2FL and SMN2Δ7 mRNA in GM03813 cells. GM03813 cells were treated with 20 nM of the indicated oligonucleotides for 72 hours. DS06-0031 and DS06-0067 are saRNA duplexes that preferentially enhance gene output of the SMN2Δ7 isoform. DAO constructs were synthesized by covalently linking ASO10-27 to the 3′-terminus of the sense (DS06-31A-27A and DS06-67A-27A) or antisense strand (DS06-31A-27B and DS06-67B-27B) in both duplexes. Mock treatments were transfected in absence of oligonucleotide. dsCon2 served as a non-specific control duplex, while DS06-332i served as a transfection control for monitoring SMN2 knockdown. SMN2FL and SMN2Δ7 splicing variants were quantified by RT-qPCR using isoform-specific primer sets. TBP was amplified as an internal reference. Shown are changes in SMN2FL and SMN2A7 expression levels relative to Mock treatment after normalization to TBP reference levels.

FIG. 10 shows the effect of DAO structure which using different linkers on the expression of SMN2FL and SMN2Δ7 mRNA in GM03813 cells. GM03813 cells were treated with the indicated concentration of saRNA, ASO and DAO with different linkers for 72 hours. Mock and dsCon2 are described as FIG. 1. mRNA levels of SMN2FL and SMNΔ7 were determined by RT-qPCR using two pairs of primers in separate PCR reactions.

In particular, the figure shows the effect of DAOs with different linkers on the expression of SMN2FL and SMN2Δ7 mRNA in GM03813 cells. GM03813 cells were treated with 25 nM of the indicated oligonucleotides including saRNA, ASO10-27 and DAOs with different linkers for 72 hours. Mock was transfected in the absence of an oligonucleotide. dsCon2 was transfected as an unrelated oligonucleotide control. mRNA levels of SMN2FL and SMNΔ7 were determined by RT-qPCR using two pairs of primers in separate PCR reactions (FIG. 10A) and by semi-quantitative RT-PCR followed by agarose gel electrophoresis (FIG. 10B). TBP gene was also amplified as a control for RNA loading. FIG. 10A shows the mRNA level of SMN2FL and SMNΔ7 determined by RT-qPCR. FIG. 10B shows SMN2FL and SMN2Δ7 levels derived from quantifying PCR product band intensity on the agarose gel.

FIG. 11 shows the effect of DAO structure which using different linkers on the expression of SMN2FL and SMN2Δ7 mRNA in GM00232 cells. GM00232 cells were treated with the indicated concentration of saRNA, ASO and DAO with different linkers for 72 hours. Mock and dsCon2 are described as FIG. 1 mRNA levels of SMN2FL and SMNΔ7 were determined by RT-qPCR using two pairs of primers in separate PCR reactions.

In particular, the figure shows the effect of DAOs with different linkers on the expression of SMN2FL and SMN2Δ7 mRNA in GM00232 cells. GM00232 cells were treated with 25 nM of the indicated oligonucleotides including saRNA, ASO10-27 and DAOs with different linkers for 72 hours. Mock was transfected in the absence of an oligonucleotide. dsCon2 was transfected as an unrelated oligonucleotide control. mRNA levels of SMN2FL and SMN2Δ7 were determined by RT-qPCR using two pairs of primers in separate PCR reactions (FIG. 11A) and by semi-quantitative RT-PCR followed by agarose gel electrophoresis (FIG. 11B). TBP gene was also amplified as a control for RNA loading. FIG. 11A shows the mRNA level of SMN2FL and SMNΔ7 determined by RT-qPCR. FIG. 11B shows SMN2FL and SMN2Δ7 levels derived from quantifying PCR product band intensity on the agarose gel.

FIG. 12 shows the effect of different DAO structure on the expression of SMN2FL and SMN2Δ7 mRNA in GM03813 and GM00232 cells. The indicated concentration of saRNA, ASO10-27 and DAO were treated for 72 hours in GM03813 and GM00232 cells respectively.

In particular, the figure shows the effects of “saRNA-saRNA” DAOs and 3-unit DAOs (i.e., bifunctional divalent saRNA with or without ASO conjugation (trifunctional DAO)) on the expression of SMN2FL and SMN2Δ7 mRNA in GM03813 and GM00232 cells. The indicated oligonucleotides were transfected at 25 nM for 72 hours into GM03813 and GM00232 cells. Mock was transfected in the absence of an oligonucleotide. dsCon2 was transfected as an unrelated oligonucleotide control. mRNA levels of SMN2FL and SMNΔ7 were determined by RT-qPCR using two pairs of primers in separate PCR reactions. FIG. 12A shows the mRNA level of SMN2FL and SMN2A7 determined by RT-qPCR in GM03813 cells. FIG. 12B shows the mRNA level of SMN2FL and SMN2Δ7 determined by RT-qPCR in GM00232 cells.

FIG. 13 shows the effect of different DAO structure on the expression of SMN protein in GM03813 and GM00232 cells. The indicated concentration of saRNA, ASO and DAO were treated for 72 hours in GM03813 and GM00232 cells respectively.

In particular, the figure shows the effects of “saRNA-saRNA” DAOs and 3-unit DAOs (i.e., bifunctional divalent saRNA with or without ASO conjugation (trifunctional DAO)) on the expression of SMN protein in GM03813 and GM00232 cells. The indicated oligonucleotides were transfected at 25 nM for 72 hours into GM03813 and GM00232 cells. Mock was transfected in the absence of an oligonucleotide. dsCon2 was transfected as an unrelated oligonucleotide control. Whole cell protein extracts were harvested for immunoblot analysis. Total SMN protein levels were detected using an indiscriminate monoclonal antibody that recognized both SMN1 and SMN2 gene product in GM03813 (FIG. 13A) and GM09677 (FIG. 13C) cells. Immunodetection of α/β-Tubulin served as a protein loading control. Scanning optical densitometry was used to quantify protein band intensity from the above immunoblots. Shown in FIG. 13B and FIG. 13D are changes in total SMN protein levels relative to Mock treatment after normalization to α/β-Tubulin in GM03813 and GM09677 cells, respectively.

FIG. 14 shows effects of DAOs with different bases number of ASO10-27 on the expression of SMN2FL and SMN2Δ7 mRNA in GM03813 and GM09677 cells. DAOs were transfected at 25 nM for 72 hours into GM03813 and GM09677 cells. Mock was transfected in the absence of an oligonucleotide. dsCon2 was transfected as an unrelated oligonucleotide control. R6-04M1-AC2(8˜18nt)-S1L1V3v was transfected as control corresponding to R6-04M1-(8˜18nt)-S1L1V3v. mRNA levels of SMN2FL and SMNΔ7 were determined by RT-qPCR using two pairs of primers in separate PCR reactions. TBP was also amplified as an internal reference. FIG. 14A shows the mRNA level of SMN2FL and SMN2A7 determined by RT-qPCR in GM03813 cells. FIG. 14B shows the mRNA level of SMN2FL and SMN2Δ7 determined by RT-qPCR in GM09677 cells.

In particular, the figure shows the effects of DAOs with varying sized SMN2 splice modulating ASO on the expression of SMN2FL and SMN2Δ7 mRNA in GM03813 and GM09677 cells. DAOs were transfected at 25 nM for 72 hours into GM03813 and GM09677 cells. Mock was transfected in the absence of an oligonucleotide. dsCon2 was transfected as an unrelated oligonucleotide control. ASO10-27 and series control DAOs (R6-04M1-AC2(8˜18nt)-S1L1V3v) were transfected as controls. mRNA levels of SMN2FL and SMNΔ7 were determined by RT-qPCR using two pairs of primers in separate PCR reactions. TBP was also amplified as an internal reference. FIG. 14A shows the mRNA level of SMN2FL and SMN2Δ7 determined by RT-qPCR in GM03813 cells. FIG. 14B shows the mRNA level of SMN2FL and SMN2Δ7 determined by RT-qPCR in GM09677 cells.

FIG. 15 shows the effect of DAOs with varying sized SMN2 splice modulating ASO on the expression of SMN protein in GM03813 and GM09677 cells. GM03813 and GM09677cells were treated with 25 nM of the indicated oligonucleotides for 72 hours. Mock was transfected in the absence of an oligonucleotide. dsCon2 was transfected as an unrelated oligonucleotide control. ASO10-27 was transfected as controls.

In particular, the figure shows the effect of DAOs with varying sized SMN2 splice modulating ASO on SMN protein levels in GM03813 and GM09677 cells. GM03813 and GM09677cells were treated with 25 nM of the indicated oligonucleotides for 72 hours. Mock was transfected in the absence of an oligonucleotide. dsCon2 was transfected as an unrelated oligonucleotide control. ASO10-27 was transfected as a positive control. Proteins were harvested from the treated cells and immunoblotted by western blotting assay using an antibody against human SMN protein. An antibody against α/β-Tubulin was also blotted to serve as a control for protein loading. FIGS. 15A & 15C shows the western blotting membrane with bands for SMN protein and α/β-Tubulin protein. FIGS. 15B & 15D shows relative fold changes of SMN protein levels derived from quantifying the band intensity of FIGS. 15A & 15C. Values (y-axis) in FIG. 15B and FIG. 15D are relative band intensity of SMN protein after being normalized to that of α/β-Tubulin.

FIG. 16 shows the effect of DAOs with varying sized SMN2 splice modulating ASO on the expression of SMN2FL and SMN2Δ7 mRNA in GM03813 and GM09677 cells. DAOs were transfected at 25 nM for 72 hours into GM03813 and GM09677 cells. Mock was transfected in the absence of an oligonucleotide. dsCon2 was transfected as an unrelated oligonucleotide control. ASO10-27 was transfected as control. mRNA levels of SMN2FL and SMNΔ7 were determined by RT-qPCR using two pairs of primers in separate PCR reactions. TBP was also amplified as an internal reference.

In particular, the figure shows the effect of DAOs with varying sized SMN2 splice modulating ASO on the expression of SMN2FL and SMN2A7 mRNA in GM03813 (FIG. 16A) and GM09677 (FIG. 16B) cells. DAOs were transfected at 25 nM for 72 hours into GM03813 and GM09677 cells. Mock was transfected in the absence of an oligonucleotide. dsCon2 was transfected as an unrelated oligonucleotide control. ASO10-27 was transfected as a positive control. mRNA levels of SMN2FL and SMNΔ7 were determined by RT-qPCR using two pairs of primers in separate PCR reactions. TBP was also amplified as an internal reference.

FIG. 17 shows the effect of DAOs with varying sized SMN2 splice modulating ASO on the expression of SMN protein in GM03813 and GM09677 cells. GM03813 and GM09677cells were treated with 25 nM of the indicated oligonucleotides for 72 hours. Mock was transfected in the absence of an oligonucleotide. dsCon2 was transfected as an unrelated oligonucleotide control. ASO10-27 was transfected as control. Proteins were harvested from the treated cells and immunoblotted by western blotting assay using an antibody against human SMN protein. An antibody against α/β-Tubulin was also blotted to serve as a control for protein loading.

In particular, the figure shows the effect of DAOs with varying sized SMN2 splice modulating ASO on SMN protein levels in GM03813 and GM09677 cells. GM03813 and GM09677 cells were treated with 25 nM of the indicated oligonucleotides for 72 hours. Mock was transfected in the absence of an oligonucleotide. dsCon2 was transfected as an unrelated oligonucleotide control. ASO10-27 was transfected as a positive control. Proteins were harvested from the treated cells and immunoblotted by western blotting assay using an antibody against human SMN protein. An antibody against α/β-Tubulin was also blotted to serve as a control for protein loading. FIGS. 17A & 17C shows the western blotting membrane with bands for SMN protein and α/β-Tubulin protein. FIGS. 17B & 17D shows relative fold changes of SMN protein levels derived from quantifying the band intensity of FIGS. 17A & 17C. Values (y-axis) FIG. 17B and FIG. 17D are relative band intensity of SMN protein after being normalized to that of α/β-Tubulin.

FIG. 18 shows the effect of DAOs with varying sized SMN2 splice modulating ASO on the expression of SMN2FL and SMN2Δ7 mRNA in PMH cells. DAOs were transfected at 25 nM for 72 hours into PMH cells. Mock was transfected in the absence of an oligonucleotide. dsCon2 was transfected as an unrelated oligonucleotide control. ASO10-27 was transfected as a positive control. mRNA levels of SMN2FL and SMNΔ7 were determined by RT-qPCR using two pairs of primers in separate PCR reactions. TBP was also amplified as an internal reference.

FIG. 19 shows the effects of “saRNA-siRNA” DAOs on the expression of SMN2FL, SMN2A7 and SOD1 mRNA in 293A (FIG. 19A) and GM03813 (FIG. 19B) cells. The indicated oligonucleotides were transfected at 25 nM for 72 hours into 293A or GM03813 cells. Mock was transfected in the absence of an oligonucleotide. dsCon2 was transfected as an unrelated oligonucleotide control. mRNA levels of SMN2FL, SMN47 and SOD1 were determined by RT-qPCR. TBP was also amplified as an internal reference.

FIG. 20 shows the effect of different DAO structures on the expression of p21 and PDL1 mRNA in PC3 (FIG. 20A) and Ku-7 (FIG. 20B) cells. The saRNA, siRNA and DAO treated with 10 nM for 48 hours in PC3 and Ku-7 cells respectively. Mock and dsCon2 are described as FIG. 1 mRNA levels of p21 and PDL1 were determined by RT-qPCR using two pairs of primers in separate PCR reactions.

In particular, the figure shows the effect of DAOs targeting two different genes on the expression of p21 and PD-L1 mRNA. PC3 and KU-7 cells were transfected with the indicated oligonucleotides at 10 nM for 48 hours. Mock was transfected in the absence of an oligonucleotide. dsCon2 was transfected as an unrelated oligonucleotide control. mRNA levels of p21 and PD-L1 were determined by RT-qPCR using gene specific primers in separate PCR reactions. GAPDH was also amplified as an internal reference. FIG. 20A shows the mRNA level of p21 and PD-L1 determined by RT-qPCR in PC3 cells. FIG. 20B shows the mRNA level of p21 and PD-L1determined by RT-qPCR in KU-7 cells.

FIG. 21 shows the effect of “ASO-ASO” DAOs on the expression of SMN2FL and SMN2Δ7 mRNA in GM03813 cells. The indicated oligonucleotides were transfected at 25 nM for 72 hours into GM03813 cells. Mock was transfected in the absence of an oligonucleotide. dsCon2 was transfected as an unrelated oligonucleotide control. mRNA levels of SMN2FL and SMNΔ7 were determined by RT-qPCR using two pairs of primers in separate PCR reactions. TBP was also amplified as an internal reference.

FIG. 22 shows the effect of “ASO-ASO” DAOs on the SMN protein in GM03813 cells. The indicated oligonucleotides were transfected at 25 nM for 72 hours into GM03813 cells. Mock was transfected in the absence of an oligonucleotide. dsCon2 was transfected as an unrelated oligonucleotide control. Proteins were harvested from the treated cells and immunoblotted by western blotting assay using an antibody against human SMN protein. In FIG. 22A, total SMN protein levels were detected using an indiscriminate monoclonal antibody that recognized both SMN1 and SMN2 gene product. Immunodetection of α/β-Tubulin served as a protein loading control. Scanning optical densitometry was used to quantify protein band intensity from the above immunoblot. Shown in FIG. 22B are changes in total SMN protein levels relative to Mock treatment after normalizing to α/β-Tubulin. Values (y-axis) is relative band intensity of SMN protein after being normalized to that of α/β-Tubulin.

FIG. 23 shows in vivo knockdown activity of “divalent” DAO siRNA on Htt mRNA expression in the brain and spinal cord of C57BL/6 pup mice (PND4). siRNAs were injected via ICV administration at 40 mg/kg dose. Saline was injected as a negative control. siHtt-S1V1 lacked DAO design and served as comparison for siHtt-S1L1 activity. Mice were sacrified 3 days (FIG. 23A) or 14 days FIG. 23B). Brain and spinal cord tissue samples were collected for analysis by RT-qPCR. Htt mRNA levels are mean values of two animals/group (n=2) relative to saline treatment after normalizing to Tbp reference levels.

FIG. 24 shows biodistribution and in vivo knockdown activity of siSOD1M2-S1L1V2v-Qu5 via SC injection in the organs of pup mice. FIG. 24A: Qu5-labeled “divalent” DAO siRNA (siSOD1M2-S1V1v-Qu5, siSOD1M2-S1L1V2v-Qu5) was administered via SC injection into C57BL/6 pup mice (PND4) at a 200 mg/kg dose. Injection of a Qu5-labeled siRNA variant siSOD1M2-S1V1v-Qu5, served as a comparative control. Mice were sacrificed 3 days following treatment and whole organ fluorescence was quantified on an IVIS Imaging System using 520 nm excitation and 570 nm emission filters. Shown are example IVIS images depicting siSOD1M2-S1L1V1v-Qu5 biodistribution via Qu5 signal in all major organs comparative to siSOD1M2-S1V1v-Qu5 following SC injection. FIG. 24B: Plotted is fluorescence signal intensity for each indicated organ within ROIs of identical size. FIG. 24C: Sod1 mRNA knockdown was quantified in the indicated organ tissue via RT-qPCR using gene specific primer sets. Tbp was amplified as an internal reference. Expression data is shown relative to mRNA levels in organs from a non-treated animal after normalizing to Tbp. The knockdown is relative to saline control.

FIG. 25 shows biodistribution and in vivo knockdown activity of siSOD1M2-S1L1V2v-Qu5 via ICV injection in the organs of pup mice. FIG. 25A: Qu5-labeled “divalent” DAO siRNA (siSOD1M2-S1V1v-Qu5, siSOD1M2-S1L1V2v-Qu5) was administered via ICV injection into C57BL/6 pup mice (PND4) at a 40 mg/kg dose. Injection of a Qu5-labeled siRNA variant siSOD1M2-S1V1v-Qu5, served as a comparative control. Mice were sacrificed 3 days following treatment and whole organ fluorescence was quantified on an IVIS Imaging System using 520 nm excitation and 570 nm emission filters. Shown are example IVIS images depicting siSOD1M2-S1L1V1v-Qu5 biodistribution via Qu5 signal in all major organs comparative to siSOD1M2-S1V1v-Qu5 following ICV injection. FIG. 25B: Plotted is fluorescence signal intensity for each indicated organ within ROIs of identical size. FIG. 25C: Sod1 mRNA knockdown was quantified in the indicated organ tissue via RT-qPCR using gene specific primer sets. Tbp was amplified as an internal reference. Expression data is shown relative to mRNA levels in organs from a non-treated animal after normalizing to Tbp. The knockdown is relative to saline control.

### 4. DETAILED DESCRIPTION OF THE INVENTION

Aspects of the present disclosure include covalently combining more than one molecule to provide improvements in efficient targeting one or more genes associated with a disease or condition, and improvements in the chemistry, manufacturing and controls (CMC) for gene therapy that could reduce manufacturing costs. The present inventors surprisingly found that covalently linking oligonucleotides targeting one or more sequences via the same or different mechanism of action into a single nucleotide molecule led to the creation of a novel class of multi-valent oligonucleotide (MVO) agents.

In some aspects, the present invention is based on investigations related to oligonucleotide agents, compositions and methods that activate/upregulate a gene expression and/or increase the amount of expression of full-length gene or protein in order to improve therapeutic effects for genetic conditions.

The present application further shows that combinatory treatment of SMA patient cells with an SMN2 saRNA and an SMN2 mRNA modulator, e.g., an ASO, such as Nusinersen, or a small pyridazine derivative including but not limited to Risdiplam and Branaplam, can achieve significantly higher levels of full-length SMN2 mRNA and SMN protein than the amount that can be achieved by either of the compounds used alone. This combination strategy for treatment can provide enhanced therapeutic benefit compared to monotherapy, for example, by improvements in the clinical symptoms of a patient diagnosed with an SMN-deficiency-related condition, or by reducing unwanted side effects in connection with monotherapy, and thus maximizing the treatment outcome of patients, such as SMA patients.

Unless otherwise defined, all the technological and scientific terms used therein have the same meanings as those generally understood by those of ordinary skill in the art covering the present invention.

In the present application, singular forms, such as “a” and “this”, include plural objects, unless otherwise specified clearly in the context.

As utilized herein, the terms “approximately,” “about,” “substantially”, and similar terms are intended to have a broad meaning in harmony with the common and accepted usage by those of ordinary skill in the art to which the subject matter of this disclosure pertains.

### 4.1. Definitions

As used herein, the term “multi-valent oligonucleotide agent”, “MVO agent” and “oligonucleotide agent with multiple functional oligonucleotide units” and the like are interchangeable and used in a broader sense to include any oligonucleotide agent of the invention comprising two or more functional oligonucleotides that are covalently linked. The MVO agent has improved functions or effects as compared to use any of the functional oligonucleotides alone or may even produce an additive effect or preferably synergistic effect. As used herein, the term “functional oligonucleotides”, also called “oligonucleotide units” or “functional oligonucleotide units”, refers to oligonucleotide units in the multi-valent oligonucleotide agent, which may be selected from single-stranded antisense oligonucleotides (ASOs, such as gapmers and mixmers) and duplex (double-stranded) RNAs (dsRNAs, such as siRNA and saRNA) and which are covalently linked to form an integrated molecule. In some aspects, the functional oligonucleotide units in the multi-valent oligonucleotide agent are of the same class or different class, are identical or different, have different targets or the same target (for example targeting the same gene or different genes), and/or connected directly or via linkers. The multi-valent oligonucleotide agents may be multi-targeting agents which have two or more targets for action and have improved activities.

As used herein, the term “gapmer” refers to a short DNA antisense oligonucleotide (ASO) structure with modified RNA segments on both sides of the central DNA structure. In some embodiments, at least one of the modified RNA segments comprises one or more of modified nucleotides selected from locked nucleic acids (LNA), and 2′—OMe or 2′-F modified nucleotides to increase affinity to the target, increase nuclease resistance, reduce immunogenicity, and/or decrease toxicity. In some embodiments, a gapmer comprises at least one nucleotide modified with a phosphorothioate (PS) group. In some embodiments, the gamper is designed to hybridize to a target piece of RNA and silence the gene transcript through the induction of RNase H cleavage. As an example, the ASO drug “Toferson” is a gapmer that knockdowns SOD1 mRNA for treatment of ALS. A possible example of a DAO with a gapmer ASO disclosed in the present application could be “siSOD1-Toferson”.

As used herein, the term “mixmer” refers to an antisense oligonucleotide (ASO) characterized as a mixture of DNA and chemically-modified nucleic acid analogs in structure. Optionally, a mixmer is composed of fully-modified nucleotides or nucleic acid analogs. In some embodiments, a mixmer is designed to bind and mask complementary RNA sequence to sterically block proteins, factors, or other RNAs from interacting with targeted RNA. In some embodiments, a mixmers is designed to alter pre-mRNA splicing by displacing the spliceosome. In some embodiments, a mixmer is deisgned to bind and sequester microRNAs (miRNAs) in which it is adopt yet another name called an “antagomir” or an “anti-miR”. In some embodiments, DAO examples combining saRNA and mixmers are described in the present application.

The terms “gapmer” and “mixmer” may used to exemplify two different sub-classes of single-stranded antisense oligonucleotide (ASO) molecules. In some embodiments, ASOs are either a “gapmer” or a “mixmer”, which performs different molecular functions. In some embodiments, the terms “gapmer” and “mixmer” refer to their chemical design.

Detailed descriptions about gapmers and mixmers can be found in such as Peter H. Hagedorn et al., Locked nucleic acid: modality, diversity, and drug discovery, Drug Discovery Today, Volume 23, Number 1, January 2018; Piotr J. Kamola et al. In silico and in vitro evaluation of exonic and intronic off-target effects form a critical element of therapeutic ASO gapmer optimization, Nucleic Acids Research, Volume 43, Issue 18, 15 Oct. 2015, Pages 8638-8650; Birte Vester et al., LNA (locked nucleic acid): high-affinity targeting of complementary RNA and DNA, Biochemistry 2004, 43, 42, 13233-13241.

As used herein, the term “SMN-deficiency-related conditions” refers to a disease caused by deficiency in SMN full-length protein due to any cause. “SMN-deficiency-related conditions” include, but are not limited to, spinal muscular atrophy (SMA), neurogenic-type arthrogryposis multiplex congenital (congenital AMC), and amyotrophic lateral sclerosis (ALS). For SMN1 (human), the GenBank gene reference is Gene ID: 6606.

The terms “spinal muscular atrophy” or “SMA” include, but are not limited to, spinal muscular atrophy (SMA) types 1 through 4; proximal spinal muscular atrophy; childhood-onset SMA Type I (Werdnig-Hoffmann disease); Type II (intermediate, chronic form), Type III (Kugelberg-Welander disease, or Juvenile Spinal Muscular Atrophy), and a relatively recently categorized adult-onset form Type IV. Meeting report: International SMA Consortium meeting. Neuromuscul Disord.; 2: 423-428. The term SMA also includes late-onset SMA (also known as SMA types 3 and 4, mild SMA, adult-onset SMA and Kugelberg-Welander disease). The term SMA also includes other forms of SMA, including X-linked disease, spinal muscular atrophy with respiratory distress (SMARD), spinal and bulbar muscular atrophy (Kennedy's disease, or Bulbo-Spinal Muscular Atrophy), and distal spinal muscular atrophy. The term SMA includes all forms of SMA described in Arnold, W. D., Kassar, D. & Kissel, J. T. Spinal muscular atrophy: Diagnosis and management in a new therapeutic era. Muscle and Nerve (2015); Butchbach, M. E. R. Copy Number Variations in the Survival Motor Neuron Genes: Implications for Spinal Muscular Atrophy and Other Neurodegenerative Diseases. Front. Mol. Biosci. (2016).

When SMA symptoms are present at birth or by the age of 6 months, the disease is called SMA type 1 (also called infantile onset or Werdnig-Hoffmann disease). Babies typically have generalized muscle weakness, a weak cry, and breathing distress. They often have difficulty swallowing and sucking, and don't reach the developmental milestone of being able to sit up unassisted. These babies have increased risk of aspiration and failure to thrive. Typically, these babies have two or three copies of the SMN2 gene. (Butchbach, M. E. R. Copy Number Variations in the Survival Motor Neuron Genes: Implications for Spinal Muscular Atrophy and Other Neurodegenerative Diseases. Front. Mol. Biosci. (2016) which is incorporated herein in its entirety)

When SMA has its onset between the ages of 3 and 15 months and before the child can stand or walk independently, it is called SMA type 2, or intermediate SMA or Dubowitz disease. Children with SMA type 2 generally have three copies of the SMN2 gene (Arnold, W. D., Kassar, D. & Kissel, J. T. Spinal muscular atrophy: Diagnosis and management in a new therapeutic era. Muscle and Nerve (2015) which is incorporated herein in its entirety). Muscle weakness is predominantly proximal (close to the center of the body) and involves the lower limbs more than the upper limbs. Usually, the face and the eye muscles are unaffected. (Butchbach, M. E. R. Copy Number Variations in the Survival Motor Neuron Genes: Implications for Spinal Muscular Atrophy and Other Neurodegenerative Diseases. Front. Mol. Biosci. (2016) which is incorporated herein in its entirety).

Late-onset SMA (also known as SMA types 3 and 4, mild SMA, adult-onset SMA and Kugelberg-Welander disease) results in variable levels of weakness. Patients with type 3 SMA have 3 to 4 copies of the SMN2 gene. SMA type 3 (juvenile onset) accounts for 30% of overall SMA cases (Arnold, W. D., Kassar, D. & Kissel, J. T. Spinal muscular atrophy: Diagnosis and management in a new therapeutic era. Muscle and Nerve (2015)). Symptoms usually appear between age 18 months and adulthood. Affected individuals achieve independent mobility. However, proximal weakness in these patients might cause falls and difficulty with climbing stairs. Over time, many lose their ability to stand and walk, so instead use a wheelchair to move around. Most of these patients develop foot deformities, scoliosis, and respiratory muscle weakness.

SMA type 4 is late-onset and accounts for less than 5% of overall SMA cases. These patients have four to eight copies of the SMN2 gene (Butchbach, M. E. R. Copy Number Variations in the Survival Motor Neuron Genes: Implications for Spinal Muscular Atrophy and Other Neurodegenerative Diseases. Front. Mol. Biosci. (2016)). Age of onset is not defined but is usually after age 30. Type 4 is a mild form of SMA and therefore lifespan remains normal. Patients can achieve motor milestones and maintain their mobility throughout life.

As used herein, the terms “subject” and “individual” are used interchangeably herein to mean any living organism that may be treated with compounds of the present disclosure. The term “patient” means a human subject or individual, including disclosure infants, children and adults.

A “therapeutically effective amount” of a composition is an amount sufficient to achieve a desired therapeutic effect, and therefore does not require cure or complete remission. In embodiments of the present disclosure, therapeutic efficacy is an improvement in any of the disease indicators, and a therapeutically effective amount is sufficient to cause an improvement in a clinically significant condition/symptom in the treated individual. The phrases “therapeutically effective amount” and “effective amount” are used herein to mean an amount sufficient to reduce by at least about 15 percent, preferably by at least 50 percent, more preferably by at least 90 percent, and most preferably prevent, a clinically significant deficit in the activity, function and response of the individual being treated.

The effective amount can vary depending on such factors as the size and weight of the subject, the type of illness, or the particular compounds of the invention. For example, the choice of the compound of the invention can affect what constitutes an “effective amount.” One of ordinary skill in the art would be able to study the factors contained herein and make the determination regarding the effective amount of the compounds of the invention without undue experimentation.

The regimen of administration can affect what constitutes an effective amount. The compound of the invention can be administered to the subject either prior to or after the disease diagnosis or condition. Further, several divided dosages, as well as staggered dosages, can be administered daily or sequentially, or the dose can be continuously infused, or can be a bolus injection. Further, the dosages of the compound(s) of the invention can be proportionally increased or decreased as indicated by the exigencies of the therapeutic or prophylactic situation.

The terms “treat,” “treated,” “treating”, or “treatment” as used herein have the meanings commonly understood in the medical arts, and therefore do not require cure or complete remission, and include any beneficial or desired clinical results. Nonlimiting examples of such beneficial or desired clinical results are prolonging survival as compared to expected survival without treatment, reduced symptoms including one or more of the followings: weakness and atrophy of proximal skeletal muscles, inability to sit or walk independently, difficulties in swallowing, breathing, etc.

As used herein, “preventing” or “delaying” a disease refers to inhibiting the full development of a disease.

The term “biological sample” refers to any tissue, cell, fluid, or other material derived from an organism (e.g., human subject). In certain embodiments, the biological sample is serum or blood.

As used herein, the terms “sequence identity” or “% identity” in the context of oligonucleotide sequence refers to the percentage of residues in the compared sequences that are the same when the sequences are aligned over a specified comparison window. For example, in the context of saRNA, the term “sequence identity” or “sequence homology” means that one oligonucleotide strand (sense or antisense) of an saRNA has at least 80% similarity with a region on the coding strand or template strand of the promoter sequence of a target gene. In some aspects, the percent identity is measured using one of the sequence comparison algorithms (e.g., BLASTP and BLASTN or other algorithms available to persons of skill) or by visual inspection.

“Target gene promoter sequence” refers to a non-coding sequence of a target gene, and in the context of the present disclosure “complementary to the promoter sequence of the target gene” refers to the coding strand of the sequence, also referred to as the non-template strand, i.e., a nucleic acid sequence that is the same sequence as the coding sequence of the gene.

As used herein, the terms “sense strand” and “sense oligonucleotide strand” are interchangeable. The sense oligonucleotide strand of dsRNA molecule can include, for example, a first nucleic acid strand comprising a coding strand of a promoter sequence of a target gene in a duplex of saRNA.

As used herein, the terms “antisense strand” and “antisense oligonucleotide strand” are interchangeable. The antisense oligonucleotide strand of a dsRNA molecule can include, for example, to a second nucleic acid strand in a duplex of saRNA that is complementary to the sense oligonucleotide strand.

As used herein, the term “first oligonucleotide strand” can be a sense strand or an antisense strand. The sense strand of a saRNA refers to an oligonucleotide strand having homology with the coding strand of the promoter DNA sequence of the target gene in the saRNA duplex. The antisense strand refers to an oligonucleotide strand complementary with the sense strand in the saRNA duplex.

As used herein, the term “second oligonucleotide strand” can also be a sense strand or an antisense strand. If the first oligonucleotide strand is a sense strand, the second oligonucleotide strand is an antisense strand; and if the first oligonucleotide strand is an antisense strand, the second oligonucleotide strand is a sense strand.

The term “promoter” as used herein refers to a nucleic acid sequence, which encodes no proteins and plays a regulatory role for the transcription of a protein-coding or RNA-coding nucleic acid sequence by associating with them spatially. Generally, a eukaryotic promoter contains 100 to 5,000 base pairs, although this length range is not intended to limit the term of “promoter” as used herein. Although the promoter sequence is generally located at the 5′ terminus of a protein-coding or RNA-coding sequence, it also exists in exon and intron sequences.

As used herein, the term “coding strand” refers to the DNA strand in the target gene that cannot be transcribed, the nucleotide sequence of which is identical to the sequence of the RNA produced by transcription (in RNA the T in DNA is replaced by U). The coding strand of the double-stranded DNA sequence of the target gene promoter described in the present disclosure refers to the promoter sequence on the same DNA strand as the DNA coding strand of the target gene.

As used herein, the term “template strand” refers to another strand of double-stranded DNA of a target gene that is complementary to the coding strand and that can be transcribed as a template into RNA that is complementary to the transcribed RNA base (A-U, G-C). During transcription, RNA polymerase binds to the template strand and moves along the 3 ‘+5’ direction of the template strand, catalyzing RNA synthesis in the 5′->3′ direction. The template strand of the double-stranded DNA sequence of the target gene promoter described in the present disclosure refers to the promoter sequence on the same DNA strand as the DNA template strand of the target gene.

As used herein, the term “transcription start site” or TSS refers to a nucleotide that marks the initiation of transcription on the template strand of a gene. The transcription start site may be present on the template strand of the promoter region. A gene may have more than one transcription start site.

As used herein, the term “overhang” refers to an oligonucleotide strand end (5′ or 3′) with non-base paired nucleotide(s) resulting from another strand extending beyond one of the strands within the double stranded oligonucleotide. Single stranded regions extending beyond the 3′and/or 5′ ends of the duplexes are referred to as overhangs. In certain embodiments, the overhang is from 0 to 6 nucleotides in length. It is understood that an overhang of 0 nucleotides means that there is no overhang.

As used herein, the terms “gene activation”, “activating gene expression”, “gene upregulation” and “upregulating gene expression” can be used interchangeably, and means an increase or upregulation in transcription, translation, expression or activity of a certain nucleic acid sequence as determined by measuring the transcription level, mRNA level, protein level, enzymatic activity, methylation state, chromatin state or configuration, translation level or the activity or state in a cell or biological system of a gene. These activities or states can be determined directly or indirectly. In addition, “gene activation” or “activating gene expression” refers to an increase in activity associated with a nucleic acid sequence, regardless the mechanism of such activation. For example, gene activation occurs at the transcriptional level to increase transcription into RNA and the RNA is translated into a protein, thereby increasing the expression of the protein.

As used herein, the terms “small activating RNA”, “saRNA” and “small activating ribonucleic acid” can be used interchangeably and refer to a ribonucleic acid molecule that can upregulate target gene expression. It can be a double-stranded nucleic acid molecule composed of a first nucleic acid strand containing a ribonucleotide sequence with sequence homology with the non-coding nucleic acid sequence (such as a promoter and an enhancer) of a target gene and a second nucleic acid strand containing a nucleotide sequence complementary with the first strand. The saRNA can also be comprised of a synthesized or vector-expressed single-stranded RNA molecule that can form a hairpin structure by two complementary regions within the molecule, wherein the first region contains a ribonucleotide sequence having sequence homology with the target sequence of a promoter of a gene, and a ribonucleotide sequence contained in the second region is complementary with the first region. The length of the duplex region of the saRNA molecule is typically about 10 to about 60, about 10 to about 50, about 12 to about 48, about 14 to about 46, about 16 to about 44, about 18 to about 42, about 20 to about 40, about 22 to about 38, about 24 to about 36, about 26 to about 34, and about 28 to about 32 base pairs, and typically about 10, about 15, about 20, about 25, about 30, about 35, about 40, about 45, about 50, about 55, or about 60 base pairs. In addition, the terms “small activating RNA”, “saRNA” and “small activating ribonucleic acid” also contain nucleic acids other than the ribonucleotide, including, but not limited to, modified nucleotides or analogues.

As used herein, the terms “small interfering RNA”, “siRNA” and “small interfering ribonucleic acid” can be used interchangeably and refer to a ribonucleic acid molecule that can downregulate or even silent target gene expression. It can be a double-stranded nucleic acid molecule composed of a first nucleic acid strand containing a ribonucleotide sequence with sequence homology with the non-coding nucleic acid sequence of a target gene and a second nucleic acid strand containing a nucleotide sequence complementary with the first strand. The siRNA can also be comprised of a synthesized or vector-expressed single-stranded RNA molecule that can form a hairpin structure by two complementary regions within the molecule, wherein the first region contains a ribonucleotide sequence having sequence homology with the target sequence of a promoter of a gene, and a ribonucleotide sequence contained in the second region is complementary with the first region. The length of the duplex region of the siRNA molecule is typically about 10 to about 60, about 10 to about 50, about 12 to about 48, about 14 to about 46, about 16 to about 44, about 18 to about 42, about 20 to about 40, about 20 to about 25 base pairs, and typically about 10, about 15, about 16, about 17, about 18, about 19, about 20, about 21, about 22, about 23, about 24, about 25, about 26, about 27, about 28, about 29, about 30, about 35, about 40, about 45, about 50 base pairs. In addition, the terms also contain nucleic acids other than the ribonucleotide, including, but not limited to, modified nucleotides or analogues.

As used herein, “covalent linker” refers to a molecule for covalently joining two molecules, e.g., two dsRNAs. As described in more detail below, the term can include, e.g., a nucleic acid linker, a peptide linker, and the like and includes disulfide linkers.

As used herein, the term “synthetic” refers to the manner in which oligonucleotides are synthesized, including any means capable of synthesizing or chemically modifying RNA, such as chemical synthesis, in vitro transcription, vector expression, and the like.

### 4.2. Structures and Constructions of Multi-Valent Oligonucleotide (MVO) Agents

4.2.1 Functional Oligonucleotides in the multi-valent oligonucleotide agent

Aspects of the present disclosure include a multi-valent oligonucleotide agent that includes two or more functional oligonucleotides that are covalently linked. In some embodiments, the two or more functional oligonucleotides are independently selected from a double stranded RNA (dsRNA) and an antisense oligonucleotide (ASO). The dsRNA are independently selected from a small interfering RNA (siRNA) and a small activating RNA (saRNA). The ASOs are independently selected from a gapmer and a mixmer.

In some embodiments, the multi-valent oligonucleotide agent comprises two or more, three or more, four or more, four or more, five or more, six or more, or seven or more oligonucleotide units. In some embodiments, the multi-valent oligonucleotide agent comprises 2˜10 functional oligonucleotides. In some embodiments, the multi-valent oligonucleotide agent is a dual-action oligonucleotide (DAO) or even multi-action oligonucleotide agent.

In some embodiments, the MVO agent may comprise: a) a first double stranded RNA (dsRNA) and a first antisense oligonucleotide (ASO); b) a first double stranded RNA (dsRNA) and a second dsRNA; c) a first antisense oligonucleotide (ASO) and a second ASO; d) a first double stranded RNA (dsRNA), a second dsRNA, and a third dsRNA; e) a first double stranded RNA (dsRNA), a second dsRNA, and a first antisense oligonucleotide (ASO); f) a first double stranded RNA (dsRNA), a first antisense oligonucleotide (ASO) and a second ASO; or g) a first antisense oligonucleotide (ASO), a second ASO, and a third ASO; and in any one of a)˜ g), the functional oligonucleotides are arranged in any order and covalently linked with or without linking component(s).

In some embodiments, the dsRNA is a small interfering RNA (siRNA). siRNA binds to target mRNA mainly in the cytoplasm to down-regulate gene expression post-transcriptionally via the RNA interference (RNAi) mechanism. siRNAs may be designed to target a gene's mRNA sequence to silence its expression via the RNAi mechanism, such as PDL-1, for maximizing treatment outcomes, e.g., for cancer patients.

siRNAs can be molecules having endogenous RNA bases or chemically modified nucleotides. The modifications do not abolish cellular activity, but rather impart increased stability and/or increased cellular potency. Examples of chemical modifications include phosphorothioate groups, 2′-deoxynucleotide, 2′—OCH.sub.3-containing ribonucleotides, 2′-F-ribonucleotides, 2′-methoxyethyl ribonucleotides, combinations thereof and the like. The siRNA can have varying lengths (e.g., 10-200 bps) and structures (e.g., hairpins, single/double strands, bulges, nicks/gaps, mismatches) and are processed in cells to provide active gene silencing. A double-stranded siRNA (dsRNA) can have the same number of nucleotides on each strand (blunt ends) or asymmetric ends (overhangs). An overhang of 1-2 nucleotides, for example, can be present on the sense and/or the antisense strand, as well as present on the S′- and/or the 3′-ends of a given strand.

In some embodiments, the dsRNA is a small activating RNA (saRNA). saRNA targets regulatory sequences in the nucleus such as gene promoters to upregulate gene expression at the transcriptional level via the RNAa (RNA activation) mechanism.

In some embodiments, at least one oligonucleotide is an ASO. An ASO can be designed to target a gene's mRNA to downregulate its expression via the RNase H activity, e.g., for maximizing treatment efficiency of cancers.

In some embodiments, at least one oligonucleotide is an ASO. An ASO can be designed to target a gene's pre-mRNA to alter its splicing via steric blocking, e.g., for maximizing the gene's functional protein expression.

In some embodiments, each of the two or more functional oligonucleotides modulates the expression of one or more genes, proteins by binding to a mRNA sequence, or noncoding regulatory nucleic acid sequences. In certain embodiments, the target noncoding regulatory nucleic acid sequence is a promoter sequence.

In some embodiments, the multi-valent oligonucleotide agent comprises a dsRNA comprising a sense strand and an antisense strand. In certain embodiments, the antisense strand of the dsRNA has partial complementarity with the sense dsRNA strand. As used herein, the term “partial complementarity” can include an antisense strand of the dsRNA that has complementarity to 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, or 60 or more contiguous nucleotides of the sense strand of the dsRNA. In certain embodiments, the dsRNA comprises an antisense strand that has at least 15 contiguous nucleotides that has complementarity to at least 15 contiguous nucleotides of a sense strand of the dsRNA. In certain embodiments, the dsRNA comprises an antisense strand that has at least 20, at least 25, at least 30, at least 35, at least 40, at least 45, at least 50, at least 55, or at least 60 contiguous nucleotides that has complementarity to at least at least 20, at least 25, at least 30, at least 35, at least 40, at least 45, at least 50, at least 55, or at least 60 contiguous nucleotides of a sense strand of the dsRNA.

In some embodiments, the multi-valent oligonucleotide agent may comprises functional oligonucleotides selected from: a) siRNA-siRNA; b) siRNA-saRNA; c) saRNA-saRNA; d) siRNA-gapmer; e) siRNA-mixmer; f) saRNA-gapmer; g) saRNA-mixmer; h) gapmer-gapmer; i) gapmer-mixmer; j) mixmer-mixmer; k) siRNA-siRNA-siRNA; 1) siRNA-siRNA-saRNA; m) siRNA-saRNA-saRNA; n) saRNA-saRNA-saRNA; o) siRNA-siRNA-gapmer; p) siRNA-siRNA-mixmer; q) siRNA-saRNA-gapmer; r) siRNA-saRNA-mixmer; s) saRNA-saRNA-gapmer; t) saRNA-saRNA-mixmer; u) siRNA-gapmer-gapmer; v) saRNA-gapmer-gapmer; w) siRNA-gapmer-mixmer; x) saRNA-gapmer-mixmer; y) siRNA-mixmer-mixmer; z) saRNA-mixmer-mixmer; aa) gapmer-gapmer-gapmer; ab) gapmer-gapmer-mixmer; ac) gapmer-mixmer-mixmer; and, ad) mixmer-mixmer-mixmer; and wherein in any one of a)˜ ad), the functional oligonucleotides are arranged in any order and covalently linked with or without linking component(s).

In some embodiments, the three or more oligonucleotides comprise three dsRNAs (e.g. a first dsRNA, a second dsRNA, and a third dsRNA). In some embodiments, the three or more oligonucleotides comprise two dsRNA and an ASO (e.g., a first dsRNA, a second dsRNA, and an ASO).

In some embodiments, the dsRNA comprises a sense strand that is at least 15 contiguous nucleotides and an antisense strand that is at least 15 contiguous nucleotides.

In some embodiments, the sense strand has a length ranging from about 10 nucleotides or more, about 15 nucleotides or more, about 20 nucleotides or more, about 25 nucleotides or more, about 30 nucleotides or more, about 35 nucleotides or more, about 40 nucleotides or more, about 45 nucleotides or more, about 50 nucleotides or more, about 55 nucleotides or more, or about 60 nucleotides or more). In some embodiments, the sense strand is 10-100 nucleotides in length (e.g., 10-20 nucleotides, 10-50 nucleotides, 10-90 nucleotides, 20-95 nucleotides, 30-70 nucleotides, 40-80 nucleotides, 50-100 nucleotides, 10-40 nucleotides, 10-30 nucleotides). In some embodiments, the sense strand is 10-60 nucleotides in length (e.g., 10-20 nucleotides, 10-50 nucleotides, 10-40 nucleotides, 10-30 nucleotides).

In some embodiments, the sense strand has a length ranging from about 10 nucleotides or more, about 15 nucleotides or more, about 20 nucleotides or more, about 25 nucleotides or more, about 30 nucleotides or more, about 35 nucleotides or more, about 40 nucleotides or more, about 45 nucleotides or more, about 50 nucleotides or more, about 55 nucleotides or more, about 60 nucleotides or more, about 65 nucleotides or more, about 70 nucleotides or more, about 75 nucleotides or more, about 80 nucleotides or more, about 85 nucleotides or more, about 90 nucleotides or more, about 95 nucleotides or more, or about 100 nucleotides or more). In some embodiments, the sense strand is 10-60 nucleotides in length (e.g., 10-20 nucleotides, 10-50 nucleotides, 10-40 nucleotides, 10-30 nucleotides).

In some embodiments, the antisense strand has a length ranging from about 10 nucleotides or more, about 15 nucleotides or more, about 20 nucleotides or more, about 25 nucleotides or more, about 30 nucleotides or more, about 35 nucleotides or more, about 40 nucleotides or more, about 45 nucleotides or more, about 50 nucleotides or more, about 55 nucleotides or more, or about 60 nucleotides or more). In some embodiments, the antisense strand is 10-100 nucleotides in length (e.g., 10-20 nucleotides, 10-50 nucleotides, 10-90 nucleotides, 20-95 nucleotides, 30-70 nucleotides, 40-80 nucleotides, 50-100 nucleotides, 10-40 nucleotides, 10-30 nucleotides). In some embodiments, the antisense strand is 10-60 nucleotides in length (e.g., 10-20 nucleotides, 10-50 nucleotides, 10-40 nucleotides, 10-30 nucleotides).

In some embodiments, the antisense strand has a length ranging from about 10 nucleotides or more, about 15 nucleotides or more, about 20 nucleotides or more, about 25 nucleotides or more, about 30 nucleotides or more, about 35 nucleotides or more, about 40 nucleotides or more, about 45 nucleotides or more, about 50 nucleotides or more, about 55 nucleotides or more, about 60 nucleotides or more, about 65 nucleotides or more, about 70 nucleotides or more, about 75 nucleotides or more, about 80 nucleotides or more, about 85 nucleotides or more, about 90 nucleotides or more, about 95 nucleotides or more, or about 100 nucleotides or more). In some embodiments, the antisense strand is 10-60 nucleotides in length (e.g., 10-20 nucleotides, 10-50 nucleotides, 10-40 nucleotides, 10-30 nucleotides).

In some embodiments, the two or more functional oligonucleotides that are covalently linked have a total nucleotide length ranging from 10 nucleotides to 500 nucleotides (e.g., 10 nucleotides to 100 nucleotides, 50 nucleotides to 100 nucleotides, 50 nucleotides to 200 nucleotides, 20 nucleotides to 100 nucleotides, 20 nucleotides to 200 nucleotides, 20 nucleotides to 300 nucleotides, 50 nucleotides to 300 nucleotides, 20 nucleotides to 80 nucleotides, 100 nucleotides to 300 nucleotides, 300 nucleotides to 500 nucleotides).

4.2.2 Nucleotide modifications

All nucleotides of the oligonucleotides described herein may be natural, i.e., non-chemically modified nucleotides or at least one nucleotide may be chemically modified. Non-limiting examples of the chemical modification can include one or a combination of the following:
(1) Modifications to phosphodiester linkages of nucleotides in the nucleotide sequence of a functional oligonucleotide (such as in the SMN2 saRNA);
(2) Modification of the 2 ‘—OH of ribose in the nucleotide sequence of a functional oligonucleotide;
(3) Modifications to bases in the nucleotide sequence of a functional oligonucleotide.

Chemical modifications of nucleotides or oligonucleotides in the present disclosure are well known to those skilled in the art, and modifications of the phosphodiester bond refer to modifications of oxygen in the phosphodiester bond, including phosphorothioate modifications and boronated phosphate modifications. Both modifications stabilize the olignucleotide structure, maintaining high specificity and high affinity for base pairing.

The ribose modification refers to a modification of the 2’—OH in a nucleotide pentose, i.e., introduction of certain substituents at the hydroxyl position of the ribose, e. g., 2′-fluoro modification, 2 ‘-oxomethyl modification, 2’-oxyethylenemethoxy modification, 2,4 ‘-dinitrophenol modification, locked nucleic acid (LNA), 2’-amino modification, 2 ‘-deoxy modification.

By base modification, it is meant a modification of the base of the nucleotide, e.g., 5’-bromouracil modification, 5′-iodouracil modification, N-methyluracil modification, 2,6-diaminopurine modification.

These modifications may increase the bioavailability of the oligonucleotides, increase affinity for the target sequence, and enhance resistance to nuclease hydrolysis in a cell.

In addition, to facilitate entry of the oligonucleotides into a cell, lipophilic groups such as cholesterol may be introduced at the ends of the sense or antisense strands of the oligonucleotides on the basis of the above modifications to facilitate action through a cell membrane composed of lipid bilayers and gene promoter regions within the nuclear membrane and nucleus.

The oligonucleotide agent of the present disclosure which, upon contact with a cell, are effective in activating or up-regulating the expression of one or more genes in the cell, preferably by at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%).

One aspect of the disclosure provides a cell comprising two or more functional oligonucleotides of the present disclosure or a nucleic acid encoding the at least two oligonucleotides of the present disclosure. In one embodiment, the cell is a mammalian cell, preferably a human cell. Such cells may be ex vivo, such as cell lines and the like, or may be present in mammalian bodies, such as humans, including infants, children or adults.

In some embodiments, at least one oligonucleotide in the at least two oligonucleotides of the multi-valent oligonucleotide agent can include at least one modified nucleotide, e.g., a 2′-O-methyl modified nucleotide, a nucleotide comprising a 5′-phosphorothioate group, a terminal nucleotide linked to a cholesteryl derivative or dodecanoic acid bisdecylamide group, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural base comprising nucleotide. In some embodiments, the first and second dsRNAs include “endo-light” modification with 2′-O-methyl modified nucleotides and nucleotides comprising a 5′-phosphorothioate group.

In some embodiments, the chemical modification of the at least one chemically modified nucleotide is an addition of a (E)-vinylphosphonate moiety at the 5′ end of the nucleotide sequence. In some embodiments, the chemical modification of the at least one chemically modified nucleotide is an addition of a 5-methyl cytosine moiety at the 5′ end of the nucleotide sequence.

In some embodiments, at least one of the oligonucleotides of the multi-valent agent is chemically modified to enhance stability or other beneficial characteristics. The nucleic acids featured in the present disclosure may be synthesized and/or modified by conventional methods, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds that can be used in this present disclosure include, but are not limited to RNAs containing modified backbones or no natural internucleoside linkages. In some embodiments, RNAs having modified backbones include, among others, those that do not have a phosphorus atom in the backbone. In some embodiments, modified RNAs that do not have a phosphorus atom in their internucleoside backbone can also be considered to be oligonucleosides. In some embodiments, the modified oligonucleotide will have a phosphorus atom in its internucleoside backbone.

Modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Non-limiting examples of preparation of the phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, which are hereby incorporated by reference in their entireties.

### 4.2.3 Covalent Linkages

Aspects of the present disclosure include a multi-valent oligonucleotide agent comprising two or more functional oligonucleotides that are covalently linked by a linking component or by a phosphodiester bond or by one or more nucleotides.

In some embodiments, the functional oligonucleotide units in the MVO agents are linked with a covalent linker. In some embodiments, the linker is a disulfide linker. Various combinations of strands can be linked, e.g., the first and second dsRNA sense strands are covalently linked or, e.g., the first and second dsRNA antisense strands are covalently linked. In some embodiments, any of the multi-valent oligonucleotide agents of the disclosure include a ligand.

In some embodiments, the linking component can include, but is not limited to:
Spacer phosphoramidite 18 (Phosphoramidous acid, N,N-bis(1-methylethyl)-, 19,19-bis(4-methoxyphenyl)-19-phenyl-3,6,9,12,15,18-hexaoxanonadec-1-yl 2-cyanoethyl ester);
Spacer-9 (3-[2-[2-[2-[bis(4-methoxyphenyl)-phenylmethoxy]ethoxy]ethoxy]ethoxy-[di(propan-2-yl)amino]phosphanyl]oxypropanenitrile); Spacer phosphoramidite C3 (6-(4,4′-Dimethoxytrityl)hexyl-1-[(2-cyanoethyl)-(N,N-diisopropyl)]-phosphoramidite);
Spacer-C6 Phosphoramidite (6-(4,4′-Dimethoxytrityl)hexyl-1-[(2-cyanoethyl)-(N,N-diisopropyl)]-phosphoramidite).

In some embodiments, the linking component comprises a compound structure shown in Table 1.

TABLE 1
Linkers used in MVO agents

| Name | Formula | Structure |
|---|---|---|
| Spacer-9 | C₃₆H₄₉N₂O₇P | 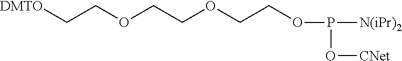 |
| Spacer-18 | C₄₂H₆₁N₂O₁₀P | 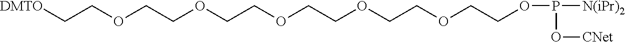 |
| Spacer-C3 | C₃₃H₄₃N₂O₅P | 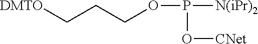 |
| Spacer-C6 | C₃₆H₄₉N₂O₅P | 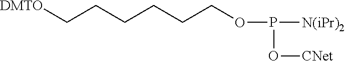 |
| Nucleotide | | 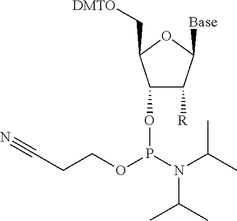 |

*n.b.: R = —H or —OH or —OMe, or —MOE, or —F, or other 2′ chemical modifications.

In some embodiments, the two or more functional oligonucleotides are covalently linked by a phosphodiester bond. In some embodiments, the two or more functional oligonucleotides are covalently linked by a phosphorothioate bond.

In some embodiments, the two or more functional oligonucleotides are covalently linked by one or more nucleotides.

Non-limiting examples of covalent linkers can be found in U.S. Patent Application Publication No.: 20200332292, which is hereby incorporated by reference in its entirety. The covalent linker can join two or more functional oligonucleotides. In some embodiments, the covalent linker can join two sense strands, two antisense strands, one sense and one antisense strand, two sense strands and one antisense strand, two antisense strands and one sense strand, two sense and two antisense strands, an antisense strand and ASO, a sense strand and ASO, and the like.

In certain embodiments, the covalent linker can include RNA and/or DNA and/or a peptide. The linker can be single stranded, double stranded, partially single strands, or partially double stranded. In some embodiments the linker includes a disulfide bond. The linker can be cleavable or non-cleavable.

In certain embodiments, the covalent linker can include, e.g., dTsdTuu=(5′-2′deoxythymidyl-3′-thiophosphate-5′-2′deoxythymidyl-3′-phosphate-5′-uridyl-3′-phosphate-5′-uridyl-3′-phosphate); rUsrU (a thiophosphate linker: 5′-uridyl-3′-thiophosphate-5′-uridyl-3′-phosphate); an rUrU linker; dTsdTaa (aadTsdT, 5′-2′deoxythymidyl-3′-thiophosphate-5′-2′deoxythymidyl-3′-phosphate-5′-adenyl-3′-phosphate-5′-adenyl-3′-phosphate); dTsdT (5′-2′deoxythyrnidyl-3′-thiophosphate-5′-2′ deoxythymidyl-3′-phosphate); dTsdTuu=uudTsdT=5′-2′deoxythymidyl-3′-thiophosphate-5′-2′deoxythymidyl-3′-phosphate-5′-uridyl-3′-phosphate-5′-uridyl-3′-phosphate.

In some embodiments, the covalent linker can include a polyRNA, such as poly(5′-adenyl-3′-phosphate-AAAAAAAA) or poly(5′-cytidyl-3′-phosphate-5′-uridyl-3′-phosphate-CUCUCUCU)), e.g., Xn single stranded poly RNA linker wherein n is an integer from 2-50 inclusive, preferable 4-15 inclusive, most preferably 7-8 inclusive. Modified nucleotides or a mixture of nucleotides can also be present in said polyRNA linker. The covalent linker can be a polyDNA, such as poly(5′-2′deoxythymidyl-3′-phosphate-TTTTTTTT), e.g., wherein n is an integer from 2-50 inclusive, preferable 4-15 inclusive, most preferably 7-8 inclusive. Modified nucleotides or a mixture of nucleotides can also be present in said polyDNA linker. a single stranded polyDNA linker wherein n is an integer from 2-50 inclusive, preferable 4-15 inclusive, most preferably 7-8 inclusive. Modified nucleotides or a mixture of nucleotides can also be present in said polyDNA linker.

In some embodiments, the covalent linker can include a disulfide bond, optionally a bis-hexyl-disulfide linker. In one embodiment, the disulfide linker is as shown below:

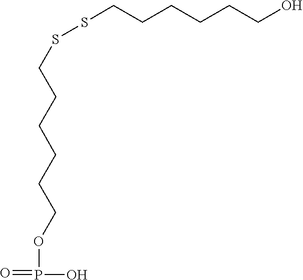

C₁₂H₂₆O₄PS₂ (Exact Mass: 329.1010; Mol. Wt.: 329.4362)

In some embodiments, the covalent linker can include a peptide bond, e.g., include amino acids. In one embodiment, the covalent linker is a 1-10 amino acid long linker, preferably comprising 4-5 amino acids, optionally X-Gly-Phe-Gly-Y wherein X and Y represent any amino acid.

In some embodiments, the covalent linker can include HEG, a hexaethylenglycol linker.

4.2.4 Orientation of covalent linkage

Aspects of the present disclosure include covalently linking two or more functional oligonucleotides to form a multi-valent oligonucleotide agent. The present inventors surprisingly found that the orientation of the linkage and positioning of the two or more functional oligonucleotides can affect the activity of multi-valent oligonucleotide agent in inducing or silencing or modulating target gene expression. In some embodiments, the orientation of the two or more functional oligonucleotides and the linker can enhance stability, oligonucleotide activity, or other beneficial characteristics, such as maximized target gene output, increased or decreased activity or expression (e.g., mRNA expression, protein expression, etc.) of one or more target genes.

In some embodiments, two adjacent functional oligonucleotids are covalently linked with the 5′ end of the first functional oligonucleotide to the 3′ end of the second functional oligonucleotide, with or without a linker therebetween. In some embodiments, two adjacent functional oligonucleotids are covalently linked with the 3′ end of the first functional oligonucleotide to the S′ end of the second functional oligonucleotide, with or without a linker therebetween.

For example, in some embodiments, the first ASO is covalently linked to a 3′ end of the sense or antisense strand of the first dsRNA; or b) the first ASO is covalently linked to a 5′ end of the sense or antisense strand of the first dsRNA. For another example, in certain embodiments, the 5′ end of the first or second ASO is conjugated to a linking component. In some embodiments, the 3′ end of the first or second ASO oligonucleotide is conjugated to a linking component.

4.2.5 Size of the ASO in the multi-valent oligonucleotide agent

Usually, an ASO may have a length of 15-25 nucleotides. The present inventors surprisingly found that the size of the single-stranded ASO in the multi-valent oligonucleotide agent can affect the activity of multi-valent oligonucleotide agent in inducing or silencing target gene expression. For example, in some cases, when the size of ASO reduced from about 20 nt to 12 nt or even to 6 nt, the activity of the whole agent may increase.

In some embodiments, multi-valent oligonucleotide agent containing certain sized ASO can enhance stability, oligonucleotide activity, or other beneficial characteristics, such as maximized target gene output, increased or decreased activity or expression (e.g., mRNA expression, protein expression, etc.) of one or more target genes.

In some embodiments, the ASO(s) in the MVO agent has a nucleotide sequence that is at least 5 contiguous nucleotides in length, such as 5-30 nucleotides in length, e.g., 5, 6, 7, 8, 9, 10, 11, 12, 13, 14, 15, 16, 17, 18, 19, 20 nucleotides in length.

The size of the ASO(s) can be selected by testing and comparing the effect or activity of the MVO agent comprising said ASO(s).

4.2.5 Subtypes of multi-valent oligonucleotide agent

Disclosed herein are various multi-valent oligonucleotide agents comprising two or more functional oligonucleotides that are covalently linked, wherein the two or more functional oligonucleotides are independently selected from: a) a double stranded RNA (dsRNA); and b) an antisense oligonucleotide (ASO).

The following are some descriptions about di- and tri-valent oligonucleotide agents comprising various functional oligonucleotides. Such embodiments do not necessarily represent the full scope of the invention, however, and reference is made therefore to the claims and herein for interpreting the scope of the invention.

4.2.5.1 Di-Valent Oligonucleotide Agent comprising dsRNA and ASO

In some aspects, disclosed herein is di-valent oligonucleotide agent comprising two functional oligonucleotides: a first double stranded RNA (dsRNA) and a first antisense oligonucleotide (ASO). In such a di-valent oligonucleotide agent, the dsRNA and the ASO may arranged in any order, such as dsRNA-ASO or ASO-dsRNA.

In some embodiments, the dsRNA is selected from a small interfering RNA (siRNA) or a small activating RNA (saRNA), and the ASO is a gapmer or a mixmer. In some embodiments, the two functional oligonucleotides independently modulate the expression of one or more genes, modulate the expression of one or more proteins (such as by binding to a mRNA sequence), or modulate non-coding regulatory nucleic acid sequences (such as a promoter sequence, enhancer, silencer, and/or transcription factor).

In some embodiments, the dsRNA comprises a sense strand that is at least 10 contiguous nucleotides and an antisense strand that is at least 10 contiguous nucleotides. In some embodiments, the dsRNA comprises a sense strand that is of 10-60 nucleotides in length and/or an antisense strand that is of 10-60 nucleotides in length. In some embodiments, the ASO has a nucleotide sequence that is at least 5 contiguous nucleotides in length. In some embodiments, the ASO has a nucleotide sequence that is 5-30 nucleotides in length. In some embodiments, the bi-valent oligonucleotide agent has a total length ranging from 15 to 100 nucleotides.

In some embodiments, the two adjacent functional oligonucleotides are covalently linked by a linking component or with no linking component. The linking component may selected from Spacer-9, Spacer-18, Spacer-C3 and Spacer-C6 or derivatives thereof, or any suitable linking components as disclosed in the present Specification or known in the art. In some embodiments, the two adjacent functional oligonucleotides are covalently linked by

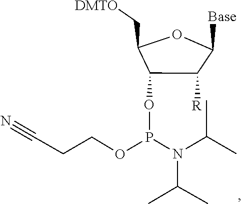

wherein R represents —H or —OH or —OMe, or -MOE, or —F, or other 2′ chemical modifications. In some embodiments, the two adjacent functional oligonucleotides are covalently linked by a phosphodiester bond or a phosphorothioate bond or by one or more nucleotides.

In some embodiments, the functional oligonucleotides comprise at least one chemically modified nucleotide. The modification to the chemically modified nucleotide may be is a 2′ sugar modification selected from one or more of: 2′-fluoro-2′-deoxynucleoside (2′-F) modification, 2′-O-methyl (2′-O—Me), modification, and 2′-O-(2-methoxyethyl) (2′-O-MOE) modification. In some embodiments, the chemical modification of the at least one chemically modified nucleotide is a Phosphorothioate (PS) backbone modification. In some embodiments, the chemical modification of the at least one chemically modified nucleotide is an addition of a 5′-phosophate moiety, an (E)-vinylphosphonate moiety, or a 5-methyl cytosine moiety at the 5′ end of the nucleotide sequence. In some embodiments, the functional oligonucleotides comprise one or more of the above modifications in one or more of the nucleotides (such as from one to up to all the nucleotides modification).

In some embodiments, the ASO in the agent is covalently linked to the adjacent dsRNA in a 3′ to S′ orientation or in a 5′ to 3′ orientation. In some embodiments, the dsRNA in the agent is covalently linked to the ASO at its 3′ end of the sense or antisense strand; or at its S′ end of the sense or antisense strand.

In some embodiments, the dsRNA is a siRNA or a saRNA, and the ASO is a gapmer and a mixmer. In some embodiments, the multi-valent oligonucleotide agent comprises functional oligonucleotides selected from: (a) siRNA-gapmer; (b) siRNA-mixmer; (c) saRNA-gapmer; and (d) saRNA-mixmer, wherein in any one of (a)˜(d), the functional oligonucleotides are arranged in any order and covalently linked with or without linking component(s). In some embodiments, the ASO targets 5′-UTR.

In some aspects, the dsRNA and/or the ASO increase the expression of a SMN2 gene or protein. In some embodiments, the dsRNA increases the expression of the SMN2 gene or protein; and/or the ASO increases the production of functional SMN protein by modulating SMN2 mRNA splicing or stability (SMN2 mRNA modulators).

In some embodiments, the dsRNA comprises a saRNA having a nucleotide sequence of a sense strand that is at least 90% identical to the nucleotide sequence selected from: a) DS06-0004 (SEQ ID NO: 5); b) DS06-0031 (SEQ ID NO: 7); c) DS06-0067 (SEQ ID NO: 9); d) DS06-4A3 (SEQ ID NO: 146); c) R6-04-S1 (SEQ ID NO: 59); and f) R6-04(20)-S1V1v(CM-4) (SEQ ID NO: 60).

In some embodiments, the dsRNA(s) comprises a saRNA having a nucleotide sequence of an antisense strand that is at least 90% identical to the nucleotide sequence selected from: a) DS06-0004 (SEQ ID NO: 6); b) DS06-0031 (SEQ ID NO: 8); c) DS06-0067 (SEQ ID NO: 10); d) DS06-4A3 (SEQ ID NO: 147); e) R6-04-S1 (SEQ ID NO: 53); and f) R6-04M1-27A-S1L1V3(CM-26) (SEQ ID NO: 17).

In some embodiments, the dsRNA comprises a saRNA having a pair of nucleotide sequences of a sense strand and an antisense strand that is at least 90% identical to the nucleotide sequence pairs selected from: a) DS06-0004: SEQ ID NO: 5 and SEQ ID NO: 6; b) DS06-0031: SEQ ID NO: 7 and SEQ ID NO: 8; c) DS06-0067: SEQ ID NO: 9 and SEQ ID NO: 10; d) DS06-4A3: SEQ ID NO: 146 and SEQ ID NO: 147; c) R6-04-S1: SEQ ID NO: 59 and SEQ ID NO: 53; and f) R6-04(20)-SIV1v(CM-4): SEQ ID NO: 60 and SEQ ID NO: 17.

In some embodiments, the dsRNA comprises a siRNA having a nucleotide sequence of a sense strand that is at least 90% identical to the nucleotide sequence of DS06-332i (SEQ ID NO: 3) or siSOD1-388-ESC (SEQ ID NO: 138). In some embodiments, the dsRNA comprises a siRNA having a nucleotide sequence of an antisense strand that is at least 90% identical to the nucleotide sequence of DS06-332i (SEQ ID NO: 4) or siSOD1-388-ESC (SEQ ID NO: 139). In some embodiments, the dsRNA comprises a siRNA having a pair of nucleotide sequences of a sense strand and an antisense strand that is at least 90% identical to the nucleotide sequence pairs selected from: DS06-332i: SEQ ID NO: 3 and SEQ ID NO: 4; siSOD1-388-ESC: SEQ ID NO: 138 and SEQ ID NO: 139.

In some embodiments, the multi-valent oligonucleotide agent has a nucleotide sequence that is at least 90% identical to the nucleotide sequences selected from:
a) DA06-4A-27A (SEQ ID NO: 14) and an antisense saRNA strand having a nucleotide sequence of SEQ ID NO: 13 that has partial complementarity with the sense saRNA strand of SEQ ID NO: 14;
b) DA06-4A-27B (SEQ ID NO: 15) and an antisense saRNA strand having a nucleotide sequence of SEQ ID NO: 13 that has partial complementarity with the sense saRNA strand of SEQ ID NO: 15;
c) R6-04M1-27A-S1L1V3 (SEQ ID NO: 18) and an antisense saRNA strand having a nucleotide sequence of SEQ ID NO: 13 that has partial complementarity with the sense saRNA strand of SEQ ID NO: 18;
d) DA06-31A-27A (SEQ ID NO: 19) and an antisense saRNA strand having a nucleotide sequence of SEQ ID NO: 8 that has partial complementarity with the sense saRNA strand of SEQ ID NO: 19;
e) DA06-31B-27A (SEQ ID NO: 20) and a sense saRNA strand having a nucleotide sequence of SEQ ID NO: 7 that has partial complementarity with the antisense saRNA strand of SEQ ID NO: 20;
f) DA06-67A-27A (SEQ ID NO: 21) and an antisense saRNA strand having a nucleotide sequence of SEQ ID NO: 10 that has partial complementarity with the sense saRNA strand of SEQ ID NO: 21;
g) DA06-67B-27A (SEQ ID NO: 22) and a sense saRNA strand having a nucleotide sequence of SEQ ID NO: 9 that has partial complementarity with the antisense saRNA strand of SEQ ID NO: 22;
h) DA6-67A3′L0-27A (SEQ ID NO: 23) and an antisense saRNA strand having a nucleotide sequence of SEQ ID NO: 10 that has partial complementarity with the sense saRNA strand of SEQ ID NO: 23;
j) DA6-67A3′L9-27A (SEQ ID NO: 24) and an antisense saRNA strand having a nucleotide sequence of SEQ ID NO: 10 that has partial complementarity with the sense saRNA strand of SEQ ID NO: 24;
k) DA6-67A3′LA-27A (SEQ ID NO: 25) and an antisense saRNA strand having a nucleotide sequence of SEQ ID NO: 10 that has partial complementarity with the sense saRNA strand of SEQ ID NO: 25;
l) DA6-67B3′L0-27A (SEQ ID NO: 26) and a sense saRNA strand having a nucleotide sequence of SEQ ID NO: 9 that has partial complementarity with the antisense saRNA strand of SEQ ID NO: 26;
m) DA6-67B5′L1-27A (SEQ ID NO: 27) and a sense saRNA strand having a nucleotide sequence of SEQ ID NO: 9 that has partial complementarity with the antisense saRNA strand of SEQ ID NO: 27;
o) DA6-67B5′L9-27A (SEQ ID NO: 29) and a sense saRNA strand having a nucleotide sequence of SEQ ID NO: 9 that has partial complementarity with the antisense saRNA strand of SEQ ID NO: 29;
p) DA6-67B5′LA-27A (SEQ ID NO: 30) and a sense saRNA strand having a nucleotide sequence of SEQ ID NO: 9 that has partial complementarity with the antisense saRNA strand of SEQ ID NO: 30;
q) DA6-67B3′L9-27A (SEQ ID NO: 31) and a sense saRNA strand having a nucleotide sequence of SEQ ID NO: 9 that has partial complementarity with the antisense saRNA strand of SEQ ID NO: 31;
r) DA6-67B3′L4-27A (SEQ ID NO: 32) and a sense saRNA strand having a nucleotide sequence of SEQ ID NO: 9 that has partial complementarity with the antisense saRNA strand of SEQ ID NO: 32;
s) DA06-67A21L1-27A (SEQ ID) NO: 33) and an antisense saRNA strand having a nucleotide sequence of SEQ ID NO: 34 that has partial complementarity with the sense saRNA strand of SEQ ID NO: 33;
t) DA06-67B21L1-27A (SEQ ID NO: 36) and sense saRNA strand of SEQ ID NO: 35 that has partial complementarity with the antisense saRNA strand of SEQ ID NO: 36;
u) DA6-04A3′L0-27A (SEQ ID NO: 37) and an antisense saRNA strand having a nucleotide sequence of SEQ ID NO: 6 that has partial complementarity with the sense saRNA strand of SEQ ID NO: 37;
v) DA6-04A5′L1-27A (SEQ ID NO: 38) and an antisense saRNA strand having a nucleotide sequence of SEQ ID NO: 6 that has partial complementarity with the sense saRNA strand of SEQ ID NO: 38;
w) DA6-04A5′L9-27A (SEQ ID NO: 39) and an antisense saRNA strand having a nucleotide sequence of SEQ ID NO: 6 that has partial complementarity with the sense saRNA strand of SEQ ID NO: 39;
x) DA6-04A5′L4-27A (SEQ ID NO: 40) and an antisense saRNA strand having a nucleotide sequence of SEQ ID NO: 6 that has partial complementarity with the sense saRNA strand of SEQ ID NO: 40;
y) DA6-04A3′L1-27A (SEQ ID) NO: 41) and an antisense saRNA strand having a nucleotide sequence of SEQ ID NO: 6 that has partial complementarity with the sense saRNA strand of SEQ ID NO: 41;
z) DA6-04A3′T.9-27A (SEQ ID NO: 42) and an antisense saRNA strand having a nucleotide sequence of SEQ ID NO: 6 that has partial complementarity with the sense saRNA strand of SEQ ID NO: 42;
aa) DA6-04A3′LA-27A (SEQ ID NO: 43) and an antisense saRNA strand having a nucleotide sequence of SEQ ID NO: 6 that has partial complementarity with the sense saRNA strand of SEQ ID NO: 43;
bb) DA6-04B3′L0-27A (SEQ ID NO: 44) and a sense saRNA strand having a nucleotide sequence of SEQ ID NO: 5 that has partial complementarity with the antisense saRNA strand of SEQ ID NO: 44;
cc) DA6-04B3′L1-27A (SEQ ID NO: 45) and a sense saRNA strand having a nucleotide sequence of SEQ ID NO: 5 that has partial complementarity with the antisense saRNA strand of SEQ ID NO: 45;
dd) DA6-04B3′T.9-27A (SEQ ID NO: 46) and a sense saRNA strand having a nucleotide sequence of SEQ ID NO: 5 that has partial complementarity with the antisense saRNA strand of SEQ ID NO: 46;
ee) DA6-04B3′L4-27A (SEQ ID NO: 47) and a sense saRNA strand having a nucleotide sequence of SEQ ID NO: 5 that has partial complementarity with the antisense saRNA strand of SEQ ID NO: 47;
ff) DA06-04A21L1-27A (SEQ ID NO: 48) and an antisense saRNA strand having a nucleotide sequence of SEQ ID NO: 49 that has partial complementarity with the sense saRNA strand of SEQ ID NO: 48;
gg) DA06-04B21L1-27A (SEQ ID NO: 51) and a sense saRNA strand of SEQ ID NO: 50 that has partial complementarity with the antisense saRNA strand of SEQ ID NO: 51;
hh) R6-04M1-16nt-S1L1V3v (SEQ ID NO: 79) and an antisense saRNA strand of SEQ ID NO: 17 that has partial complementarity with the sense saRNA strand of SEQ ID NO: 79;
ii) R6-04M1-15nt-S1L1V3v (SEQ ID NO: 80) and an antisense saRNA strand of SEQ ID NO: 17 that has partial complementarity with the sense saRNA strand of SEQ ID NO: 80;
jj) R6-04M1-14nt-S1L1V3v (SEQ ID NO: 81) and an antisense saRNA strand of SEQ ID NO: 17 that has partial complementarity with the sense saRNA strand of SEQ ID NO: 81;
kk) R6-04M1-13nt-S1L1V3v (SEQ ID NO: 82) and an antisense saRNA strand of SEQ ID NO: 17 that has partial complementarity with the sense saRNA strand of SEQ ID NO: 82;
11) R6-04M1-(12nt-B)-SIL1V3v (SEQ ID NO: 83) and an antisense saRNA strand of SEQ ID NO: 17 that has partial complementarity with the sense saRNA strand of SEQ ID NO: 83;
mm) R6-04M1-11nt-S1L1V3v (SEQ ID NO: 84) and an antisense saRNA strand of SEQ ID NO: 17 that has partial complementarity with the antisense saRNA strand of SEQ ID NO: 84;
nn) R6-04M1-10nt-S1L1V3v (SEQ ID NO: 85) and an antisense saRNA strand of SEQ ID NO: 17 that has partial complementarity with the antisense saRNA strand of SEQ ID NO: 85;
oo) R6-04M1-9nt-S1L1V3v (SEQ ID NO: 86) and an antisense saRNA strand of SEQ ID NO: 17 that has partial complementarity with the sense saRNA strand of SEQ ID NO: 86;
pp) R6-04M1-8nt-S1L1V3v (SEQ ID NO: 87) and an antisense saRNA strand of SEQ ID NO: 17 that has partial complementarity with the sense saRNA strand of SEQ ID NO: 87;
qq) R6-04M1-7nt-S1L1V3v (SEQ ID NO: 88) and an antisense saRNA strand of SEQ ID NO: 17 that has partial complementarity with the sense saRNA strand of SEQ ID NO: 88;
rr) R6-04M1-6nt-S1L1V3v (SEQ ID NO: 89) and an antisense saRNA strand of SEQ ID NO: 17 that has partial complementarity with the sense saRNA strand of SEQ ID NO: 89;
ss) DS06-4A-S2LSV (SEQ ID NO: 128) and an antisense saRNA strand having a nucleotide sequence of SEQ ID NO: 17 that has partial complementarity with the sense saRNA strand of SEQ ID NO: 128;
ss′) DS06-4A-S2L1v (SEQ ID NO: 16) and an antisense saRNA strand having a nucleotide sequence of SEQ ID NO: 17 that has partial complementarity with the sense saRNA strand of SEQ ID NO: 16;
tt) DA6-27A-5′UTR (SE
tt) DA6-27A-5′UTR (SEQ ID NO: 143);
uu) DA6-5′UTR-27A (SEQ ID NO: 144);
vv) R6-67M3-27A-SIL1V3 (SEQ ID NO: 129) and an antisense saRNA strand having a nucleotide sequence of SEQ ID NO: 130 that has partial complementarity with the sense saRNA strand of SEQ ID NO: 129;
ww) R6-67M3-16nt-S1L1V3 (SEQ ID) NO: 129) and an antisense saRNA strand having a nucleotide sequence of SEQ ID NO: 131 that has partial complementarity with the sense saRNA strand of SEQ ID NO: 129;
xx) R6-67M3-15nt-S1L1V3 (SEQ ID NO: 129) and an antisense saRNA strand having a nucleotide sequence of SEQ ID NO: 132 that has partial complementarity with the sense saRNA strand of SEQ ID NO: 129;
yy) R6-67M3-14nt-S1L1V3 (SEQ ID NO: 129) and an antisense saRNA strand having a nucleotide sequence of SEQ ID NO: 133 that has partial complementarity with the sense saRNA strand of SEQ ID NO: 129;
zz) R6-67M3-13nt-S1L1V3 (SEQ ID NO: 129) and an antisense saRNA strand having a nucleotide sequence of SEQ ID NO: 134 that has partial complementarity with the sense saRNA strand of SEQ ID NO: 129;
aaa) R6-67M3-12nt-S1L1V3 (SEQ ID NO: 129) and an antisense saRNA strand having a nucleotide sequence of SEQ ID NO: 135 that has partial complementarity with the sense saRNA strand of SEQ ID NO: 129;
bbb) R6-67M3-9nt-S1L1V3 (SEQ ID NO: 129) and an antisense saRNA strand having a nucleotide sequence of SEQ ID NO: 136 that has partial complementarity with the sense saRNA strand of SEQ ID NO: 129;
ccc) R6-67M3-8nt-S1L1V3 (SEQ ID NO: 129) and an antisense saRNA strand having a nucleotide sequence of SEQ ID NO:137 that has partial complementarity with the sense saRNA strand of SEQ ID NO: 129;
wherein linkers selected from the group consisting of L1, L4 and L9 is present or absent, wherein L1 represents spacer-18; L4 represents spacer-C6; and L9 represents spacer-9.

In some embodiments, the multi-valent oligonucleotide agents are as listed in Tables 7-11 and 13-14. In some embodiments, the linking components and/or linkage bonds and/or orientation of the above mentioned multi-valent oligonucleotide agents are changeable.

In some aspects, one or more of the functional oligonucleotides increase the expression of CDKN1A/p21 gene or protein and/or decreases the expression CD274/PDL-1. In some embodiments, the dsRNA is a saRNA that increases the expression of the CDKN1A/p21 gene or protein; and/or a siRNA that decreases the expression CD274/PDL-1. In some embodiments, the ASO is an ASO that increases the expression of CDKN1A/p21 gene or protein and/or decreases the expression CD274/PDL-1.

In some embodiments, the dsRNA is a saRNA having a nucleotide sequence of a sense strand that is at least 90% identical to the nucleotide sequence of RAG1-40 (SEQ ID NO: 62). In some embodiments, the dsRNA is a saRNA having a nucleotide sequence of an antisense strand that is at least 90% identical to the nucleotide sequence of RAG1-40 (SEQ ID NO: 63). In some embodiments, the dsRNA is a saRNA having a nucleotide sequence of a sense strand that is at least 90% identical to the nucleotide sequence of RAG1-40 (SEQ ID NO: 62) and a nucleotide sequence of an antisense strand that is at least 90% identical to the nucleotide sequence of RAG1-40 (SEQ ID NO: 63).

In some embodiments, the dsRNA is a siRNA having a nucleotide sequence of a sense strand that is at least 90% identical to the nucleotide sequence selected from: a) siPDL1-2 (SEQ ID NO: 64); and b) siPDL1-3 (SEQ ID NO: 66). In some embodiments, the dsRNA is a siRNA having a nucleotide sequence of an antisense strand that is at least 90% identical to the nucleotide sequence selected from: a) siPDL1-2 (SEQ ID NO: 65); and b) siPDL1-3 (SEQ ID NO: 67). In some embodiments, the dsRNA is a siRNA selected from: a) a siRNA having a nucleotide sequence of a sense strand that is at least 90% identical to the nucleotide sequence siPDL1-2 (SEQ ID NO: 64) and a nucleotide sequence of an antisense strand that is at least 90% identical to the nucleotide sequence of siPDL1-2 (SEQ ID NO: 65); and b) a siRNA having a nucleotide sequence of a sense strand that is at least 90% identical to the nucleotide sequence siPDL1-2 (SEQ ID NO: 66) and a nucleotide sequence of an antisense strand that is at least 90% identical to the nucleotide sequence of siPDL1-2 (SEQ ID NO: 67).

In some embodiments, the ASO has a nucleotide sequence that is at least 90% identical to the nucleotide sequence selected from: a) aPDL1-1 (SEQ ID NO: 68); b) aPDL1-2 (SEQ ID NO: 69); and c) aPDL1-3 (SEQ ID NO: 70).

In some embodiments, the multi-valent oligonucleotide agent has a nucleotide sequence that is at least 90% identical to the nucleotide sequences selected from:
a) saP21-40/aPDL1-1 (SEQ ID) NO: 72) and an antisense saRNA strand having a nucleotide sequence of SEQ ID NO: 63 that has partial complementarity with the sense saRNA strand of SEQ ID NO: 72;
b) saP21-40/aPDL1-2 (SEQ ID NO: 73) and an antisense saRNA strand having a nucleotide sequence of SEQ ID NO: 63 that has partial complementarity with the sense saRNA strand of SEQ ID NO: 73;
c) saP21-40/aPDL1-3 (SEQ ID NO: 74) and an antisense saRNA strand having a nucleotide sequence of SEQ ID NO: 63 that has partial complementarity with the sense saRNA strand of SEQ ID NO: 74;
d) saP21-40/aPDL1-1R (SEQ ID NO: 75) and an antisense saRNA strand having a nucleotide sequence of SEQ ID NO: 63 that has partial complementarity with the sense saRNA strand of SEQ ID NO: 75;
e) saP21-40/aPDL1-2R (SEQ ID NO: 76) and an antisense saRNA strand having a nucleotide sequence of SEQ ID NO: 63 that has partial complementarity with the sense saRNA strand of SEQ ID NO: 76; and
f) saP21-40/aPDL1-3R (SEQ ID NO: 77) and an antisense saRNA strand having a nucleotide sequence of SEQ ID NO: 63 that has partial complementarity with the sense saRNA strand of SEQ ID NO: 77.

In some embodiments, the multi-valent oligonucleotide agent is selected from or has at least 90% sequence identity to those shown in Table 16. In some embodiments, the linking components and/or linkage bonds and/or orientation of those multi-valent oligonucleotide agents are changeable.

4.2.5.2 Di-Valent Oligonucleotide Agent comprising dsRNA and dsRNA

In some aspects, disclosed herein is di-valent oligonucleotide agent comprising two functional oligonucleotides: a first double stranded RNA (dsRNA) and a second double stranded RNA (dsRNA). In such a di-valent oligonucleotide agent, the dsRNAs may be arranged in any order, such as dsRNA1-dsRNA2 or dsRNA2-dsRNA1; and the two functional oligonucleotides may be covalently connected via a linker or a bond.

In some embodiments, the dsRNA is selected from a small interfering RNA (siRNA) or a small activating RNA (saRNA), and the two functional oligonucleotides independently modulate the expression of one or more genes, modulate the expression of one or more proteins (such as by binding to a mRNA sequence), or modulate non-coding regulatory nucleic acid sequences (such as a promoter sequence, enhancer, silencer, and/or transcription factor).

In some embodiments, the dsRNA comprises a sense strand that is at least 10 contiguous nucleotides and an antisense strand that is at least 10 contiguous nucleotides. In some embodiments, the dsRNA comprises a sense strand that is of 10-60 nucleotides in length and/or an antisense strand that is of 10-60 nucleotides in length. In some embodiments, the bi-valent oligonucleotide agent has a total length ranging from 20 to 200 nucleotides.

In some embodiments, the two adjacent functional oligonucleotides are covalently linked by a linking component or with no linking component. The linking component may selected from Spacer-9, Spacer-18, Spacer-C3 and Spacer-C6 or derivatives thereof, or any suitable linking components as disclosed in the present Specification or known in the art. In some embodiments, the two adjacent functional oligonucleotides are covalently linked by

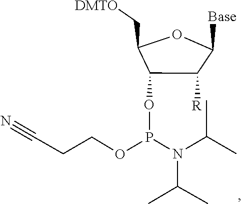

wherein R represents —H or —OH or —OMe, or -MOE, or —F, or other 2′ chemical modifications. In some embodiments, the two adjacent functional oligonucleotides are covalently linked by a phosphodiester bond or a phosphorothioate bond or by one or more nucleotides.

In some embodiments, the functional oligonucleotides comprise at least one chemically modified nucleotide. The modification to the chemically modified nucleotide may be is a 2′ sugar modification selected from one or more of: 2′-fluoro-2′-deoxynucleoside (2′-F) modification, 2′-O-methyl (2′-O—Me), modification, and 2′-O-(2-methoxyethyl) (2′-O-MOE) modification. In some embodiments, the chemical modification of the at least one chemically modified nucleotide is a Phosphorothioate (PS) backbone modification. In some embodiments, the chemical modification of the at least one chemically modified nucleotide is an addition of a 5′-phosophate moiety, an (E)-vinylphosphonate moiety, or a 5-methyl cytosine moiety at the 5′ end of the nucleotide sequence. In some embodiments, the functional oligonucleotides comprise one or more of the above modifications in one or more of the nucleotides (such as from one to up to all the nucleotides modification).

In some embodiments, the dsRNAs in the agent are covalently linked in a 3′ to 5′ orientation or in a 5′ to 3′ orientation. In some embodiments, the first dsRNA in the agent is covalently linked to the second dsRNA at its 3′ end of the sense or antisense strand; or at its 5′ end of the sense or antisense strand.

In some embodiments, the dsRNA is a siRNA or a saRNA. In some embodiments, the multi-valent oligonucleotide agent comprises functional oligonucleotides selected from: (a) siRNA-siRNA; (b) siRNA-saRNA; (c) saRNA-saRNA, wherein in any one of (a)˜ (c), the functional oligonucleotides are arranged in any order and covalently linked with or without linking component(s). In some embodiments, the functional oligonucleotides in the same agent can be identical or different.

In some aspects, the dsRNA(s) increase/inhibit the expression of a SMN2 gene or protein.

In some embodiments, the dsRNA comprises a saRNA having a nucleotide sequence of a sense strand that is at least 90% identical to the nucleotide sequence selected from: a) DS06-0004 (SEQ ID NO: 5); b) DS06-0031 (SEQ ID NO: 7); c) DS06-0067 (SEQ ID NO: 9); d) DS06-4A3 (SEQ ID NO: 146); c) R6-04-S1 (SEQ ID NO: 59); and f) R6-04(20)-S1V1v(CM-4) (SEQ ID NO: 60).

In some embodiments, the dsRNA(s) comprises a saRNA having a nucleotide sequence of an antisense strand that is at least 90% identical to the nucleotide sequence selected from: a) DS06-0004 (SEQ ID NO: 6); b) DS06-0031 (SEQ ID NO: 8); c) DS06-0067 (SEQ ID NO: 10); d) DS06-4A3 (SEQ ID NO: 147); e) R6-04-S1 (SEQ ID NO: 53); and f) R6-04M1-27A-S1L1V3(CM-26) (SEQ ID NO: 17).

In some embodiments, the dsRNA comprises a saRNA having a pair of nucleotide sequences of a sense strand and an antisense strand that is at least 90% identical to the nucleotide sequence pairs selected from: a) DS06-0004: SEQ ID NO: 5 and SEQ ID NO: 6; b) DS06-0031: SEQ ID NO: 7 and SEQ ID NO: 8; c) DS06-0067: SEQ ID NO: 9 and SEQ ID NO: 10; d) DS06-4A3: SEQ ID NO: 146 and SEQ ID NO: 147; c) R6-04-S1: SEQ ID NO: 59 and SEQ ID NO: 53; and f) R6-04(20)-S1V1v(CM-4): SEQ ID NO: 60 and SEQ ID NO: 17.

In some embodiments, the dsRNA comprises a siRNA having a nucleotide sequence of a sense strand that is at least 90% identical to the nucleotide sequence of DS06-332i (SEQ ID NO: 3) or siSOD1-388-ESC (SEQ ID NO: 138). In some embodiments, the dsRNA comprises a siRNA having a nucleotide sequence of an antisense strand that is at least 90% identical to the nucleotide sequence of DS06-332i (SEQ ID NO: 4) or siSOD1-388-ESC (SEQ ID NO: 139). In some embodiments, the dsRNA comprises a siRNA having a pair of nucleotide sequences of a sense strand and an antisense strand that is at least 90% identical to the nucleotide sequence pairs selected from: DS06-332i: SEQ ID NO: 3 and SEQ ID NO: 4; siSOD1-388-ESC: SEQ ID NO: 138 and SEQ ID NO: 139.

In some embodiments, the multi-valent oligonucleotide agent has a nucleotide sequence that is at least 90% identical to the nucleotide sequences selected from:
a) R6-04S1&67S1R-L1V2 (SEQ ID NO: 52) and an antisense saRNA strand having a nucleotide sequence of SEQ ID NO: 53 that has partial complementarity with the strand of SEQ ID NO: 52 and an antisense saRNA strand having a nucleotide sequence of SEQ ID NO: 78 that has partial complementarity with the strand of SEQ ID NO: 52;
b) R6-04S1&67S5-L1V2 (SEQ ID NO: 56) and an antisense saRNA strand having a nucleotide sequence of SEQ ID NO: 53 that has partial complementarity with the strand of SEQ ID NO: 56 and an antisense saRNA strand having a nucleotide sequence of SEQ ID NO: 78 that has partial complementarity with the strand of SEQ ID NO: 56;
c) R6-04M1&R17-388E-L1V2 (SEQ ID NO: 140) and an antisense saRNA strand of SEQ ID NO: 17 that has partial complementarity with the strand of SEQ ID NO: 99 and an antisense siRNA strand of SEQ ID NO: 141 that has partial complementarity with the strand of SEQ ID NO: 99;
d) DS06-4A-S2L1v (SEQ ID NO: 16) and an antisense saRNA strand of SEQ ID NO: 17 that has partial complementarity with the strand of SEQ ID NO: 16; and
e) DS06-4A-S2L5V (SEQ ID NO: 128) and an antisense saRNA strand having a nucleotide sequence of SEQ ID NO: 17 that has partial complementarity with the sense saRNA strand of SEQ ID NO: 128.

In some embodiments, the multi-valent oligonucleotide agents are as listed in Tables 12 and 15. In some embodiments, the linking components and/or linkage bonds and/or orientation of the above mentioned multi-valent oligonucleotide agents are changeable.

In some aspects, one or more of the functional oligonucleotides increase the expression of CDKN1A/p21 gene or protein and/or decreases the expression CD274/PDL-1. In some embodiments, the dsRNA is a saRNA that increases the expression of the CDKN1A/p21 gene or protein; and/or a siRNA that decreases the expression CD274/PDL-1.

In some embodiments, the dsRNA is a saRNA having a nucleotide sequence of a sense strand that is at least 90% identical to the nucleotide sequence of RAG1-40 (SEQ ID NO: 62). In some embodiments, the dsRNA is a saRNA having a nucleotide sequence of an antisense strand that is at least 90% identical to the nucleotide sequence of RAG1-40 (SEQ ID NO: 63). In some embodiments, the dsRNA is a saRNA having a nucleotide sequence of a sense strand that is at least 90% identical to the nucleotide sequence of RAG1-40 (SEQ ID NO: 62) and a nucleotide sequence of an antisense strand that is at least 90% identical to the nucleotide sequence of RAG1-40 (SEQ ID NO: 63).

In some embodiments, the dsRNA is a siRNA having a nucleotide sequence of a sense strand that is at least 90% identical to the nucleotide sequence selected from: a) siPDL1-2 (SEQ ID NO: 64); and b) siPDL1-3 (SEQ ID NO: 66). In some embodiments, the dsRNA is a siRNA having a nucleotide sequence of an antisense strand that is at least 90% identical to the nucleotide sequence selected from: a) siPDL1-2 (SEQ ID NO: 65); and b) siPDL1-3 (SEQ ID NO: 67). In some embodiments, the dsRNA is a siRNA selected from: a) a siRNA having a nucleotide sequence of a sense strand that is at least 90% identical to the nucleotide sequence siPDL1-2 (SEQ ID NO: 64) and a nucleotide sequence of an antisense strand that is at least 90% identical to the nucleotide sequence of siPDL1-2 (SEQ ID NO: 65); and b) a siRNA having a nucleotide sequence of a sense strand that is at least 90% identical to the nucleotide sequence siPDL1-2 (SEQ ID NO: 66) and a nucleotide sequence of an antisense strand that is at least 90% identical to the nucleotide sequence of siPDL1-2 (SEQ ID NO: 67).

In some embodiments, the multi-valent oligonucleotide agent has a nucleotide sequence that is at least 90% identical to the nucleotide sequences selected from
a) saP21-40/siPDL1-2 (SEQ ID NO: 71) and an antisense saRNA strand having a nucleotide sequence of SEQ ID NO: 63 that has partial complementarity with the strand of SEQ ID NO: 71 and an antisense siRNA strand having a nucleotide sequence of SEQ ID NO: 65 that has partial complementarity with the strand of SEQ ID NO: 71; and
b) saP21-40/siPDL1-3 (SEQ ID NO: 100) and an antisense saRNA strand having a nucleotide sequence of SEQ ID NO: 63 that has partial complementarity with the sense saRNA strand of SEQ ID NO: 100 and an antisense saRNA strand having a nucleotide sequence of SEQ ID NO: 65 that has partial complementarity with the strand of SEQ ID NO: 100.

In some embodiments, the multi-valent oligonucleotide agent is selected from or has at least 90% sequence identity to those shown in Table 16. In some embodiments, the linking components and/or linkage bonds and/or orientation of those multi-valent oligonucleotide agents are changeable.

4.2.5.3 Di-Valent Oligonucleotide Agent comprising ASO and ASO

In some aspects, disclosed herein is di-valent oligonucleotide agent comprising two functional oligonucleotides: a first antisense oligonucleotide (ASO1) and a second antisense oligonucleotide (ASO2). In such a di-valent oligonucleotide agent, the dsRNA and the ASO may be arranged in any order, such as ASO1-ASO2 or ASO2-ASO1. The two ASOs may be identical or different.

In some embodiments, the ASO is independently selected from a gapmer or a mixmer. In some embodiments, the two functional oligonucleotides independently modulate the expression of one or more genes, modulate the expression of one or more proteins (such as by binding to a mRNA sequence), or modulate non-coding regulatory nucleic acid sequences (such as a promoter sequence, enhancer, silencer, and/or transcription factor).

In some embodiments, the ASO has a nucleotide sequence that is at least 5 contiguous nucleotides in length. In some embodiments, the ASO has a nucleotide sequence that is 5-30 nucleotides in length. In some embodiments, the bi-valent oligonucleotide agent has a total length ranging from 10 to 100 nucleotides.

In some embodiments, the two adjacent functional oligonucleotides are covalently linked by a linking component or with no linking component. The linking component may selected from Spacer-9, Spacer-18, Spacer-C3 and Spacer-C6 or derivatives thereof, or any suitable linking components as disclosed in the present Specification or known in the art. In some embodiments, the two adjacent functional oligonucleotides are covalently linked by

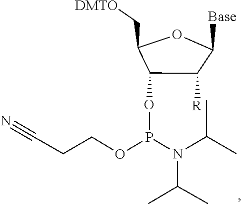

wherein R represents —H or —OH or —OMe, or -MOE, or —F, or other 2′ chemical modifications. In some embodiments, the two adjacent functional oligonucleotides are covalently linked by a phosphodiester bond or a phosphorothioate bond or by one or more nucleotides.

In some embodiments, the functional oligonucleotides comprise at least one chemically modified nucleotide. The modification to the chemically modified nucleotide may be is a 2′ sugar modification selected from one or more of: 2′-fluoro-2′-deoxynucleoside (2′-F) modification, 2′-O-methyl (2′-O—Me), modification, and 2′-O-(2-methoxyethyl) (2′-O-MOE) modification. In some embodiments, the chemical modification of the at least one chemically modified nucleotide is a Phosphorothioate (PS) backbone modification. In some embodiments, the chemical modification of the at least one chemically modified nucleotide is an addition of a 5′-phosophate moiety, an (E)-vinylphosphonate moiety, or a 5-methyl cytosine moiety at the 5′ end of the nucleotide sequence. In some embodiments, the functional oligonucleotides comprise one or more of the above modifications in one or more of the nucleotides (such as from one to up to all the nucleotides modification).

In some embodiments, the first ASO in the agent is covalently linked to the second ASO in a 3′ to 5′ orientation or in a 5′ to 3′ orientation. In some embodiments, the first ASO in the agent is covalently linked to the second ASO at its 3′ end; or at its 5′ end.

In some embodiments, the ASO is selected from a gapmer and a mixmer. In some embodiments, the multi-valent oligonucleotide agent comprises functional oligonucleotides selected from: (a) mixer-mixer; (b) gapmer-mixmer; (c) gapmer-gapmer, wherein in any one of (a)˜(c), the functional oligonucleotides are arranged in any order and covalently linked with or without linking component(s). In some embodiments, the ASO targets 5′-UTR.

In some aspects, the ASO increase the expression of a SMN2 gene or protein. In some embodiments, the ASO increases the production of functional SMN protein by modulating SMN2 mRNA splicing or stability (SMN2 mRNA modulators).

In some embodiments, the ASO has a nucleotide sequence that is at least 90% identical to the nucleotide sequence of ASO10-27 (SEQ ID NO: 11) or S′UTR ASO (SEQ ID NO: 142).

In some embodiments, the multivalent nucleotide agent has a nucleotide sequence that is at least 90% identical to the nucleotide sequences selected from:
a) DA6-27A-5′UTR (SEQ ID NO: 143); and
b) DA6-S′UTR-27A (SEQ ID NO: 144).

In some embodiments, the multi-valent oligonucleotide agent is selected from or has at least 90% sequence identity to those shown in Table 17. In some embodiments, the linking components and/or linkage bonds and/or orientation of those multi-valent oligonucleotide agents are changeable.

4.2.5.4 Tri-Valent Oligonucleotide Agent comprising 2 dsRNAs and 1 ASO

In some aspects, disclosed herein is tri-valent oligonucleotide agent comprising three functional oligonucleotides: a first double stranded RNA (dsRNA), a second dsRNA, and a first antisense oligonucleotide (ASO). In such a tri-valent oligonucleotide agent, the dsRNAs and the ASO may arranged in any order, such as dsRNA1-dsRNA2-ASO, dsRNA2-dsRNA1-ASO, dsRNA1-ASO-dsRNA2, dsRNA2-ASO-dsRNA1, ASO-dsRNA1-dsRNA2, ASO-dsRNA2-dsRNA1.

In some embodiments, the dsRNA is selected from a small interfering RNA (siRNA) or a small activating RNA (saRNA), and the ASO is a gapmer or a mixmer. In some embodiments, the two functional oligonucleotides independently modulate the expression of one or more genes, modulate the expression of one or more proteins (such as by binding to a mRNA sequence), or modulate non-coding regulatory nucleic acid sequences (such as a promoter sequence, enhancer, silencer, and/or transcription factor).

In some embodiments, the dsRNA comprises a sense strand that is at least 10 contiguous nucleotides and an antisense strand that is at least 10 contiguous nucleotides. In some embodiments, the dsRNA comprises a sense strand that is of 10-60 nucleotides in length and/or an antisense strand that is of 10-60 nucleotides in length. In some embodiments, the ASO has a nucleotide sequence that is at least 5 contiguous nucleotides in length. In some embodiments, the ASO has a nucleotide sequence that is 5-30 nucleotides in length. In some embodiments, the tri-valent oligonucleotide agent has a total length ranging from 15 to 100 nucleotides.

In some embodiments, the two adjacent functional oligonucleotides are covalently linked by a linking component or with no linking component. The linking component may selected from Spacer-9, Spacer-18, Spacer-C3 and Spacer-C6 or derivatives thereof, or any suitable linking components as disclosed in the present Specification or known in the art. In some embodiments, the two adjacent functional oligonucleotides are covalently linked by

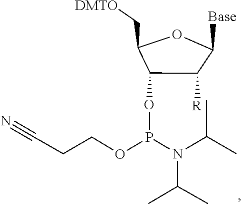

wherein R represents —H or —OH or —OMe, or -MOE, or —F, or other 2′ chemical modifications. In some embodiments, the two adjacent functional oligonucleotides are covalently linked by a phosphodiester bond or a phosphorothioate bond or by one or more nucleotides.

In some embodiments, the functional oligonucleotides comprise at least one chemically modified nucleotide. The modification to the chemically modified nucleotide may be is a 2′ sugar modification selected from one or more of: 2′-fluoro-2′-deoxynucleoside (2′-F) modification, 2′-O-methyl (2′-O—Me), modification, and 2′-O-(2-methoxyethyl) (2′-O-MOE) modification. In some embodiments, the chemical modification of the at least one chemically modified nucleotide is a Phosphorothioate (PS) backbone modification. In some embodiments, the chemical modification of the at least one chemically modified nucleotide is an addition of a 5′-phosophate moiety, an (E)-vinylphosphonate moiety, or a 5-methyl cytosine moiety at the 5′ end of the nucleotide sequence. In some embodiments, the functional oligonucleotides comprise one or more of the above modifications in one or more of the nucleotides (such as from one to up to all the nucleotides modification).

In some embodiments, the ASO in the agent is covalently linked to the adjacent dsRNA in a 3′ to 5′ orientation or in a 5′ to 3′ orientation. In some embodiments, the dsRNA in the agent is covalently linked to the ASO at its 3′ end of the sense or antisense strand; or at its 5′ end of the sense or antisense strand.

In some embodiments, the dsRNA is a siRNA or a saRNA, and the ASO is a gapmer and a mixmer. In some embodiments, the multi-valent oligonucleotide agent comprises functional oligonucleotides selected from: a) siRNA-siRNA-gapmer; b) siRNA-siRNA-mixmer; c) siRNA-saRNA-gapmer; d) siRNA-saRNA-mixmer; e) saRNA-saRNA-gapmer; f) saRNA-saRNA-mixmer, wherein in any one of a)˜ f), the functional oligonucleotides are arranged in any order and covalently linked with or without linking component(s). In some embodiments, the ASO targets 5′-UTR.

In some aspects, the dsRNA(s) and/or the ASO increase the expression of a SMN2 gene or protein. In some embodiments, the dsRNA(s) increases the expression of the SMN2 gene or protein; and/or the ASO increases the production of functional SMN protein by modulating SMN2 mRNA splicing or stability (SMN2 mRNA modulators).

In some embodiments, the dsRNA comprises a saRNA having a nucleotide sequence of a sense strand that is at least 90% identical to the nucleotide sequence selected from: a) DS06-0004 (SEQ ID) NO: 5); b) DS06-0031 (SEQ ID NO: 7); c) DS06-0067 (SEQ ID NO: 9); d) DS06-4A3 (SEQ ID NO: 146); e) R6-04-S1 (SEQ ID NO: 59); and f) R6-04(20)-S1V1v(CM-4) (SEQ ID NO: 60).

In some embodiments, the dsRNA(s) comprises a saRNA having a nucleotide sequence of an antisense strand that is at least 90% identical to the nucleotide sequence selected from: a) DS06-0004 (SEQ ID NO: 6); b) DS06-0031 (SEQ ID NO: 8); c) DS06-0067 (SEQ ID NO: 10); d) DS06-4A3 (SEQ ID NO: 147); e) R6-04-S1 (SEQ ID NO: 53); and f) R6-04M1-27A-SIL1V3(CM-26) (SEQ ID NO: 17).

In some embodiments, the dsRNA comprises a saRNA having a pair of nucleotide sequences of a sense strand and an antisense strand that is at least 90% identical to the nucleotide sequence pairs selected from: a) DS06-0004: SEQ ID NO: 5 and SEQ ID NO: 6; b) DS06-0031: SEQ ID NO: 7 and SEQ ID NO: 8; c) DS06-0067: SEQ ID NO: 9 and SEQ ID NO: 10; d) DS06-4A3: SEQ ID NO: 146 and SEQ ID NO: 147; e) R6-04-S1: SEQ ID NO: 59 and SEQ ID NO: 53; f) R6-04(20)-S1V1v(CM-4): SEQ ID NO: 60 and SEQ ID NO: 17.

In some embodiments, the dsRNA comprises a siRNA having a nucleotide sequence of a sense strand that is at least 90% identical to the nucleotide sequence of DS06-332i (SEQ ID NO: 3) or siSOD1-388-ESC (SEQ ID NO: 138). In some embodiments, the dsRNA comprises a siRNA having a nucleotide sequence of an antisense strand that is at least 90% identical to the nucleotide sequence of DS06-332i (SEQ ID NO: 4) or siSOD1-388-ESC (SEQ ID NO: 139). In some embodiments, the dsRNA comprises a siRNA having a pair of nucleotide sequences of a sense strand and an antisense strand that is at least 90% identical to the nucleotide sequence pairs selected from: DS06-332i: SEQ ID NO: 3 and SEQ ID NO: 4; siSOD1-388-ESC: SEQ ID NO: 138 and SEQ ID NO: 139.

In some embodiments, the multi-valent oligonucleotide agent has a nucleotide sequence that is at least 90% identical to the nucleotide sequences selected from:
a) R6-04S1&27A&67S1R-L1V2 (SEQ ID NO: 54) and an antisense saRNA strand having a nucleotide sequence of SEQ ID NO: 53 that has partial complementarity with the strand of SEQ ID NO: 54 and an antisense saRNA strand having a nucleotide sequence of SEQ ID NO: 78 that has partial complementarity with the strand of SEQ ID NO: 54;
b) R6-04S1&67S1R&27A-L1V2 (SEQ ID NO: 55) and an antisense saRNA strand having a nucleotide sequence of SEQ ID NO: 53 that has partial complementarity with the strand of SEQ ID NO: 55 and an antisense saRNA strand having a nucleotide sequence of SEQ ID NO: 78 that has partial complementarity with the strand of SEQ ID NO: 55;
c) R6-04S1&27A&67S5-L1V2 (SEQ ID NO: 57) and an antisense saRNA strand having a nucleotide sequence of SEQ ID NO: 53 that has partial complementarity with the strand of SEQ ID NO: 57 and an antisense saRNA strand having a nucleotide sequence of SEQ ID NO: 78 that has partial complementarity with the strand of SEQ ID NO: 57; and
d) R6-04S1&67S5&27A-L1V2 (SEQ ID NO: 58) and an antisense saRNA strand having a nucleotide sequence of SEQ ID NO: 53 that has partial complementarity with the strand of SEQ ID NO: 58 and an antisense saRNA strand having a nucleotide sequence of SEQ ID NO: 78 that has partial complementarity with the strand of SEQ ID NO: 58, wherein L1 represents spacer-18; L4 represents spacer-C6; and L9 represents spacer-9.

In some embodiments, the multi-valent oligonucleotide agents are as listed in Table 12. In some embodiments, the linking components and/or linkage bonds and/or orientation of the above mentioned multi-valent oligonucleotide agents are changeable.

In some aspects, one or more of the functional oligonucleotides increase the expression of CDKN1A/p21 gene or protein and/or decreases the expression CD274/PDL-1. In some embodiments, the dsRNA is a saRNA that increases the expression of the CDKN1A/p21 gene or protein; and/or a siRNA that decreases the expression CD274/PDL-1. In some embodiments, the ASO is an ASO that increases the expression of CDKN1A/p21 gene or protein and/or decreases the expression CD274/PDL-1.

In some embodiments, the dsRNA is a saRNA having a nucleotide sequence of a sense strand that is at least 90% identical to the nucleotide sequence of RAG1-40 (SEQ ID NO: 62). In some embodiments, the dsRNA is a saRNA having a nucleotide sequence of an antisense strand that is at least 90% identical to the nucleotide sequence of RAG1-40 (SEQ ID NO: 63). In some embodiments, the dsRNA is a saRNA having a nucleotide sequence of a sense strand that is at least 90% identical to the nucleotide sequence of RAG1-40 (SEQ ID NO: 62) and a nucleotide sequence of an antisense strand that is at least 90% identical to the nucleotide sequence of RAG1-40 (SEQ ID NO: 63).

In some embodiments, the dsRNA is a siRNA having a nucleotide sequence of a sense strand that is at least 90% identical to the nucleotide sequence selected from: a) siPDL1-2 (SEQ ID NO: 64); and b) siPDL1-3 (SEQ ID NO: 66). In some embodiments, the dsRNA is a siRNA having a nucleotide sequence of an antisense strand that is at least 90% identical to the nucleotide sequence selected from: a) siPDL1-2 (SEQ ID NO: 65); and b) siPDL1-3 (SEQ ID NO: 67). In some embodiments, the dsRNA is a siRNA selected from: a) a siRNA having a nucleotide sequence of a sense strand that is at least 90% identical to the nucleotide sequence siPDL1-2 (SEQ ID NO: 64) and a nucleotide sequence of an antisense strand that is at least 90% identical to the nucleotide sequence of siPDL1-2 (SEQ ID NO: 65); and b) a siRNA having a nucleotide sequence of a sense strand that is at least 90% identical to the nucleotide sequence siPDL1-2 (SEQ ID NO: 66) and a nucleotide sequence of an antisense strand that is at least 90% identical to the nucleotide sequence of siPDL1-2 (SEQ ID NO: 67).

In some embodiments, the ASO has a nucleotide sequence that is at least 90% identical to the nucleotide sequence selected from: a) aPDL1-1 (SEQ ID NO: 68); b) aPDL1-2 (SEQ ID NO: 69); and c) aPDL1-3 (SEQ ID NO: 70).

In some embodiments, the linking components and/or linkage bonds and/or orientation of those multi-valent oligonucleotide agents are changeable.

In some embodiments, the tri-valent oligonucleotide agent can be those produced by adding one dsRNA or ASO to a bi-valent oligonucleotide agent, such as the bi-valent oligonucleotide agents disclosed in 4.2.5.1 or 4.2.5.2.

4.2.5.5 Tri-Valent Oligonucleotide Agent comprising 1 dsRNA and 2 ASOs

In some aspects, disclosed herein is tri-valent oligonucleotide agent comprising three functional oligonucleotides: a first double stranded RNA (dsRNA), a first antisense oligonucleotide (ASO) and a second ASO. In such a tri-valent oligonucleotide agent, the dsRNA and the ASOs may be arranged in any order, such as dsRNA-ASO1-ASO2, dsRNA-ASO2-ASO1, ASO1-dsRNA-ASO2, ASO1-ASO2-dsRNA, ASO2-dsRNA-ASO1, ASO2-ASO1-dsRNA.

In some embodiments, the dsRNA is selected from a small interfering RNA (siRNA) or a small activating RNA (saRNA), and the ASO is a gapmer or a mixmer. In some embodiments, the two functional oligonucleotides independently modulate the expression of one or more genes, modulate the expression of one or more proteins (such as by binding to a mRNA sequence), or modulate non-coding regulatory nucleic acid sequences (such as a promoter sequence, enhancer, silencer, and/or transcription factor).

In some embodiments, the dsRNA comprises a sense strand that is at least 10 contiguous nucleotides and an antisense strand that is at least 10 contiguous nucleotides. In some embodiments, the dsRNA comprises a sense strand that is of 10-60 nucleotides in length and/or an antisense strand that is of 10-60 nucleotides in length. In some embodiments, the ASO has a nucleotide sequence that is at least 5 contiguous nucleotides in length. In some embodiments, the ASO has a nucleotide sequence that is 5-30 nucleotides in length. In some embodiments, the tri-valent oligonucleotide agent has a total length ranging from 15 to 100 nucleotides.

In some embodiments, the two adjacent functional oligonucleotides are covalently linked by a linking component or with no linking component. The linking component may selected from Spacer-9, Spacer-18, Spacer-C3 and Spacer-C6 or derivatives thereof, or any suitable linking components as disclosed in the present Specification or known in the art. In some embodiments, the two adjacent functional oligonucleotides are covalently linked by

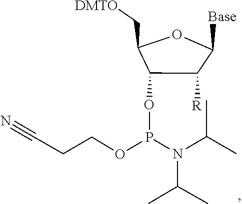

wherein R represents —H or —OH or —OMe, or -MOE, or —F, or other 2′ chemical modifications. In some embodiments, the two adjacent functional oligonucleotides are covalently linked by a phosphodiester bond or a phosphorothioate bond or by one or more nucleotides.

In some embodiments, the functional oligonucleotides comprise at least one chemically modified nucleotide. The modification to the chemically modified nucleotide may be is a 2′ sugar modification selected from one or more of: 2′-fluoro-2′-deoxynucleoside (2′-F) modification, 2′-O-methyl (2′-O—Me), modification, and 2′-( )(2-methoxyethyl) (2′-( )MOE) modification. In some embodiments, the chemical modification of the at least one chemically modified nucleotide is a Phosphorothioate (PS) backbone modification. In some embodiments, the chemical modification of the at least one chemically modified nucleotide is an addition of a 5′-phosophate moiety, an (E)-vinylphosphonate moiety, or a 5-methyl cytosine moiety at the 5′ end of the nucleotide sequence. In some embodiments, the functional oligonucleotides comprise one or more of the above modifications in one or more of the nucleotides (such as from one to up to all the nucleotides modification).

In some embodiments, the ASO in the agent is covalently linked to the adjacent dsRNA or ASO in a 3′ to 5′ orientation or in a 5′ to 3′ orientation. In some embodiments, the dsRNA in the agent is covalently linked to the ASO or dsRNA at its 3′ end of the sense or antisense strand; or at its 5′ end of the sense or antisense strand.

In some embodiments, the dsRNA is a siRNA or a saRNA, and the ASO is a gapmer and a mixmer. In some embodiments, the multi-valent oligonucleotide agent comprises functional oligonucleotides selected from: a) siRNA-gapmer-gapmer; b) saRNA-gapmer-gapmer; c) siRNA-gapmer-mixmer; d) saRNA-gapmer-mixmer; e) siRNA-mixmer-mixmer; f) saRNA-mixmer-mixmer, wherein in any one of a)˜f), the functional oligonucleotides are arranged in any order and covalently linked with or without linking component(s). In some embodiments, the ASO targets S′-UTR.

In some aspects, the dsRNA and/or the ASO(s) increase the expression of a SMN2 gene or protein. In some embodiments, the dsRNA(s) increases the expression of the SMN2 gene or protein; and/or the ASO(s) increases the production of functional SMN protein by modulating SMN2 mRNA splicing or stability (SMN2 mRNA modulators).

In some embodiments, the dsRNA comprises a saRNA having a nucleotide sequence of a sense strand that is at least 90% identical to the nucleotide sequence selected from: a) DS06-0004 (SEQ ID NO: 5); b) DS06-0031 (SEQ ID NO: 7); c) DS06-0067 (SEQ ID NO: 9); d) DS06-4A3 (SEQ ID NO: 146); c) R6-04-S1 (SEQ ID NO: 59); and f) R6-04(20)-S1V1v(CM-4) (SEQ ID NO: 60).

In some embodiments, the dsRNA(s) comprises a saRNA having a nucleotide sequence of an antisense strand that is at least 90% identical to the nucleotide sequence selected from: a) DS06-0004 (SEQ ID NO: 6); b) DS06-0031 (SEQ ID NO: 8); c) DS06-0067 (SEQ ID NO: 10); d) DS06-4A3 (SEQ ID NO: 147); e) R6-04-S1 (SEQ ID NO: 53); and f) R6-04M1-27A-S1L1V3(CM-26) (SEQ ID NO: 17).

In some embodiments, the dsRNA comprises a saRNA having a pair of nucleotide sequences of a sense strand and an antisense strand that is at least 90% identical to the nucleotide sequence pairs selected from: a) DS06-0004: SEQ ID NO: 5 and SEQ ID NO: 6; b) DS06-0031: SEQ ID NO: 7 and SEQ ID NO: 8; c) DS06-0067: SEQ ID NO: 9 and SEQ ID NO: 10; d) DS06-4A3: SEQ ID NO: 146 and SEQ ID NO: 147; e) R6-04-S1: SEQ ID NO: 59 and SEQ ID NO: 53; f) R6-04(20)-S1V1v(CM-4): SEQ ID NO: 60 and SEQ ID NO: 17.

In some embodiments, the dsRNA comprises a siRNA having a nucleotide sequence of a sense strand that is at least 90% identical to the nucleotide sequence of DS06-332i (SEQ ID NO: 3) or siSOD1-388-ESC (SEQ ID NO: 138). In some embodiments, the dsRNA comprises a siRNA having a nucleotide sequence of an antisense strand that is at least 90% identical to the nucleotide sequence of DS06-332i (SEQ ID NO: 4) or siSOD1-388-ESC (SEQ ID NO: 139). In some embodiments, the dsRNA comprises a siRNA having a pair of nucleotide sequences of a sense strand and an antisense strand that is at least 90% identical to the nucleotide sequence pairs selected from: DS06-332i: SEQ ID NO: 3 and SEQ ID NO: 4; siSOD1-388-ESC: SEQ ID NO: 138 and SEQ ID NO: 139.

In some embodiments, the linking components and/or linkage bonds and/or orientation of the above mentioned multi-valent oligonucleotide agents are changeable.

In some aspects, one or more of the functional oligonucleotides increase the expression of CDKN1A/p21 gene or protein and/or decreases the expression CD274/PDL-1. In some embodiments, the dsRNA is a saRNA that increases the expression of the CDKN1A/p21 gene or protein; and/or a siRNA that decreases the expression CD274/PDL-1. In some embodiments, the ASO is an ASO that increases the expression of CDKN1A/p21 gene or protein and/or decreases the expression CD274/PDL-1.

In some embodiments, the dsRNA is a saRNA having a nucleotide sequence of a sense strand that is at least 90% identical to the nucleotide sequence of RAG1-40 (SEQ ID NO: 62). In some embodiments, the dsRNA is a saRNA having a nucleotide sequence of an antisense strand that is at least 90% identical to the nucleotide sequence of RAG1-40 (SEQ ID NO: 63). In some embodiments, the dsRNA is a saRNA having a nucleotide sequence of a sense strand that is at least 90% identical to the nucleotide sequence of RAG1-40 (SEQ ID NO: 62) and a nucleotide sequence of an antisense strand that is at least 90% identical to the nucleotide sequence of RAG1-40 (SEQ ID NO: 63).

In some embodiments, the dsRNA is a siRNA having a nucleotide sequence of a sense strand that is at least 90% identical to the nucleotide sequence selected from: a) siPDL1-2 (SEQ ID NO: 64); and b) siPDL1-3 (SEQ ID NO: 66). In some embodiments, the dsRNA is a siRNA having a nucleotide sequence of an antisense strand that is at least 90% identical to the nucleotide sequence selected from: a) siPDL1-2 (SEQ ID NO: 65); and b) siPDL1-3 (SEQ ID NO: 67). In some embodiments, the dsRNA is a siRNA selected from: a) a siRNA having a nucleotide sequence of a sense strand that is at least 90% identical to the nucleotide sequence siPDL1-2 (SEQ ID NO: 64) and a nucleotide sequence of an antisense strand that is at least 90% identical to the nucleotide sequence of siPDL1-2 (SEQ ID NO: 65); and b) a siRNA having a nucleotide sequence of a sense strand that is at least 90% identical to the nucleotide sequence siPDL1-2 (SEQ ID NO: 66) and a nucleotide sequence of an antisense strand that is at least 90% identical to the nucleotide sequence of siPDL1-2 (SEQ ID NO: 67).

In some embodiments, the ASO has a nucleotide sequence that is at least 90% identical to the nucleotide sequence selected from: a) aPDL1-1 (SEQ ID NO: 68); b) aPDL1-2 (SEQ ID NO: 69); and c) aPDL1-3 (SEQ ID NO: 70).

In some embodiments, the linking components and/or linkage bonds and/or orientation of those multi-valent oligonucleotide agents are changeable.

In some embodiments, the tri-valent oligonucleotide agent can be those produced by adding one dsRNA or ASO to a bi-valent oligonucleotide agent, such as the bi-valent oligonucleotide agents disclosed in 4.2.5.1 or 4.2.5.3.

4.2.5.6 Tri-Valent Oligonucleotide Agent comprising 3 dsRNAs

In some aspects, disclosed herein is tri-valent oligonucleotide agent comprising three functional oligonucleotides: a first double stranded RNA (dsRNA), a second dsRNA and a third dsRNA. In such a tri-valent oligonucleotide agent, the dsRNAs may be arranged in any order, such as dsRNA1-dsRNA2-dsRNA3, dsRNA1-dsRNA3-dsRNA2, dsRNA2-dsRNA1-dsRNA3, dsRNA2-dsRNA3-dsRNA1, dsRNA3-dsRNA1-dsRNA2, dsRNA3-dsRNA2-dsRNA1. In some embodiments, the three functional oligonucleotides may be covalently connected via a linker or a bond.

In some embodiments, the dsRNA is selected from a small interfering RNA (siRNA) or a small activating RNA (saRNA), and the three functional oligonucleotides independently modulate the expression of one or more genes, modulate the expression of one or more proteins (such as by binding to a mRNA sequence), or modulate non-coding regulatory nucleic acid sequences (such as a promoter sequence, enhancer, silencer, and/or transcription factor).

In some embodiments, each dsRNA comprises a sense strand that is at least 10 contiguous nucleotides and an antisense strand that is at least 10 contiguous nucleotides. In some embodiments, each dsRNA comprises a sense strand that is of 10-60 nucleotides in length and/or an antisense strand that is of 10-60 nucleotides in length. In some embodiments, the tri-valent oligonucleotide agent has a total length ranging from 30 to 250 nucleotides.

In some embodiments, the two adjacent functional oligonucleotides are covalently linked by a linking component or with no linking component. The linking component may selected from Spacer-9, Spacer-18, Spacer-C3 and Spacer-C6 or derivatives thereof, or any suitable linking components as disclosed in the present Specification or known in the art. In some embodiments, the two adjacent functional oligonucleotides are covalently linked by

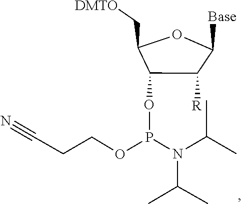

wherein R represents —H or —OH or —OMe, or -MOE, or —F, or other 2′ chemical modifications. In some embodiments, the two adjacent functional oligonucleotides are covalently linked by a phosphodiester bond or a phosphorothioate bond or by one or more nucleotides.

In some embodiments, the functional oligonucleotides comprise at least one chemically modified nucleotide. The modification to the chemically modified nucleotide may be is a 2′ sugar modification selected from one or more of: 2′-fluoro-2′-deoxynucleoside (2′-F) modification, 2′-O-methyl (2′-O—Me), modification, and 2′-O-(2-methoxyethyl) (2′-O-MOE) modification. In some embodiments, the chemical modification of the at least one chemically modified nucleotide is a Phosphorothioate (PS) backbone modification. In some embodiments, the chemical modification of the at least one chemically modified nucleotide is an addition of a 5′-phosophate moiety, an (E)-vinylphosphonate moiety, or a 5-methyl cytosine moiety at the 5′ end of the nucleotide sequence. In some embodiments, the functional oligonucleotides comprise one or more of the above modifications in one or more of the nucleotides (such as from one to up to all the nucleotides modification).

In some embodiments, the dsRNAs in the agent are covalently linked in a 3′ to 5′ orientation or in a 5′ to 3′ orientation. In some embodiments, one dsRNA in the agent is covalently linked to the other dsRNA at its 3′ end of the sense or antisense strand; or at its 5′ end of the sense or antisense strand.

In some embodiments, the dsRNA is a siRNA or a saRNA. In some embodiments, the multi-valent oligonucleotide agent comprises functional oligonucleotides selected from: (a) siRNA-siRNA-siRNA; b) siRNA-siRNA-saRNA; c) siRNA-saRNA-saRNA; d) saRNA-saRNA-saRNA, wherein in any one of (a)˜ (d), the functional oligonucleotides are arranged in any order and covalently linked with or without linking component(s). In some embodiments, two or three functional oligonucleotides in the same agent can be identical or different.

In some aspects, the dsRNA(s) increase/inhibit the expression of a SMN2 gene or protein.

In some embodiments, the dsRNA comprises a saRNA having a nucleotide sequence of a sense strand that is at least 90% identical to the nucleotide sequence selected from: a) DS06-0004 (SEQ ID NO: 5); b) DS06-0031 (SEQ ID NO: 7); c) DS06-0067 (SEQ ID NO: 9); d) DS06-4A3 (SEQ ID NO: 146); e) R6-04-S1 (SEQ ID NO: 59); and f) R6-04(20)-S1V1v(CM-4) (SEQ ID NO: 60).

In some embodiments, the dsRNA(s) comprises a saRNA having a nucleotide sequence of an antisense strand that is at least 90% identical to the nucleotide sequence selected from: a) DS06-0004 (SEQ ID NO: 6); b) DS06-0031 (SEQ ID NO: 8); c) DS06-0067 (SEQ ID NO: 10); d) DS06-4A3 (SEQ ID NO: 147); e) R6-04-S1 (SEQ ID NO: 53); and f) R6-04M1-27A-S1L1V3(CM-26) (SEQ ID NO: 17).

In some embodiments, the dsRNA comprises a saRNA having a pair of nucleotide sequences of a sense strand and an antisense strand that is at least 90% identical to the nucleotide sequence pairs selected from: a) DS06-0004: SEQ ID NO: 5 and SEQ ID NO: 6; b) DS06-0031: SEQ ID NO: 7 and SEQ ID NO: 8; c) DS06-0067: SEQ ID NO: 9 and SEQ ID NO: 10; d) DS06-4A3: SEQ ID NO: 146 and SEQ ID NO: 147; e) R6-04-S1: SEQ ID NO: 59 and SEQ ID NO: 53; f) R6-04(20)-S1V1v(CM-4): SEQ ID NO: 60 and SEQ ID NO: 17.

In some embodiments, the dsRNA comprises a siRNA having a nucleotide sequence of a sense strand that is at least 90% identical to the nucleotide sequence of DS06-332i (SEQ ID NO: 3) or siSOD1-388-ESC (SEQ ID NO: 138). In some embodiments, the dsRNA comprises a siRNA having a nucleotide sequence of an antisense strand that is at least 90% identical to the nucleotide sequence of DS06-332i (SEQ ID) NO: 4) or siSOD1-388-ESC (SEQ ID NO: 139). In some embodiments, the dsRNA comprises a siRNA having a pair of nucleotide sequences of a sense strand and an antisense strand that is at least 90% identical to the nucleotide sequence pairs selected from: DS06-332i: SEQ ID NO: 3 and SEQ ID NO: 4; siSOD1-388-ESC: SEQ ID NO: 138 and SEQ ID NO: 139.

In some aspects, one or more of the functional oligonucleotides increase the expression of CDKN1A/p21 gene or protein and/or decreases the expression CD274/PDL-1. In some embodiments, the dsRNA is a saRNA that increases the expression of the CDKN1A/p21 gene or protein; and/or a siRNA that decreases the expression CD274/PDL-1.

In some embodiments, the dsRNA is a saRNA having a nucleotide sequence of a sense strand that is at least 90% identical to the nucleotide sequence of RAG1-40 (SEQ ID NO: 62). In some embodiments, the dsRNA is a saRNA having a nucleotide sequence of an antisense strand that is at least 90% identical to the nucleotide sequence of RAG1-40 (SEQ ID NO: 63). In some embodiments, the dsRNA is a saRNA having a nucleotide sequence of a sense strand that is at least 90% identical to the nucleotide sequence of RAG1-40 (SEQ ID NO: 62) and a nucleotide sequence of an antisense strand that is at least 90% identical to the nucleotide sequence of RAG1-40 (SEQ ID NO: 63).

In some embodiments, the dsRNA is a siRNA having a nucleotide sequence of a sense strand that is at least 90% identical to the nucleotide sequence selected from: a) siPDL1-2 (SEQ ID NO: 64); and b) siPDL1-3 (SEQ ID NO: 66). In some embodiments, the dsRNA is a siRNA having a nucleotide sequence of an antisense strand that is at least 90% identical to the nucleotide sequence selected from: a) siPDL1-2 (SEQ ID NO: 65); and b) siPDL1-3 (SEQ ID NO: 67). In some embodiments, the dsRNA is a siRNA selected from: a) a siRNA having a nucleotide sequence of a sense strand that is at least 90% identical to the nucleotide sequence siPDL1-2 (SEQ ID NO: 64) and a nucleotide sequence of an antisense strand that is at least 90% identical to the nucleotide sequence of siPDL1-2 (SEQ ID NO: 65); and b) a siRNA having a nucleotide sequence of a sense strand that is at least 90% identical to the nucleotide sequence siPDL1-2 (SEQ ID NO: 66) and a nucleotide sequence of an antisense strand that is at least 90% identical to the nucleotide sequence of siPDL1-2 (SEQ ID NO: 67).

In some embodiments, the linking components and/or linkage bonds and/or orientation of those multi-valent oligonucleotide agents are changeable. In some embodiments, the tri-valent oligonucleotide agent can be those produced by adding one dsRNA to a bi-valent oligonucleotide agent, such as the bi-valent oligonucleotide agents disclosed in 4.2.5.2.

4.2.5.7 Tri-Valent Oligonucleotide Agent comprising 3 ASOs

In some aspects, disclosed herein is tri-valent oligonucleotide agent comprising three functional oligonucleotides: a first antisense oligonucleotide (ASO), a second ASO and a third ASO. In such a tri-valent oligonucleotide agent, the ASOs may be arranged in any order, such as ASO1-ASO2-ASO3, ASO1-ASO3-AS02, ASO2-ASO1-ASO3, ASO2-ASO3-ASO1, ASO3-ASO1-ASO2, ASO3-ASO2-ASO1. Two or three of the ASOs may be identical or different.

In some embodiments, the ASO is independently selected from a gapmer or a mixmer. In some embodiments, the three functional oligonucleotides independently modulate the expression of one or more genes, modulate the expression of one or more proteins (such as by binding to a mRNA sequence), or modulate non-coding regulatory nucleic acid sequences (such as a promoter sequence, enhancer, silencer, and/or transcription factor).

In some embodiments, each ASO has a nucleotide sequence that is at least 5 contiguous nucleotides in length. In some embodiments, the ASO has a nucleotide sequence that is 5-30 nucleotides in length. In some embodiments, the tri-valent oligonucleotide agent has a total length ranging from 15 to 150 nucleotides.

In some embodiments, the two adjacent functional oligonucleotides are covalently linked by a linking component or with no linking component. The linking component may selected from Spacer-9, Spacer-18, Spacer-C3 and Spacer-C6 or derivatives thereof, or any suitable linking components as disclosed in the present Specification or known in the art. In some embodiments, the two adjacent functional oligonucleotides are covalently linked by

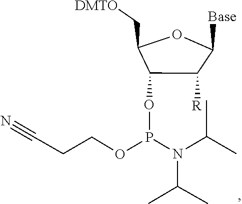

wherein R represents —H or —OH or —OMe, or -MOE, or —F, or other 2′ chemical modifications. In some embodiments, the two adjacent functional oligonucleotides are covalently linked by a phosphodiester bond or a phosphorothioate bond or by one or more nucleotides.

In some embodiments, the functional oligonucleotides comprise at least one chemically modified nucleotide. The modification to the chemically modified nucleotide may be is a 2′ sugar modification selected from one or more of: 2′-fluoro-2′-deoxynucleoside (2′-F) modification, 2′-O-methyl (2′-O—Me), modification, and 2′-( )(2-methoxyethyl) (2′-O)-MOE) modification. In some embodiments, the chemical modification of the at least one chemically modified nucleotide is a Phosphorothioate (PS) backbone modification. In some embodiments, the chemical modification of the at least one chemically modified nucleotide is an addition of a 5′-phosophate moiety, an (E)-vinylphosphonate moiety, or a 5-methyl cytosine moiety at the 5′ end of the nucleotide sequence. In some embodiments, the functional oligonucleotides comprise one or more of the above modifications in one or more of the nucleotides (such as from one to up to all the nucleotides modification).

In some embodiments, one ASO in the agent is covalently linked to another ASO in a 3′ to 5′ orientation or in a 5′ to 3′ orientation. In some embodiments, one ASO in the agent is covalently linked to another ASO at its 3′ end; or at its 5′ end.

In some embodiments, the ASO is selecte from a gapmer and a mixmer. In some embodiments, the multi-valent oligonucleotide agent comprises functional oligonucleotides selected from: a) gapmer-gapmer-gapmer; b) gapmer-gapmer-mixmer; c) gapmer-mixmer-mixmer; d) mixmer-mixmer-mixmer, wherein in any one of (a)˜ (d), the functional oligonucleotides are arranged in any order and covalently linked with or without linking component(s). In some embodiments, the ASO targets 5′-UTR.

In some aspects, the ASO increase the expression of a SMN2 gene or protein. In some embodiments, the ASO increases the production of functional SMN protein by modulating SMN2 mRNA splicing or stability (SMN2 mRNA modulators).

In some embodiments, the ASO has a nucleotide sequence that is at least 90% identical to the nucleotide sequence of ASO10-27 (SEQ ID NO: 11) or 5′UTR ASO (SEQ ID NO: 142).

In some embodiments, the linking components and/or linkage bonds and/or orientation of those multi-valent oligonucleotide agents are changeable. In some embodiments, the tri-valent oligonucleotide agent can be those produced by adding one dsRNA to a bi-valent oligonucleotide agent, such as the bi-valent oligonucleotide agents disclosed in 4.2.5.3.

4.2.5.8 Multi-Valent Oligonucleotide Agent comprising more than 3 functional oligonucleotides

In some aspects, disclosed herein are multi-valent oligonucleotide agent comprising more than 3 functional oligonucleotides that are covalently linked, wherein the four or more functional oligonucleotides are independently selected from: a) a double stranded RNA (dsRNA); and b) an antisense oligonucleotide (ASO). In such a multi-valent oligonucleotide agent, the dsRNA(s) and the ASO(s) may be arranged in any order.

In some embodiments, the number of the functional oligonucleotides comprised in the multi-valent oligonucleotide agent is ranged from 4 to X, wherein X is an integer ranged from 5 to 10. In some embodiments, the number of dsRNA comprised in the agent is from 0 to X, with the rest functional oligonucleotides being ASO(s).

In some embodiments, the dsRNA is selected from a small interfering RNA (siRNA) or a small activating RNA (saRNA), and the ASO is a gapmer or a mixmer. In some embodiments, the two functional oligonucleotides independently modulate the expression of one or more genes, modulate the expression of one or more proteins (such as by binding to a mRNA sequence), or modulate non-coding regulatory nucleic acid sequences (such as a promoter sequence, enhancer, silencer, and/or transcription factor).

In some embodiments, each dsRNA comprises a sense strand that is at least 10 contiguous nucleotides and an antisense strand that is at least 10 contiguous nucleotides. In some embodiments, each dsRNA comprises a sense strand that is of 10-60 nucleotides in length and/or an antisense strand that is of 10-60 nucleotides in length. In some embodiments, each ASO has a nucleotide sequence that is at least 5 contiguous nucleotides in length. In some embodiments, each ASO has a nucleotide sequence that is 5-30 nucleotides in length. In some embodiments, the multi-valent oligonucleotide agent has a total length ranging from 20 to 200 nucleotides.

In some embodiments, two adjacent functional oligonucleotides are covalently linked by a linking component or with no linking component. The linking component may selected from Spacer-9, Spacer-18, Spacer-C3 and Spacer-C6 or derivatives thereof, or any suitable linking components as disclosed in the present Specification or known in the art. In some embodiments, the two adjacent functional oligonucleotides are covalently linked by, wherein R represents —H or —OH or —OMe, or -MOE, or —F, or other 2′ chemical modifications. In some embodiments, the two adjacent functional oligonucleotides are covalently linked by a phosphodiester bond or a phosphorothioate bond or by one or more nucleotides.

In some embodiments, the functional oligonucleotides comprise at least one chemically modified nucleotide. The modification to the chemically modified nucleotide may be is a 2′ sugar modification selected from one or more of: 2′-fluoro-2′-deoxynucleoside (2′-F) modification, 2′-O-methyl (2′-O—Me), modification, and 2′-( )(2-methoxyethyl) (2′-( )MOE) modification. In some embodiments, the chemical modification of the at least one chemically modified nucleotide is a Phosphorothioate (PS) backbone modification. In some embodiments, the chemical modification of the at least one chemically modified nucleotide is an addition of a 5′-phosophate moiety, an (E)-vinylphosphonate moiety, or a 5-methyl cytosine moiety at the 5′ end of the nucleotide sequence. In some embodiments, the functional oligonucleotides comprise one or more of the above modifications in one or more of the nucleotides (such as from one to up to all the nucleotides modification).

In some embodiments, the sequences of two or more functional oligonucleotides in the multi-valent nucleotide agent are identical or different; and/or the functions of the two or more functional oligonucleotides are identical or different.

In some embodiments, the linking components and/or linkage bonds and/or orientation of those multi-valent oligonucleotide agents are changeable. In some embodiments, the multi-valent oligonucleotide agent can be those produced by adding one or more dsRNAs and/or ASO(s) (such as one or more disclosed herein) to a bi-valent oligonucleotide agent (such as the bi-valent oligonucleotide agents disclosed in 4.2.5.1-4.2.5.3) or to a tri-valent oligonucleotide agent (such as the tri-valent oligonucleotide agents disclosed in 4.2.5.4˜4.2.5.7).

### 4.3 Exemplary Functions of the Multi-Valent Oligonucleotide Agents

Also disclosed herein are multi-valent oligonucleotide agents of various functions. In some embodiments, depending on the functional oligonucleotides in the multi-valent oligonucleotide agents, the agents may be used to target desired gene(s) which are assocated to particular diseases so as to produce therapeutic effects. Accordingly, methods for treatment of diseases using the multi-valent oligonucleotide agents and products comprising multi-valent oligonucleotide agents for disease treatment are also provided.

The multi-valent oligonucleotide agents can be used to adjust and/or regulate the expression and/or activity of target(s) of interest (which may be associated with certain diseases). Based on the disclosure and spirit of the present disclosure, one may design suitable multi-valent oligonucleotide agents specically binding, adjusting and/or regulating the expression and/or activity of target(s) of interest and may assess the effects of the multi-valent oligonucleotide agents via conventional experiments.

The following are some exemplary aspects and embodiments of the invention. Such aspects and embodiments do not necessarily represent the full scope of the invention, however, and reference is made therefore to the claims and herein for interpreting the scope of the invention.

4.3.1 multi-valent oligonucleotide agents that increase the expression of the SMN2 gene or protein

In some embodiments, the two or more functional oligonucleotides increase the expression of an SMN2 gene or protein. Administration of the multi-valent oligonucleotide agent to a patient treats or delays the onset of an SMN-deficiency-related condition, such as spinal muscular atrophy. In certain embodiments, the described multi-valent oligonucleotide agent increases the amount of a full-length SMN protein by, for example, activating/up-regulating SMN2 transcription in conjunction with modulating splicing for exon 7 inclusion to increase the amount of full-length SMN2 mRNA. In certain embodiments, full-length SMN protein is increased in an amount sufficient to reduce the symptoms associated with an SMN-deficiency-related condition. In certain embodiments, full-length SMN protein is increased by at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%).

In certain embodiments, at least one of the two or more functional oligonucleotides of the multi-valent oligonucleotide agent that increases the expression of the SMN2 gene or protein is an saRNA. The SMN2 saRNA activates or upregulates the expression of an SMN2 gene in a cell in which the SMN2 gene is normally expressed.

In typical embodiments, a first strand of the SMN2 saRNA comprises a segment that has at least 75% sequence identity or sequence complementarity to a 16-35 nucleotide fragment of the promoter region of the SMN2 gene thereby effecting activation or upregulation of expression of the gene.

In particular, the first strand of the SMN2 saRNA has homology or complementarity with a region of the SMN2 gene promoter from a region of the SMN2 gene promoter from −1639 to −1481 (SEQ ID NO: 124), a region of the SMN2 gene promoter from −1090 to −1008 (SEQ ID NO: 125), a region of the SMN2 gene promoter from −994 to −180 regions (SEQ ID NO: 126, or a region of the SMN2 gene promoter from −144 to −37 (SEQ ID NO: 127), and have a homology or complementarity of at least 75%, such as at least about 79%, about 80%, about 85%, about 90%, about 95%, or about 99%. More specifically, one strand of the SMN2 saRNA has at least 75%, e.g., at least about 79%, or about 99% homology or complementarity with any nucleotide sequence selected from the group consisting of SEQ ID NO: 315-471.

In the present disclosure, the SMN2 saRNA comprises a sense nucleic acid fragment and an antisense nucleic acid fragment. The sense nucleic acid fragment and the antisense nucleic acid fragment comprise complementary regions capable of forming a double-stranded nucleic acid structure that facilitates expression of the SMN2 gene in a cell by the RNA activation mechanism. Sense nucleic acid fragments and antisense nucleic acid fragments of saRNAs may be present on two different nucleic acid strands or may be present on the same nucleic acid strand. When the sense and antisense nucleic acid fragments are present on two strands at least one strand of the saRNA has a 3′ overhang of 0-6 nucleotides in length, preferably both strands have a 3′ overhang of 2 or 3 nucleotides in length, and preferably the nucleotides of the overhang are deoxythymine (dT). When a sense nucleic acid fragment and an antisense nucleic acid fragment of an saRNA are present on the same nucleic acid strand, preferably the saRNA is a single-stranded hairpin-structured nucleic acid molecule, wherein the complementary regions of the sense nucleic acid fragment and the antisense nucleic acid fragment form a double-stranded nucleic acid structure. In such an saRNA, the sense nucleic acid fragment and antisense nucleic acid fragment are 10-60 nucleotides in length and may be 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides.

In certain embodiments of the present disclosure, the SMN2 saRNA comprises a sense nucleic acid strand and an antisense nucleic acid strand, the sense nucleic acid strand comprising at least one region that is complementary to at least one region on the antisense nucleic acid strand to form a double-stranded nucleic acid structure capable of activating expression of the SMN2 gene in a cell.

In certain embodiments of the present disclosure, the sense nucleic acid strand and the antisense nucleic acid strand are located on two different nucleic acid strands.

In certain embodiments of the present disclosure, the sense nucleic acid fragment and the antisense nucleic acid fragment are located on the same nucleic acid strand, forming a hairpin single-stranded nucleic acid molecule, wherein the complementary regions of the sense nucleic acid fragment and the antisense nucleic acid fragment form a double-stranded nucleic acid structure.

In certain embodiments of the present disclosure, at least one of the nucleic acid strands has a 3′overhang of 0 to 6 nucleotides in length. In certain embodiments of the present disclosure, both of the nucleic acid strands have 3′overhangs of 2-3 nucleotides in length. In certain embodiments of the present disclosure, the sense and antisense nucleic acid strands are 16 to 35 nucleotides in length, respectively.

In some embodiments, a first, second, and/or third dsRNA increases the expression of the SMN2 gene or protein and the ASO increases the production of functional SMN protein by modulating SMN2 mRNA splicing or stability (SMN2 mRNA modulators).

The term “SMN2 mRNA modulator” as used herein refers to a modulator of SMN2 mRNA splicing or stability that increases the production of functional SMN2 mRNA and functional SMN protein. The term “SMN2 mRNA modulator” includes an agent that changes the way the SMN2 pre-mRNA is spliced so that it contains all the information necessary to make functional full-length SMN protein, for example, by blocking the effect of the intronic inhibitory splicing region of intron 7 of the SMN2 gene. An SMN2 mRNA modulator includes those agents that increase the desired splicing and subsequent protein production by stabilizing the interaction between the spliceosome and SMN2 pre-mRNA (J Med Chem, 2018 Dec. 27; 61(24): 11021-11036), and those agents that enhance stabilization of the transient double-strand RNA structure formed by the SMN2 pre-mRNA and Ul small nuclear ribonucleic protein (snRNP) complex (Nat Chem Biol, 2015 July;11(7):511-7). In certain examples, an SMN2 mRNA modulator will modulate the splicing of SMN2 pre-mRNA to include exon 7 in the processed transcript. Alternatively, SMN2 mRNA modulators of the present disclosure include agents which possess the ability to increase functional SMN protein levels by preventing exon7 from being spliced out of the mature SMN mRNA during splicing. The SMN2 mRNA modulator in accordance with the present disclosure also includes those described in U.S. Pat. Nos. 10,436,802 and 10,420,753, the entirety of each of which are incorporated herein by reference.

Examples of SMN2 mRNA modulators in accordance with the present disclosure include pyridazine derivatives, for example those described in WO2014028459A1, the entire contents of which are incorporated herein by reference. Specific examples of SMN2 mRNA modulators in include Branaplam (also known as LMI070) and Risdiplam (also known as RG7916, or RO7034067).

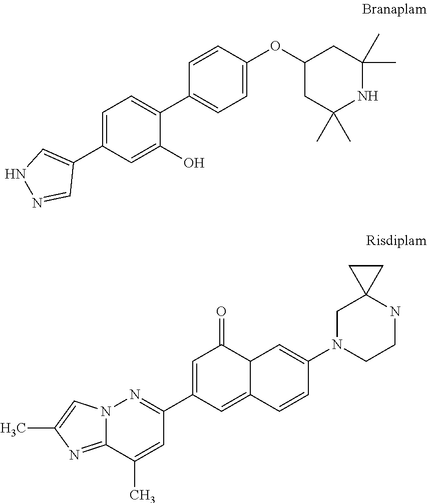

Further examples of SMN2 mRNA modulators in accordance with the present disclosure include antisense oligonucleotides such as those capable of antisense targeting, displacement and/or disruption of an intronic sequence in the SMN2 gene to enhance the production of SMN2 full-length (SMN2FL) transcripts (transcripts containing exon 7) during splicing. In certain embodiments, Nusinersen, marketed as SPINRAZA®, is suitable for use in accordance with the disclosed combinations.

In some embodiments, the dsRNA is a small activating RNA (saRNA). saRNA targets regulatory sequences in the nucleus such as gene promoters to upregulate gene expression at the transcriptional level via the RNAa (RNA activation) mechanism. For example, small activating ribonucleic acids (saRNAs) that activate or upregulate the expression of an SMN2 gene (also referred to as “SMN2 saRNAs” herein) in a cell, and may be covalently linked to one or more modulators of SMN2 mRNA splicing or stability (also referred to as “SMN2 mRNA modulators” herein) that increase the production of functional SMN2 mRNA, to achieve a significant increase in the level of full-length SMN2 mRNA and full-length SMN protein. Covalently linking multi-valent oligonucleotides as provided herein can provide enhanced therapeutic benefit compared to monotherapy and can thus maximize treatment outcomes, e.g., for SMA patients.

In some aspects, the present disclosure provides an isolated SMN2 gene saRNA targeting site having any contiguous 16-35 nucleotide sequence on the promoter region (UCSC Genome Browser coordinates: chr5:70,044,612-70,049,522) of the SMN2 gene (NCBI GeneID: 6607).

In some embodiments, an saRNA can include a nucleotide sequence of a sense strand that is at least 60% (e.g., at least 65%, at least 70%, at least 75%, at least 80%, at least 85%, at least 90%, at least 95%, at least 97%, at least 99% or 100%) identical to the nucleotide sequence selected from: DS06-0004 (SEQ ID NO: 5); b) DS06-0031 (SEQ ID NO: 7); c) DS06-0067 (SEQ ID NO: 9); d) DS06-4A3 (SEQ ID NO: 146); e) R6-04-S1 (SEQ ID) NO: 59); and f) R6-04(20)-S1V1v(CM-4) (SEQ ID NO: 60). In some embodiments, an saRNA can include a nucleotide sequence of a sense strand that is at least 60% (e.g., at least 65%, at least 70%, at least 75%, at least 80%, at least 85%, at least 90%, at least 95%, at least 97%, at least 99% or 100%) identical to the nucleotide sequence of DS06-0004 (SEQ ID NO: 5). In some embodiments, an saRNA can include a nucleotide sequence of a sense strand that is at least 60% (e.g., at least 65%, at least 70%, at least 75%, at least 80%, at least 85%, at least 90%, at least 95%, at least 97%, at least 99% or 100%) identical to the nucleotide sequence of DS06-0031 (SEQ ID NO: 7. In some embodiments, an saRNA can include a nucleotide sequence of a sense strand that is at least 60% (e.g., at least 65%, at least 70%, at least 75%, at least 80%, at least 85%, at least 90%, at least 95%, at least 97%, at least 99% or 100%) identical to the nucleotide sequence of DS06-0067 (SEQ ID NO: 9). In some embodiments, an saRNA can include a nucleotide sequence of a sense strand that is at least 60% (e.g., at least 65%, at least 70%, at least 75%, at least 80%, at least 85%, at least 90%, at least 95%, at least 97%, at least 99% or 100%) identical to the nucleotide sequence of DS06-4A3 (SEQ ID NO: 12. In some embodiments, an saRNA can include a nucleotide sequence of a sense strand that is at least 60% (e.g., at least 65%, at least 70%, at least 75%, at least 80%, at least 85%, at least 90%, at least 95%, at least 97%, at least 99% or 100%) identical to the nucleotide sequence of R6-04-S1 (SEQ ID NO: 59. In some embodiments, an saRNA can include a nucleotide sequence of a sense strand that is at least 60% (e.g., at least 65%, at least 70%, at least 75%, at least 80%, at least 85%, at least 90%, at least 95%, at least 97%, at least 99% or 100%) identical to the nucleotide sequence of R6-04(20)-S1V1v(CM-4) (SEQ ID NO: 60).

In some embodiments, an saRNA can include a nucleotide sequence of an antisense strand that is at least 60% (e.g., at least 65%, at least 70%, at least 75%, at least 80%, at least 85%, at least 90%, at least 95%, at least 97%, at least 99% or 100%) identical to the nucleotide sequence selected from: a) DS06-0004 (SEQ ID NO: 6); b) DS06-0031 (SEQ ID NO: 8); c) DS06-0067 (SEQ ID NO: 10); d) DS06-4A3 (SEQ ID NO: 147); e) R6-04-S1 (SEQ ID NO: 53); and f) R6-04M1-27A-SIL1V3(CM-26) (SEQ ID NO: 17). In certain embodiments, an saRNA can include a nucleotide sequence of a antisense strand that is at least 60% (e.g., at least 65%, at least 70%, at least 75%, at least 80%, at least 85%, at least 90%, at least 95%, at least 97%, at least 99% or 100%) identical to the nucleotide sequence of a) DS06-0004 (SEQ ID NO: 6). In certain embodiments, an saRNA can include a nucleotide sequence of a antisense strand that is at least 60% (e.g., at least 65%, at least 70%, at least 75%, at least 80%, at least 85%, at least 90%, at least 95%, at least 97%, at least 99% or 100%) identical to the nucleotide sequence of DS06-0031 (SEQ ID NO: 8). In certain embodiments, an saRNA can include a nucleotide sequence of a antisense strand that is at least 60% (e.g., at least 65%, at least 70%, at least 75%, at least 80%, at least 85%, at least 90%, at least 95%, at least 97%, at least 99% or 100%) identical to the nucleotide sequence of DS06-0067 (SEQ ID NO: 10). In certain embodiments, an saRNA can include a nucleotide sequence of an antisense strand that is at least 60% (e.g., at least 65%, at least 70%, at least 75%, at least 80%, at least 85%, at least 90%, at least 95%, at least 97%, at least 99% or 100%) identical to the nucleotide sequence of DS06-4A3 (SEQ ID NO: 147). In certain embodiments, an saRNA can include a nucleotide sequence of a antisense strand that is at least 60% (e.g., at least 65%, at least 70%, at least 75%, at least 80%, at least 85%, at least 90%, at least 95%, at least 97%, at least 99% or 100%) identical to the nucleotide sequence of R6-04-S1 (SEQ ID NO: 53). In certain embodiments, an saRNA can include a nucleotide sequence of an antisense strand that is at least 60% (e.g., at least 65%, at least 70%, at least 75%, at least 80%, at least 85%, at least 90%, at least 95%, at least 97%, at least 99% or 100%) identical to the nucleotide sequence of R6-04M1-27A-S1L1V3(CM-26) (SEQ ID NO: 17).

In some embodiments, the dsRNA is an siRNA. an siRNA can include a nucleotide sequence of a sense strand that is at least 60% (e.g., at least 65%, at least 70%, at least 75%, at least 80%, at least 85%, at least 90%, at least 95%, at least 97%, at least 99% or 100%) identical to the nucleotide sequence selected from: DS06-332i (SEQ ID NO: 3).

In some embodiments, an siRNA can include a nucleotide sequence of an antisense strand that is at least 60% (e.g., at least 65%, at least 70%, at least 75%, at least 80%, at least 85%, at least 90%, at least 95%, at least 97%, at least 99% or 100%) identical to the nucleotide sequence selected from: DS06-332i (SEQ ID NO: 4).

In some embodiments, at least one oligonucleotide is an ASO. An ASO can be designed to target a gene's mRNA to downregulate its expression via the RNase H activity, e.g., for maximizing treatment efficiency of cancers.

In some embodiments, an ASO can include a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequence of ASO10-27 (SEQ ID NO: 11).

In some embodiments, the two or more functional oligonucleotides comprises a first dsRNA and a second dsRNA. In certain embodiments, the first and second dsRNA are each an siRNA.

In some embodiments, the first and second dsRNA are each an saRNA. An saRNA can be designed to target a gene's promoter sequence to induce its transcription via the RNAa mechanism, e.g., for treatment of various cancers.

In some embodiments, the first dsRNA is an saRNA and the second dsRNA is an siRNA. In certain embodiments, the second dsRNA is an siRNA and the second dsRNA is an saRNA.

In some embodiments, the two or more functional oligonucleotides comprises a first ASO and a second ASO.

In some embodiments, the first dsRNA is the siRNA and the second oligonucleotide is the ASO.

In some embodiments, the first dsRNA is the saRNA and the second oligonucleotide is the ASO. In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA06-4A-27A (SEQ ID NO:14) and an antisense saRNA strand having a nucleotide sequence of SEQ ID NO: 13 that has partial complementarity with the sense saRNA strand of SEQ ID NO: 14. In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA06-4A-27B (SEQ ID NO: 15) and an antisense saRNA strand having a nucleotide sequence of SEQ ID NO: 13 that has partial complementarity with the sense saRNA strand of SEQ ID NO: 15. In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-27A-S1L1V3 (SEQ ID NO: 18) and an antisense saRNA strand having a nucleotide sequence of SEQ ID NO: 13 that has partial complementarity with the sense saRNA strand of SEQ ID NO: 18. In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA06-31A-27A (SEQ ID NO: 19) and an antisense saRNA strand having a nucleotide sequence of SEQ ID NO: 8 that has partial complementarity with the sense saRNA strand of SEQ ID NO: 19. In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA06-31B-27A (SEQ ID NO: 20) and a sense saRNA strand having a nucleotide sequence of SEQ ID NO: 7 that has partial complementarity with the antisense saRNA strand of SEQ ID NO: 20.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA06-67A-27A (SEQ ID NO: 21) and an antisense saRNA strand having a nucleotide sequence of SEQ ID NO: 10 that has partial complementarity with the sense saRNA strand of SEQ ID NO: 21;

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA06-67B-27A (SEQ ID NO: 22) and a sense saRNA strand having a nucleotide sequence of SEQ ID NO: 9 that has partial complementarity with the antisense saRNA strand of SEQ ID NO: 22.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-67A3′L0-27A (SEQ ID NO: 23) and an antisense saRNA strand having a nucleotide sequence of SEQ ID NO: 10 that has partial complementarity with the sense saRNA strand of SEQ ID NO: 23.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-67A3′L9-27A (SEQ ID NO: 24) and an antisense saRNA strand having a nucleotide sequence of SEQ ID NO: 10 that has partial complementarity with the sense saRNA strand of SEQ ID NO: 24.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-67A3′L4-27A (SEQ ID NO: 25) and an antisense saRNA strand having a nucleotide sequence of SEQ ID NO: 10 that has partial complementarity with the sense saRNA strand of SEQ ID NO: 25.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-67B3′L0-27A (SEQ ID NO: 26) and a sense saRNA strand having a nucleotide sequence of SEQ ID NO: 9 that has partial complementarity with the antisense saRNA strand of SEQ ID NO: 26.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-67B5′L1-27A (SEQ ID NO: 27) and a sense saRNA strand having a nucleotide sequence of SEQ ID NO: 9 that has partial complementarity with the antisense saRNA strand of SEQ ID NO: 27.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-67B3′L1-27A (SEQ ID NO: 28) and a sense saRNA strand having a nucleotide sequence of SEQ ID NO: 9 that has partial complementarity with the antisense saRNA strand of SEQ ID NO: 28.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-67B5′L9-27A (SEQ ID NO: 29) and a sense saRNA strand having a nucleotide sequence of SEQ ID NO: 9 that has partial complementarity with the antisense saRNA strand of SEQ ID NO: 29.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-67B5′LA-27A (SEQ ID NO: 30) and a sense saRNA strand having a nucleotide sequence of SEQ ID NO: 9 that has partial complementarity with the antisense saRNA strand of SEQ ID NO: 30.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-67B3′L9-27A (SEQ ID NO: 31) and a sense saRNA strand having a nucleotide sequence of SEQ ID NO: 9 that has partial complementarity with the antisense saRNA strand of SEQ ID NO: 31.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-67B3′LA-27A (SEQ ID NO: 32) and a sense saRNA strand having a nucleotide sequence of SEQ ID NO: 9 that has partial complementarity with the antisense saRNA strand of SEQ ID NO: 32.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA06-67A21L1-27A (SEQ ID NO: 33) and an antisense saRNA strand having a nucleotide sequence of SEQ ID NO: 34 that has partial complementarity with the sense saRNA strand of SEQ ID NO: 33.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA06-67B21L1-27A (SEQ ID NO: 36) and sense saRNA strand of SEQ ID NO: 35 that has partial complementarity with the antisense saRNA strand of SEQ ID NO: 36.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-04A3′L0-27A (SEQ ID NO: 37) and an antisense saRNA strand having a nucleotide sequence of SEQ ID NO: 6 that has partial complementarity with the sense saRNA strand of SEQ ID NO: 37.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-04A5′L1-27A (SEQ ID NO: 38) and an antisense saRNA strand having a nucleotide sequence of SEQ ID NO: 6 that has partial complementarity with the sense saRNA strand of SEQ ID NO: 38.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-04A5′L9-27A (SEQ ID NO: 39) and an antisense saRNA strand having a nucleotide sequence of SEQ ID NO: 6 that has partial complementarity with the sense saRNA strand of SEQ ID NO: 39.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-04A5′L4-27A (SEQ ID NO: 40) and an antisense saRNA strand having a nucleotide sequence of SEQ ID NO: 6 that has partial complementarity with the sense saRNA strand of SEQ ID NO: 40.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-04A3′L1-27A (SEQ ID NO: 41) and an antisense saRNA strand having a nucleotide sequence of SEQ ID NO: 6 that has partial complementarity with the sense saRNA strand of SEQ ID NO: 41.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-04A3′L9-27A (SEQ ID NO: 42) and an antisense saRNA strand having a nucleotide sequence of SEQ ID NO: 6 that has partial complementarity with the sense saRNA strand of SEQ ID NO: 42.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-04A3′L4-27A (SEQ ID NO: 43) and an antisense saRNA strand having a nucleotide sequence of SEQ ID NO: 6 that has partial complementarity with the sense saRNA strand of SEQ ID NO: 43.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-04B3′L0-27A (SEQ ID NO: 44) and a sense saRNA strand having a nucleotide sequence of SEQ ID NO: 5 that has partial complementarity with the antisense saRNA strand of SEQ ID NO: 44.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-04B3′L1-27A (SEQ ID NO: 45) and a sense saRNA strand having a nucleotide sequence of SEQ ID NO: 5 that has partial complementarity with the antisense saRNA strand of SEQ ID NO: 45.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-04B3′L9-27A (SEQ ID NO: 46) and a sense saRNA strand having a nucleotide sequence of SEQ ID NO: 5 that has partial complementarity with the antisense saRNA strand of SEQ ID NO: 46.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA6-04B3′LA-27A (SEQ ID NO: 47) and a sense saRNA strand having a nucleotide sequence of SEQ ID NO: 5 that has partial complementarity with the antisense saRNA strand of SEQ ID NO: 47.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA06-04A21L1-27A (SEQ ID NO: 48) and an antisense saRNA strand having a nucleotide sequence of SEQ ID NO: 49 that has partial complementarity with the sense saRNA strand of SEQ ID NO: 48.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of DA06-04B21L1-27A (SEQ ID NO: 51) and a sense saRNA strand of SEQ ID NO: 50 that has partial complementarity with the antisense saRNA strand of SEQ ID NO: 51.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-27A-S1L1V3(CM-26) (SEQ ID NO: 61) and an antisense saRNA strand of SEQ ID NO: 17 that has partial complementarity with the sense saRNA strand of SEQ ID NO: 61.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-16nt-S1L1V3v(CM-27) (SEQ ID NO: 79) and an antisense saRNA strand of SEQ ID NO: 17 that has partial complementarity with the sense saRNA strand of SEQ ID NO: 79.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-15nt-S1L1V3v (SEQ ID NO: 80) and an antisense saRNA strand of SEQ ID NO: 17 that has partial complementarity with the sense saRNA strand of SEQ ID NO: 80.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-14nt-S1L1V3v (SEQ ID NO: 81) and an antisense saRNA strand of SEQ ID NO: 17 that has partial complementarity with the sense saRNA strand of SEQ ID NO: 81.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-13nt-S1L1V3 (SEQ ID NO: 82) and an antisense saRNA strand of SEQ ID NO: 17 that has partial complementarity with the sense saRNA strand of SEQ ID NO: 82.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-(12nt-B)-S1L1V3v (SEQ ID NO: 83) and an antisense saRNA strand of SEQ ID NO: 17 that has partial complementarity with the sense saRNA strand of SEQ ID NO: 83.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-11nt-S1L1V3v (SEQ ID NO: 84) and an antisense saRNA strand of SEQ ID NO: 17 that has partial complementarity with the sense saRNA strand of SEQ ID NO: 84.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-10nt-S1L1V3v (SEQ ID NO: 85) and an antisense saRNA strand of SEQ ID NO: 17 that has partial complementarity with the sense saRNA strand of SEQ ID NO: 85.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-9nt-S1L1V3v (SEQ ID NO: 86) and a sense saRNA strand of SEQ ID NO: 17 that has partial complementarity with the sense saRNA strand of SEQ ID NO: 86.

In certain embodiments, the multi-valent oligonucleotide agent has a nucleotide sequence has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-8nt-S1L1V3v (SEQ ID NO: 87) and a sense saRNA strand of SEQ ID NO: 17 that has partial complementarity with the sense saRNA strand of SEQ ID NO: 87.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-7nt-S1L1V3v (SEQ ID NO: 88) and an antisense saRNA strand of SEQ ID NO: 17 that has partial complementarity with the sense saRNA strand of SEQ ID NO: 88.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-6nt-S1L1V3v (SEQ ID NO: 89) and an antisense saRNA strand of SEQ ID NO: 17 that has partial complementarity with the sense saRNA strand of SEQ ID NO: 89;

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(18)-S1L1V3v (SEQ ID NO: 90) and an antisense saRNA strand of SEQ ID NO: 17 that has partial complementarity with the sense saRNA strand of SEQ ID NO: 90.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(16)-S1L1V3v (SEQ ID NO: 91) and an antisense saRNA strand of SEQ ID NO: 17 that has partial complementarity with the sense saRNA strand of SEQ ID NO: 91.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(15)-S1L1V3v (SEQ ID NO: 92) and an antisense saRNA strand of SEQ ID NO: 17 that has partial complementarity with the sense saRNA strand of SEQ ID NO: 92.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(14)-S1L1V3v (SEQ ID NO: 93) and an antisense saRNA strand of SEQ ID NO: 17 that has partial complementarity with the sense saRNA strand of SEQ ID NO: 93;

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(13)-S1L1V3v (SEQ ID NO: 94) and an antisense saRNA strand of SEQ ID NO: 17 that has partial complementarity with the sense saRNA strand of SEQ ID NO: 94;

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(12)-S1L1V3v (SEQ ID NO: 95) and an antisense saRNA strand of SEQ ID NO: 17 that has partial complementarity with the sense saRNA strand of SEQ ID NO: 95;

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(11)-S1L1V3v (SEQ ID NO: 96) and an antisense saRNA strand of SEQ ID NO: 17 that has partial complementarity with the sense saRNA strand of SEQ ID NO: 96;

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(10)-S1L1V3v (SEQ ID NO: 97) and an antisense saRNA strand of SEQ ID NO: 17 that has partial complementarity with the sense saRNA strand of SEQ ID NO: 97;

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(9)-S1L1V3v (SEQ ID NO: 98) and an antisense saRNA strand of SEQ ID NO: 17 that has partial complementarity with the sense saRNA strand of SEQ ID NO: 98; and

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04M1-AC2(8)-S1L1V3v (SEQ ID NO: 99) and an antisense saRNA strand of SEQ ID NO: 17 that has partial complementarity with the sense saRNA strand of SEQ ID NO: 99.

In some embodiments, the multi-valent agent comprises a third oligonucleotide. In certain embodiments, the third oligonucleotide is selected from an siRNA, an saRNA, and an ASO. In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04S1&27A&67SIR-L1V2 (SEQ ID NO: 54) and an antisense saRNA strand having a nucleotide sequence of SEQ ID NO: 53 that has partial complementarity with the DS06-0004 sense saRNA strand of SEQ ID NO: 54.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04S1&67SIR&27A-L1V2 (SEQ ID NO: 55) and an antisense saRNA strand having a nucleotide sequence of SEQ ID NO: 53 that has partial complementarity with the DS06-0004 sense saRNA strand of SEQ ID NO: 55.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04S1&27A&67S5-L1V2 (SEQ ID NO: 57) and an antisense saRNA strand having a nucleotide sequence of SEQ ID NO: 53 that has partial complementarity with the DS06-0004 sense saRNA strand of SEQ ID NO: 57.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of R6-04S1&67S5&27A-L1V2 (SEQ ID NO: 58) and an antisense saRNA strand having a nucleotide sequence of SEQ ID NO: 53 that has partial complementarity with the DS06-0004 sense saRNA strand of SEQ ID NO: 58.

4.3.2 Agents that increase the expression of the p21 gene or protein and decrease the expression of a PDL-1 gene or protein

Aspects of the present disclosure include multi-valent oligonucleotide agents that increase the expression of the p21 gene or protein and increase the expression of a PDL-1 gene or protein.

CDKN1A (p21) and CD274 (PD-L1, programmed death-ligand 1) are two important genes implicated in tumorigenesis. p21 is a negative cell cycle regulator and a putative tumor suppressor and its activation by saRNA can lead to tumor inhibition. PD-L1 is an important target in cancer treatment and blocking PD-L1 can promote T-cell-mediated immunosurveillance against cancer and has shown huge clinical benefit in cancer patients.

In one aspect, multi-valent oligonucleotide agents of the present disclosure include two or more functional oligonucleotides that are covalently linked to combine tumor inhibitory effects of p21 activation and PD-L1 blockage into a single agent.

In some embodiments, the two or more functional oligonucleotides comprise a first dsRNA and a second dsRNA. In certain embodiments, the first dsRNA is an saRNA that that increases the expression of the CDKN1A/p21 gene or protein.

In certain embodiments, the second dsRNA comprises an siRNA that decreases the expression CD274/PDL-1. In other embodiments, the second dsRNA comprises an siRNA that decreases the expression CD274/PDL-1.

In some embodiments, the two or more functional oligonucleotide comprises the first dsRNA and the first ASO. In certain embodiments, the second dsRNA comprises the first ASO that decreases the expression CD274/PDL-1.

In some embodiments, the dsRNA is an siRNA. In certain embodiments, the siRNA has a nucleotide sequence of an sense strand that is at least 60% (e.g., at least 65%, at least 70%, at least 75%, at least 80%, at least 85%, at least 90%, at least 95%, at least 97%, at least 99% or 100%) identical to the nucleotide sequence selected from: siPDL1-2 (SEQ ID NO: 64) and siPDL1-3 (SEQ ID NO:66). In some embodiments, the dsRNA is an siRNA. In certain embodiments, the siRNA has a nucleotide sequence of an sense strand that is at least 60% (e.g., at least 65%, at least 70%, at least 75%, at least 80%, at least 85%, at least 90%, at least 95%, at least 97%, at least 99% or 100%) identical to the nucleotide sequence of siPDL1-2 (SEQ ID NO: 64). In some embodiments, the dsRNA is an siRNA. In certain embodiments, the siRNA has a nucleotide sequence of an sense strand that is at least 60% (e.g., at least 65%, at least 70%, at least 75%, at least 80%, at least 85%, at least 90%, at least 95%, at least 97%, at least 99% or 100%) identical to the nucleotide sequence of siPDL1-3 (SEQ ID NO: 66).

In some embodiments, the dsRNA is an siRNA. In certain embodiments, the siRNA has a nucleotide sequence of an antisense strand that is at least 60% (e.g., at least 65%, at least 70%, at least 75%, at least 80%, at least 85%, at least 90%, at least 95%, at least 97%, at least 99% or 100%) identical to the nucleotide sequence selected from: siPDL1-2 (SEQ ID NO: 65) and siPDL1-3 (SEQ ID NO:67). In some embodiments, the dsRNA is an siRNA. In certain embodiments, the siRNA has a nucleotide sequence of an antisense strand that is at least 60% (e.g., at least 65%, at least 70%, at least 75%, at least 80%, at least 85%, at least 90%, at least 95%, at least 97%, at least 99% or 100%) identical to the nucleotide sequence of siPDL1-2 (SEQ ID NO: 65). In some embodiments, the dsRNA is an siRNA. In certain embodiments, the siRNA has a nucleotide sequence of an antisense strand that is at least 60% (e.g., at least 65%, at least 70%, at least 75%, at least 80%, at least 85%, at least 90%, at least 95%, at least 97%, at least 99% or 100%) identical to the nucleotide sequence of siPDL1-3 (SEQ ID NO: 67).

In some embodiments, the second oligonucleotide of the at least two oligonucleotides comprises an ASO. In certain embodiments, the ASO has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequence selected from: a) aPDL1-1 (SEQ ID NO: 72); b) aPDL1-2 (SEQ ID NO:73); and c) aPDL1-3 (SEQ ID NO: 74). In certain embodiments, the ASO has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequence of aPDL1-1 (SEQ ID NO: 72). In certain embodiments, the ASO has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequence of aPDL1-2 (SEQ ID NO:73). In certain embodiments, the ASO has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequence of aPDL1-3 (SEQ ID NO:74).

In some embodiments, the multi-valent oligonucleotide agent comprises a saRNA and an siRNA. In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences selected from: a) saP21-40/siPDL1-2 (SEQ ID NO: 71) and an antisense saRNA strand having a nucleotide sequence of SEQ ID NO: 63 that has partial complementarity with the sense saRNA strand of SEQ ID NO: 71; and b) saP21-40/siPDL1-3 (SEQ ID NO: 100) and an antisense saRNA strand having a nucleotide sequence of SEQ ID NO: 63 that has partial complementarity with the sense saRNA strand of SEQ ID NO: 100.

In some embodiments, the multi-valent oligonucleotide agent comprises a saRNA and an ASO. In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences selected from: a) saP21-40/aPDL1-1 (SEQ ID NO: 72) and an antisense saRNA strand having a nucleotide sequence of SEQ ID NO: 63 that has partial complementarity with the sense saRNA strand of SEQ ID NO: 72; b) saP21-40/aPDL1-2 (SEQ ID NO: 73) and an antisense saRNA strand having a nucleotide sequence of SEQ ID NO: 63 that has partial complementarity with the sense saRNA strand of SEQ ID NO: 73; c) saP21-40/aPDL1-3 (SEQ ID NO: 74) and an antisense saRNA strand having a nucleotide sequence of SEQ ID NO: 63 that has partial complementarity with the sense saRNA strand of SEQ ID NO: 74; d) saP21-40/aPDL1-1R (SEQ ID NO: 75) and an antisense saRNA strand having a nucleotide sequence of SEQ ID NO: 63 that has partial complementarity with the sense saRNA strand of SEQ ID NO: 75; e) saP21-40/aPDL1-2R (SEQ ID NO: 76) and an antisense saRNA strand having a nucleotide sequence of SEQ ID NO: 63 that has partial complementarity with the sense saRNA strand of SEQ ID NO: 76; and f) saP21-40/aPDL1-3R (SEQ ID NO: 77) and an antisense saRNA strand having a nucleotide sequence of SEQ ID NO: 63 that has partial complementarity with the sense saRNA strand of SEQ ID NO: 77.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of saP21-40/aPDL1-1 (SEQ ID NO: 72) and an antisense saRNA strand having a nucleotide sequence of SEQ ID NO: 63 that has partial complementarity with the sense saRNA strand of SEQ ID NO: 72.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of saP21-40/aPDL1-2 (SEQ ID NO: 73) and an antisense saRNA strand having a nucleotide sequence of SEQ ID NO: 63 that has partial complementarity with the sense saRNA strand of SEQ ID NO: 73.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of saP21-40/aPDL1-3 (SEQ ID NO: 74) and an antisense saRNA strand having a nucleotide sequence of SEQ ID NO: 63 that has partial complementarity with the sense saRNA strand of SEQ ID NO: 74.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of saP21-40/aPDL1-IR (SEQ ID NO: 75) and an antisense saRNA strand having a nucleotide sequence of SEQ ID NO: 63 that has partial complementarity with the sense saRNA strand of SEQ ID NO: 75.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of saP21-40/aPDL1-2R (SEQ ID NO: 76) and an antisense saRNA strand having a nucleotide sequence of SEQ ID NO: 63 that has partial complementarity with the sense saRNA strand of SEQ ID NO: 76.

In certain embodiments, the multi-valent oligonucleotide agent has a nucleotide sequence that is at least 60% (e.g., at least 65%, at least 70%, at least 75%, at least 80%, at least 85%, at least 90%, at least 95%, at least 97%, at least 99% or 100%) identical to the nucleotide sequences of saP21-40/aPDL1-3R (SEQ ID NO: 77) and an antisense saRNA strand having a nucleotide sequence of SEQ ID NO: 63 that has partial complementarity with the sense saRNA strand of SEQ ID NO: 77.

### 4.4 Compositions of Multi-Valent Oligonucleotide Agent

Another aspect of the present disclosure provides a pharmaceutical composition comprising one or more multi-valent oligonucleotide agent comprising two or more functional oligonucleotides as described in the present disclosure.

In some embodiments, the pharmaceutical composition further comprises at least one pharmaceutically acceptable carrier.

In one embodiment, the pharmaceutically acceptable carrier includes one or more of an aqueous carrier, liposome, polymeric polymer, and polypeptide. In one embodiment, the pharmaceutically acceptable carrier includes one or more of aqueous carriers, liposomes, polymeric polymers, or polypeptides. In one embodiment, the aqueous carrier may be, for example, RNase-free water, or RNase-free buffer. The composition may contain 1-150 nM, for example 1-100 nM, for example 1-50 nM, for example 1-20 nM, for example 10-100 nM, 10-50 nM, 20-50 nM, 20-100 nM, for example 50 nM of the aforementioned oligonucleotides or nucleic acid encoding the oligonucleotides according to the present disclosure.

In some embodiments, the composition comprises 1-150 nM of the first dsRNA comprising a first siRNA and 1-150 nM of a second dsRNA comprising a second siRNA. In some embodiments, the composition comprises 1-150 nM of the first dsRNA comprising a first saRNA and 1-150 nM of a second dsRNA comprising a second saRNA. In some embodiments, the composition comprises 1-150 nM of the first dsRNA comprising the siRNA and 1-150 nM of a second dsRNA comprising a saRNA. In some embodiments, the composition comprises 1-150 nM of the first dsRNA comprising an saRNA and 1-150 nM of the first ASO. In some embodiments, the composition comprises 1-150 nM of the first dsRNA comprising an siRNA and 1-150 nM of the first ASO. In some embodiments, the composition comprises 1-150 nM of the first ASO and 1-150 nM of the second ASO. In some embodiments, the composition comprises 1-150 nM of the first dsRNA comprising a first saRNA, 1-150 nM of the second dsRNA comprising a second saRNA, and 1-150 nM of the third ASO.

Another aspect of the present disclosure relates to the use of two or more functional oligonucleotides as described herein, a nucleic acid encoding two or more functional oligonucleotides as described herein, or a composition comprising such two or more functional oligonucleotides or a nucleic acid encoding two or more functional oligonucleotides as described herein, where the two or more functional oligonucleotides are covalently linked, for the preparation of one or more compositions for modulate the expression of one or more genes or proteins expressed by a cell.

Another embodiment provides pharmaceutical compositions or medicaments comprising the compounds of the present disclosure and a therapeutically inert carrier, diluent or pharmaceutically acceptable excipient, as well as methods of using the compounds of the present disclosure to prepare such compositions and medicaments. In certain embodiments, the two or more functional oligonucleotides of the present disclosure are in the same pharmaceutical compositions, since the two or more functional oligonucleotides are covalently linked.

Compositions of the present disclosure are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

Compositions comprising any of the small molecule compounds described herein, for example, Risdiplam or Branaplam, may be administered separately from the multi-valent oligonucleotide composition by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. For multi-valent oligonucleotide compositions, the delivery can be through parenteral infusions including intrathecal, intramuscular, intravenous, intraarterial, intraperitoneal, or subcutaneous administration.

The small molecule compounds described herein, such as Risdiplam and Branaplam, may be administered in any convenient administrative form, e.g., tablets, powders, capsules, solutions, dispersions, suspensions, syrups, sprays, suppositories, gels, emulsions, patches, etc. Such compositions may comprise components conventional in pharmaceutical preparations, e.g., diluents, carriers, pH modifiers, preservatives, solubilizers, stabilizers, wetting agents, emulsifiers, sweeteners, colorants, flavorants, salts for varying the osmotic pressure, buffers, masking agents, antioxidants, and further active agents. Such compositions can also comprise still other therapeutically valuable substances.

A typical formulation is prepared by mixing a compound of the present invention and a carrier or excipient. Suitable carriers and excipients are well known to those skilled in the art and are described in detail in, e.g., Ansel H. C. et al., Ansel's Pharmaceutical Dosage Forms and Drug Delivery Systems (2004) Lippincott, Williams & Wilkins, Philadelphia; Gennaro A. R. et al., Remington: The Science and Practice of Pharmacy (2000) Lippincott, Williams & Wilkins, Philadelphia; and Rowe R. C, Handbook of Pharmaceutical Excipients (2005) Pharmaceutical Press, Chicago. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

In another aspect, the invention provides use of the combination of two or more functional oligonucleotides of the multi-valent oligonucleotide agent that are covalently linked, according to any one of the embodiments described herein, or a composition according to any one of the embodiments described herein, in the manufacture of a medicament for the treatment of gene or protein-related condition in an individual. The use according to certain embodiments, the condition can include a SMN-deficiency-related condition that comprises a hereditary neuromuscular disease, preferably spinal muscular atrophy. In other embodiments, the condition can include an immune-related condition, such as cancer. Also provided is the use according to certain embodiments wherein the individual is a mammal, preferably a human.

### 4.5 Kits Comprising Multi-Valent Oligonucleotide Agent

In another aspect, any of the multi-valent oligonucleotide agents or compositions described herein can be provided in one or more kits, optionally including instructions for use of the agents or compositions. That is, the kit can include a description of use of a multi-valent oligonucleotide agent or composition in any method described herein. A “kit,” as used herein, typically defines a package, assembly, or container (such as an insulated container) including one or more of the components or embodiments of the invention, and/or other components associated with the invention, for example, as previously described. Any of the antes or components of the kit may be provided in liquid form (e.g., in solution), or in solid form (e.g., a dried powder, frozen, etc.).

In some cases, the kit includes one or more components, which may be within the same or in two or more receptacles, and/or in any combination thereof. The receptacle is able to contain a liquid, and non-limiting examples include bottles, vials, jars, tubes, flasks, beakers, or the like. In some cases, the receptacle is spill-proof (when closed, liquid cannot exit the receptacle, regardless of orientation of the receptacle).

Examples of other compositions or components associated with the agents, compounds and methods described herein include, but are not limited to: diluents, salts, buffers, chelating agents, preservatives, drying agents, antimicrobials, needles, syringes, packaging materials, tubes, bottles, flasks, beakers, and the like, for example, for using, modifying, assembling, storing, packaging, preparing, mixing, diluting, and/or preserving the components for a particular use. In embodiments where liquid forms of any of the components are used, the liquid form may be concentrated or ready to use.

In additional embodiments, a kit can include instructions or instructions to a website or other source in any form that are provided for using the kit in connection with the components and/or methods described herein. For instance, the instructions may include instructions for the use, modification, mixing, diluting, preserving, assembly, storage, packaging, and/or preparation of the components and/or other components associated with the kit. In some cases, the instructions may also include instructions for the delivery of the components, for example, for shipping or storage at room temperature, sub-zero temperatures, cryogenic temperatures, etc. The instructions may be provided in any form that is useful to the user of the kit, such as written or oral (e.g., telephonic), digital, optical, visual (e.g., videotape, DVD, etc.) and/or electronic communications (including Internet or web-based communications), provided in any manner.

### 4.5 Use of/Method for Using the Multi-Valent Oligonucleotide Agent

Multi-valent oligonucleotide agents of the present disclosure are useful for therapeutic approaches to treating diseases associated with the target(s) of the agents.

Provided herein is a method for the treatment of disease comprising administering one or more multi-valent oligonucleotide agent, composition or kit of the present disclosure to a subject in need of the treatment, wherein the target(s) of the multi-valent oligonucleotide agent is associated with the disease.

The following are some exemplary aspects and embodiments of the invention. Such aspects and embodiments do not necessarily represent the full scope of the invention, however, and reference is made therefore to the claims and herein for interpreting the scope of the invention.

4.5.1 Methods of modulating splicing and gene expression

Multi-valent oligonucleotides of the present disclosure are useful for therapeutic approaches to treating diseases such as spinal muscular atrophy (SMA), an autosomal recessive disorder affecting approximately 1 in 6000-8000 newborns and is the leading hereditary cause of mortality in infants. SMA is caused by reduced levels of survival motor neuron (SMN) protein as a result of a homozygous deletion or mutation of the telomeric copy of the survival of motor neuron gene (SMN1) on chromosome 5q13.4.

The SMN protein is encoded by two SMN genes (SMN1 and SMN2), which essentially differ in their coding sequence by one nucleotide in exon 7 in that a cytosine (C) is changed to a thymine (T) in SMN2 gene (Coovert, D. D., et al. The survival motor neuron protein in spinal muscular atrophy. Human Mol Genet (1997)). This critical difference creates a cryptic splicing site and leads to exon 7 skipping in ˜90% of mature SMN mRNA transcribed from SMN2 gene. SMN2 mRNA lacking exon 7 (SMN2Δ7) gives rise to a truncated SMN protein that is unstable and rapidly degraded. In SMA patients, the SMN1 gene no longer produces any SMN protein, and the amount of full length SMN protein produced by SMN2 is not sufficient to compensate for the loss of SMN1, leading to the apoptotic death of the motor neuron in the anterior horn of the spinal cord, atrophy of skeletal muscles, and consequent weakness (Monani, U. R., et al. The human centromeric survival motor neuron gene (SMN2) rescues embryonic lethality in Smn(−/−) mice and results in a mouse with spinal muscular atrophy. Human Mol Genet (2000)). The severity of the symptoms for SMA patients depends on the copy number of the SMN2 gene in a patient's cells—a larger number of copies results in less severe symptoms (Harada, Y., et al. Correlation between SMN2 copy number and clinical phenotype of spinal muscular atrophy: three SMN2 copies fail to rescue some patients from the disease severity. J Neurol (2002)).

To develop therapeutics for SMA, one strategy is to use splicing modulators (SMs) to stimulate exon 7 inclusion during the splicing process. In this regard, an antisense oligonucleotide (ASO) drug SPINRAZA® has already been approved for marketing by the U.S. Food and Drug Administration (FDA) (Hua, Y. and A. R. Krainer. Antisense-mediated exon inclusion. Methods Mol Biol (2012) and Stein, C. A. and D. Castanotto. FDA-Approved Oligonucleotide Therapies in 2017. Mol Thera (2017)). Another drug, Risdiplam (RG7916), an investigational, orally administered small molecule drug, is under New Drug Application (NDA) with the FDA (Ramdas, S. and L. Servais. New treatments in spinal muscular atrophy: an overview of currently available data. Expert Opin Pharmaco (2020)). These two drugs have changed the treatment of SMA by significantly extending patients' survival and improving motor milestones. Despite those improvements, treated patients, especially children, are far from living normal lives. Several plausible reasons could explain the inadequate efficacy of SMs. One is the ceiling effect that limits the maximum achievable level of full-length SMN protein restored by the treatments. SMs do not have an effect on SMN2 transcription, and so do not increase the amount of available SMN2 pre-mRNA. To restore SMN protein to its normal physiological level, SMs optimally would achieve a 100% in vivo efficiency in converting SMN2Δ7 mRNA to full-length mRNA, an ideal effect that is unlikely to occur in reality. Thus, the maximal efficacy SMs can offer to patients is limited by the availability of SMN2 pre-mRNA.

Another therapeutic approach to treating SMA involves stimulating SMN2 transcription to increase the levels of full-length SMN protein. Previously, various epigenetic modifying agents, such as histone deacetylase (HDAC) inhibitors (e.g., sodium butyrate, valproic acid) and non HDAC inhibitors (e.g., hydroxyurea, celecoxib, albuterol, etc.), have been tested in vitro and in mouse SMA models, but these failed to demonstrate noticeable clinical efficacy (Lunke, S. and A. El-Osta. The emerging role of epigenetic modifications and chromatin remodeling in spinal muscular atrophy. J Neurochem (2009)). An explanation for the failure is the lack of target specificity of epigenetic modifying agents. There exists a need for improved methods and compositions for treating SMN-deficiency-related conditions, such as spinal muscular atrophy.

Another aspect of the present disclosure relates to a method of treating or delaying the onset of an SMN-deficiency-related condition in an individual comprising administering to the subject a therapeutically effective amount of an multi-valent oligonucleotide agent as described herein, a nucleic acid encoding a multi-valent oligonucleotide agent as described herein, or a composition comprising a multi-valent oligonucleotide agent of the invention or a nucleic acid encoding a multi-valent oligonucleotide agent as described herein. The subject may be a mammal, such as a human. The subject may be an infant, a child or an adult. In one embodiment, the disease caused by insufficient SMN full-length protein expression or SMN1 gene mutation may include, for example, SMA. In one embodiment, the disease caused by under-expression of the SMN full-length protein, mutation or deletion of the SMN1 gene, and/or under-expression of the full-length SMN protein is SMA. In one embodiment, the SMA of the present invention includes SMA Type I, SMA Type II, SMA Type III, and SMA Type IV.

In some aspects, methods of the present disclosure include a method of increasing the expression of an SMN2 gene or protein while also increasing functional SMN protein levels in a subject in need thereof, comprising administering to the subject a pharmaceutical composition comprising a multi-valent agent of the present disclosure and a pharmaceutically acceptable carrier.

In certain embodiments, the increase in production of functional SMN protein occurs by modulating SMN2 mRNA splicing or stability (SMN2 mRNA modulators).

In some embodiments, the methods of the present disclosure further include administering at least one or more SMN2 mRNA modulators selected from the group consisting of Nusinersen, Risdiplam and Branaplam.

Another aspect of the invention relates to the use of a multi-valent oligonucleotide agent of the present disclosure, a nucleic acid encoding two or more functional oligonucleotides of the multi-valent oligonucleotide agent of the present disclosure or a composition comprising the multi-valent oligonucleotide agent of the present disclosure or a nucleic acid encoding two or more functional oligonucleotides of the multi-valent oligonucleotide agent of the present for the preparation of a medicament for the treatment or delaying the onset of an SMN-deficiency-related condition. The subject may be a mammal, such as a human. The subject may be an infant, a child or an adult. In one embodiment, the an SMN-deficiency-related condition may include, for example, SMA. In one embodiment, the SMA of the present invention includes SMA Type I, SMA Type II, SMA Type III, and SMA Type IV.

Also provided is the use according to any one of the combinations of an SMN2 dsRNA and an SMN2 mRNA modulator described herein, or a composition according to any one of the combinations of an SMN2 dsRNA and an SMN2 mRNA modulator described herein, in the manufacture of a preparation for increasing the amount of full-length SMN protein in a cell. In certain embodiments, the cell is a mammalian cell, preferably a human cell. In certain embodiments, the cell is present in a human. In certain embodiments, the human is a patient suffering from symptoms caused by an SMN-deficiency-related condition. In certain embodiments, the combinations, or the compositions thereof, is administered in an amount an amount effective to treat the SMN-deficiency-related condition. In certain embodiments, the symptoms caused by SMN-deficiency-related condition are those associated with hereditary neuromuscular diseases, preferably spinal muscular atrophy.

In certain embodiments, the covalently linked two or more functional oligonucleotides achieves an increase in full-length SMN protein that is greater than the amount achieved by administration of the same amount of either substance used individually, with reduced toxicity or unwanted side effects. In certain embodiments, the two or more functional oligonucleotides achieves an increase in full-length SMN protein that is greater than the additive effect of treatment with the same amount of either oligonucleotide used individually. In certain embodiments, amount of each of the oligonucleotides administered in an amount that is less than the amount used for conventional treatment when used in an embodiment of the covalently linked combination described herein. In certain embodiments, the multi-valent oligonucleotide agent optionally comprises an SMN2 mRNA modulator.

In certain embodiments, the effect of the combination of two or more functional oligonucleotides achieves a greater clinical improvement compared to the effect of the same amount of either substance used individually. In certain embodiments, the effect of the covalently linked two or more functional oligonucleotides achieves a greater than additive clinical improvement compared to the effect of the same amount of either substance used individually.

The present disclosure also relates to a method of increasing the amount of full-length SMN protein in a cell comprising administering to the cell a multi-valent oligonucleotide comprising two or more functional oligonucleotides as described herein, a nucleic acid encoding two or more functional oligonucleotides as described herein, or a composition comprising the two or more functional oligonucleotides or a nucleic acid encoding the two or more functional oligonucleotides as described herein.

In any of the embodiments provided herein, such multi-valent agent, nucleic acids encoding the multi-valent agent of the present disclosure, or compositions comprising such multi-valent agent or nucleic acids encoding multi-valent agent of the present disclosure may be introduced directly into a cell, or may be produced intracellularly upon introduction of a nucleotide sequence encoding the multi-valent agent into a cell, preferably a mammalian cell, more preferably a human cell. Such cells may be ex vivo, such as cell lines, and the like, or may be present in mammalian bodies, such as humans. In some embodiments, the human is a patient or individual suffering from a SMN-deficiency-related condition. In certain embodiments, a nucleic acid encoding a multi-valent agent or a composition comprising the aforementioned multi-valent agent or a nucleic acid encoding a multi-valent agent of the invention, in respective amounts sufficient to effect treatment of the SMN-deficiency-related condition. In one embodiment, the SMN-deficiency-related condition is SMA. In one embodiment, the SMA of the present disclosure includes SMA Type I, SMA Type II, SMA Type III, and SMA Type IV.

In certain embodiments, the combination of SMN2 dsRNA and SMN2 mRNA modulator, when covalently linked, achieves an increase in full-length SMN protein that is greater than the amount achieved by administration of the same amount of either substance used individually. In certain embodiments, the combination of two or more functional oligonucleotides of the multi-valent agent has reduced toxicity and/or reduced unwanted side effects compared to treatment by monotherapy. In certain embodiments, the two or more functional oligonucleotides of the multi-valent agent achieves an increase in full-length SMN protein that is greater than the additive effect of treatment with the same amount of either substance used individually. In certain embodiments, each of the oligonucleotides, when covalently linked, are administered in an amount less than the amount that would be used for conventional monotherapy treatment.

In certain embodiments, the two or more functional oligonucleotides of the multi-valent agent achieves a greater clinical improvement compared to the effect of the same amount of either substance used individually. In certain embodiments, the two or more functional oligonucleotides of the multi-valent agent achieves a greater than additive clinical improvement compared to the effect of the same amount of either substance used individually.

In certain embodiments, the baseline measurement is obtained from a biological sample, as defined herein, obtained from an individual prior to administering the therapy described herein. In certain embodiments, the biological sample is peripheral blood mononuclear cells, blood plasma, serum, skin tissue, cerebrospinal fluid (CSF). In certain embodiments, increases in SMN protein levels in peripheral blood mononuclear cells and skin correlate with those in neurons of the central nervous system (CNS), indicating that a change of these levels in blood or skin can be used as a non-invasive surrogate to determine changes of SMN protein levels in the CNS. In further embodiments, the combination provided herein increases the amount of full-length SMN protein as compared to the baseline measurement, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 115%, by at least 120%, by at least 125%, by at least 130%, by at least 135%, by at least 140%, by at least 145%, by at least 150%, by at least 155%, by at least 160%, by at least 165%, by at least 170%, by at least 175%, by at least 180%, by at least 185%, by at least 190%, by at least 195%, by at least 200%, by at least 210%, by at least 215%, by at least 220%, by at least 225%, by at least 230%, by at least 235%, by at least 240%, by at least 245%, by at least 250%, by at least 255%, by at least 260%, by at least 265%, by at least 270%, by at least 275%, by at least 280%, by at least 285%, by at least 290%, by at least 295%, by at least 300%, by at least 310%, by at least 315%, by at least 320%, by at least 325%, by at least 330%, by at least 335%, by at least 340%, by at least 345%, by at least 350%, by at least 355%, by at least 360%, by at least 365%, by at least 370%, by at least 375%, by at least 380%, by at least 385%, by at least 390%, by at least 395%, by at least 400%.

The dosage at which compositions of the present disclosure can be administered can vary within wide limits and will, of course, be fitted to the individual requirements in each case.

In a particular embodiment, the multi-valent agent show a greater than additive effect or synergy in the treatment, prevention, delaying progression and/or amelioration of diseases caused by an inactivating mutation or deletion in the SMN1 gene and/or associated with loss or defect of SMN1 gene function, and additionally for the protection of cells implicated in the pathophysiology of the disease, particularly for the treatment, prevention, delaying progression and/or amelioration of spinal muscular atrophy (SMA).

In certain embodiments, a first dose of a pharmaceutical composition according to the present disclosure is administered when the subject is less than one week old, less than one month old, less than 3 months old, less than 6 months old, less than one year old, less than 2 years old, less than 15 years old, or older than 15 years old.

In certain embodiments, at least one pharmaceutical composition comprising the multi-valent agent comprises two or more functional oligonucleotides. The single dose of the multi-valent agent can be a single 0.1 to 15 milligram dose, a single 1 milligram dose, a single 2 milligram dose, a single 3 milligram dose, a single 4 milligram dose, a single 5 milligram dose, a single 6 milligram dose, single 7 milligram dose, a single 8 milligram dose, a single 9 milligram dose, a single 10 milligram dose, a single 11 milligram dose, a single 12 milligram dose, a single 13 milligram dose, a single 14 milligram dose, a single 15 milligram dose, a single 16 milligram dose, a single 17 milligram dose, a single 18 milligram dose, a single 19 milligram dose, a single 20 milligram dose, a single 21 milligram dose, a single 22 milligram dose, a single 23 milligram dose, a single 24 milligram dose, a single 25 milligram dose, a single 26 milligram dose, a single 27 milligram dose, a single 28 milligram dose, a single 29 milligram dose, a single 30 milligram dose, a single 35 milligram dose, a single 40 milligram dose, a single 45 milligram dose, or a single 50 milligram dose. The doses described herein may contain two or more multi-valent oligonucleotide agents of the present disclosure.

In certain embodiments, the multi-valent oligonucleotide agent is administered as an intrathecal injection, e.g., by lumbar puncture, subcutaneous or intravenous injections.

In certain embodiments, where a dose of the multi-valent oligonucleotide agent is administered as an intrathecal injection by lumbar puncture, the use of a smaller gauge needle may reduce or ameliorate one or more symptoms associated with a lumbar puncture procedure. In certain embodiments, symptoms associated with a lumbar puncture include, but are not limited to, post-lumbar puncture syndrome, headache, back pain, pyrexia, constipation, nausea, vomiting, and puncture site pain. In certain embodiments, use of a 24- or 25-gauge needle for the lumbar puncture reduces or ameliorates one or more post lumbar puncture symptoms. In certain embodiments, use of a 21-, 22-, 23-, 24- or 25-gauge needle for the lumbar puncture reduces or ameliorates post-lumbar puncture syndrome, headache, back pain, pyrexia, constipation, nausea, vomiting, and/or puncture site pain.

Proposed dose frequency is approximate, for example, in certain embodiments if the proposed dose frequency is a dose at day 1 and a second dose at day 29, an SMA patient may receive a second dose 25, 26, 27, 28, 29, 30, 31, 32, 33, or 34 days after receipt of the first dose. In certain embodiments, if the proposed dose frequency is a dose at day 1 and a second dose at day 15, an SMA patient may receive a second dose 10, 11, 12, 13, 14, 15, 16, 17, 18, 19, or 20 days after receipt of the first dose. In certain embodiments, if the proposed dose frequency is a dose at day 1 and a second dose at day 85, an SMA patient may receive a second dose 80, 81, 82, 83, 84, 85, 86, 87, 88, 89, or 90 days after receipt of the first dose.

In certain embodiments, the dose and/or the volume of the injection will be adjusted based on the patient's age, the patient's CSF volume, or the patient's age and/or estimated CSF volume. (For example, see Matsuzawa J, Matsui M, Konishi T, Noguchi K, Gur R C, Bilker W, Miyawaki T. Age-related volumetric changes of brain gray and white matter in healthy infants and children. Cereb Cortex 2001 April; 11(4):335-342, which is hereby incorporated by reference in its entirety).

In some embodiments, the multi-valent oligonucleotide agent can be delivered or administered via a vector. Any vectors that may be used for gene delivery may be used. In some variations, a viral vector (such as AAV, adenovirus, lentivirus, a retrovirus) may be used. Non-limiting examples of vectors that may be used in the present disclosure include, but are not limited to human immunodeficiency virus; HSV, herpes simplex virus; MMSV, Moloney murine sarcoma virus; MSCV, murine stem cell virus; SFV, Semliki Forest virus; SIN, Sindbis virus; VEE, Venezuelan equine encephalitis virus; VSV, vesicular stomatitis virus; VV, and vaccinia virus.

In some embodiments, the vector is a recombinant AAV vector. AAV vectors are DNA viruses of relatively small size that can integrate, in a stable and site specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

AAV vectors may be prepared using standard methods in the art. Adeno-associated viruses of any serotype are suitable (see, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p 5-14, Hudder Arnold, London, U K (2006); and DE Bowles, J E Rabinowitz, R J Samulski “The Genus Dependovirus” (J R Kerr, S F Cotmore. M E Bloom, R M Linden, C R Parrish, Eds.) p 15-23, Hudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the invention may include a recombinant virus particle (recombinant because it contains a recombinant polynucleotide) comprising any of the vectors described herein. Methods of producing such particles are known in the art and are described in U.S. Pat. No. 6,596,535.

4.5.2 Methods of modulating an immune response

Aspects of the present disclosure include methods of modulating an immune response comprising administering to a subject a pharmaceutical composition comprising any multi-valent oligonucleotide as described in the present disclosure.

In some embodiments, modulating an immune response comprises increasing the expression of a CDKN1A/p21 gene or protein, and decreasing the expression CD274/PDL-1.

In some embodiments, the pharmaceutical composition comprises a first dsRNA that increases the expression of a CDKN1A/p21 gene or protein and a second oligonucleotide that decreases the expression CD274/PDL-1.

Aspects of the present methods further include a method of increasing the expression of p21 and decreasing the expression of a PDL-1 gene or protein in an subject in need thereof, comprising: administering to the subject a pharmaceutical composition of the present disclosure including any multi-valent oligonucleotide agent.

4.5.3 Methods of treating cancer

Aspects of the present methods include methods of treating a solid tumor, cancer, or malignancy in a subject comprising administering to the subject a pharmaceutical composition comprising a multi-valent oligonucleotide agent of the present disclosure and a pharmaceutically acceptable carrier.

In some embodiments, treating the solid tumor comprises inhibiting the tumor and associated growth effectors.

In some embodiments, the subject has cancer. In certain embodiments, the subject has a malignant tumor.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

Cancers that may be treated include tumors that are not vascularized, or not yet substantially vascularized, as well as vascularized tumors. Types of cancers to be treated with the recombinant dendritic cells described herein include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pincaloma, hemangioblastoma, acoustic neuroma, oligodendroglioma, menangioma, neuroblastoma, retinoblastoma and brain metastases).

In some embodiments, the subject is suffering from a cancer selected from the group consisting of colon carcinoma, breast cancer, pancreatic cancer, ovarian cancer, prostate cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, merkel cell carcinoma, craniopharyngioma, ependymoma, pinealoma, hemangioblastoma, acoustic neuroma, oligodendroglioma, meningioma, melanoma, neuroblastoma, retinoblastoma, acute lymphocytic leukemia, acute myelocytic leukemia, chronic leukemia, polycythemia vera, lymphoma, multiple myeloma, Waldenstrom's macroglobulinemia, heavy chain disease, and combinations thereof.

In additional embodiments, the cancer is a solid tumor selected from the group consisting of fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

In certain embodiments, the cancer is a solid tumor selected from the group consisting of fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma/colorectal cancer, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

In some embodiments, the method further comprises administering one or more additional agents commonly used as standard of care to treat cancer. In certain embodiments, the additional agent is selected from radiation, chemotherapy, an antibody drug conjugate, and an immune modulating antibody.

In some embodiments, the chemotherapy is of cisplatin, carboplatin, paclitaxel, docetaxel, gemcitabine, vinorelbine, vinblastine, irinotecan, etoposide, or pemetrexed, or combinations thereof, or a pharmaceutically acceptable salt thereof.

In some embodiments, the immune modulating antibody is an anti-PD-1 antibody, an anti-PD-L1 antibody, an anti-CD40 antibody, an anti-CTLA-4 antibody, or an anti-OX40 antibody, or any combination thereof.

In some embodiments, the antibody drug conjugate targets c-Met kinase, LRRC15, EGFR, or CS1, or any combination thereof.

In additional embodiments, the method further comprises administering to the patient an immune check-point inhibitor, including any one or combination of two check point inhibitors, including an inhibitor of PD-1 or PD-L1 (B7—H1), such as an anti-PD-1 antibody, including nivolumab (Nivolumab from Bristol-Myers Squibb), pembrolizumab/lambrolizumab, also known as MK-3475 (Keytruda from Merck), pidilizumab (Curetech), AMP-224 (Amplimmune), or an anti-PD-L1 antibody, including MPDL3280A (Roche), MDX-1105 (Bristol Myer Squibb), MEDI-4736 (AstraZeneca) and MSB-0010718 C (Merck), an antagonist of CTLA-4, such as an anti-CTLA-4 antibody including anti-CTLA4 antibody Yervoy.TM. (ipilimumab, Bristol-Myers Squibb), tremelimumab (Pfizer), Ticilimumab (AstraZeneca) or AMGP-224 (Glaxo Smith Kline), or a tumor specific antibody trastuzumab (Herceptin) for breast cancer, rituximab (Rituxan) for lymphoma, or cetuximab (Erbitux).

In some embodiments, the route of administration is via intratumoral, peritumoral, intradermal, subcutaneous, intramuscular, intraperitoneal injection. The compositions are administered to stimulate an immune response, and can be given by bolus injection, continuous infusion, sustained release from implants, or other suitable technique.

In some aspects, any of the methods of treatment described herein can further comprise administering one or more additional anti-cancer therapies to the individual. Various classes of anti-cancer agents can be used. Non-limiting examples include: radiation therapy, alkylating agents (e.g. cisplatin, carboplatin, or oxaliplatin), antimetabolites (e.g., azathioprine or mercaptopurine), anthracyclines, plant alkaloids (including, e.g. vinca alkaloids (such as, vincristine, vinblastine, vinorelbine, or vindesine) and taxanes (such as, paclitaxel, taxol, or docetaxel)), topoisomerase inhibitors (e.g., camptothecins, irinotecan, topotecan, amsacrine, etoposide, etoposide phosphate, or teniposide), podophyllotoxin (and derivatives thereof, such as etoposide and teniposide), antibodies (e.g., monoclonal or polyclonal), tyrosine kinase inhibitors (e.g., imatinib mesylate (Gleevec.RTM. or Glivec.RTM.)), hormone treatments, soluble receptors and other antineoplastics (e.g., dactinomycin, doxorubicin, epirubicin, bleomycin, mechlorethamine, cyclophosphamide, chlorambucil, or ifosfamide).

Agents that may be used adjunctively with anti-PD-1 antibodies include, but are not limited to, alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, and the like, as well as combinations of one or more of these agents.

BiTE antibodies are bispecific antibodies that direct T-cells to attack cancer cells by simultaneously binding the two cells. The T-cell then attacks the target cancer cell. Examples of BiTE antibodies include adecatumumab (Micromet MT201), blinatumomab (Micromet MT103) and the like. Without being limited by theory, one of the mechanisms by which T-cells elicit apoptosis of the target cancer cell is by exocytosis of cytolytic granule components, which include perforin and granzyme B.

Multivalent binding proteins are binding proteins comprising two or more antigen binding sites. Multivalent binding proteins are engineered to have the three or more antigen binding sites and are generally not naturally occurring antibodies. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific (i.e., capable of binding one antigen) or multispecific (i.e., capable of binding two or more antigens). DVD binding proteins comprising two heavy chain DVD polypeptides and two light chain DVD polypeptides are referred to as DVD Ig's. Each half of a DVD Ig comprises a heavy chain DVD polypeptide, a light chain DVD polypeptide, and two antigen binding sites. Each binding site comprises a heavy chain variable domain and a light chain variable domain with a total of 6 CDRs involved in antigen binding per antigen binding site.

Alkylating agents include, but are not limited to, altretamine, AMD-473, AP-5280, apaziquone, bendamustine, brostallicin, busulfan, carboquone, carmustine (BCNU), chlorambucil, CLORETAZINE.RTM. (laromustine, VNP 40101M), cyclophosphamide, dacarbazine, estramustine, fotemustine, glufosfamide, ifosfamide, KW-2170, lomustine (CCNU), mafosfamide, melphalan, mitobronitol, mitolactol, nimustine, nitrogen mustard N-oxide, ranimustine, temozolomide, thiotepa, TREANDA.RTM. (bendamustine), treosulfan, and trofosfamide.

Angiogenesis inhibitors include, but are not limited to, endothelial-specific receptor tyrosine kinase (Tie-2) inhibitors, epidermal growth factor receptor (EGFR) inhibitors, vascular endothelial growth factor receptor (VEGF) inhibitors, delta-like ligand 4 (DLLA) inhibitors, insulin growth factor-2 receptor (IGFR-2) inhibitors, matrix metalloproteinase-2 (MMP-2) inhibitors, matrix metalloproteinase-9 (MMP-9) inhibitors, platelet-derived growth factor receptor (PDGFR) inhibitors, thrombospondin analogs, and vascular endothelial growth factor receptor tyrosine kinase (VEGFR) inhibitors.

Antibody drug conjugates include, but are not limited to, those that target c-Met kinase (e.g., ADCs described in U.S. Pat. No. 7,615,529), LRRC15, CD30 (e.g., ADCETRIS.RTM. (brentuximab vedotin)), CS1 (e.g., ADCs described in US publication no. 20160122430), DLL3 (e.g., rovalpituzumab tesirine (ROVA-T)), HER2 (e.g., KADCYLA.RTM. (trastuzumab emtansine)), EGFR (e.g., ADCs described in US publication no. 20150337042), and prolactin receptor (e.g., ADCs described in US publication no. 20140227294).

Antimetabolites include, but are not limited to, ALIMTA.RTM. (pemetrexed disodium, LY231514, MTA), 5-azacitidine, XELODA.RTM. (capecitabine), carmofur, LEUSTAT.RTM. (cladribine), clofarabine, cytarabine, cytarabine ocfosfate, cytosine arabinoside, decitabine, deferoxamine, doxifluridine, eflornithine, EICAR (5-ethynyl-1-. beta.-D-ribofuranosylimidazole-4-carboxamide), enocitabine, ethnylcytidine, fludarabine, 5-fluorouracil alone or in combination with leucovorin, GEMZAR.RTM. (gemcitabine), hydroxyurea, ALKERAN.RTM. (melphalan), mercaptopurine, 6-mercaptopurine riboside, methotrexate, mycophenolic acid, nelarabine, nolatrexed, ocfosfate, pelitrexol, pentostatin, raltitrexed, ribavirin, triapine, trimetrexate, S-1, tiazofurin, tegafur, TS-1, vidarabine, and UFT.

Antivirals include, but are not limited to, ritonavir, acyclovir, cidofovir, ganciclovir, foscarnet, zidovudine, ribavirin, and hydroxychloroquine.

Aurora kinase inhibitors include, but are not limited to, ABT-348, AZD-1152, MLN-8054, VX-680, Aurora A-specific kinase inhibitors, Aurora B-specific kinase inhibitors and pan-Aurora kinase inhibitors.

]Bcl-2 protein inhibitors include, but are not limited to, ABT-263 (navitoclax), AT-101 ((-)gossypol), GENASENSE.RTM. (G3139 or oblimersen (Bcl-2-targeting antisense oligonucleotide)), IPI-194, IPI-565, N-(4-(4-((4′-chloro(1,1′-biphenyl)-2-yl)methyl)piperazin-1-yl)benzoyl)-4-(((1R)-3-(dimethylamino)-1-((phenyl sulfanyl)methyl)propyl)amino)-3-nitrobenzene sulfonamide), N-(4-(4-((2-(4-chlorophenyl)-5,5-dimethyl-1-cyclohex-1-en-1-yl)methyl)pip-erazin-1-yl)benzoyl)-4-(((1R)-3-(morpholin-4-yl)-1-((phenylsulfanyl)methyl--) propyl)amino)-3-((trifluoromethyl)sulfonyl)benzenesulfonamide, venetoclax and GX-070 (obatoclax).

Bcr-Abl kinase inhibitors include, but are not limited to, DASATINIB.RTM. (BMS-354825) and GLEEVEC.RTM. (imatinib).

BTK inhibitors include, but are not limited to, ibrutinib and acalabrutinib.

CDK inhibitors include, but are not limited to, AZD-5438, BMI-1040, BMS-032, BMS-387, CVT-2584, flavopyridol, GPC-286199, MCS-5A, PD0332991, PHA-690509, seliciclib (CYC-202, R-roscovitine), abemaciclib, palbociclib. and ZK-304709.

COX-2 inhibitors include, but are not limited to, ABT-963, ARCOXIA.RTM. (etoricoxib), BEXTRA.RTM. (valdecoxib), BMS347070, CELEBREX.RTM. (celecoxib), COX-189 (lumiracoxib), CT-3, DERAIVIAXX.RTM. (deracoxib), JTE-522, 4-methyl-2-(3,4-dimethylphenyl)-1-(4-sulfamoylphenyl-1H-pyrrole)-, MK-663 (etoricoxib), NS-398, parecoxib, RS-57067, SC-58125, SD-8381, SVT-2016, S-2474, T-614, and VIOXX.RTM. (rofecoxib).

EGFR inhibitors include, but are not limited to, ABX-EGF, anti-EGFR immunoliposomes, EGF-vaccine, EMD-7200, ERBITUX.RTM. (cetuximab), HR3, IgA antibodies, IRESSA.RTM. (gefitinib), TARCEVA.RTM. (erlotinib or OSI-774), TAGRISSO.RTM. (osimertinib), TP-38, EGFR fusion protein, and TYKERB.RTM. (lapatinib).

ErbB2 receptor inhibitors include, but are not limited to, CP-724-714, CI-1033 (canertinib), HERCEPTIN.RTM. (trastuzumab), TYKERB.RTM. (lapatinib), OMNITARG.RTM. (2C4, pertuzumab), TAK-165, GW-572016 (ionafarnib), GW-282974, EKB-569, PI-166, dHER2 (HER2 vaccine), APC-8024 (HER-2 vaccine), anti-HER/2neu bispecific antibody, B7.her2IgG3, AS HER2 trifunctional bispecific antibodies, mAB AR-209, and mAB 2B-1.

Histone deacetylase inhibitors include, but are not limited to, depsipeptide, LAQ-824, MS-275, trapoxin, suberoylanilide hydroxamic acid (SAHA), TSA, and valproic acid.

HSP-90 inhibitors include, but are not limited to, 17-AAG-nab, 17-AAG, CNF-101, CNF-1010, CNF-2024, 17-DMAG, geldanamycin, IPI-146, KOS-953, MYCOGRAB.RTM. (human recombinant antibody to HSP-90), NCS-683664, PU24FC1, PU-3, radicicol, SNX-2112, STA-9090, and VER49009.

Inhibitors of apoptosis proteins include, but are not limited to, HGS1029, GDC-0145, GDC-0152, LCL-161, and LBW-242.

Activators of death receptor pathway include, but are not limited to, TRAIL, antibodies or other agents that target TRAIL or death receptors (e.g., DR4 and DR5) such as Apomab, conatumumab, ETR2-ST01, GDC0145 (lexatumumab), HGS-1029, LBY-135, PRO-1762 and trastuzumab.

Kinesin inhibitors include, but are not limited to, Eg5 inhibitors such as AZD4877, ARRY-520; and CENPE inhibitors such as GSK923295A.

JAK-2 inhibitors include, but are not limited to, CEP-701 (lesaurtinib), XL019 and INCB018424.

MEK inhibitors include, but are not limited to, ARRY-142886, ARRY-438162, PD-325901, and PD-98059.

mTOR inhibitors include, but are not limited to, AP-23573, CCI-779, everolimus, RAD-001, rapamycin, temsirolimus, ATP-competitive TORC1/TORC2 inhibitors, including PI-103, PP242, PP30, and Torin 1.

Non-steroidal anti-inflammatory drugs include, but are not limited to, AMIGESIC.RTM. (salsalate), DOLOBID.RTM. (diflunisal), MOTRIN.RTM. (ibuprofen), ORUDIS.RTM. (ketoprofen), RELAFEN.RTM. (nabumetone), FELDENE.RTM. (piroxicam), ibuprofen cream, ALEVE.RTM. (naproxen) and NAPROSYN.RTM. (naproxen), VOLTAREN.RTM. (diclofenac), INDOCIN.RTM. (indomethacin), CLINORIL.RTM. (sulindac), TOLECTIN.RTM. (tolmetin), LODINE.RTM. (etodolac), TORADOL.RTM. (ketorolac), and DAYPRO.RTM. (oxaprozin).

PDGFR inhibitors include, but are not limited to, C-451, CP-673 and CP-868596.

Platinum chemotherapeutics include, but are not limited to, cisplatin, ELOXATIN.RTM. (oxaliplatin) eptaplatin, lobaplatin, nedaplatin, PARAPLATIN.RTM. (carboplatin), satraplatin, and picoplatin.

Polo-like kinase inhibitors include, but are not limited to, BI-2536.

Phosphoinositide-3 kinase (PI3K) inhibitors include, but are not limited to, wortmannin, LY294002, XL-147, CAL-120, ONC-21, AEZS-127, ETP-45658, PX-866, GDC-0941, BGT226, BEZ235, and XL765.

Thrombospondin analogs include, but are not limited to, ABT-510, ABT-567, ABT-898, and TSP-1.

VEGFR inhibitors include, but are not limited to, ABT-869, AEE-788, ANGIOZYME.TM. (a ribozyme that inhibits angiogenesis (Ribozyme Pharmaceuticals (Boulder, Colo.) and Chiron (Emeryville, Calif.)), axitinib (AG-13736), AZD-2171, CP-547,632, CYRAMZA.RTM. (ramucirumab), IM-862, MACUGEN.RTM. (pegaptamib), NEXAVAR.RTM. (sorafenib, BAY43-9006), pazopanib (GW-786034), vatalanib (PTK-787, ZK-222584), SUTENT.RTM. (sunitinib, SU-11248), STIVARGA.RTM. (regorafenib), VEGF trap, and ZACTIMA.TM. (vandetanib, ZD-6474).

Antibiotics include, but are not limited to, intercalating antibiotics aclarubicin, actinomycin D, amrubicin, annamycin, adriamycin, BLENOXANE.RTM. (bleomycin), daunorubicin, CAELYX.RTM. or MYOCET.RTM. (liposomal doxorubicin), elsamitrucin, epirbucin, glarbuicin, ZAVEDOS.RTM. (idarubicin), mitomycin C, nemorubicin, neocarzinostatin, peplomycin, pirarubicin, rebeccamycin, stimalamer, streptozocin, VALSTAR.RTM. (valrubicin), and zinostatin.

Topoisomerase inhibitors include, but are not limited to, aclarubicin, 9-aminocamptothecin, amonafide, amsacrine, becatecarin, belotecan, BN-80915, CAMPTOSAR.RTM. (irinotecan hydrochloride), camptothecin, CARDIOXANE.RTM. (dexrazoxine), diflomotecan, edotecarin, ELLENCE.RTM. or PHARMORUBICIN.RTM. (epirubicin), etoposide, exatecan, 10-hydroxycamptothecin, gimatecan, lurtotecan, mitoxantrone, Onivyde.TM. (liposomal irinotecan), orathecin, pirarbucin, pixantrone, rubitecan, sobuzoxane, SN-38, tafluposide, and topotecan.

Antibodies include, but are not limited to, AVASTIN.RTM. (bevacizumab), CD40-specific antibodies, chTNT-1/B, denosumab, ERBITUX.RTM. (cetuximab), HUMAX-CD4.RTM. (zanolimumab), IGF1R-specific antibodies, lintuzumab, OX-40 specific antibodies, PANOREX.RTM. (edrecolomab), RENCAREX.RTM. (WX G250), RITUXAN.RTM. (rituximab), ticilimumab, trastuzumab, pertuzumab, VECTIBIX.RTM. (panitumumab) and CD20 antibodies types I and II.

Hormonal therapies include, but are not limited to, ARIMIDEX.RTM. (anastrozole), AROMASIN.RTM. (exemestane), arzoxifene, CASODEX.RTM. (bicalutamide), CETROTIDE.RTM. (cetrorelix), degarelix, deslorelin, DESOPAN.RTM. (trilostane), dexamethasone, DROGENIL.RTM. (flutamide), EVISTA.RTM. (raloxifene), AFEMA.TM. (fadrozole), FARESTON.RTM. (toremifene), FASLODEX.RTM. (fulvestrant), FEMARA.RTM. (letrozole), formestane, glucocorticoids, HECTOROL.RTM. (doxercalciferol), RENAGEL.RTM. (sevelamer carbonate), lasofoxifene, leuprolide acetate, MEGACE.RTM. (megesterol), MIFEPREX.RTM. (mifepristone), NILANDRON.TM. (nilutamide), NOLVADEX.RTM. (tamoxifen citrate), PLENAXIS.TM. (abarelix), prednisone, PROPECIA.RTM. (finasteride), rilostane, SUPREFACT.RTM. (buserelin), TRELSTAR.RTM. (luteinizing hormone releasing hormone (LHRH)), VANTAS.RTM. (Histrelin implant), VETORYL.RTM. (trilostane or modrastane), and ZOLADEX.RTM. (fosrelin, goserelin).

Deltoids and retinoids include, but are not limited to, seocalcitol (EB1089, CB1093), lexacalcitrol (KH1060), fenretinide, PANRETIN.RTM. (aliretinoin), ATRAGEN.RTM. (liposomal tretinoin), TARGRETIN.RTM. (bexarotene), and LGD-1550.

PARP inhibitors include, but are not limited to, ABT-888 (veliparib), KU-59436, AZD-2281 (olaparib), AG-014699 (rucaparib), MK4827 (niraparib), BMN-673 (talazoparib), iniparib, BSI-201, BGP-15, INO-1001, and ONO-2231.

Plant alkaloids include, but are not limited to, vincristine, vinblastine, vindesine, and vinorelbine.

Proteasome inhibitors include, but are not limited to, VELCADE.RTM. (bortezomib), KYPROLIS.RTM. (carfilzomib), MG132, NPI-0052, and PR-171.

Non-limiting examples of immunologicals include, but are not limited to, interferons, immune checkpoint inhibitors, co-stimulatory agents, and other immune-enhancing agents. Interferons include interferon alpha, interferon alpha-2a, interferon alpha-2b, interferon beta, interferon gamma-la, ACTIMMUNE.RTM. (interferon gamma-1b) or interferon gamma-n1, combinations thereof and the like. Immune check point inhibitors include antibodies that target PD-L1 (e.g., durvalumab, atezolizumab, avelumab, MEDI4736, MSB0010718C and MPDL3280A), and CTLA4 (cytotoxic lymphocyte antigen 4; e.g., ipilimumab, tremelimumab). Co-stimulatory agents include, but are not limited to, antibodies against CD3, CD40, CD40L, CD27, CD28, CSF1R, CD137 (e.g., urelumab), B7H1, GITR, ICOS, CD80, CD86, OX40, OX40L, CD70, HLA-DR, LIGHT, LIGHT-R, TIM3, A2AR, NKG2A, KIR (e.g., lirilumab), TGF-. beta. (e.g., fresolimumab) and combinations thereof.

Other agents include, but are not limited to, ALFAFERONE.RTM. (IFN−. alpha.), BAM-002 (oxidized glutathione), BEROMUN.RTM. (tasonermin), BEXXAR.RTM. (tositumomab), CAMPATH.RTM. (alemtuzumab), dacarbazine, denileukin, epratuzumab, GRANOCYTE.RTM. (lenograstim), lentinan, leukocyte alpha interferon, imiquimod, melanoma vaccine, mitumomab, molgramostim, MYLOTARG.TM. (gemtuzumab ozogamicin), NEUPOGEN.RTM. (filgrastim), OncoVAC-CL, OVAREX.RTM. (oregovomab), pemtumomab (Y-muHMFG1), PROVENGE.RTM. (sipuleucel-T), sargaramostim, sizofilan, teceleukin, THERACYS.RTM. (Bacillus Calmette-Guerin), ubenimex, VIRULIZIN.RTM. (immunotherapeutic, Lorus Pharmaceuticals), Z-100 (Specific Substance of Maruyama (SSM)), WF-10 (Tetrachlorodecaoxide (TCDO)), PROLEUKIN.RTM. (aldesleukin), ZADAXIN.RTM. (thymalfasin), ZINBRYTA.RTM. (daclizumab high-yield process), and ZEVALIN.RTM. (.sup.90Y-Ibritumomab tiuxetan).

Biological response modifiers are agents that modify defense mechanisms of living organisms or biological responses, such as survival, growth or differentiation of tissue cells to direct them to have anti-tumor activity and include, but are not limited to, krestin, lentinan, sizofiran, picibanil PF-3512676 (CpG-8954), and ubenimex.

Pyrimidine analogs include, but are not limited to, cytarabine (ara C or Arabinoside C), cytosine arabinoside, doxifluridine, FLUDARA.RTM. (fludarabine), 5-FU (5-fluorouracil), floxuridine, GEMZAR.RTM. (gemcitabine), TOMUDEX.RTM. (ratitrexed), and TROXATYL.TM. (triacetyluridine troxacitabine).

Purine analogs include, but are not limited to, LANVIS.RTM. (thioguanine) and PURINETHOL.RTM. (mercaptopurine).

Antimitotic agents include, but are not limited to, batabulin, epothilone D (KOS-862), N-(2-((4-hydroxyphenyl)amino)pyridin-3-yl)-4-methoxybenzenesulfonamide, ixabepilone (BMS 247550), TAXOL.RTM. (paclitaxel), TAXOTERE.RTM. (docetaxel), PNU100940 (109881), patupilone, XRP-9881 (larotaxel), vinflunine, and ZK-EPO (synthetic epothilone).

Ubiquitin ligase inhibitors include, but are not limited to, MDM2 inhibitors, such as nutlins, and NEDD8 inhibitors such as MLN4924.

The multi-valent agents and related compositions may also be used to enhance the efficacy of radiation therapy. Examples of radiation therapy include external beam radiation therapy, internal radiation therapy (i.e., brachytherapy) and systemic radiation therapy.

The multi-valent agents and related compositions may be administered adjunctive to or with other chemotherapeutic agents such as ABRAXANE.TM. (ABI-007), ABT-100 (farnesyl transferase inhibitor), ADVEXIN.RTM. (Ad5CMV-p53 vaccine), ALTOCOR.RTM. or MEVACOR.RTM. (lovastatin), AMPLIGEN.RTM. (poly I:poly C12U, a synthetic RNA), APTOSYN.RTM. (exisulind), AREDIA.RTM. (pamidronic acid), arglabin, L-asparaginase, atamestane (1-methyl-3,17-dione-androsta-1,4-diene), AVAGE.RTM. (tazarotene), AVE-8062 (combreastatin derivative) BEC2 (mitumomab), cachectin or cachexin (tumor necrosis factor), canvaxin (vaccine), CEAVAC.RTM. (cancer vaccine), CELEUK.RTM. (celmoleukin), CEPLENE.RTM. (histamine dihydrochloride), CERVARIX.RTM. (human papillomavirus vaccine), CHOP.RTM. (C: CYTOXAN.RTM. (cyclophosphamide); H: ADRIAMYCIN.RTM. (hydroxydoxorubicin); O: Vincristine (ONCOVIN.RTM.); P: prednisone), CYPAT.TM. (cyproterone acetate), combrestatin A4P, DAB(389)EGF (catalytic and translocation domains of diphtheria toxin fused via a His-Ala linker to human epidermal growth factor) or TransMID-107R.TM. (diphtheria toxins), dacarbazine, dactinomycin, 5,6-dimethylxanthenone-4-acetic acid (DMXAA), eniluracil, EVIZON.TM. (squalamine lactate), DIMERICINE.RTM. (T4N5 liposome lotion), discodermolide, DX-8951f (exatecan mesylate), enzastaurin, EP0906 (epithilone B), GARDASIL.RTM. (quadrivalent human papillomavirus (Types 6, 11, 16, 18) recombinant vaccine), GASTRIMMUNE.RTM., GENASENSE.RTM., GMK (ganglioside conjugate vaccine), GVAX.RTM. (prostate cancer vaccine), halofuginone, hi strelin, hydroxycarbamide, ibandronic acid, IGN-101, IL-13-PE38, IL-13-PE38QQR (cintredekin besudotox), IL-13-pseudomonas exotoxin, interferon-. alpha., interferon-. gamma., JUNOVAN.TM. or MEPACT.TM. (mifamurtide), lonafarnib, 5,10-methylenetetrahydrofolate, miltefosine (hexadecylphosphocholine), NEOVASTAT.RTM. (AE-941), NEUTREXIN.RTM. (trimetrexate glucuronate), NIPENT.RTM. (pentostatin), ONCONASE.RTM. (a ribonuclease enzyme), ONCOPHAGE.RTM. (melanoma vaccine treatment), ONCOVAX.RTM. (IL-2 Vaccine), ORATHECIN.TM. (rubitecan), OSIDEM.RTM. (antibody-based cell drug), OVAREX.RTM. MAb (murine monoclonal antibody), paclitaxel, PANDIMEX.TM. (aglycone saponins from ginseng comprising 20(S)protopanaxadiol (aPPD) and 20(S)protopanaxatriol (aPPT)), panitumumab, PANVAC.RTM.-VF (investigational cancer vaccine), pegaspargase, PEG Interferon A, phenoxodiol, procarbazine, rebimastat, REMOVAB.RTM. (catumaxomab), REVLIMID.RTM. (lenalidomide), RSR13 (efaproxiral), SOMATULINE.RTM. LA (lanreotide), SORIATANE.RTM. (acitretin), staurosporine (Streptomyces staurospores), talabostat (PT100), TARGRETIN.RTM. (bexarotene), TAXOPREXIN.RTM. (DHA-paclitaxel), TELCYTA.RTM. (canfosfamide, TLK286), temilifene, TEMODAR.RTM. (temozolomide), tesmilifene, thalidomide, THERATOPE.RTM. (STn-KLH), thymitaq (2-amino-3,4-dihydro-6-methyl-4-oxo-5-(4-pyridylthio)quinazoline dihydrochloride), TNFERADE.TM. (adenovector: DNA carrier containing the gene for tumor necrosis factor-.alpha.), TRACLEER.RTM. or ZAVESCA.RTM. (bosentan), tretinoin (Retin-A), tetrandrine, TRISENOX.RTM. (arsenic trioxide), VIRULIZIN.RTM., ukrain (derivative of alkaloids from the greater celandine plant), vitaxin (anti-alphavbeta3 antibody), XCYTRIN.RTM. (motexafin gadolinium), XINLAY.TM. (atrasentan), XYOTAX.TM. (paclitaxel poliglumex), YONDELIS.RTM. (trabectedin), ZD-6126, ZINECARD.RTM. (dexrazoxane), ZOMETA.RTM. (zolendronic acid), and zorubicin, as well as combinations of any of these agents.

### 4.6 Single Oligonucleotide Agents and Use Thereof

Aspects of the present disclosure include single functional oligonucleotide. Aspects of the present disclosure also include pharmaceutical compositions comprising single oligonucleotide agents and a pharmaceutically acceptable carrier.

In some embodiments, the pharmaceutical composition comprises an oligonucleotide having a nucleotide sequence of a saRNA sense strand that is at least 60% (e.g., at least 65%, at least 70%, at least 75%, at least 80%, at least 85%, at least 90%, at least 95%, at least 97%, at least 99% or 100%) to the nucleotide sequence selected from one or more of: a) DS06-4A3 (SEQ ID NO: 146); b) R6-04-S1 (SEQ ID NO: 59); d) R6-04(20)-S1V1v(CM-4) (SEQ ID NO: 60); and c) RAG1-40 (SEQ ID NO: 62). In certain embodiments, the pharmaceutical composition comprises an oligonucleotide having a nucleotide sequence of a saRNA sense strand that is at least 60% (e.g., at least 65%, at least 70%, at least 75%, at least 80%, at least 85%, at least 90%, at least 95%, at least 97%, at least 99% or 100%) to the nucleotide sequence of DS06-4A3 (SEQ ID NO: 146). In certain embodiments, the pharmaceutical composition comprises an oligonucleotide having a nucleotide sequence of a saRNA sense strand that is at least 60% (e.g., at least 65%, at least 70%, at least 75%, at least 80%, at least 85%, at least 90%, at least 95%, at least 97%, at least 99% or 100%) to the nucleotide sequence of R6-04-S1 (SEQ ID NO: 59). In certain embodiments, the pharmaceutical composition comprises an oligonucleotide having a nucleotide sequence of a saRNA sense strand that is at least 60% (e.g., at least 65%, at least 70%, at least 75%, at least 80%, at least 85%, at least 90%, at least 95%, at least 97%, at least 99% or 100%) to the nucleotide sequence of R6-04(20)-S1V1v(CM-4) (SEQ ID NO: 60). In certain embodiments, the pharmaceutical composition comprises an oligonucleotide having a nucleotide sequence of a saRNA sense strand that is at least 60% (e.g., at least 65%, at least 70%, at least 75%, at least 80%, at least 85%, at least 90%, at least 95%, at least 97%, at least 99% or 100%) to the nucleotide sequence of RAG1-40 (SEQ ID NO: 62).

In some embodiments, the pharmaceutical composition further has a nucleotide sequence of an antisense strand of the saRNA that is complementary to the sense strand, and is at least 60% (e.g., at least 65%, at least 70%, at least 75%, at least 80%, at least 85%, at least 90%, at least 95%, at least 97%, at least 99% or 100%) identical to the nucleotide sequence selected from: a) DS06-4A3 (SEQ ID NO: 147);b) R6-04-S1 (SEQ ID NO: 53); c) R6-04(20)-S1VIv(CM-4) (SEQ ID NO: 17); d) RAG1-40 (SEQ TD NO: 63); and e) R6-04M1-27A-S1L1V3(CM-26) (SEQ ID NO: 17).

In certain embodiments, the pharmaceutical composition further has a nucleotide sequence of an antisense strand of the saRNA that is complementary to the sense strand, and is at least 60% (e.g., at least 65%, at least 70%, at least 75%, at least 80%, at least 85%, at least 90%, at least 95%, at least 97%, at least 99% or 100%) identical to the nucleotide sequence of: DS06-4A3 (SEQ ID NO: 147).

In certain embodiments, the pharmaceutical composition further has a nucleotide sequence of an antisense strand of the saRNA that is complementary to the sense strand, and is at least 60% (e.g., at least 65%, at least 70%, at least 75%, at least 80%, at least 85%, at least 90%, at least 95%, at least 97%, at least 99% or 100%) identical to the nucleotide sequence of: DS06-4A-S2L5V (SEQ ID NO: 17).

In certain embodiments, the pharmaceutical composition further has a nucleotide sequence of an antisense strand of the saRNA that is complementary to the sense strand, and is at least 60% (e.g., at least 65%, at least 70%, at least 75%, at least 80%, at least 85%, at least 90%, at least 95%, at least 97%, at least 99% or 100%) identical to the nucleotide sequence of: R6-04-S1 (SEQ ID NO: 53).

In certain embodiments, the pharmaceutical composition further has a nucleotide sequence of an antisense strand of the saRNA that is complementary to the sense strand, and is at least 60% (e.g., at least 65%, at least 70%, at least 75%, at least 80%, at least 85%, at least 90%, at least 95%, at least 97%, at least 99% or 100%) identical to the nucleotide sequence of: R6-04(20)-S1V1v(CM-4) (SEQ ID NO: 17).

In certain embodiments, the pharmaceutical composition further has a nucleotide sequence of an antisense strand of the saRNA that is complementary to the sense strand, and is at least 60% (e.g., at least 65%, at least 70%, at least 75%, at least 80%, at least 85%, at least 90%, at least 95%, at least 97%, at least 99% or 100%) identical to the nucleotide sequence of: RAG1-40 (SEQ ID NO: 63).

In certain embodiments, the pharmaceutical composition further has a nucleotide sequence of an antisense strand of the saRNA that is complementary to the sense strand, and is at least 60% (e.g., at least 65%, at least 70%, at least 75%, at least 80%, at least 85%, at least 90%, at least 95%, at least 97%, at least 99% or 100%) identical to the nucleotide sequence of: R6-04M1-27A-S1L1V3(CM-26) (SEQ ID NO: 17).

In some aspects, provided herein is an isolated or synthesized oligonucleotide comprising: a nucleotide sequence of a saRNA sense strand that is at least 90% identical to the nucleotide sequence of SEQ ID NO: 62. In some embodiments, the isolated or synthesized oligonucleotide further comprises an antisense strand that has partial complementarity with the above sense saRNA strand. In some embodiments, the isolated or synthesized oligonucleotide further comprises an antisense strand that is at least 90% identical to the nucleotide sequence of SEQ ID NO: 63.

In some embodiments, the isolated or synthesized oligonucleotide comprising: a nucleotide sequence of a saRNA sense strand of SEQ ID NO: 62 and a saRNA antisense strand of SEQ ID NO: 63.

In some aspects, provide herein is a pharmaceutical composition or kit, comprising the isolated or synthesized oligonucleotide of the disclosure.

In some aspects, provide herein is a method for disease treatment, comprising administering sufficient amount of one or more of the isolated or synthesized oligonucleotide or a pharmaceutical composition or kit of the present disclosure to a subject in need of such treatment.

### 5. EXAMPLES

The present invention is further illustrated by the following examples. These examples are provided merely for illustration purposes and shall not be interpreted to limit the scope or content of the present invention in any way.

### 5.1. Example 1: Design of Multi-Targeting Oligonucleotide Agents

5.1.1. 2-unit DAOs (dual-acting oligonucleotides)

2-unit DAOs (e.g., multi-targeting oligonucleotide agents with two oligonucleotides covalently linked) were created by covalently connecting two units of functional oligonucleotides with different mechanism of action (MOA) or targeting two different target genes. The functional oligonucleotides include single-stranded oligonucleotide (SSO) (e.g., gapmer ASO and steric block ASO) and double-stranded oligonucleotide (DSO) (e.g., siRNA and saRNA) (Table 3). The two units were joined covalently by any of the following linkers: ethylene glycol chain, an alkyl chain, a peptide, RNA, DNA, a phosphodiester, a phosphorothioate, a phosphoramidate, an amide, a carbamate, or absent. Table 4 lists some exemplary linkers. The structures of 8 DAOs (DAO-1˜ DAO-8) are schematically displayed in Table 5.

TABLE 3
List of 2-unit DAOs with their functional
units within their molecule

| DAO category | Unit A | Unit B |
|---|---|---|
| siRNA-siRNA | siRNA | siRNA |
| saRNA-saRNA | saRNA | saRNA |
| saRNA-siRNA | saRNA | siRNA |
| siRNA-ASO | siRNA | ASO |
| saRNA-ASO | saRNA | ASO |
| ASO-ASO | ASO | ASO |

n.b.: The two units, either different or the same, may be arranged in desired order, for example, Unit A-Unit B or Unit B-Unit A.

TABLE 4
Linkers used in DAOs

| Name | Formula | Structure |
|---|---|---|
| Spacer-9 | C₃₆H₄₉N₂O₇P | 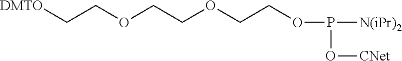 |
| Spacer-18 | C₄₂H₆₁N₂O₁₀P | 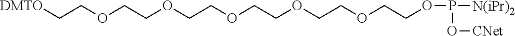 |
| Spacer-C3 | C₃₃H₄₃N₂O₅P | 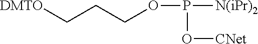 |
| Spacer-C6 | C₃₆H₄₉N₂O₅P | 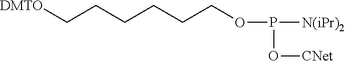 |
| Nucleotide | | 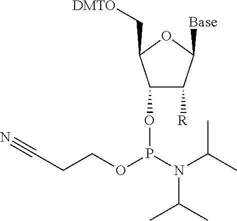 |

*n.b .: R = —H or —OH or —OMe, or —MOE, or -F, or other 2' chemical modification.

TABLE 5
Schematic of 2-unit DAO structures*

| Name | Strcture | Design |
|---|---|---|
| DAO-1 | 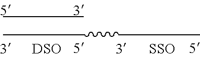 | DSO and SSO covalently joined by a linker |
| DAO-2 | 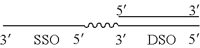 | DSO and SSO covalently joined by a linker |
| DAO-3 | 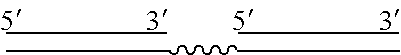 | DSO and DSO covalently joined by a linker |
| DAO-4 | 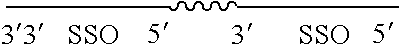 | SSO and SSO covalently joined by a linker |
| DAO-5 | 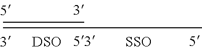 | DSO and SSO joined by a phosphodiester bond |
| DAO-6 | 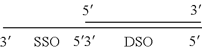 | SSO and DSO joined by a phosphodiester bond |
| DAO-7 | 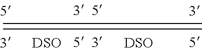 | DSO and DSO joined by a phosphodiester bond on both strands |
| DAO-8 | 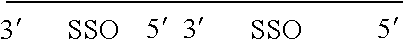 | SSO and SSO joined by a phosphodiester bond |

*key: DSO, double-stranded oligonucleotide; SSO, single-stranded oligonucleotide; [symbol] , linker

5.1.2. 3-unit DAOs

Similar to the DAO structures listed in Table 5, 3-unit DAOs (e.g. multi-targeting oligonucleotide agents with 3 oligonucleotides covalently linked) were created by adding a third unit to the 2-unit DAOs, resulting 6 DAO structures (DAO-9˜ DAO-14, Table 6). These DAOs may be one of the combinations: 3 duplex units, 2 duplex units and an ASO, a duplex and 2 ASO units, or 3 ASOs (Table 6).

TABLE 6
Schematic of 3-unit DAO structures*

| Name | Strcture |
|---|---|
| DAO-9 | 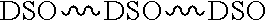 |
| DAO-10 | 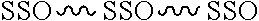 |
| DAO-11 | 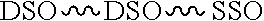 |
| DAO-12 | 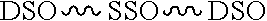 |
| DAO-13 | 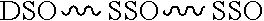 |
| DAO-14 | 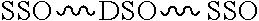 |
| DAO-15 | 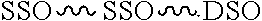 |
| DAO-16 | 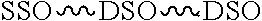 |

*key: DSO, double-stranded oligonucleotide; SSO, single-stranded oligonucleotide; [symbol] , linker or a PS bond (with no linker)

### 5.2. Example 2: Design of DAOs to Increase Target Gene Expression by Combining a Promoter-Targeted saRNA and a Pre-mRNA Targeted Splice Modulating ASO

Spinal muscular atrophy (SMA) is caused by reduced levels of survival motor neuron (SMN) protein as a result of a homozygous deletion or mutation of the telomeric copy of the survival of motor neuron gene (SMN1) on chromosome 5q13.4. SMN protein is also encoded by SMN2 gene, which essentially differ in their coding sequence by one nucleotide in exon 7 in that a cytosine (C) is changed to a thymine (T) in SMN2 gene (Coovert et al. 1997). This critical difference creates a cryptic splicing site and leads to exon 7 skipping in ˜90% of mature SMN mRNA transcribed from SMN2 gene. SMN2 mRNA lacking exon 7 (SMN2Δ7) gives rise to a truncated SMN protein that is unstable and rapidly degraded.

An ASO drug Nusinersen (SPINRAZA®) has been approved to treat SMA patients (Hua, Y. and A. R. Krainer. Antisense-mediated exon inclusion. Methods Mol Biol (2012) and Stein, C. A. and D. Castanotto. FDA-Approved Oligonucleotide Therapies in 2017. Mol Thera (2017)). SPINRAZA® targets and blocks intronic splicing silencer N1 (ISS-N1) in intron 7 of SMN2 pre-mRNA to increase exon 7 inclusion in mature mRNA, leading to increased full-length mature mRNA and then full-length SMN protein. However, the room for increase of full-length SMN2 mRNA is limited by the available SMN2 pre-mRNA which is not altered by the ASO.

To develop DAOs which can maximize full-length SMN2 expression, a series of DAO molecules consisting of a SMN2 saRNA which can activate SMN2 transcription and a splice modulating ASO was created.

### 5.3. Example 3. DAOs Composed of a saRNA and a Splice Modulating ASO Increase the Level of Full Length SMN2 mRNA in SMA Patient-Derived Fibroblasts

In order to determine the effect of two DAOs (DA06-4A-27A and DA06-4A-27B) which were comprised of a SMN2 saRNA (DS06-0004) and a SMN2 splice modulating ASO (ASO10-27), DA06-4A-27A and DA06-4A-27B were individually transfected at 20 nM for 72 hours into GM03813 cells which were derived from a SMA type II patient. DS06-0004 and ASO10-27, DS06-0004 in combination with ASO10-27 were also transfected. Oligonucleotides used in this experiment include the following and are listed in Table 7.

DS06-0004 is a duplex saRNA targeting the promoter region of SMN2 gene to increase the mRNA expression of full-length SMN2 (SMN2FL) and SMN2Δ7 (Table 7).

DS06-4A3 is a duplex saRNA derived from DS06-0004 with structure optimization and chemical modification to increase its in vivo stability and activity (Table 7).

ASO10-27 is a single-stranded ASO which is known to increase the levels of SMN2FL by converting SMN2Δ7 (Table 7).

DA06-4A-27A is DAO created by connecting the 5′ end of the ASO to the 3′ end of the sense strand of DS06-4A3 via a spacer-18 (S18) linker (Table 7).

DA06-4A-27B are DAOs created by connecting the 3′ end of the ASO to the 3′ end of the sense strand of DS06-4A3 via a spacer-18 (S18) linker (Table 7).

“GM03813 cells” refers to fibroblast cells provided by Coriell Institute for Medical Research. This cell line is described as SPINAL MUSCULAR ATROPHY, TYPE II; SMA2 SURVIVAL OF MOTOR NEURON 1, TELOMERIC; SMN1. The relevant gene is SMN1; the chromosomal location is 5q12.2-q13.3, the allelic variant is described as 1 exons 7 and 8 deleted, SPINAL MUSCULAR ATROPHY, TYPE I; and the identified mutation: is EX7-8DEL. The phenotype data derived from a fibroblast from skin (arm) of the following subject characterized as: clinically affected; born after full term uncomplicated pregnancy; rolled over at 6 months old; began babbling at 9 months old; by 12 months old, there was marked muscle atrophy and weakness; absent deep tendon reflexes; constipation; donor subject has 3 copies of the SMN2 gene; PCR analysis showed that this donor subject is homozygous for the deletion of exons 7 and 8 in the SMN1 gene; similarly affected brother (not in repository); mother is GM03814 (Fibroblast)/GM24474 (iPSC); father is GM03815 (Fibroblast); see GM23240 (iPSC-lentiviral) and GM24468 (iPSC-episomal); previously classified as SMA I, but data such as onset features and SMN2 dosage in the proband supported re-classification to SMA II.

Seventy-two hours later, total cellular RNA was isolated from the treated cells and reverse transcribed into cDNA. SMN2 mRNA expression was assessed with RT-qPCR using primer pairs specific for SMN2FL or SMN2A7. SMN2 mRNA expression was also assessed by semi-quantitative RT-PCR using a primer pair that amplifies both SMN2FL and SMN2Δ7 followed by DdeI digestion (RT-PCR/digestion). The PCR resulted in two product bands: 507 bp (SMN2FL) and 453 bp (SMN2A7). After digestion, both bands were reduced by 115 bp, resulting in two products: 392 bp (SMN2FL) and 338 bp (SMN2A7) as shown on the gel of FIG. 1B.

As shown in FIG. 1A, treating cells with 20 nM of DS06-0004, DS06-4A-S2L1v and ASO10-27 induced SMN2FL by 2.1-, 1.9- and 2.1-fold, respectively and SMN2Δ7 by 2.1-, 1.6- and 0.02-fold, respectively. Combining DS06-0004 and ASO-27A in transfection increased SMN2FL mRNA by 3.5-fold and reduced SMN2A7 by 97%. Very similar to the activity of the combinatory treatment, one of the DAOs DA06-4A-27A increased SMN2FL mRNA by 3.2-fold and reduced SMN2A7 by 96%, indicating that the DAO structure successfully retained the activity of both oligonucleotide units in its molecule and demonstrated an additive effect in inducing SMN2FL contributed by both units. This result was further confirmed by RT-PCR/DdeI digestion experiments (FIG. 1B & 1C).

Interestingly, DAO DA06-4A-27B in which the orientation of the ASO unit was flipped in opposition to DA06-4A-27A did not show an obvious increase of its activity in inducing SMN2FL compared to saRNA or ASO used alone, suggesting that direction of the ASO in the DAO structure may affect the activity.

Together, these results reveal that: 1) by covalently conjugating two oligonucleotide units of different MOAs, novel DAO molecules can be created to possess the activity of both units; 2) When applied to DAOs comprising saRNA and splice modulating ASO for the same gene, this conjugation method can result in DAOs to maximize target gene output. 3) The orientation of the ASO unit in DAO may affect the ASO's activity for example the comparison between DA06-4A-27A vs. DAJun. 4, 2027B shows ASO unit linked to the duplex unit in the orientation of 5′→3′ end may possess higher activity.

TABLE 7
Representative oligonucleotide sequences tested in Example 3 and 4

| | | | | SEQ | | | |
|---|---|---|---|---|---|---|---|
| | | | Target | ID | | Size | |
| Name | Category | MOA | gene | NO: | Sequence (5′-3′) | (nt) | Design |
| dsCon2 | n/a | n/a | n/a | 1 | ACUACUGAGUGACAGUAGATT | 21 | n/a |
| | | | | 2 | UCUACUGUCACUCAGUAGUTT | 21 | |
| DS06- | siRNA | RNAi | SMN2 | 3 | GGUGACAUUUGUGAAACUUTT | 21 | siRNA |
| 332i | | | | 4 | AAGUUUCACAAAUGUCACCTT | 21 | |
| DS06- | saRNA | RNAa | SMN2 | 5 | AGACGAGGCCUAAGCAACATT | 21 | saRNA |
| 0004 | | | | 6 | UGUUGCUUAGGCCUCGUCUTT | 21 | |
| DS06- | saRNA | RNAa | SMN2 | 146 | fG*mA*fGmGfCmCfUmAfAmGfCmAfA*mC*fA | 15 | saRNA |
| 4A3 | | | | 147 | mU*fG*mUfUmGfCmUfUmAfGmGfCmC*fU*mC*fG*mU* | 20 | |
| | | | | | fC*mU*fC | | |
| DS06- | DAO | RNAa + | SMN2 | 16 | fG*mA*fGmGfCmCfUmAfAmGfCmAfA*mC*fA-L1- | 30 | two saRNAs linked by S18 |
| 4A- | | RNAa | | | fG*mA*fGmGfCmCfUmAfAmGfCmAfA*mC*fA | | |
| S2L1v | | | | 17 | VPmU*fG*mUfUmGfCmUfUmAfGmGfCmC*fU*mC*fG*m | 20 | |
| | | | | | U*fC*mU*fC | | |
| ASO10- | ASO | steric | SMN2 | 11 | meU*meC*meA*meC*meU*meU*meU*meC*meA*meU*m | 18 | ASO |
| 27 | | block | | | eA*meA*meU*meG*meC*meU*meG*meG | | |
| DA06- | DAO | RNAa + | SMN2+S | 14 | fG*mA*fGmGfCmCfUmAfAmGfCmAfA*mC*fA-L1- | 33 | DS06-4A3 and ASO10-27 |
| 4A- | | steric | | | meU*meC*meA*meC*meU*meU*meU*meC*meA*meU*m | | linked by S18 and with |
| 27A | | block | | | eA*meA*meU*meG*meC*meU*meG*meG | | the ASO linked in 5′-3′ |
| | | | MN2 | 13 | pmU*fG*mUfUmGfCmUfUmAfGmGfCmC*fU*mC*fG*mU | 20 | orientation |
| | | | | | *fC*mU*fC | | |
| DA06- | DAO | RNAa + | SMN2+S | 15 | fG*mA*fGmGfCmCfUmAfAmGfCmAfA*mC*fA-L1- | 33 | DS06-4A3 and ASO10-27 |
| 4A- | | steric | MN2 | 13 | meG*meG*meU*meC*meG*meU*meA*meA*meU*meA*m | 20 | linked by S18, with the |
| 27B | | block | | | eC*meU*meU*meU*meC*meA*meC*meU | | ASO linked in 3′-5′ |
| | | | | | pmU*fG*mUfUmGfCmUfUmAfGmGfCmC*fU*mC*fG*mU | | orientation |
| | | | | | *fC*mU*fC | | |

Keys:
*, phosphorothioate (PS); f, 2′-fluoro; m, 2′-OMe; me, 2′-MOE; p, 5′-phosophate; VP, 5′-(E)-vinylphosphonate; C, 5-methyl cytosine; U, 5-methyl uracil; L1, spacer-18 (S18 linker); L4, spacer-C6 (C6 linker); L9, spacer-9 (S9 linker)

### 5.4. Example 4: Dose-Dependent Effects of DAO (DA06-4A-27A) on the Expression of SMN2 mRNA and SMN Protein in GM03813 Cells

To further assess the dose-dependent effect of DA06-4A-27A on SMN2 expression, DA06-4A-27A was transfected into GM03813 cells at concentrations ranging from 0.1 nM to 50 nM for 72 hours and SMN2 mRNA and protein levels were detected. As shown in FIG. 2A, DA06-4A-27A at 0.1, 1, 5, 10, 25 and 50 nM caused a 1.6-, 2.8-, 4.3-, 4.8-, 3.8- and 3.5-fold increase in SMN2FL with concurrent decrease of SMN2Δ7 (by a 10%, 90%, 99%, 99%, 99% and 99% respectively) with peak SMN2FL induction (4.8 fold) occurred at 10 nM. The result was further verified by RT-PCR/DdeI digestion (FIGS. 2B & 2C). Similarly, SMN protein was also induced in a dose-dependent manner. DA06-4A-27A at 0.1, 1, 5, 10, 25 and 50 nM respectively caused a 1.4-, 2.2-, 2.5-, 2.8-, 2.6- and 2.1-fold increase in SMN protein and caused a peak induction at 10 nM (FIGS. 3A & 3B).

### 5.5. Example 5: DAO (DA06-4A-27A) Induces the Expression of SMN2FL and SMN2A7 mRNA In Vivo

To further verify the activity of DAOs, DA06-4A-27A (10 μg or 40 μg) was injected into heterozygous SMA (Het-SMA) mice carrying human SMN2 gene (with the genotype Smn1^{+/-}, SMN2^{+/-}) at postnatal day 1 (P1) via ICV injection, and the brain, spinal cord and quadriceps femoris muscle were collected 72 hours later for mRNA expression analysis by RT-PCR/DdeI digestion. For comparison, included were mice receiving ICV injection of 20 μg of ASO10-27 and DS06-4A-S2L1v. N.B., the calculated molecular weight of ASO10-27, DS06-4A-S2L1v and DA06-4A-27A is 7500, 12749 and 20249 (1:1.7:2.7) respectively, as such, 10 μg and 40 μg of DA06-4A-27A is equivalent to 0.49 nM and 2.0 nM in molar mass, 20 μg of ASO10-27 and DS06-4A-S2L1v has a molar mass of 2.7 nM and 1.6 nM.

As shown in FIGS. 4A and 4B, DS06-4A-S2L1v (20 μg), DS06-4A-27A (10 μg, low dose) and DS06-4A-27A (40 μg, high dose) treatment caused a 1.1-, 1.4-, 1.6-fold increase in SMN2FL respectively compared to PBS controls. When the total amount SMN2 mRNA (SMN2FL+SMN2Δ7) was evaluated, DS06-4A-27A at 10 μg and 40 μg caused a 2.3- and 2.3-fold increase compared to a 2.0-fold increase caused by DS06-4A-S2L1v at the dosing level of 20 μg.

Similarly, in the spinal cords of Het-SMA mice, DS06-4A-S2L1v (20 μg), DS06-4A-27A (10 μg, low dose) and DS06-4A-27A (40 μg, high dose) treatment caused a 1.2-, 2.5-, 3.5-fold increase in SMN2FL respectively (FIG. 5). DS06-4A-27A (40 μg, high dose) caused a 4.1-fold increase in SMN2FL compared to a 2.2-fold increase caused by DS06-4A-S2L1v alone.

In the muscle, ASO10-27 (20 μg) and DS06-4A-S2L1v (20 μg) did not show any activity in inducing SMN2 mRNA (either SMN2FL or SMN2A7) compared to PBS control. In contrast, DAO DA06-4A-27A (10 μg, low dose) treatment caused a 1.9- and a 1.2-fold increase respectively in SMN2FL and SMN2Δ7 mRNA, resulting a 3.2-fold increase in total SMN2 mRNA compared with PBS control (FIGS. 6A and 6B).

Together, these results indicate the DAO oligonucleotide DS06-4A-27A possesses higher activity in the CNS and peripheral tissues, in particular in the muscle, in boosting SMN2 mRNA levels than does any of its functional unit within its molecule used alone. Although in some examples the benefit of the DAO manifested more by an increase in SMN2Δ7 than by SMN2FL, this benefit could be important for clinical efficacy, since it has been reported that SMN2Δ7 protein also plays an important role in ameliorating SMA phenotype and extending SMA mouse survival by associating with SMN2FL protein to form stable oligomeric SMN protein (Le et al. 2005). Another obvious benefit of a DAO is that the reduced manufacturing cost of the DAO drug compared to the combined manufacturing cost of two oligonucleotide drugs.

### 5.6. Example 6: Effect of DAOs on the Expression of SMN2FL and SMN2A7 mRNA in SMA GM03813 and GM09677 Cells

To further optimize the structure of DA06-4A-27A, a new DAO structure R6-04M1-27A-S1L1V3 was created (Table 8) and tested in SMA GM03813 (type II) and SMA GM09677 (type I) cells.

TABLE 8
Representative oligonucleotide sequences tested in Example 6

| | | | | SEQ | | | |
|---|---|---|---|---|---|---|---|
| | Cat- | | Target | ID | | Size | |
| Name | egory | MOA | gene | NO: | Sequence (5′-3′) | (nt) | Design |
| ASO10- | ASO | steric | SMN2 | 11 | meU*meC*meA*meC*meU*meU*meU*meC*meA*meU*meA* | 18 | steric |
| 27 | | block | | | meA*meU*meG*meC*meU*meG*meG | | block |
| | | | | | | | ASO |
| R6-04- | saRNA | RNAa | SMN2 | 145/ | GACGAGGCCUAAGCAACA | 18 | saRNA |
| S1 | | | | 59 | | | |
| | | | | 53 | UGUUGCUUAGGCCUCGUCUC | 20 | |
| DS06- | DAO | RNAa + | SMN2 | 128 | fG*mA*fGmGfCmCfUmAfAmGfCmAfA*mC*fA-L5 | 18 | DIO |
| 4A- | | RNAa | | 17 | VPmU*fG*mUfUmGfCmUfUmAfGmGfCmC*fU*mC*fG*mU* | | linker |
| S2L5V | | | | | fC*mU*fC | | is used |
| | | | | | | | to make |
| | | | | | | 15 | a |
| | | | | | | | divalent |
| | | | | | | | structure. |
| R6- | DAO | RNAa + | SMN2 + | 18 | mG*fA*mCfGmAfGmGfCfCfUfAfAmGfCmAfA*mC*fA-L1- | 36 | R6- |
| 04M1- | | steric | SMN2 | | meU*meC*meA*meC*meU*meU*meU*meC*meA*meU*meA* | | 04 (20)- |
| 27A- | | block | | | meA*meU*meG*meC*meU*meG*meG | | S1V1v (CM- |
| S1L1V3 | | | | 13 | pmU*fG*mUfUmGfCmUfUmAfGmGfCmC*fU*mC*fG*mU*fC* | | 4) and |
| | | | | | mU*fC | 20 | ASO10-27 |
| | | | | | | | linked |
| | | | | | | | by S18 |

Keys:
*, phosphorothioate (PS); f, 2′-fluoro; m, 2′-OMe; me, 2′-MOE; p, 5′-phosophate; VP, 5′-(E)-vinylphosphonate; C, 5-methyl cytosine; U, 5-methyl uracil; L1, spacer-18 (S18 linker); L4, spacer-C6 (C6 linker); L5, DIO linker; L9, spacer-9 (S9 linker)

R6-04M1-27A-S1L1V3 contains SMN2 saRNA R6-04(20)-S1V1v(CM-4) and ASO10-27 conjugated with a S18 linker.

R6-04(20)-S1V1v(CM-4) was derived from R6-04-S1 with chemical modification. R6-04-S1 was structurally optimized DS06-0004 saRNA.

Also included in the test is a SMN2 saRNA (DS06-4A-S2L5V) which is derived from DS06-0004 with structural optimization and chemical modification. DS06-4A-S2L5V is the same as DS06-4A-S2L1v except that the phosphonate modification on the first nucleotide of the antisense strand counted from the 5′ end was replaced by 5′-(E)-vinylphosphonate (5′(E)Vp) modification.

As shown in FIG. 7A, treating GM03813 cells with 25 nM of ASO10-27, DS06-4A-S2L5V and R6-04M1-27A-S1L1V3 induced total SMN2 mRNA by 1.7, 2.8 and 4.4-fold respectively. When the levels of total SMN2 (SMN2FL+SMN2A7) was assessed, its increase caused by DAO R6-04M1-27A-S1L1V3 was the highest (4.4-fold) compared to 1.7- and 2.8-fold increase caused by ASO10-27 and DS06-4A-S2L5V respectively. As shown in FIG. 7B, treating GM09677 cells with 25 nM of ASO10-27, DS06-4A-S2L5V and R6-04M1-27A-S1L1V3 induced SMN2FL by 2.0-, 2.8- and 4.8-fold, respectively. Consistent with the result in GM03813 cells, DAO R6-04M1-27A-S1L1V3 possesses higher activity than any of its oligonucleotide components within its molecule used alone.

### 5.7. Example 7: Effect of DAOs on the Expression of SMN Protein in SMA GM03813 and GM09677 Cells

To further verify SMN protein level induction by DAO R6-04M1-27A-S1L1V3, SMN protein was assessed by western blotting in GM03813 and GM09677 cells treated with the DAO, saRNA R6-04-S1, saRNA R6-04(20)-S1V1v(CM-4) or ASO10-27.

As shown in FIGS. 8A & 8B, treating GM03813 cells with 25 nM of ASO10-27, R6-04-S1, R6-04(20)-S1V1v(CM-4) and R6-04M1-27A-S1L1V3 caused a 1.7-, 2.1-, 1.8- and 2.7-fold increase in SMN protein respectively, with DAO R6-04M1-27A-S1L1V3 giving the highest induction (2.7-fold). Similarly, in GM09677 cells (FIGS. 8C & 8D), 25 nM of ASO10-27, R6-04-S1, R6-04(20)-S1V1v(CM-4) and R6-04M1-27A-S1L1V3 caused a 2.2-, 3.0-, 3.5- and 3.9-fold increase in SMN protein respectively. Consistent with the result in GM03813 cells, DAO R6-04M1-27A-S1L1V3 possesses higher activity than any of its oligonucleotide components within its molecule used alone.

### 5.8. Example 8: Effects of Different DAO Structure on the Expression of SMN2FL and SMN2Δ7 mRNA in GM03813 Cells

In order to fully prove the advantages of DAO structures, two more SMN2 saRNAs (DS06-0031 and DS06-0067) were selected for inclusion in DAO design. These two saRNAs were selected because they possess robust activity in inducing SMN2 transcription which, however, mainly manifested as an increase in SMN2A7 instead of SMN2FL caused by DS06-0004 (FIG. 9). Two DAOs (A and B version) were created by connecting ASO10-27 to DS06-0031 and DS06-0067, resulting in two DAOs for each of them. The two DAOs differ in the duplex strand conjugated with the ASO in the DAO structure. In the A version (DA06-31A-27A and DA06-67A-27A), the ASO's 5′ end was linked to the 3′end of the sense strand of the duplex via a linker, whereas in the B version (DA06-31B-27A and DA06-67B-27A), the ASO's 5′ end was linked to the 3′ end of the antisense strand of the duplex via a linker (Table 9).

TABLE 9
Representative oligonucleotide sequences tested in Example 8

| | | | | SEQ | | | |
|---|---|---|---|---|---|---|---|
| | | | Target | ID | | Size | |
| Name | Category | MOA | gene | NO: | Sequence (5′ -3′) | (nt) | Design |
| DS06- | siRNA | RNAi | SMN2 | 3 | GGUGACAUUUGUGAAACUUTT | 21 | siRNA |
| 332i | | | | 4 | AAGUUUCACAAAUGUCACCTT | 21 | |
| ASO10- | ASO | steric | SMN2 | 11 | meU*meC*meA*meC*meU*meU*meU*meC*meA*meU*meA*meA* | 18 | steric block ASO |
| 27 | | block | | | meU*meG*meC*meU*meG*meG | | |
| DS06- | saRNA | RNAa | SMN2 | 7 | UUGUACACUUGGUCAACAUTT | 21 | saRNA |
| 0031 | | | | 8 | AUGUUGACCAAGUGUACAATT | 21 | |
| DS06- | saRNA | RNAa | SMN2 | 9 | CACUGGAGUUCGAGACGAGTT | 21 | saRNA |
| 0067 | | | | 10 | CUCGUCUCGAACUCCAGUGTT | 21 | |
| DA06- | DAO | RNAa + | SMN2 + | 19 | UUGUACACUUGGUCAACAUTT-L1- | 39 | DAO in the form of |
| 31A- | | steric | SMN2 | | meU*meC*meA*meC*meU*meU*meU*meC*meA*meU*meA*meA* | | AS010-27 conjugated |
| 27A | | block | | | meU*meG*meC*meU*meG*meG | | with DS06-0031 at 3′- |
| | | | | 8 | AUGUUGACCAAGUGUACAATT | 21 | end of its sense |
| | | | | | | | strand by S18 |
| DA06- | DAO | RNAa + | SMN2 + | 7 | UUGUACACUUGGUCAACAUTT | 21 | DAO in the form of |
| 31B- | | steric | SMN2 | 20 | AUGUUGACCAAGUGUACAATT-L1- | | ASO10-27 conjugated |
| 27A | | block | | | meU*meC*meA*meC*meU*meU*meU*meC*meA*meU*meA*meA* | | with DS06-0031 at 3′- |
| | | | | | meU*meG*meC*meU*meG*meG | 39 | end of its antisense |
| | | | | | | | strand by S18 |
| DA06- | DAO | RNAa + | SMN2 + | 21 | CACUGGAGUUCGAGACGAGTT-L1- | 39 | DAO in the form of |
| 67A- | | steric | SMN2 | | meU*meC*meA*meC*meU*meU*meU*meC*meA*meU*meA*meA* | | ASO10-27 conjugated |
| 27A | | block | | | meU*meG*meC*meU*meG*meG | | with DS06-0067 at 3′- |
| | | | | 10 | CUCGUCUCGAACUCCAGUGTT | 21 | end of its sense |
| | | | | | | | strand by S18 |
| DA06- | DAO | RNAa + | SMN2 + | 9 | CACUGGAGUUCGAGACGAGTT | 21 | DAO in the form of |
| 67B- | | steric | SMN2 | 22 | CUCGUCUCGAACUCCAGUGTT-L1- | | ASO10-27 conjugated |
| 27A | | block | | | meU*meC*meA*meC*meU*meU*meU*meC*meA*meU*meA*meA* | | with DS06-0067 at 3′- |
| | | | | | meU*meG*meC*meU*meG*meG | 39 | end of its antisense |
| | | | | | | | strand by S18 |

Keys:
*, phosphorothioate (PS); f, 2′-fluoro; m, 2′-OMe; me, 2′-MOE; p, 5′-phosophate; VP, 5′-(E)-vinylphosphonate; C, 5-methyl cytosine; U, 5-methyl uracil; L1, spacer-18 (S18 linker); L4, spacer-C6 (C6 linker); L9, spacer-9 (S9 linker)

As shown in FIG. 9, DS06-0031 and DS06-0067 treatment of GM03813 cells at 20 nM increased dramatically SMN2A7 by 4.0- and 2.7-fold respectively, although the levels of SMN2FL was either unaltered (by DS06-0031) or induced to a less magnitude (2.3 fold by DS06-0067). This result indicates that these two saRNAs possess strong RNAa activity despite the activity mainly manifested as an increase in SMN2A7. Indeed, connecting ASO10-27 to either of DS06-0031 or DS06-0067 resulted in similar or mostly increased activity in inducing SMN2FL compared to the activity of either the saRNA or the ASO used alone, especially for the DAO containing DS06-0067. As shown in FIG. 9, DA06-31A-27A and DA06-31B-27A increased SMN2FL by 2.1 and 1.8 fold, while DA06-67A-27A and DA06-67B-27A increased SMN2FL by 3.0 and 2.3 fold respectively.

### 5.9. Example 9: Effects of DAOs with Different Conjugation Positions and Linkers on the Expression of SMN2FL and SMN2Δ7 mRNA in SMA GM03813 Cells

Based on saRNAs DS06-0004 and DS06-0067, additional DAOs were created by connecting them to ASO10-27 using different linkers, including no linker (L0) between two functional oligonucleotide units, spacer 18 (L1), spacer C6 (LA), spacer 9 (L9). These DAOs were transfected into GM03813 cells at 25 nM and SMN2 expression was assessed by RT-qPCR and RT-PCR followed by DdeI digestion. These DAOs are listed in Table 10 and Table 11.

TABLE 10
Composition of additonal SMN2 DAOs

| | | | | |
|---|---|---|---|---|
| | | | The duplex strand and terminus to | |
| Name of DAOs | saRNA unit | ASO unit | which the ASO is conjugated | Linker |

RAG6-0067 derivatives

| | | | | |
|---|---|---|---|---|
| DA6-67A3′L0-27A | RAG6-0067 | ASO10-27 | Sense, 3′ end | None |
| DA06-67A-27A | RAG6-0067 | ASO10-27 | Sense, 3′ end | Spacer 18 |
| DA6-67A3′L9-27A | RAG6-0067 | ASO10-27 | Sense, 3′ end | Spacer 9 |
| DA6-67A3′L4-27A | RAG6-0067 | ASO10-27 | Sense, 3′ end | Spacer C6 |
| DA6-67B3′L0-27A | RAG6-0067 | ASO10-27 | Antisense, 3′ end | None |
| DA6-67B5′L1-27A | RAG6-0067 | ASO10-27 | Antisense, 5′ end | Spacer 18 |
| DA06-67B-27A | RAG6-0067 | ASO10-27 | Antisense, 3′ end | Spacer 18 |
| DA6-67B5′L9-27A | RAG6-0067 | ASO10-27 | Antisense, 5′ end | Spacer 9 |
| DA6-67B5′L4-27A | RAG6-0067 | ASO10-27 | Antisense, 5′ end | Spacer C6 |
| DA6-67B3′L9-27A | RAG6-0067 | ASO10-27 | Antisense, 3′ end | Spacer 9 |
| DA6-67B3′L4-27A | RAG6-0067 | ASO10-27 | Antisense, 3′ end | Spacer C6 |
| DA06-67A21L1-27A | RAG6-0067, extended toward the duplex's 5′ end by 2 nt | ASO10-27 | Sense, 3′ end | Spacer 18 |
| DA06-67B21L1-27A | RAG6-0067, extended toward the duplex's 5′ end by 2 nt | ASO10-27 | Antisense, 3′ end | Spacer 18 |

RAG6-0004 derivatives

| | | | | |
|---|---|---|---|---|
| DA6-04A3′L0-27A | RAG6-0004 | ASO10-27 | Sense, 3′ end | None |
| DA6-04A5′L1-27A | RAG6-0004 | ASO10-27 | Sense, 5′ end | Spacer 18 |
| DA6-04A5′L9-27A | RAG6-0004 | ASO10-27 | Sense, 5′ end | Spacer 9 |
| DA6-04A5′L4-27A | RAG6-0004 | ASO10-27 | Sense, 5′ end | Spacer C6 |
| DA6-04A3′L1-27A | RAG6-0004 | ASO10-27 | Sense, 3′ end | Spacer 18 |
| DA6-04A3′L9-27A | RAG6-0004 | ASO10-27 | Sense, 3′ end | Spacer 9 |
| DA6-04A3′L4-27A | RAG6-0004 | ASO10-27 | Sense, 3′ end | Spacer C6 |
| DA6-04B3′L0-27A | RAG6-0004 | ASO10-27 | Antisense, 3′ end | None |
| DA6-04B3′L1-27A | RAG6-0004 | ASO10-27 | Antisense, 3′ end | Spacer 18 |
| DA6-04B3′L9-27A | RAG6-0004 | ASO10-27 | Antisense, 3′ end | Spacer 9 |
| DA6-04B3′L4-27A | RAG6-0004 | ASO10-27 | Antisense, 3′ end | Spacer C6 |
| DA06-04A21L1-27A | RAG6-0004, extended toward the duplex's 3′ end by 2 nt | ASO10-27 | Sense, 3′ end | Spacer 18 |
| DA06-04B21L1-27A | RAG6-0004, extended toward the duplex's 3′ end by 2 nt | ASO10-27 | Antisense, 3′ end | Spacer 18 |

TABLE 11
Representative oligonucleotide sequences tested in Example 9

| | | | | SEQ | | | |
|---|---|---|---|---|---|---|---|
| | | | Target | ID | | Size | |
| Name | Category | MOA | gene | NO: | Sequence (5′-3′) | (nt) | Design |
| ASO10- | ASO | steric | SMN2 | 11 | meU*meC*meA*meC*meU*meU*meU*meC*meA*meU | 18 | steric block ASO |
| 27 | | block | | | *meA*meA*meU*meG*meC*meU*meG*meG | | |
| DS06- | saRNA | RNAa | SMN2 | 9 | CACUGGAGUUCGAGACGAGTT | 21 | saRNA |
| 0067 | | | | 10 | CUCGUCUCGAACUCCAGUGTT | 21 | |
| DA6- | DAO | RNAa + | SMN2 + | 23 | CACUGGAGUUCGAGACGAG-TT- | 39 | DAO in the form of ASO-10-27 |
| 67A3′L | | steric | SMN2 | | meU*meC*meA*meC*meU*meU*meU*meC*meA*meU | | conjugated with DS06-0067 |
| 0-27A | | block | | | *meA*meA*meU*meG*meC*meU*meG*meG | | duplex at 3′-end of its |
| | | | | 10 | CUCGUCUCGAACUCCAGUGTT | 21 | sense strand by dTdT linker |
| DA06- | DAO | RNAa + | SMN2 + | 21 | CACUGGAGUUCGAGACGAGTT-L1- | 39 | DAO in the form of ASO-10-27 |
| 67A- | | steric | SMN2 | | meU*meC*meA*meC*meU*meU*meU*meC*meA*meU | | conjugated with DS06-0067 |
| 27A | | block | | | *meA*meA*meU*meG*meC*meU*meG*meG | | duplex at 3′-end of its |
| | | | | 10 | CUCGUCUCGAACUCCAGUGTT | 21 | sense strand by S18 linker |
| DA6- | DAO | RNAa + | SMN2 + | 24 | CACUGGAGUUCGAGACGAGTT-L9- | 39 | DAO in the form of ASO-10-27 |
| 67A3′L | | steric | SMN2 | | meU*meC*meA*meC*meU*meU*meU*meC*meA*meU | | conjugated with DS06-0067 |
| 9-27A | | block | | | *meA*meA*meU*meG*meC*meU*meG*meG | | duplex at 3′-end of its |
| | | | | 10 | CUCGUCUCGAACUCCAGUGTT | 21 | sense strand by dTdT linker |
| DA6- | DAO | RNAa + | SMN2 + | 25 | CACUGGAGUUCGAGACGAGTT-L4- | 39 | DAO in the form of ASO-10-27 |
| 67A3′L | | steric | SMN2 | | meU*meC*meA*meC*meU*meU*meU*meC*meA*meU | | conjugated with DS06-0004 at |
| 4-27A | | block | | | *meA*meA*meU*meG*meC*meU*meG*meG | | 3′-end of its sense strand |
| | | | | 10 | CUCGUCUCGAACUCCAGUGTT | 21 | by C6 linker |
| DA6- | DAO | RNAa + | SMN2 + | 9 | CACUGGAGUUCGAGACGAGTT | 21 | DAO in the form of ASO-10-27 |
| 67B3′L | | steric | SMN2 | 26 | CUCGUCUCGAACUCCAGUG-TT- | | conjugated with DS06-0067 at |
| 0-27A | | block | | | meU*meC*meA*meC*meU*meU*meU*meC*meA*meU | 39 | 3′end of its antisense |
| | | | | | *meA*meA*meU*meG*meC*meU*meG*meG | | strand by dTdT |
| DA6- | DAO | RNAa + | SMN2 + | 9 | CACUGGAGUUCGAGACGAGTT | 21 | DAO in the form of ASO-10-27 |
| 67B5′L | | steric | SMN2 | 27 | meU*meC*meA*meC*meU*meU*meU*meC*meA*meU | | conjugated with DS06-0067 at |
| 1-27A | | block | | | *meA*meA*meU*meG*meC*meU*meG*meG-L1- | 39 | 5′end of its antisense |
| | | | | | CUCGUCUCGAACUCCAGUGTT | | strand by S18 linker |
| DA06- | DAO | RNAa + | SMN2 + | 9 | CACUGGAGUUCGAGACGAGTT | 21 | DAO in the form of ASO-10-27 |
| 67B- | | steric | SMN2 | 22 | CUCGUCUCGAACUCCAGUGTT-L1- | | conjugated with DS06-0067 |
| 27A | | block | | | meU*meC*meA*meC*meU*meU*meU*meC*meA*meU | | duplex at 3′-end of its |
| | | | | | *meA*meA*meU*meG*meC*meU*meG*meG | 39 | antisense strand by S18 |
| | | | | | | | linker |
| DA6- | DAO | RNAa + | SMN2 + | 9 | CACUGGAGUUCGAGACGAGTT | 21 | DAO in the form of ASO-10-27 |
| 67B5′L | | steric | SMN2 | 29 | meU*meC*meA*meC*meU*meU*meU*meC*meA*meU | | conjugated with DS06-0067 at |
| 9-27A | | block | | | *meA*meA*meU*meG*meC*meU*meG*meG-L9- | 39 | 5′ end of its antisense |
| | | | | | CUCGUCUCGAACUCCAGUGTT | | strand by S9 linker |
| DA6- | DAO | RNAa + | SMN2 + | 9 | CACUGGAGUUCGAGACGAGTT | 21 | DAO in the form of ASO-10-27 |
| 67B5′L | | steric | SMN2 | 30 | meU*meC*meA*meC*meU*meU*meU*meC*meA*meU | | conjugated with DS06-0067 at |
| 4-27A | | block | | | *meA*meA*meU*meG*meC*meU*meG*meG-L4- | 39 | 5′ end of its antisense |
| | | | | | CUCGUCUCGAACUCCAGUGTT | | strand by C6 linker |
| DA6- | DAO | RNAa + | SMN2 + | 9 | CACUGGAGUUCGAGACGAGTT | 21 | DAO in the form of ASO-10-27 |
| 67B3′L | | steric | SMN2 | 31 | CUCGUCUCGAACUCCAGUGTT-L9- | | conjugated with DS06-0067 at |
| 9-27A | | block | | | meU*meC*meA*meC*meU*meU*meU*meC*meA*meU | 39 | 3′end of its antisense |
| | | | | | *meA*meA*meU*meG*meC*meU*meG*meG | | strand by S9 linker |
| DA6- | DAO | RNAa + | SMN2 + | 9 | CACUGGAGUUCGAGACGAGTT | 21 | DAO in the form of ASO-10-27 |
| 67B3′L | | steric | SMN2 | 32 | CUCGUCUCGAACUCCAGUGTT-L4- | | conjugated with DS06-0067 at |
| 4-27A | | block | | | meU*meC*meA*meC*meU*meU*meU*meC*meA*meU | 39 | 3′end of its antisense |
| | | | | | *meA*meA*meU*meG*meC*meU*meG*meG | | strand by C6 linker |
| DA06- | DAO | RNAa + | SMN2 + | 33 | CACUGGAGUUCGAGACGAGGCTT-L1- | 41 | DAO in the form of ASO-10-27 |
| 67A21L | | steric | SMN2 | | meU*meC*meA*meC*meU*meU*meU*meC*meA*meU | | conjugated with DS06-0067 at |
| 1-27A | | block | | | *meA*meA*meU*meG*meC*meU*meG*meG | | 3′-end of its sense strand |
| | | | | 34 | GCCUCGUCUCGAACUCCAGUGTT | 23 | by S9 linker, the duplex is |
| | | | | | | | extended by 2 nt at 3′ end |
| DA06- | DAO | RNAa + | SMN2 + | 35 | CACUGGAGUUCGAGACGAGGCTT | 23 | DAO in the form of ASO-10-27 |
| 67B21L | | steric | SMN2 | 36 | GCCUCGUCUCGAACUCCAGUGTT-L1- | | conjugated with DS06-0067 at |
| 1-27A | | block | | | meU*meC*meA*meC*meU*meU*meU*meC*meA*meU | | 3′end of its antisense |
| | | | | | *meA*meA*meU*meG*meC*meU*meG*meG | 41 | strand by S18 linker, with |
| | | | | | | | the duplex expand 2-nt at |
| | | | | | | | its 3′end |
| DS06- | saRNA | RNAa | SMN2 | 5 | AGACGAGGCCUAAGCAACATT | 21 | saRNA |
| 0004 | | | | 6 | UGUUGCUUAGGCCUCGUCUTT | 21 | |
| DA6- | DAO | RNAa + | SMN2 + | 37 | AGACGAGGCCUAAGCAACA-TT- | 39 | DAO in the form of ASO-10-27 |
| 04A3′L | | steric | SMN2 | | meU*meC*meA*meC*meU*meU*meU*meC*meA*meU | | conjugated with DS06-0004 |
| 0-27A | | block | | | *meA*meA*meU*meG*meC*meU*meG*meG | | duplex at 3′-end of its |
| | | | | 6 | UGUUGCUUAGGCCUCGUCUTT | 21 | sense strand by dTdT linker |
| DA6- | DAO | RNAa + | SMN2 + | 38 | meU*meC*meA*meC*meU*meU*meU*meC*meA*meU | 39 | DAO in the form of ASO-10-27 |
| 04A5′L | | steric | SMN2 | | *meA*meA*meU*meG*meC*meU*meG*meG-L1- | | conjugated with DS06-0004 |
| 1-27A | | block | | | AGACGAGGCCUAAGCAACATT | | duplex at 5′-end of its |
| | | | | 6 | UGUUGCUUAGGCCUCGUCUTT | 21 | sense strand by TT linker |
| DA6- | DAO | RNAa + | SMN2 + | 39 | meU*meC*meA*meC*meU*meU*meU*meC*meA*meU | 39 | DAO in the form of ASO-10-27 |
| 04A5′L | | steric | SMN2 | | *meA*meA*meU*meG*meC*meU*meG*meG-L9- | | conjugated with DS06-0004 at |
| 9-27A | | block | | | AGACGAGGCCUAAGCAACATT | | 5′-end of its sense strand |
| | | | | 6 | UGUUGCUUAGGCCUCGUCUTT | 21 | by S9 linker |
| DA6- | DAO | RNAa + | SMN2 + | 40 | meU*meC*meA*meC*meU*meU*meU*meC*meA*meU | 39 | DAO in the form of ASO-10-27 |
| 04A5′L | | steric | SMN2 | | *meA*meA*meU*meG*meC*meU*meG*meG-L4- | | conjugated with DS06-0004 at |
| 4-27A | | block | | | AGACGAGGCCUAAGCAACATT | | 5′-end of its sense strand |
| | | | | 6 | UGUUGCUUAGGCCUCGUCUTT | 21 | by C6 linker |
| DA6- | DAO | RNAa + | SMN2 + | 41 | AGACGAGGCCUAAGCAACATT-L1- | 39 | DAO in the form of ASO-10-27 |
| 04A3′L | | steric | SMN2 | | meU*meC*meA*meC*meU*meU*meU*meC*meA*meU | | conjugated with DS06-0004 |
| 1-27A | | block | | | *meA*meA*meU*meG*meC*meU*meG*meG | | duplex at 3′-end of its |
| | | | | 6 | UGUUGCUUAGGCCUCGUCUTT | 21 | sense strand by S18 linker |
| DA6- | DAO | RNAa + | SMN2 + | 42 | AGACGAGGCCUAAGCAACATT-L9- | 39 | DAO in the form of ASO-10-27 |
| 04A3′L | | steric | SMN2 | | meU*meC*meA*meC*meU*meU*meU*meC*meA*meU | | conjugated with DS06-0004 at |
| 9-27A | | block | | | *meA*meA*meU*meG*meC*meU*meG*meG | | 3′-end of its sense strand |
| | | | | 6 | UGUUGCUUAGGCCUCGUCUTT | 21 | by S9 linker |
| DA6- | DAO | RNAa + | SMN2 + | 43 | AGACGAGGCCUAAGCAACATT-L4- | 39 | DAO in the form of ASO-10-27 |
| 04A3′L | | steric | SMN2 | | meU*meC*meA*meC*meU*meU*meU*meC*meA*meU | | conjugated with DS06-0004 at |
| 4-27A | | block | | | *meA*meA*meU*meG*meC*meU*meG*meG | | 3′-end of its sense strand |
| | | | | 6 | UGUUGCUUAGGCCUCGUCUTT | 21 | by C6 linker |
| DA6- | DAO | RNAa + | SMN2 + | 5 | AGACGAGGCCUAAGCAACATT | 21 | DAO in the form of ASO-10-27 |
| 04B3′L | | steric | SMN2 | 44 | UGUUGCUUAGGCCUCGUCUTT- | | conjugated with DS06-0004 at |
| 0-27A | | block | | | meU*meC*meA*meC*meU*meU*meU*meC*meA*meU | 39 | 3′end of its antisense |
| | | | | | *meA*meA*meU*meG*meC*meU*meG*meG | | strand by dTdT |
| DA6- | DAO | RNAa + | SMN2 + | 5 | AGACGAGGCCUAAGCAACATT | 21 | DAO in the form of ASO-10-27 |
| 04B3′L | | steric | SMN2 | 45 | UGUUGCUUAGGCCUCGUCUTT-L1- | | conjugated with DS06-0004 at |
| 1-27A | | block | | | meU*meC*meA*meC*meU*meU*meU*meC*meA*meU | 39 | 3′end of its antisense |
| | | | | | *meA*meA*meU*meG*meC*meU*meG*meG | | strand by S18 linker |
| DA6- | DAO | RNAa + | SMN2 + | 5 | AGACGAGGCCUAAGCAACATT | 21 | DAO in the form of ASO-10-27 |
| 04B3′L | | steric | SMN2 | 46 | UGUUGCUUAGGCCUCGUCUTT-L9- | | conjugated with DS06-0004 at |
| 9-27A | | block | | | meU*meC*meA*meC*meU*meU*meU*meC*meA*meU | 39 | 3′end of its antisense |
| | | | | | *meA*meA*meU*meG*meC*meU*meG*meG | | strand by S9 linker |
| DA6- | DAO | RNAa + | SMN2 + | 5 | AGACGAGGCCUAAGCAACATT | 21 | DAO in the form of ASO-10-27 |
| 04B3′L | | steric | SMN2 | 47 | UGUUGCUUAGGCCUCGUCUTT-L4- | | conjugated with DS06-0004 at |
| 4-27A | | block | | | meU*meC*meA*meC*meU*meU*meU*meC*meA*meU | 39 | 3′end of its antisense |
| | | | | | *meA*meA*meU*meG*meC*meU*meG*meG | | strand by C6 linker |
| DA06- | DAO | RNAa + | SMN2 + | 48 | CGAGACGAGGCCUAAGCAACATT-L1- | 41 | DAO in the form of ASO-10-27 |
| 04A21L | | steric | SMN2 | | meU*meC*meA*meC*meU*meU*meU*meC*meA*meU | | conjugated with DS06-0004 at |
| 1-27A | | block | | | *meA*meA*meU*meG*meC*meU*meG*meG | | 3′-end of its sense strand |
| | | | | 49 | UGUUGCUUAGGCCUCGUCUCGTT | 23 | by S9 linker, the duplex is |
| | | | | | | | extended by 2 nt at 3′ end |
| DA06- | DAO | RNAa + | SMN2 + | 50 | CGAGACGAGGCCUAAGCAACATT | 23 | DAO in the form of ASO-10-27 |
| 04B21L | | steric | SMN2 | 51 | UGUUGCUUAGGCCUCGUCUCGTT-L1- | | conjugated with DS06-0004 at |
| 1-27A | | block | | | meU*meC*meA*meC*meU*meU*meU*meC*meA*meU | | 3′end of its antisense |
| | | | | | *meA*meA*meU*meG*meC*meU*meG*meG | 41 | strand by S18 linker, with |
| | | | | | | | the duplex expand 2-nt at |
| | | | | | | | its 3′end |

Keys:
*, phosphorothioate (PS); f, 2′-fluoro; m, 2′-OMe; me, 2′-MOE; p, 5′-phosophate; VP, 5′-(E)-vinylphosphonate; C, 5-methyl cytosine; U, 5-methyl uracil; L1, spacer-18 (S18 linker); LA, spacer-C6 (C6 linker); L9, spacer-9 (S9 linker)

As shown in FIG. 10A, ASO10-27, DS06-0067 and DS06-0004 induced a 1.9-, 1.6- and 2.8-fold increase of SMN2FL mRNA and a 0.0-, 3.1- and 2.3-fold increase of SMN2A7 mRNA. Compared to DS06-0067, all 13 DS06-0067-containing DAOs had higher activity in inducing SMN2FL than either ASO10-27 or DS06-0067 used alone, with the highest activity (3.5 fold) exhibited by DA6-67A-27A in which the ASO was conjugated to the 3′end of the sense strand of the saRNA duplex by a space 18 linker. Among 13 DS06-0004-containing DAOs, 3 (DA6-04A3′-L1-27A, DA6-04A3′-L9-27A and DA6-04A3′-LA-27A) exhibited higher activity in inducing SMN2FL, and in them, the ASO was conjugated to the sense strand of the saRNA duplex by a space 18, space 9 and spacer C6 respectively.

The RT-qPCR result presented in FIG. 10A was further verified by semi-quantitative RT-PCR followed by DdeI digestion in FIG. 10B. Consistent with RT-qPCR result, most of the DAOs increased SMNFL by over 2.0 fold.

Together, results from this experiment suggest that the conjugation location in the duplex affect activity of the resulted DAOs. In general, conjugating the ASO to the 3′end of the sense strand in the duplex yields DAOs with good activity whereas the type of linkers in the conjugation has less impact on activity.

### 5.10. Example 10: Effects of DAOs with Different Conjugation Positions and Linkers on the Expression of SMN2FL and SMN2Δ7 mRNA in SMA GM00232 Cells

The DAOs assessed in Example 9 were also tested in SMA GM00232 cells for their activity in inducing SMN2 mRNA expression. As shown in FIG. 11, ASO10-27, DS06-0067 and DS06-0004 induced a 2.3-, 1.9- and 2.4-fold increase of SMN2FL mRNA respectively. Compared to DS06-0067, all 13 DS06-0067-containing DAOs had higher activity in inducing SMN2FL than either ASO10-27 or DS06-0067 used alone, with the highest activity (4.5 fold) exhibited by DA06-67A-27A. Among 13 DS06-0004-containing DAOs, all except one (DA6-04A5′-L9-27A) exhibited higher activity in inducing SMN2FL than DS06-0004 or ASO10-27 used alone with DA6-04A3′L1-27A having the highest activity (3.7 fold). Consistent with the RT-qPCR result, most of the DAOs increased SMN2FL by more than 2.0-fold. Together, the data presented in FIG. 10 and FIG. 11 from two SMA cell lines consistently demonstrates that DAOs with a variety of conjugation positions and linkers are functional and possesses activity contributed by the two oligonucleotide components within their molecule.

“GM00232 cells” refers to fibroblast cells provided by Coriell Institute for Medical Research. This cell line is described as SPINAL MUSCULAR ATROPHY I; SMA1. The donor subject has 2 copies of the SMN2 gene (data from several sources including Stabley et al. 2015, PMID 26247043) and is homozygous for deletion of exons 7 and 8 of the SMN1 gene. The relevant gene is SMN1; the chromosomal location is 5q12.2-q13.3, the allelic variant is described as exons 7 and 8 deleted; SPINAL MUSCULAR ATROPHY, TYPE I; and the identified mutation: is EX7-8DEL. The phenotype data derived from a fibroblast from skin (arm) of the following subject characterized as: Progressive muscular atrophy; absent deep tendon reflexes; abnormal EMG; donor subject has 2 copies of the SMN2 gene (data from several sources including Stabley et al. 2015, PMID 26247043) and is homozygous for deletion of exons 7 and 8 of the SMN1 gene.

### 5.11. Example 11: Effects of “saRNA-saRNA” DAOs and 3-Unit DAOs on the Expression of SMN2 mRNA and Protein in GM03813 and GM00232 Cells

Additional DAOs were created by connecting two saRNAs (“saRNA-saRNA” DAOs) and by further adding an ASO unit into the “saRNA-saRNA” DAOs, giving rise to 3-unit DAOs. These DAOs were tested in GM03813 and GM00232 cells for their activity in inducing SMN2 mRNA and protein expression.

R6-04S1&67S1R-L1V2 and R6-04S1&67S5R-L1V2 are “saRNA-saRNA” DAOs with 2 saRNAs linked by a spacer 18 linker (Table 12).

R6-04S1&27A&67S1R-L1V2 and R6-04S1&27A&67S5-L1V2 are 3-unit DAOs with an ASO linked to an “saRNA-saRNA” DAO and positioned between the two saRNAs (Table 12).

R6-04S1&67S1R&27A-L1V2 and R6-04S1&67S5&27A-L1V2 are 3-unit DAOs with a ASO linked to an “saRNA-saRNA” DAO and positioned at one side of the two saRNAs (Table 12)

TABLE 12
Representative oligonucleotide sequences tested in Example 11

| | | | | SEQ | | | |
|---|---|---|---|---|---|---|---|
| | Cat- | | Target | ID | | Size | |
| Name | egory | MOA | gene | NO: | Sequence (5′-3′) | (nt) | Design |
| ASO10- | ASO | steric | SMN2 | 11 | meU*meC*meA*meC*meU*meU*meU*meC*meA* | 18 | steric |
| 27 | | block | | | meU*meA*meA*meU*meG*meC*meU*meG*meG | | block ASO |
| R6- | DAO | RNAa + | SMN2 + | 52 | GACGAGGCCUAAGCAACA-L1- | 35 | 2 saRNAs |
| 04S1&67 | | RNAa | SMN2 | | CGUCUCGAACUCCAGUG | | linked by |
| S1R- | | | | 53 | UGUUGCUUAGGCCUCGUCUC | 20 | S18 |
| L1V2 | | | | 78 | CACUGGAGUUCGAGACGAG | 19 | |
| R6- | 3- | RNAa + | SMN2 + | 54 | GACGAGGCCUAAGCAACA-L1- | 53 | 2 saRNAs |
| 04S1&27 | unit | steric | SMN2 + | | meU*meC*meA*meC*meU*meU*meU*meC*meA* | | (R6-04S1 |
| A&67S1R- | DAO | block + | SMN2 | | meU*meA*meA*meU*meG*meC*meU*meG*meG- | | and 67S1R) |
| L1V2 | | RNAa | | | L1-CGUCUCGAACUCCAGUG | | and AS010- |
| | | | | 53 | UGUUGCUUAGGCCUCGUCUC | 20 | 27 linked |
| | | | | 78 | CACUGGAGUUCGAGACGAG | 19 | by S18 and |
| | | | | | | | with ASO |
| | | | | | | | in between |
| R6- | 3- | RNAa + | SMN2 + | 55 | GACGAGGCCUAAGCAACA-L1- | 53 | 2 saRNA |
| 04S1&67 | unit | RNAa + | SMN2 + | | CGUCUCGAACUCCAGUG-L1- | | (R6-04S1 |
| S1R&27A- | DAO | steric | SMN2 | | meU*meC*meA*meC*meU*meU*meU*meC*meA* | | and 67S1R) |
| L1V2 | | block | | | meU*meA*meA*meU*meG*meC*meU*meG*meG | | and |
| | | | | 53 | UGUUGCUUAGGCCUCGUCUC | 20 | ASO10-27 |
| | | | | 78 | CACUGGAGUUCGAGACGAG | 19 | linked by |
| | | | | | | | S18 and |
| | | | | | | | with ASO |
| | | | | | | | located |
| | | | | | | | outside |
| R6- | DAO | RNAa + | SMN2 + | 56 | GACGAGGCCUAAGCAACA-L1- | 37 | 2 saRNAs |
| 04S1&67 | | RNAa | SMN2 | | CUCGUCUCGAACUCCAGUG | | (R6-04S1 |
| S5-L1V2 | | | | 53 | UGUUGCUUAGGCCUCGUCUC | 20 | and 67S5) |
| | | | | 78 | CACUGGAGUUCGAGACGAG | 19 | linked |
| | | | | | | | by S18 |
| R6- | 3- | RNAa + | SMN2 + | 57 | GACGAGGCCUAAGCAACA-L1- | 55 | 2 saRNAs |
| 04S1&27 | unit | steric | SMN2 + | | meU*meC*meA*meC*meU*meU*meU*meC*meA* | | (R6-04S1 |
| A&67S5- | DAO | block + | SMN2 | | meU*meA*meA*meU*meG*meC*meU*meG*meG- | | and 67S5) |
| L1V2 | | RNAa | | | L1-CUCGUCUCGAACUCCAGUG | | and |
| | | | | 53 | UGUUGCUUAGGCCUCGUCUC | 20 | ASO10-27 |
| | | | | 78 | CACUGGAGUUCGAGACGAG | 19 | linked by |
| | | | | | | | S18 and |
| | | | | | | | with ASO |
| | | | | | | | in between |
| R6- | 3- | RNAa + | SMN2 + | 58 | GACGAGGCCUAAGCAACA-L1- | 55 | 2 saRNAs |
| 04S1&67 | unit | RNAa + | SMN2 + | | CUCGUCUCGAACUCCAGUG-L1- | | (R6-04S1 |
| S5&27A- | DAO | steric | SMN2 | | meU*meC*meA*meC*meU*meU*meU*meC*meA* | | and 67S5 |
| L1V2 | | block | | | meU*meA*meA*meU*meG*meC*meU*meG*meG | | and |
| | | | | 53 | UGUUGCUUAGGCCUCGUCUC | 20 | ASO10-27 |
| | | | | 78 | CACUGGAGUUCGAGACGAG | 19 | linked by |
| | | | | | | | S18 and |
| | | | | | | | with the |
| | | | | | | | ASO located |
| | | | | | | | outside |

Keys:
*, phosphorothioate (PS); f, 2′-fluoro; m, 2′-OMe; me, 2′-MOE; p, 5′-phosophate; VP, 5′-(E)-vinylphosphonate; C, 5-methyl cytosine; U, 5-methyl uracil; L1, spacer-18 (S18 linker); L4, spacer-C6 (C6 linker); L9, spacer-9 (S9 linker)

As shown in FIG. 12A, ASO10-27 and DAOs were transfected at 25 nM into GM03813 cells. ASO10-27 induced a 1.9-fold increase of SMN2FL mRNA, whereas the two “saRNA-saRNA” DAOs R6-04S1&67S1R-L1V2 and R6-04S1&67S5-L1V2 induced a 2.6- and 2.6-fold increase of SMN2FL mRNA and a 1.9- and 2.0-fold increase of SMN2Δ7mRNA, with total SMN2 mRNA reaching 4.5 and 4.6 fold respectively, indicating additive activity from the two connected saRNAs.

Compared to ASO10-27 or “saRNA-saRNA” DAOs, all 3-unit DAOs R6-04S1&27A&67S1R-L1V2, R6-04S1&67S1R&27A-L1V2, R6-04S1&27A&67S5-L1V2 and R6-04S1&67S5&27A-L1V2 exhibited higher activity in inducing SMN2FL mRNA and caused a 3.7-,4.3-, 3.8- and 4.4-fold increase of SMN2FL mRNA respectively with concurrent disappearance of SMN2Δ7, indicating activities contributed by both the saRNAs and the ASO.

In comparison with DAOs with ASO positioned between the two saRNAs (i.e., R6-04S1&27A&67S1R-L1V2 and R6-04S1&27A&67S5-L1V2), DAOs with the ASO positioned at the side of the two saRNAs (i.e., R6-04S1&67S1R&27A-L1V2 and R6-04S1&67S5&27A-L1V2) exhibited stronger activity in inducing SMN2FL mRNA (FIG. 12A). Very consistent result was obtained from repeating the same experiment in GM00232 cells as shown in FIG. 12B.

Further, SMN protein levels were detected by western blotting in both GM03813 and GM00232 cells transfected with ASO10-27 or DAOs tested in FIG. 12. As shown in FIGS. 13A and 13B, ASO10-27, R6-04S1&67S1R-L1V2 and R6-04S1&67S5-L1V2 induced a 5.8-, 6.9- and 4.8-fold increase of SMN protein in GM03813 cells. R6-04S1&27A&67S1R-L1V2, R6-04S1&67S1R&27A-L1V2, R6-04S1&27A&67S5-L1V2 and R6-04S1&67S5&27A-L1V2 induced a 8.2-, 9.8-, 6.7- and 6.8-fold increase of SMN protein respectively. The activity 3-unit DAOs in inducing SMN protein was much higher than ASO10-27 or “saRNA-saRNA” DAOs.

In GM00232 cells, ASO10-27, R6-04S1&67S1R-L1V2 and R6-04S1&67S5-L1V2 induced a 4.4-, 4.6- and 4.7-fold increase of SMN protein (FIGS. 13C and 13D). 3-unit DAOs R6-04S1&27A&67S1R-L1V2, R6-04S1&67S1R&27A-L1V2, R6-04S1&27A&67S5-L1V2 and R6-04S1&67S5&27A-L1V2 induced a 7.8-, 10.7-, 3.7- and 7.5-fold increase of SMN protein respectively, with R6-04S1&67S1R&27A-L1V2 and R6-04S1&67S5&27A-L1V2 achieving over a 7-fold increase in SMN protein.

These results further demonstrate that DAOs composed of oligonucleotide units of the same MOA can have additive effect and warrant the use of such DAOs in disease treatment to maximize gene output with reduced cost in drug manufacturing compared to simply combining two oligonucleotide drugs.

Furthermore, these results prove the feasibility and activity of novel 3-unit DAOs. As listed in Table 6, 3-unit DAOs could be composed of different oligonucleotide units with different MOAs to achieve a wide variety of potential by simultaneously manipulating the expression of 2 or more target genes.

### 5.12. Example 12: Effects of DAOs with Varying Sized ASOs on the Expression of SMN2FL and SMN2Δ7 mRNA in GM03813 and GM09677 Cells

To further optimize the structure of SMN2 DAOs, a series of new DAOs were created by conjugating R6-04(20)-S1V1v(CM-4), which was derived from RAG06-0004 with chemical modification, with different sized SMN2 splice modulating ASOs. These new ASOs were based on 18-nt ASO10-27 and have a size ranging from 6, 7, 8, 9, 10, 11, 12, 13, 14, 15 to 16 nt. These ASOs were conjugated with R6-04M1 by a S18 linker, giving rise to R6-04M1-6nt-S1L1V3v, R6-04M1-7nt-S1L1V3v, R6-04M1-8nt-S1L1V3v, R6-04M1-9nt-S1L1V3v, R6-04M1-10nt-S1L1V3v, R6-04M1-11nt-S1L1V3v, R6-04M1-12nt-S1L1V3v, R6-04M1-13nt-S1L1V3v, R6-04M1-14nt-S1L1V3v, R6-04M1-15nt-S1L1V3v, R6-04M1-16nt-S1L1V3v (Table 13).

A series of controls were also created and contain the saRNA duplex R6-04(20)-S1V1v(CM-4) linked by a S18 linker to a control ASO sequence (AC2) ranging from 8 to 18 nt. They are R6-04M1-AC2(8)-S1L1V3v, R6-04M1-AC2(9)-S1L1V3v, R6-04M1-AC2(10)-S1L1V3v, R6-04M1-AC2(11)-S1L1V3v, R6-04M1-AC2(12)-S1L1V3v, R6-04M1-AC2(13)-S1L1V3v, R6-04M1-AC2(14)-S1L1V3v, R6-04M1-AC2(15)-S1L1V3v, R6-04M1-AC2(16)-S1L1V3v, R6-04M1-AC2(18)-S1L1V3v. These controls differ from their correspondingly sized DAOs only by the single-stranded ASO part (Table 13).

TABLE 13
Representative oligonucleotide sequences tested in Example 12

| | | | | SEQ | | | |
|---|---|---|---|---|---|---|---|
| | | | Target | ID | | Size | |
| Name | Category | MOA | gene | NO: | Sequence (5′-3′) | (nt) | Design |
| AS010-27 | ASO | steric | SMN2 | 11 | meU*meC*meA*meC*meU*meU*meU*meC*meA*me | 18 | steric block ASO |
| | | block | | | U*meA*meA*meU*meG*meC*meU*meG*meG | | |
| R6- | saRNA | RNAa | SMN2 | 60 | mG*fA*mCfGmAfGmGfCfCfUfAfAmGfCmAfA*mC* | 18 | chemically modified R6-04- |
| 04(20)- | | | | | fA | | S1 |
| S1V1v | | | | 17 | VPmU*fG*mUfUmGfCmUfUmAfGmGfCmC*fU*mC*f | 20 | |
| (CM-4) | | | | | G*mU*fC*mU*fC | | |
| R6-04M1- | DAO | RNAa + | SMN2 + | 61 | mG*fA*mCfGmAfGmGfCfCfUfAfAmGfCmAfA*mC* | 36 | DAO composed of R6-04(20)- |
| 27A- | | steric | SMN2 | | fA-L1- | | S1V1v (CM-4) and ASO10-27 |
| S1L1V3v | | block | | | meU*meC*meA*meC*meU*meU*meU*meC*meA*me | | linked by S18 linker |
| | | | | | U*meA*meA*meU*meG*meC*meU*meG*meG | | |
| | | | | 17 | VPmU*fG*mUfUmGfCmUfUmAfGmGfCmC*fU*mC*f | 20 | |
| | | | | | G*mU*fC*mU*fC | | |
| R6-04M1- | DAO | RNAa + | SMN2 + | 79 | mG*fA*mCfGmAfGmGfCfCfUfAfAmGfCmAfA*mC* | 34 | DAO in the form of 16-nt |
| 16 nt- | | steric | SMN2 | | fA-L1- | | ASO conjugated with saRNA |
| S1L1V3v | | block | | | meA*meC*meU*meU*meU*meC*meA*meU*meA*me | | R6-04M1 duplex at 3′-end of |
| | | | | | A*meU*meG*meC*meU*meG*meG | | its sense strand by S18 |
| | | | | 17 | VPmU*fG*mUfUmGfCmUfUmAfGmGfCmC*fU*mC*f | 20 | |
| | | | | | G*mU*fC*mU*fC | | |
| R6-04M1- | DAO | RNAa + | SMN2 + | 80 | mG*fA*mCfGmAfGmGfCfCfUfAfAmGfCmAfA*mC* | 33 | DAO in the form of 15-nt |
| 15 nt- | | steric | SMN2 | | fA-L1- | | ASO conjugated with saRNA |
| S1L1V3v | | block | | | meU*meU*meU*meC*meA*meU*meA*meA*meU*me | | R6-04M1 duplex at 3′-end of |
| | | | | | G*meC*meU*meG*meG*meC | | its sense strand by S18 |
| | | | | 17 | VPmU*fG*mUfUmGfCmUfUmAfGmGfCmC*fU*mC*f | 20 | |
| | | | | | G*mU*fC*mU*fC | | |
| R6-04M1- | DAO | RNAa + | SMN2 + | 81 | mG*fA*mCfGmAfGmGfCfCfUfAfAmGfCmAfA*mC* | 32 | DAO in the form of 14-nt |
| 14 nt- | | steric | SMN2 | | fA-L1- | | ASO conjugated with saRNA |
| S1L1V3v | | block | | | meU*meU*meC*meA*meU*meA*meA*meU*meG*me | | R6-04M1 duplex at 3′-end of |
| | | | | | C*meU*meG*meG*meC | | its sense strand by S18 |
| | | | | 17 | VPmU*fG*mUfUmGfCmUfUmAfGmGfCmC*fU*mC*f | 20 | |
| | | | | | G*mU*fC*mU*fC | | |
| R6-04M1- | DAO | RNAa + | SMN2 + | 82 | mG*fA*mCfGmAfGmGfCfCfUfAfAmGfCmAfA*mC* | 31 | DAO in the form of 13-nt |
| 13 nt- | | steric | SMN2 | | fA-L1- | | ASO conjugated with saRNA |
| S1L1V3v | | block | | | meU*meC*meA*meU*meA*meA*meU*meG*meC*me | | R6-04M1 duplex at 3′-end of |
| | | | | | U*meG*meG*meC | | its sense strand by S18 |
| | | | | 17 | VPmU*fG*mUfUmGfCmUfUmAfGmGfCmC*fU*mC*f | 20 | |
| | | | | | G*mU*fC*mU*fC | | |
| R6-04M1- | DAO | RNAa + | SMN2 + | 83 | mG*fA*mCfGmAfGmGfCfCfUfAfAmGfCmAfA*mC* | 30 | DAO in the form of 12-nt |
| (12 nt-B)- | | steric | SMN2 | | fA-L1- | | ASO conjugated with saRNA |
| S1L1V3v | | block | | | meA*meU*meA*meA*meU*meG*meC*meU*meG*me | | R6-04M1 duplex at 3′-end of |
| | | | | | G*meC*meA | | its sense strand by S18 |
| | | | | 17 | VPmU*fG*mUfUmGfCmUfUmAfGmGfCmC*fU*mC*f | 20 | |
| | | | | | G*mU*fC*mU*fC | | |
| R6-04M1- | DAO | RNAa + | SMN2 + | 84 | mG*fA*mCfGmAfGmGfCfCfUfAfAmGfCmAfA*mC* | 29 | DAO in the form of 11-nt |
| 11 nt- | | steric | SMN2 | | fA-L1- | | ASO conjugated with saRNA |
| S1L1V3v | | block | | | meA*meA*meU*meG*meC*meU*meG*meG*meC*me | | R6-04M1 duplex at 3′-end of |
| | | | | | A*meG | | its sense strand by S18 |
| | | | | 17 | VPmU*fG*mUfUmGfCmUfUmAfGmGfCmC*fU*mC*f | 20 | |
| | | | | | G*mU*fC*mU*fC | | |
| R6-04M1- | DAO | RNAa + | SMN2 + | 85 | mG*fA*mCfGmAfGmGfCfCfUfAfAmGfCmAfA*mC* | 29 | DAO in the form of 10-nt |
| 10 nt- | | steric | SMN2 | | fA-L1- | | ASO conjugated with saRNA |
| S1L1V3v | | block | | | meA*meA*meU*meG*meC*meU*meG*meG*meC*me | | R6-04M1 duplex at 3′-end of |
| | | | | | A*meG | | its sense strand by S18 |
| | | | | 17 | VPmU*fG*mUfUmGfCmUfUmAfGmGfCmC*fU*mC*f | 20 | |
| | | | | | G*mU*fC*mU*fC | | |
| R6-04M1- | DAO | RNAa + | SMN2 + | 86 | mG*fA*mCfGmAfGmGfCfCfUfAfAmGfCmAfA*mC* | 27 | DAO in the form of 9-nt ASO |
| 9 nt- | | steric | SMN2 | | fA-L1- | | conjugated with saRNA R6- |
| S1L1V3v | | block | | | meU*meG*meC*meU*meG*meG*meC*meA*meG | | 04M1 duplex at 3′-end of |
| | | | | 17 | VPmU*fG*mUfUmGfCmUfUmAfGmGfCmC*fU*mC*f | 20 | its sense strand by S18 |
| | | | | | G*mU*fC*mU*fC | | |
| R6-04M1- | DAO | RNAa + | SMN2 + | 87 | mG*fA*mCfGmAfGmGfCfCfUfAfAmGfCmAfA*mC* | 26 | DAO in the form of 8-nt ASO |
| 8 nt- | | steric | SMN2 | | fA-L1-meG*meC*meU*meG*meG*meC*meA*meG | | conjugated with saRNA R6- |
| S1L1V3v | | block | | 17 | VPmU*fG*mUfUmGfCmUfUmAfGmGfCmC*fU*mC*f | 20 | 04M1 duplex at 3′-end of |
| | | | | | G*mU*fC*mU*fC | | its sense strand by S18 |
| R6-04M1- | DAO | RNAa + | SMN2 + | 88 | mG*fA*mCfGmAfGmGfCfCfUfAfAmGfCmAfA*mC* | 25 | DAO in the form of 7-nt ASO |
| 7 nt- | | steric | SMN2 | | fA-L1-meG*meC*meU*meG*meG*meC*meA | | conjugated with saRNA R6- |
| S1L1V3v | | block | | 17 | VPmU*fG*mUfUmGfCmUfUmAfGmGfCmC*fU*mC*f | 20 | 04M1 duplex at 3′-end of |
| | | | | | G*mU*fC*mU*fC | | its sense strand by S18 |
| R6-04M1- | DAO | RNAa + | SMN2 + | 89 | mG*fA*mCfGmAfGmGfCfCfUfAfAmGfCmAfA*mC* | 24 | DAO in the form of 6-nt ASO |
| 6 nt- | | steric | SMN2 | | fA-L1-meG*meC*meU*meG*meG*meC | | conjugated with saRNA R6- |
| S1L1V3v | | block | | 17 | VPmU*FG*mUfUmGfCmUfUmAfGmGfCmC*fU*mC*f | 20 | 04M1 duplex at 3′-end of |
| | | | | | G*mU*fC*mU*fC | | its sense strand by S18 |
| R6-04M1- | Ctrl | RNAa + | SMN2 | 90 | mG*fA*mCfGmAfGmGfCfCfUfAfAmGfCmAfA*mC* | 36 | DAO in the form of |
| AC2(18)- | DAO | # | | | fA-L1- | | nonfunctional AC2 (18-nt) |
| S1L1V3v | | | | | meU*meA*meG*meA*meC*meU*meA*meG*meA*me | | ASO conjugated with saRNA |
| | | | | | U*meC*meA*meU*meA*meU*meG*meA*meG | | R6-04M1 duplex at 3′-end of |
| | | | | 17 | VPmU*fG*mUfUmGfCmUfUmAfGmGfCmC*fU*mC*f | 20 | its sense strand by S18 |
| | | | | | G*mU*fC*mU*fC | | |
| R6-04M1- | Ctrl | RNAa + | SMN2 | 91 | mG*fA*mCfGmAfGmGfCfCfUfAfAmGfCmAfA*mC* | 34 | DAO in the form of AC2 (16- |
| AC2(16)- | DAO | # | | | fA-L1- | | nt) ASO conjugated with |
| S1L1V3v | | | | | meU*meA*meG*meA*meC*meU*meA*meG*meA*me | | saRNA R6-04M1 duplex at 3′- |
| | | | | | U*meC*meA*meU*meA*meU*meG | | end of its sense strand by |
| | | | | 17 | VPmU*fG*mUfUmGfCmUfUmAfGmGfCmC*fU*mC*f | 20 | S18 |
| | | | | | G*mU*fC*mU*fC | | |
| R6-04M1- | Ctrl | RNAa + | SMN2 | 92 | mG*fA*mCfGmAfGmGfCfCfUfAfAmGfCmAfA*mC* | 33 | DAO in the form of |
| AC2(15)- | DAO | # | | | fA-L1- | | nonfunctional AC2 (15-nt) |
| S1L1V3v | | | | | meU*meA*meG*meA*meC*meU*meA*meG*meA*me | | ASO conjugated with saRNA |
| | | | | | U*meC*meA*meU*meA*meU | | R6-04M1 duplex at 3′-end of |
| | | | | 17 | VPmU*fG*mUfUmGfCmUfUmAfGmGfCmC*fU*mC*f | 20 | its sense strand by S18 |
| | | | | | G*mU*fC*mU*fC | | |
| R6-04M1- | Ctrl | RNAa + | SMN2 | 93 | mG*fA*mCfGmAfGmGfCfCfUfAfAmGfCmAfA*mC* | 32 | DAO in the form of |
| AC2(14)- | DAO | # | | | fA-L1- | | nonfunctional AC2 (14-nt) |
| S1L1V3v | | | | | meU*meA*meG*meA*meC*meU*meA*meG*meA*me | | ASO conjugated with saRNA |
| | | | | | U*meC*meA*meU*meA | | R6-04M1 duplex at 3′-end of |
| | | | | 17 | VPmU*fG*mUfUmGfCmUfUmAfGmGfCmC*fU*mC*f | 20 | its sense strand by S18 |
| | | | | | G*mU*fC*mU*fC | | |
| R6-04M1- | Ctrl | RNAa + | SMN2 | 94 | mG*fA*mCfGmAfGmGfCfCfUfAfAmGfCmAfA*mC* | 31 | DAO in the form of |
| AC2(13)- | DAO | # | | | fA-L1- | | nonfunctional AC2 (13-nt) |
| S1L1V3v | | | | | meU*meA*meG*meA*meC*meU*meA*meG*meA*me | | ASO conjugated with saRNA |
| | | | | | U*meC*meA*meU | | R6-04M1 duplex at 3′-end of |
| | | | | 17 | VPmU*fG*mUfUmGfCmUfUmAfGmGfCmC*fU*mC*f | 20 | its sense strand by S18 |
| | | | | | G*mU*fC*mU*fC | | |
| R6-04M1- | Ctrl | RNAa + | SMN2 | 95 | mG*fA*mCfGmAfGmGfCfCfUfAfAmGfCmAfA*mC* | 30 | DAO in the form of |
| AC2(12)- | DAO | # | | | fA-L1- | | nonfunctional AC2 (12-nt) |
| S1L1V3v | | | | | meU*meA*meG*meA*meC*meU*meA*meG*meA*me | | ASO conjugated with saRNA |
| | | | | | U*meC*meA | | R6-04M1 duplex at 3′-end of |
| | | | | 17 | VPmU*fG*mUfUmGfCmUfUmAfGmGfCmC*fU*mC*f | 20 | its sense strand by S18 |
| | | | | | G*mU*fC*mU*fC | | |
| R6-04M1- | Ctrl | RNAa + | SMN2 | 96 | mG*fA*mCfGmAfGmGfCfCfUfAfAmGfCmAfA*mC* | 20 | DAO in the form of |
| AC2(11)- | DAO | # | | | fA-L1- | | nonfunctional AC2 (11-nt) |
| S1L1V3v | | | | | meU*meA*meG*meA*meC*meU*meA*meG*meA*me | | ASO conjugated with saRNA |
| | | | | | U*meC | | R6-04M1 duplex at 3′-end of |
| | | | | 17 | VPmU*fG*mUfUmGfCmUfUmAfGmGfCmC*fU*mC*f | | its sense strand by S18 |
| | | | | | G*mU*fC*mU*fC | | |
| R6-04M1- | Ctrl | RNAa + | SMN2 | 97 | mG*fA*mCfGmAfGmGfCfCfUfAfAmGfCmAfA*mC* | 28 | DAO in the form of |
| AC2(10)- | DAO | # | | | fA-L1- | | nonfunctional AC2 (10-nt) |
| S1L1V3v | | | | | meU*meA*meG*meA*meC*meU*meA*meG*meA*me | | ASO conjugated with saRNA |
| | | | | | U | | R6-04M1 duplex at 3′-end of |
| | | | | 17 | VPmU*fG*mUfUmGfCmUfUmAfGmGfCmC*fU*mC*f | 20 | its sense strand by S18 |
| | | | | | G*mU*fC*mU*fC | | |
| R6-04M1- | Ctrl | RNAa + | SMN2 | 98 | mG*fA*mCfGmAfGmGfCfCfUfAfAmGfCmAfA*mC* | 27 | DAO in the form of |
| AC2(9)- | DAO | # | | | fA-L1- | | nonfunctional AC2 (9-nt) ASO |
| S1L1V3v | | | | | meU*meA*meG*meA*meC*meU*meA*meG*meA | | conjugated with saRNA R6- |
| | | | | 17 | VPmU*fG*mUfUmGfCmUfUmAfGmGfCmC*fU*mC*f | 20 | 04M1 duplex at 3′-end of |
| | | | | | G*mU*fC*mU*fC | | its sense strand by S18 |
| R6-04M1- | Ctrl | RNAa + | SMN2 | 99 | mG*fA*mCfGmAfGmGfCfCfUfAfAmGfCmAfA*mC* | 26 | DAO in the form of |
| AC2(8)- | DAO | # | | | fA-L1-meU*meA*meG*meA*meC*meU*meA*meG | | nonfunctional AC2 (8-nt) ASO |
| S1L1V3v | | | | 17 | VPmU*fG*mUfUmGfCmUfUmAfGmGfCmC*fU*mC | | conjugated with saRNA R6- |
| | | | | | *FG*mU*fC*mU*fC | 20 | 04M1 duplex at 3′-end of |
| | | | | | | | its sense strand by S18 |

Keys:
*, phosphorothioate (PS); f, 2′-fluoro; m, 2′-OMe; me, 2′-MOE; p, 5′-phosphate; VP, 5′-(E)-vinylphosphonate; C, 5-methyl cytosine; U, 5-methyl uracil; L1, spacer-18 (S18 linker); L4, spacer-C6 (C6 linker); L9, spacer-9 (S9 linker); Ctrl, control; RNAa + #, a saRNA + a control ASO; #, a control ASO with no function.

These DAOs and their controls were transfected into GM03813 and GM09677 cells at 25 nM for 72 hours. As additional controls, ASO ASO10-27 and saRNA R6-04(20)-S1V1v(CM-4) were also transfected.

As shown in FIG. 14A, in GM03813 cells, all DAOs caused a higher (at least 4.2 fold) increase in SMN2FL mRNA compared to only a 2.0 and 2.6-fold increase by ASO10-27 and R6-04(20)-S1V1v(CM-4) respectively, with the highest induction (7.3 fold) occurring in R6-04M1-7nt-S1L1V3v treated cells. In contrast, the control DAOs only exhibited an activity in inducing SMN2FL and SMN2Δ7 comparable to the saRNA duplex R6-04(20)-S1V1v(CM-4), suggesting their activity came from the saRNA unit.

Further, a concurrent reduction in the level of SMN2A7 was evident, indicating that the increased activity of DAOs in inducing SMN2FL was contributed by both the saRNA and the splicing modulating ASO components. Furthermore, the activity of DAOs gradually increased when the ASO's size was decreased from 18 nt to 13 nt, but reduced when the ASOs size was further decreased from 12 nt to 11 nt, and came back when the ASO was further decreased to 6 nt.

When these DAOs were transfected into GM09677 cells, very consistent result was obtained (FIG. 14B).

The data demonstrated that the ASO unit in DAOs can be optimized independently by varying its length and higher activity in inducing the expression of SMN2FL can be achieved by using a shorter (12 nt˜15 nt, 6 nt˜9 nt) than typical sized (18 nt) ASO in their structures.

### 5.13. Example 13: Effects of DAOs with Varying Sized ASOs on the Expression of SMN Protein in GM03813 and GM09677 Cells

To further verify protein level induction of these DAOs described in Example 12 (Table 13), SMN protein was assessed by western blotting. All DAOs were individually transfected into GM03813 and GM09677 cells at 25 nM for 72 hours. As additional controls, ASO10-27 and saRNA R6-04(20)-S1V1v(CM-4) were also transfected.

As shown in FIGS. 15A & 15B, all DAOs in GM03813 cells caused a higher (at least 3.5 fold) increase in SMN protein compared to only a 2.9- and 2.4-fold increase by ASO10-27 and R6-04(20)-S1V1v(CM-4) respectively, with the highest induction (6.0 fold) occurring in R6-04M1-15nt-S1L1V3v treated cells. The activity of DAOs gradually increased when the ASO's size was decreased from 18 nt to 15 nt, but reduced when the ASOs size was further decreased from 14 nt to 11 nt, and remain unchanged when the ASO was further decreased from 10 to 8 nt. Consistent with the GM03813 cells results, all DAOs caused a higher (at least 2.4 fold) increase in SMN protein compared to only a 1.6- and 1.1-fold increase by ASO10-27 and R6-04(20)-S1V1v(CM-4) respectively, with the highest induction (4.6 fold) occurring in R6-04M1-(12nt-B)-S1L1V3v treated GM09677 cells (FIG. 15C & 15D). The activity of DAOs gradually increased when the ASO's size was decreased from 18 nt to 12 nt, but dramatically reduced when the ASOs size was further decreased to 11 nt, and remained unchanged when the ASO was further decreased from 10 to 8 nt.

The results of SMN protein induction also demonstrated that the ASO unit in DAOs can be optimized independently by varying its length and higher activity in inducing SMN protein expression can be achieved by using a shorter (12 nt˜15 nt) than typical sized (18 nt) ASO in the DAO structures.

### 5.14. Example 14: Effects of DAOs with Varying Sized ASOs on the Expression of SMN2FL and SMN2Δ7 mRNA in GM03813 and GM09677 Cells

To further optimize the structure of SMN2 DAOs with varying sized ASOs, additional DAOs were created which was derived from chemically modified RAG06-0067 with different sized SMN2 splice modulating ASOs. These new ASOs were based on 18-nt ASO10-27 and have a size ranging from 8, 9, 12, 13, 14, 15 and 16 nt. These ASOs were conjugated with R6-67M3 by a S18 linker, giving rise to R6-67M3-8nt-S1L1V3, R6-67M3-9nt-S1L1V3, R6-67M3-(12nt)-SIL1V3, R6-67M3-13nt-S1L1V3, R6-67M3-14nt-S1L1V3, R6-67M3-15nt-S1L1V3, R6-67M3-16nt-S1L1V3 (Table 14).

TABLE 14
Representative oligonucleotide sequences tested in Example 14

| | | | | SEQ | | | |
|---|---|---|---|---|---|---|---|
| | | | Target | ID | | Size | |
| Name | Category | MOA | gene | NO: | Sequence (5′-3′) | (nt) | Design |
| ASO10- | ASO | steric | SMN2 | 11 | meU*meC*meA*meC*meU*meU*meU*meC*meA*meU*meA | 18 | steric block ASO |
| 27 | | block | | | *meA*meU*meG*meC*meU*meG*meG | | |
| R6- | DAO | RNAa + | SMN2 | 129 | mC*fA*mCfUmGfGmAfGmUfUmCfGmAfGmA*fC*mG*fA*m | 22 | DAO composed of R6-67M3 and |
| 67M3- | | steric | + | | G*fG*mC*fC | | AS010-27 linked by S18 |
| 27A- | | block | SMN2 | 130 | mC*fC*mUfCmGfUmCfUfCfGfAfAmCfUmCfCmAfGmUfG- | 38 | |
| S1L1V3 | | | | | L1- | | |
| | | | | | meU*meC*meA*meC*meU*meU*meU*meC*meA*meU*meA | | |
| | | | | | *meA*meU*meG*meC*meU*meG*meG | | |
| R6- | DAO | RNAa + | SMN2 | 129 | mC*fA*mCfUmGfGmAfGmUfUmCfGmAfGmA*fC*mG*fA*m | 22 | DAO in the form of 16-nt ASO |
| 67M3- | | steric | + | | G*fG*mC*fC | | conjugated with saRNA R6-67M3 |
| 16 nt- | | block | SMN2 | 131 | mC*fC*mUfCmGfUmCfUfCfGfAfAmCfUmCfCmAfGmUfG- | 36 | duplex at 3′-end of its sense |
| S1L1V3 | | | | | L1- | | strand by S18 |
| | | | | | meA*meC*meU*meU*meU*meC*meA*meU*meA*meA*meU | | |
| | | | | | *meG*meC*meU*meG*meG | | |
| R6- | DAO | RNAa + | SMN2 | 129 | mC*fA*mCfUmGfGmAfGmUfUmCfGmAfGmA*fC*mG*fA*m | 22 | DAO in the form of 15-nt ASO |
| 67M3- | | steric | + | | G*FG*mC*fC | | conjugated with saRNA R6-67M3 |
| 15 nt- | | block | SMN2 | 132 | mC*fC*mUfCmGfUmCfUfCfGfAfAmCfUmCfCmAfGmUfG- | 35 | duplex at 3′-end of its sense |
| S1L1V3 | | | | | L1- | | strand by S18 |
| | | | | | meU*meU*meU*meC*meA*meU*meA*meA*meU*meG*meC | | |
| | | | | | *meU*meG*meG*meC | | |
| R6- | DAO | RNAa + | SMN2 | 129 | mC*fA*mCfUmGfGmAfGmUfUmCfGmAfGmA*fC*mG*fA*m | 22 | DAO in the form of 14-nt ASO |
| 67M3- | | steric | + | | G*fG*mC*FC | | conjugated with saRNA R6-67M3 |
| 14 nt- | | block | SMN2 | 133 | mC*fC*mUfCmGfUmCfUfCfGfAfAmCfUmCfCmAfGmUfG- | 34 | duplex at 3′-end of its sense |
| S1L1V3 | | | | | L1- | | strand by S18 |
| | | | | | meU*meU*meC*meA*meU*meA*meA*meU*meG*meC*meU | | |
| | | | | | *meG*meG*meC | | |
| R6- | DAO | RNAa + | SMN2 | 129 | mC*fA*mCfUmGfGmAfGmUfUmCfGmAfGmA*fC*mG*fA*m | 22 | DAO in the form of 13-nt ASO |
| 67M3- | | steric | + | | G*fG*mC*fC | | conjugated with saRNA R6-67M3 |
| 13 nt- | | block | SMN2 | 134 | mC*fC*mUfCmGfUmCfUfCfGfAfAmCfUmCfCmAfGmUfG- | | duplex at 3′-end of its sense |
| S1L1V3 | | | | | L1- | 33 | strand by S18 |
| | | | | | meU*meC*meA*meU*meA*meA*meU*meG*meC*meU*meG | | |
| | | | | | *meG*meC | | |
| R6- | DAO | RNAa + | SMN2 | 129 | mC*fA*mCfUmGfGmAfGmUfUmCfGmAfGmA*fC*mG*fA*m | 22 | DAO with 12-nt ASO conjugated |
| 67M3- | | steric | + | | G*FG*mC*fC | | with saRNA R6-67M3 duplex at |
| 12 nt- | | block | SMN2 | 135 | mC*fC*mUfCmGfUmCfUfCfGfAfAmCfUmCfCmAfGmUfG- | | 3′-end of its sense strand by |
| S1L1V3 | | | | | L1- | 32 | S18 |
| | | | | | meC*meA*meU*meA*meA*meU*meG*meC*meU*meG*meG | | |
| | | | | | *meC | | |
| R6- | DAO | RNAa + | SMN2 | 129 | mC*fA*mCfUmGfGmAfGmUfUmCfGmAfGmA*fC*mG*fA*m | 22 | DAO in the form of 9-nt ASO |
| 67M3- | | steric | + | | G*fG*mC*fC | | conjugated with saRNA R6-67M3 |
| 9 nt- | | block | SMN2 | 136 | mC*fC*mUfCmGfUmCfUfCfGfAfAmCfUmCfCmAfGmUfG- | 29 | duplex at 3′-end of its sense |
| S1L1V3 | | | | | L1-meU*meG*meC*meU*meG*meG*meC*meA*meG | | strand by S18 |
| R6- | DAO | RNAa + | SMN2 | 129 | mC*fA*mCfUmGfGmAfGmUfUmCfGmAfGmA*fC*mG*fA*m | 22 | DAO in the form of 8-nt ASO |
| 67M3- | | steric | + | | G*fG*mC*fC | | conjugated with saRNA R6-67M3 |
| 8 nt- | | block | SMN2 | 137 | mC*fC*mUfCmGfUmCfUfCfGfAfAmCfUmCfCmAfGmUfG- | | duplex at 3′-end of its sense |
| S1L1V3 | | | | | L1-meG*meC*meU*meG*meG*meC*meA*meG | 28 | strand by S18 |

Keys:
*, phosphorothioate (PS); f, 2′-fluoro; m, 2′-OMe; me, 2′-MOE; p, 5′-phosophate; vp, 5′-(E)-vinylphosphonate; C, 5-methyl cytosine; U, 5-methyl uracil; L1, spacer-18 (S18 linker); L4, spacer-C6 (C6 linker); L9, spacer-9 (S9 linker)

As shown in FIG. 16A, all DAOs were transfected at 25 nM in GM03813 cells caused a higher (at least 2.6 fold) increase in SMN2FL mRNA compared to only a 1.9-fold increase by ASO10-27 alone, with the highest induction (3.5 fold) occurring in R6-67M3-15nt-S1L1V3 treated cells. Further, the activity of DAOs gradually increased when the ASO's size was decreased from 18 nt to 15 nt, but reduced when the ASOs size was further decreased (14nt˜ 12nt, 9nt˜ 8nt) and remained unchanged. Similarly, all DAOs in GM09677 cells caused a higher (at least 2.5 fold) increase in SMN2FL mRNA compared to only a 1.9-fold increase by ASO10-27 alone, with the highest induction (3.7 fold) occurring in R6-67M3-8nt-S1L1V3 treated cells (FIG. 16B). The activity of DAOs gradually increased when the ASO's size was decreased from 18 nt to 13 nt, but reduced when the ASOs size was further decreased from 12nt to 9nt, and came back when the ASO was decreased to 8nt. Furthermore, a concurrent reduction in the level of SMN2A7 was evident, indicating that the increased activity of DAOs in inducing SMN2FL was contributed by the splicing modulating ASO components (FIG. 16B).

This data demonstrated that the ASO unit in DAOs can be optimized independently by varying its length and higher activity in inducing the expression of SMN2FL can be achieved by using a shorter (13 nt, 15 nt and 8 nt) than typical sized (18 nt) ASO in their structures.

### 5.15. Example 15: Effects of DAOs with Varying Sized ASOs on the Expression of SMN Protein in GM03813 and GM09677 Cells

To further verify protein level induction of these DAOs which described as Example 14 (Table 14), SMN protein was assessed by western blotting. All DAOs were transfected into GM03813 and GM09677 cells at 25 nM for 72 hours. ASO10-27 was also transfected as a positive control.

As shown in FIGS. 17A & 17B, in GM03813 cells, all DAOs caused a higher (at least 2.0 fold) increase in SMN protein compared to only a 1.6-fold increase by ASO10-27 alone, with the highest induction (3.6 fold) occurring in R6-67M3-15nt-S1L1V3 treated cells. Consistent with the GM03813 cells results, all DAOs caused a higher (at least 3.4 fold) increase in SMN protein in GM09677 cells compared to only a 2.2-fold increase by ASO10-27 alone, with the highest induction (4.6 fold) occurring in R6-67M3-15nt-S1L1V3 treated cells (FIGS. 17C & 17D).

Further, the activity of DAOs gradually increased when the ASO's size was decreased from 18 nt to 15 nt, but reduced when the ASOs size was further decreased from 14 nt to 9 nt, and remain unchanged when the ASO was further decreased to 8 nt (FIGS. 17A-17D).

The data demonstrated that the ASO unit in DAOs can be optimized independently by varying its length and higher activity in inducing SMN protein expression can be achieved by using a shorter (e.g., 15 nt) than typical sized (18 nt) ASO in their structures.

### 5.16. Example 16: Effects of DAOs with Varying Sized ASOs on the Expression of SMN2FL and SMN2Δ7 mRNA in PMH Cells

To further verify the function of DAOs in primary mouse hepatocytes (PMHs) isolated from type III SMA mice carrying two copies human SMN2 gene, the DAOs tested in Example 12 (Table 13) were transfected at 25 nM for 72 hours in PMH cells. As additional controls, ASO10-27 and saRNA R6-04(20)-S1V1v(CM-4) were also transfected.

As shown in FIG. 18, all DAOs caused a higher (at least 3.4 fold) increase in SMN2FL mRNA compared to a 2.7- and 2.2-fold increase by ASO10-27 and R6-04(20)-S1V1v(CM-4) respectively, with the highest induction (4.3 fold) occurring in R6-04M1-13nt-S1L1V3v and R6-04M1-14nt-S1L1V3v treated cells. The activity of DAOs gradually increased when the ASO's size was decreased from 18 nt to 13 nt with a little bit reduced when the ASOs size was further decreased to 12 nt, and came back when the ASO was decreased to 10 nt to 8 nt. Further, all DAOs were effective both in SMA-patient cells and PMH cells, which supports further testing in animal models and human patients.

### 5.17. Example 17: Effect of “saRNA-siRNA” DAOs Two Different Genes on the Expression of SMN2FL, SMN2Δ7 and SOD1 mRNA in 293A and GM03813 Cells

Based on “saRNA-saRNA” DAOs, a DAO was created by connecting a saRNA and a siRNA (“saRNA-siRNA” DAO) covalently via a S18 linker. In this DAO structure (R6-04M1&R17-388E-L1V2), the saRNA unit was designed to activate SMN2 gene and the siRNA unit was designed to knock down the superoxide dismutase 1 (SOD1) gene (Table 15).

Amyotrophic lateral sclerosis (ALS) and SMA are the most frequent motor neuron disorders in adulthood and infancy, respectively. Both diseases share common pathophysiological patterns and can co-occur within a family (Coreia P et al. Phenotypic and genotypic studies of ALS cases in ALS-SMA families. Amyotroph Lateral Scler Frontotemporal Degener 2018 August;19(5-6):432-437). Mutation of SOD1 gene accounts for 20% of familial ALS and the mutated SOD1 protein is believed to exert a toxic action on motor neurons can disrupt SMN protein complex. A deficiency of SMN protein is also reported to be an exacerbating factor in the development of ALS (Kariya S, et al. Mutant superoxide dismutase 1 (SOD1), a cause of amyotrophic lateral sclerosis, disrupts the recruitment of SMN, the spinal muscular atrophy protein to nuclear Cajal bodies. Hum Mol Genet. 2012 Aug. 1;21(15):3421-34). Therefore, simultaneous activation of SMN protein and knockdown of mutated SOD1 may be therapeutically beneficial.

These DAOs were transfected into HEK293A or GM03813 cells. The saRNA unit R6-04(20)-S1V1v(CM-4) and the siRNA unit siSOD1-388-ESC were also transfected either individually or in combination. The transfected cells were harvested for mRNA expression analysis 72 hours later.

As shown in FIG. 19A, R6-04(20)-S1V1v(CM-4) and siSOD1-388-ESC induced SMN2FL expression (3.18 fold) and reduced SOD1 expression (0.01 fold) respectively. Their combined treatment caused a 3.19 fold increase in SMN2FL and a 0.025 fold downregulation of SOD1, whereas the DAO R6-04M1&R17-388E-L1V2 induced SMN2FL by 1.95 fold and reduced SOD1 by 0.01 fold. Compared to combo treatment, the DAO actually exhibited higher SOD1 knockdown efficiency.

Consistent with the result in 293A cells, in GM03813 cells (FIG. 19B), R6-04(20)-S1V1v(CM-4) and siSOD1-388-ESC induced SMN2FL expression (1.85 fold) and reduced SOD1 expression (0.01 fold) respectively. Their combined treatment caused a 1.71 fold increase in SMN2FL and a 0.02 fold downregulation of SOD1, whereas the DAO R6-04M1&R17-388E-L1V2 induced SMN2FL by 1.57 fold and reduced SOD1 by 0.01 fold. Compared to combo treatment, the DAO exhibited higher SOD1 knockdown efficiency.

From testing DAO R6-04M1&R17-388E-L1V2, it is indicated that combining a saRNA with a siRNA into a DAO can largely retain the activity of each unit and sometime give rise to enhanced activity for the siRNA unit.

TABLE 15
Representative oligonucleotide sequences tested in Example 17

| | | | | SEQ | | | |
|---|---|---|---|---|---|---|---|
| | Cat- | | Target | ID | | Size | |
| Name | egory | MOA | gene | NO: | Sequence (5′-3′ ) | (nt) | Design |
| R6- | saRNA | RNAa | SMN2 | 60 | mG*fA*mCfGmAfGmGfCfCfUfAfAmGfCmAfA*mC*fA | 18 | Chemically |
| 04(20)- | | | | 17 | VPmU*fG*mUfUmGfCmUfUmAfGmGfCmC*fU*mC*FG* | 20 | modified R6-04 |
| S1V1V | | | | | mU*fC*mU*fC | | |
| (CM-4) | | | | | | | |
| siSOD1- | siRNA | steric | SOD1 | 138 | fG*mG*fUmGfGmAfAmAfUfGfAmAfGmAfAmAf*GmU* | 19 | Chemically |
| 388- | | block | | | fA | | modified siRNA |
| ESC | | | | 139 | mU*fA*mCfUmUfUmCfUmUfCmAfUmUfUmCfCmAfC | 21 | |
| | | | | | mC*fU*mU | | |
| R6- | DAO | RNAa + | SMN2 | 140 | mG*fA*mCfGmAfGmGfCfCfUfAfAmGfCmAfA*mC*f | 37 | DAO composed of |
| 04M1&R | | RNAi | + | | A-L1- | | R6-04(20)- |
| 17- | | | SOD1 | | fG*mG*fUmGfGmAfAmAfUfGfAmAfGmAfAmAf*GmU | | S1V1 (CM-4) and |
| 388E- | | | | | *fA | | siSOD1-388-ESC |
| L1V2 | | | | 17 | VPmU*fG*mUfUmGfCmUfUmAfGmGfCmC*fU*mC*fG* | 20 | with the 2 |
| | | | | | mU*fC*mU*fC | | antisense |
| | | | | 141 | VPmU*fA*mCfUmUfUmCfUmUfCmAfUmUfUmCfCmAf | | stands in |
| | | | | | CmC*fU*mU | 21 | each duplex |
| | | | | | | | linked by S18 |

Keys:
*, phosphorothioate (PS); f, 2′-fluoro; m, 2′-OMe; me, 2′-MOE; p, 5′-phosophate; vp, 5′-(E)-vinylphosphonate; C, 5-methyl cytosine; U, 5-methyl uracil; L1, spacer-18 (S18 linker); L4, spacer-C6 (C6 linker); L9, spacer-9 (S9 linker)

### 5.18. Example 18: Effect of DAOs Targeting Two Different Genes on the Expression of p21 and PD-L1 mRNA

CDKN1A (p21) and CD274 (PD-L1, programmed death-ligand 1) are two important genes implicated in tumorigenesis. p21 is a negative cell cycle regulator and a putative tumor suppressor and its activation by saRNA can lead to tumor inhibition (Kang et al. 2017). PD-L1 is an important target in cancer treatment and blocking PD-L1 can promote T-cell-mediated immunosurveillance against cancer and has shown huge clinical benefit in cancer patients.

To combine tumor inhibitory effects of p21 activation and PD-L1 blockage into a single oligonucleotide molecule, DAOs were designed by connecting a p21 saRNA (RAG1-40) and PD-L1 siRNAs (siPDL1-2, siPDL1-3) or gapmer ASOs (aPDL1-1, aPDL1-2, aPDL1-3) (Table 16). RAG1-40 was designed to target p21 gene promoter sequence to induce its transcription via the RNAa mechanism; siPDL1-2 and siPDL1-3 were designed to target PD-L1 gene's mRNA sequence to silence its expression via the RNAi mechanism and aPDL1-1, aPDL1-2 and aPDL1-3 are gapmer ASOs designed to target PD-L1 mRNA to downregulate its expression via the RNase H activity.

saP21-40/siPDL1-2 and saP21-40/siPDL1-3 are DAOs composed of two duplexes RAG1-40 and siPDL1-2 or siPDL1-3 conjugated via a linker.

saP21-40/aPDL1-1, saP21-40/aPDL1-2 and saP21-40/aPDL1-3 are DAOs composed of duplex RAG1-40 and aPDL1-1, aPDL1-2, aPDL1-3 gapmer ASOs conjugated via a linker with the 5′ end of the ASO conjugated to the linker.

saP21-40/aPDL1-1R, saP21-40/aPDL1-2R and saP21-40/aPDL1-3R are DAOs composed of duplex RAG1-40 and aPDL1-1, aPDL1-2, aPDL1-3 gapmer ASOs respectively conjugated via a linker with the 3′ end of the ASO conjugated to the linker.

TABLE 16
Representative oligonucleotide sequences tested in Example 18

| | | | | SEQ | | | |
|---|---|---|---|---|---|---|---|
| | Cat- | | Target | ID | | Size | |
| Name | egory | MOA | gene | NO: | Sequence (5′-3′) | (nt) | Design |
| dsCon2 | n/a | n/a | n/a | 1 | ACUACUGAGUGACAGUAGATT | 21 | n/a |
| | | | | 2 | UCUACUGUCACUCAGUAGUTT | 21 | |
| RAG1-40 | saRNA | RNAa | CDKN1A | 62 | CCAACUCAUUCUCCAAGUC | 19 | saRNA |
| saRNA | | | | 63 | UACUUGGAGAAUGAGUUGGCA | 21 | |
| siPDL1-2 | siRNA | RNAi | CD274 | 64 | CCUAUAUGUGGUAGAGUAUTT | 21 | siRNA |
| siRNAs | | | | 65 | AUACUCUACCACAUAUAGGTT | 21 | |
| siPDL1-3 | siRNA | RNAi | CD274 | 66 | GCCGAAGUCAUCUGGACAATT | 21 | siRNA |
| siRNAs | | | | 67 | UUGUCCAGAUGACUUCGGCTT | 21 | |
| aPDL1-1 | ASO | Rnase | CD274 | 68 | meC*meA*meU*meA*meU*a*g*g*u*c* | 20 | ASO gapmer |
| ASOS | gapmer | H | | | c*u*u*g*g*meG*meA*meA*meC*meC | | |
| aPDL1-2 | ASO | Rnase | CD274 | 69 | meC*meA*meU*meA*meC*u*c*u*a*c* | 20 | ASO gapmer |
| ASOs | gapmer | H | | | c*a*c*a*u*meA*meU*meA*meG*meG | | |
| aPDL1-3 | ASO | Rnase | CD274 | 70 | meC*meA*meU*meA*meC*u*c*u*a*c* | 20 | ASO gapmer |
| ASOs | gapmer | H | | | c*a*c*a*u*meA*meU*meA*meG*meG | | |
| saP21-40/ | DAO | RNAa | CDKN1A | 71 | CCAACUCAUUCUCCAAGUC-L1- | 40 | RAG1-40 and |
| siPDL1-2 | | + | + | | CCUAUAUGUGGUAGAGUAUTT | | siPDL1-2 |
| | | Rnase | CD274 | 63 | UACUUGGAGAAUGAGUUGGCA | 21 | conjugated by |
| | | H | | 65 | AUACUCUACCACAUAUAGGTT | 21 | S18 |
| saP21-40/ | DAO | RNAa | CDKN1A | 100 | CCAACUCAUUCUCCAAGUC-L1- | 40 | RAG1-40 and |
| siPDL1-3 | | + | + | | GCCGAAGUCAUCUGGACAATT | | siPDL1-3 |
| | | Rnase | CD274 | 63 | UACUUGGAGAAUGAGUUGGCA | 21 | conjugated by |
| | | H | | 65 | AUACUCUACCACAUAUAGGTT | 21 | S18 |
| saP21-40/ | DAO | RNAa | CDKN1A | 72 | CCAACUCAUUCUCCAAGUC-L1- | 39 | RAG1-40 and |
| aPDL1-1 | | + | + | | meC*meA*meU*meA*meU*a*g*g*u*c* | | aPDL1-1 |
| | | Rnase | CD274 | | c*u*u*g*g*meG*meA*meA*meC*meC | | conjugated by |
| | | H | | 63 | UACUUGGAGAAUGAGUUGGCA | 21 | S18 |
| saP21-40/ | DAO | RNAa | CDKN1A | 73 | CCAACUCAUUCUCCAAGUC-L1- | 39 | RAG1-40 and |
| aPDL1-2 | | + | + | | meC*meA*meU*meA*meC*u*c*u*a*c* | | aPDL1-2 |
| | | Rnase | CD274 | | c*a*c*a*u*meA*meU*meA*meG*meG | | conjugated |
| | | H | | 63 | UACUUGGAGAAUGAGUUGGCA | 21 | by S18 |
| saP21-40/ | DAO | RNAa | CDKN1A | 74 | CCAACUCAUUCUCCAAGUC-L1- | 39 | RAG1-40 and |
| aPDL1-3 | | + | + | | meC*meU*meU*meG*meU*c*c*a*g*a* | | aPDL1-3 |
| | | Rnase | CD274 | | u*g*a*c*u*meU*meC*meG*meG*meC | | conjugated |
| | | H | | 63 | UACUUGGAGAAUGAGUUGGCA | 21 | by S18 |
| saP21-40/ | DAO | RNAa | CDKN1A | 75 | CCAACUCAUUCUCCAAGUC-L1- | 39 | RAG1-40 and |
| aPDL1-1R | | + | + | | meC*meC*meA*meA*meG*g*g*u*u*c* | | aPDL1-1 |
| | | Rnase | CD274 | | c*u*g*g*a*meU*meA*meU*meA*meC | | conjugated by |
| | | H | | 63 | UACUUGGAGAAUGAGUUGGCA | 21 | S18 with the |
| | | | | | | | ASO linked to |
| | | | | | | | the duplex in |
| | | | | | | | from 3′ to 5′ |
| | | | | | | | end |
| saP21-40/ | DAO | RNAa | CDKN1A | 76 | CCAACUCAUUCUCCAAGUC-L1- | 39 | RAG1-40 and |
| aPDL1-2R | | + | + | | meG*meG*meA*meU*meA*u*a*c*a*c* | | aPDL1-2 |
| | | Rnase | CD274 | | c*a*u*c*u*meC*meA*meU*meA*meC | | conjugated by |
| | | H | | 63 | UACUUGGAGAAUGAGUUGGCA | 21 | S18 with the |
| | | | | | | | ASO linked to |
| | | | | | | | the duplex from |
| | | | | | | | 3′ to 5′ end |
| saP21-40/ | DAO | RNAa | CDKN1A | 77 | CCAACUCAUUCUCCAAGUC-L1- | 39 | RAG1-40 and |
| aPDL1-3R | | + | + | | meC*meG*meG*meC*meU*u*c*a*g*u* | | aPDL1-3 |
| | | Rnase | CD274 | | a*g*a*c*c*meU*meG*meU*meU*meC | | conjugated by |
| | | H | | 63 | UACUUGGAGAAUGAGUUGGCA | 21 | S18 with the |
| | | | | | | | ASO linked to |
| | | | | | | | the duplex from |
| | | | | | | | 3′ to 5′ end |

Keys:
*, phosphorothioate (PS); f, 2′-fluoro; m, 2′-OMe; me, 2′-MOE; p, 5′-phosophate; vp, 5′-(E)-vinylphosphonate; C, 5-methyl cytosine; U, 5-methyl uracil; L1, spacer-18 (S18 linker); L4, spacer-C6 (C6 linker); L9, spacer-9 (S9 linker)

As shown in FIG. 20A, p21 saRNA (RAG1-40), PD-L1 siRNA (siPDL1-3), PD-L1 ASOs (aPLD1-1, aPLD1-2, aPLD1-3) and DAOs were transfected at 10 nM into PC3 cells for 72 hours. RAG1-40 induced the expression of p21 by 10.5-fold and unexpectedly also increased the expression of PD-L1 by 13.2 fold. siPDL1-3 alone decreased PD-L1 expression by 62% and but also decreased p21 mRNA by 33%. aPDL1-1, aPDL1-2 and aPDL1-3 caused a 14%, 25% and 28% decrease in PD-L1 mRNA.

Compared with Mock treatment, DAOs saP21-40/siPDL1-3, saP21-40/aPDL1-1, saP21-40/aPDL1-2, saP21-40/aPDL1-3, saP21-40/aPDL1-1R, saP21-40/aPDL1-2R and saP21-40/aPDL1-3R caused a 3.3-, 4.0-, 5.8-, 8.4-, 10.3-, 11.0- and 8.2-fold increase in p21 mRNA. Compared with RAG1-40 treatment, DAOs saP21-40/siPDL1-3, saP21-40/aPDL1-1, saP21-40/aPDL1-2, saP21-40/aPDL1-3, saP21-40/aPDL1-1R, saP21-40/aPDL1-2R and saP21-40/aPDL1-3R caused an 84%, 93%, 91%, 86%, 27%, 36% and 60% decrease of PD-L1 mRNA (FIG. 20A). Subtracting the activating effect of p21 upregulation on PD-L1 expression and the inhibitory effect of PD-L1 downregulation on p21 expression, all except 3 DAOs (saP21-40/aPDL1-1R, saP21-40/aPDL1-2R and saP21-40/aPDL1-3R) with the ASO's 3′ end conjugated to the linker exhibited both RNAa activity in inducing p21 and gapmer ASO's activity in inhibiting PD-L1.

As shown in FIG. 20B, in similarly treated KU-7 cells, RAG1-40 induced p21 mRNA by 8.3 fold and unexpectedly also induced PD-L1 expression by 20.8 fold. siPDL1-2 caused a 45% decrease in PDL1 mRNA. Compared with Mock control, DAOs saP21-40/siPDL1-2, saP21-40/aPDL1-1, saP21-40/aPDL1-2, saP21-40/aPDL1-3, saP21-40/aPDL1-1R, saP21-40/aPDL1-2R and saP21-40/aPDL1-3R caused a 7.4-, 2.8-, 3.1-, 4.1-, 5.4-, 5.5- and 5.3-fold increase in p21 mRNA. Compared with RAG1-40 treatment, saP21-40/siPDL1-2, saP21-40/aPDL1-1, saP21-40/aPDL1-2, saP21-40/aPDL1-3, saP21-40/aPDL1-1R, saP21-40/aPDL1-2R and saP21-40/aPDL1-3R caused a 78%, 95%, 96%, 94%, 37%, 58% and 55% decrease of PD-L1 mRNA. Subtracting the activating effect of p21 upregulation on PD-L1 expression, all DAOs exhibited both RNAa activity in inducing p21 and gapmer ASO's activity in inhibiting PD-L1.

### 5.19. Example 19: Effect of “ASO-ASO” DAOs on the Expression of SMN2FL and SMN2Δ7 in GM03813 Cells

It has been reported that ASOs complementary to the 5′ end of SMN2 gene increase SMN mRNA and protein levels via a mechanism of inhibition of SMN2 mRNA decay by the ASO, and use of the 5′ UTR ASO in combination with a splice-modulating ASO increases SMN levels above those attained with the latter alone [PMID: 33575118, PMCID: PMC7851419]. DAOs were created by covalently linking a 5′UTR ASO and ASO10-27 in different order via the spacer 18 linker, resulting two DAOs with one (DA6-27A-5′UTR) in the orientation of 5′-ASO10-27-linker-5′UTR ASO-3′, and the other (DA6-5′UTR-27A) in the orientation of 5′-5′UTR ASO-linker-ASO10-27-3′. These two DAOs are called “ASO-ASO” DAOs (Table 17).

These DAOs were transfected into GM03813 cells to evaluate their activity in inducing SMN2 mRNA expression. As shown in FIG. 21, ASO10-27 and 5′UTR ASO at 25 nM caused a 1.6- and 1.1-fold increase in SMN2FL with concurrent decrease of SMN2Δ7 (by a 97%, 32%) respectively. DA6-5′UTR-27A DAO showed higher potency in inducing SMN2FL mRNA expression (1.8 fold) compared with ASO10-27 (1.6 fold) or 5′UTR ASO (1.1 fold) alone, suggesting that the ASO-ASO DAO possesses added activity from its component ASOs. DA6-27A-S′UTR showed higher potency in inducing SMN2A7 expression, and thus increasing the total expression of SMN2FL+SMN2A7. This study further indicates that linking two ASOs is also able to create DAOs with added activity and the way how the two ASOs are connected dictates activity of the resulted DAOs.

TABLE 17
Representative oligonucleotide sequences tested in Example 19

| | | | | SEQ | | | |
|---|---|---|---|---|---|---|---|
| | Cat- | | Target | ID | | Size | |
| Name | egory | MOA | gene | NO: | Sequence (5′-3′) | (nt) | Design |
| dsCon2 | n/a | n/a | n/a | 1 | ACUACUGAGUGACAGUAGATT | 21 | n/a |
| | | | | 2 | UCUACUGUCACUCAGUAGUTT | 21 | |
| ASO10- | ASO | steric | SMN2 | 11 | meU*meC*meA*meC*meU*meU*meU*meC*meA*meU* | 18 | steric |
| 27 | | block | | | meA*meA*meU*meG*meC*meU*meG*meG | | block ASO |
| 5′UTR | ASO | steric | SMN2 + | 142 | meG*meU*meU*meA*meU*meC*meG*meC*meC*m | 24 | ASO |
| ASO | | block + | SMN2 | | eC*meU*meC*meC*meC*meA*meC*meA*meU*meU | | targeting |
| | | steric | | | *meU*meG*meU*meG*meG | | 5′UTR |
| | | block | | | | | |
| DA6- | DAO | steric | SMN2 + | 143 | meU*meC*meA*meC*meU*meU*meU*meC*meA*m | 42 | ″ASO10-27″- |
| 27A- | (ASO- | block + | SMN2 | | eU*meA*meA*meU*meG*meC*meU*meG*meG-L1- | | S18-″5′UTR |
| 5′UTR | ASO) | steric | | | meG*meU*meU*meA*meU*meC*meG*meC*meC*m | | ASO″ |
| | | block | | | eC*meU*meC*meC*meC*meA*meC*meA*meU*meU | | |
| | | | | | *meU*meG*meU*meG*meG | | |
| DA6- | DAO | steric | SMN2 + | 144 | meG*meU*meU*meA*meU*meC*meG*meC*meC*m | 42 | ″5′UTR |
| 5′UTR- | (ASO- | block + | SMN2 | | eC*meU*meC*meC*meC*meA*meC*meA*meU*meU | | ASO″-S18-″ |
| 27A | ASO) | steric | | | *meU*meG*meU*meG*meG-L1- | | ASO10-27″ |
| | | block | | | meU*meC*meA*meC*meU*meU*meU*meC*meA*m | | |
| | | | | | eU*meA*meA*meU*meG*meC*meU*meG*meG | | |

Keys:
*, phosphorothioate (PS); f, 2′-fluoro; m, 2′-OMe; me, 2′-MOE; p, 5′-phosophate; VP, 5′-(E)-vinylphosphonate; C, 5-methyl cytosine; U, 5-methyl uracil; L1, spacer-18 (S18 linker); L4, spacer-C6 (C6 linker); L9, spacer-9 (S9 linker)

### 5.20. Example 20: Effect of “ASO-ASO” DAOs on SMN Protein in GM03813 Cells

To further verify protein level induction of “ASO-ASO” DAOs, SMN protein was assessed by western blotting. All DAOs were transfected into GM03813 cells at 25 nM for 72 hours. As shown in FIGS. 22A and 22B, ASO10-27 and 5′UTR ASO at 25 nM caused a 2.1- and 2.0-fold increase in SMN protein. Consistent with RT-qPCR result, DA6-5′UTR-27A DAO showed higher potency in inducing SMN protein (3.0 fold) compared with ASO10-27 (2.1 fold) or 5′UTR ASO (2.0 fold) alone, suggesting that the ASO-ASO DAO possesses added activity from its component ASOs. However, DA6-27A-5′UTR failed to show added activity (2.0 fold). The study further indicates that linking two ASOs is also able to create DAOs with added activity on SMN protein level and the way how the two ASOs are connected dictates activity of the resulted DAOs.

### 5.21. Example 21: Effect of “Divalent” DAOs on Htt Gene Expression in CNS Tissues of Pup Mice

As a special case of DAO design, “divalent” DAO is to covalently connect two duplexes sharing the same sequence. With “divalent” DAO design, the additional phosphorothioate backbone modification introduced in the “divalent” DAO molecule increased the in vivo biodistribution and cell uptake. To verify the enhanced delivery efficiency of “divalent” DAO designed oligonucleotides, siHtt-S1L1, the “divalent” DAO variant of siHtt duplex (siHtt-S1V1) is created by connecting two siHtt-SIV1 covalently. After ICV injection of the oligonucleotides in the pup mice, Htt gene expression knockdown level was assessed by RT-qPCR.

As shown in FIG. 23A, the “divalent” DAO variant siHtt-SIL1 caused Htt mRNA expression knockdown at a level of 30% in brain and 20% in spinal cord 3 days after ICV administration, while the siHtt-SIV1 without “divalent” DAO design caused Htt mRNA knockdown at a level of 2% in brain and 12% in spinal cord. As shown in FIG. 23B, 14 days after the ICV administration, the “divalent” DAO variant siHtt-SIL1 caused 47% (in brain) and 39% (in spinal cord) knockdown of Htt mRNA expression which is superior comparing to 40% (in brain) and 29% caused by siHtt-S1V1. This data provides evidence that DAO designed duplex possesses better in vivo activity in the CNS via local injection.

### 5.22. Example 22: Effect of “Divalent” DAOs on Sod1 Gene Expression in Major Tissues of Pup Mice by Subcutaneous Administration

To further verify the enhanced delivery efficiency of “divalent” DAO designed oligonucleotides, “divalent” DAO designed siRNA targeting Sod1 gene (siSOD1M2-S1L1V2v-Qu5) is synthesized by connecting two siSOD1M2-S1V1v-Qu5 covalently. After SC injection of the oligonucleotides in the pup mice, biodistribution of Qu5 signal revealed siSOD1M2-S1L1V2v-Qu5 was enriched in almost all peripheral tissues especially in muscle, liver, and kidney (FIGS. 24A & 24B). The Sod1 gene expression knockdown level was assessed by RT-qPCR. As shown in FIG. 24C, the “divalent” DAO variant siSOD1-S1L1V2v-Qu5 caused Sod1 mRNA expression knockdown at a level of 84% in liver, while the siSOD1M2-S1V1v-Qu5 without “divalent” DAO design caused Sod1 mRNA knockdown at a level of 65%. This data provides evidence that DAO designed duplex possesses better in vivo activity in the liver via systemic injection.

### 5.23. Example 23: Effect of “Divalent” DAOs on Sod1 Gene Expression in CNS Tissues of Pup Mice

To further verify the enhanced delivery efficiency of siSODIM2-S1L1V2v-Qus in CNS tissues, siSODIM2-S1L1V2v-Qu5 and siSOD1M2-S1V1v-QuS were administrated in pup mice by ICV injection. Three days after ICV injection, biodistribution of Qu5 signal revealed siSODIM2-S1L1V2v-Qu5 was enriched across CNS tissues (FIGS. 25A & 25B). The Sod1 gene expression knockdown level was assessed by RT-qPCR. As shown in FIG. 25C, the “divalent” DAO variant siSOD1-S1L1V2v-Qu5 caused Sod1 mRNA expression knockdown at a level of 75% in brain and 88% in spinal cord, while the siSODIM2-S1V1v-Qu5 without “divalent” DAO design caused Sod1 mRNA knockdown at a level of 61% in brain and 54% in spinal cord. This data provides evidence that DAO designed duplex possesses better in vivo activity in the CNS tissues via local injection.

### 5.24. Materials and Methods

Oligonucleotide design and synthesis

The phosphoramidites 2′-OMEO-N6-Bz-A (HR-00207001), 2′-O-MOE-N2-ibu-G (HR-00207003), 2′-O-MOE-N4-Bz-5-Me-C(HR-00207006), 2′-O-MOE-5-Me—U (HR-00207005), Bz-rA (HR-00202001), Ac-rC (HR-00202002), ibu-rG (HR-00202003), rU (HR-00202003), 2′-F-Bz-dA (HR-00204001), 2′-F-Ac-dC (HR-00204004), 2′-F-dU (HR-00204005), 2′-F-ibu-dG (HR-00204003), dT (HR-00201004), Spacer-18 (HR-00214005), Spacer-9 (HR-00214009), and Spacer-C6 (HR-00214019) were purchased from Wuhu Huaren Science and Technology (Wuhu, Anhui Province, China). The oligonucleotides used were synthesized on a K&A DNA synthesizer (K&A Laborgeraete GbR, Schaafheim, Germany) by using solid phase technique. Briefly, during solid phase synthesis, phosphoramidite monomers, including various linkers and conjugates, were added sequentially onto a solid support to generate the desired full-length oligonucleotide. Each cycle of base addition consisted of four chemical reactions, detritylation, coupling, oxidation/thiolation and capping. Following synthesis, the C&D (cleavage and deprotection) step releases the oligonucleotide from the solid-support and removed the protecting groups from bases and phosphates. After synthesis, the solid support was then transferred to a screw-cap microcentrifuge tube. For a 1 μM synthesis scale, a mixture of 33% methylamine in ethanol and 1 ml of ammonium hydroxide was added. The tube containing the solid support was then heated in an oven at 60° C. to 65° C. for 2 hours and then allowed to cool to room temperature. The cleavage solution was collected and evaporated to dryness in a speedvac. The crude RNA oligonucleotide, still carrying the 2′-TBDMS groups, was dissolved in 0.1 ml of DMSO. After adding 1 ml of Triethylamine 3HF, the tube was capped, and the mixture was shaken vigorously to ensure complete dissolution. The bottle was heated in an oven at 60° C. to 65° C. for 3 to 3.5 hours. The tube was removed from the oven and cooled to room temperature. The solution containing the completely desilylated oligonucleotide was cooled on dry ice. Two milliliter of ice-cold n-butanol (−20° C.) was carefully added in 0.5 ml portions to precipitate the oligonucleotide. The precipitate was filtered and washed with 1 ml ice-cold n-butanol and the precipitate was then dissolved in 1 M TEAA (triethylammonium acetate). The crude oligonucleotides were then purified by exchange (IEX) HPLC using a source 15Q column. The purity of the fractions was analyzed by ion exchange (IEX) HPLC using Column DNA Pac™ PA100. Following the generation of desalted purified single strand solutions, a duplex was made by annealing two complimentary single-stranded oligonucleotides and was lyophilized to powder.

ASO synthesis

Antisense oligonucleotide (ASO) including ASO10-27 (also known as Nusinersen (SPINRAZA®) was synthesized using the same technique described above except the omission of the final annealing step. ASO10-27 is a single stranded and 2′-O-2-methoxyethyl (MOE)-modified ASO and induces exon 7 inclusion by targeting an intronic splicing silencer (ISS) at intron 7 of SMN2 gene (Hua et al. 2008). The sequence for ASO10-27 is: meU*meC*meA*meC*meU*meU*meU*meC*meA*meU*meA*meA*meU*meG*meC*m eU*meG*meG (SEQ ID NO: 11), in which, me=2′-MOE, *=phosphorothioate (PS) backbone modification, and all cytosines (C) are 5-methyl cytosine, and all uracils (U) are 5-methyl uracil. Lyophilized oligonucleotides were suspended in RNase free water for cell transfection or diluted with saline to the appropriate concentration for in vivo injection.

Cell culture and treatment

SMA patient derived fibroblasts were obtained from Coriell Institute (Camden, NJ, USA), including GM00232 (SMA type I with 2 copies of SMN2 gene), GM09677 (SMA type with 3 copies of SMN2 gene) and GM03813 (SMA type II with 3 copies of SMN2 gene). These cells were cultured at 5% CO2 and 37° C. in modified MEM medium (Gibco, Thermo Fisher Scientific, Carlsbad, CA) supplemented with 15% bovine calf serum (Sigma-Aldrich), 1% NEAA (Gibco) and 1% penicillin/streptomycin (Gibco).

The human prostate cancer cell line, PC3 cells were cultured in RPMI-1640 (Gibco) medium supplemented with 10% bovine calf serum (Sigma-Aldrich) and 1% penicillin/streptomycin (Gibco). The human bladder cancer cell line, KU-7 was cultured in McCoy's 5A (modified) medium supplemented with 10% bovine calf serum (Sigma-Aldrich) and 1% penicillin/streptomycin (Gibco). These cells were cultured at 5% CO2 and 37° C. incubator.

Primary mouse hepatocytes (PMH) were isolated from the liver of Type III SMA mice (smn1^{+/-}, SMN2^{+/-}) and cultured at 5% CO₂ and 37° C. in modified DMEM medium (Gibco, Thermo Fisher Scientific, Carlsbad, CA) supplemented with 10% bovine calf serum (Sigma-Aldrich) and 1% penicillin/streptomycin (Gibco).

To transfect oligonucleotides including saRNA(s), siRNA(s), ASO(s) and DAO(s), cells were seeded into 6-well plates at a density of 1˜2×10⁵ cells/well and were transfected with oligonucleotides at different concentrations using RNAiMax (Invitrogen, Carlsbad, CA) according to the reverse transfection protocol provided by the manufacturer for 72 hours (unless otherwise specified). The sequences for saRNAs, an SMN2 siRNA (DS06-332i), a control dsRNA (dsCon2), an ASO and different DAO structures were listed in Table 7.

PBMC cells (PB004F, ALLCELLS, US) were cultured at 5% CO₂ and 37° C. in RPMI-1640 (Gibco, Thermo Fisher Scientific) medium supplemented with 10% bovine calf serum (Sigma-Aldrich) and 1% penicillin/streptomycin (Gibco).

### RNA Isolation and Reverse Transcription-Quantitative Polymerase Chain Reaction (RT-qPCR)

For RNA isolation from cultured cells, total cellular RNA was isolated from treated cells using an RNeasy Plus Mini kit (Qiagen, Hilden, Germany) according to its manual. To isolated RNA from animal tissues, tissues were harvested and stored at RNA later (AM7021, Thermo Fisher, Carlsbad, CA, USA). Total RNA was then isolated using MagPure Total RNA Micro LQ kit (Magen, R6621, Guangzhou, Guangdong, China) by auto-pure96 machine (ALLSHENG, Hangzhou, Zhejiang, China). The resultant RNA (1 μg) was reverse transcribed into cDNA by using a PrimeScript RT kit containing gDNA Eraser (Takara, Shlga, Japan). The resultant cDNA was amplified in an ABI 7500 Fast Real-time PCR System (Applied Biosystems; Foster City, CA) using SYBR Premix Ex Taq II (Takara, Shlga, Japan) reagents and primers which specifically amplified target gene of interest. The reaction conditions were: 95° C. for 3 seconds (1 cycle) and 60° C. for 30 seconds (40 cycles). Amplification of TBP, Tbp and Gapdh genes was served as an internal control. All primer sequences are listed in Table 19. The RT and RT-qPCR reactions are shown in Table 20 and Table 21.

TABLE 19
Primer sequences for RT-qPCR assay

| | | SEQ ID | | Product |
|---|---|---|---|---|
| Primer Title | Gene | NO | Sequence (5′-3′) | size |
| SMN2FL F | Human | 101 | TCATACTGGCTATTATATGGGTTTT | 95 bp |
| SMN2FL R | SMN2 | 102 | TGCTCTATGCCAGCATTTCTC | |
| SMNΔ7 F | Human | 103 | GCTATTATATGGAAATGCTGGCATAG | 73 bp |
| SMNΔ7 R | SMN2 | 104 | TTCCAGATCTGTCTGATCGTTTCT | |
| SMN-exon6 F | Human | 105 | CCCCCACCACCTCCCATATG | 507 bp |
| SMN-exon8 R | SMN2 | 106 | CCCTTCTCACAGCTCATAAAATTAC | 453 bp |
| p21 F | Human | 107 | GGAAGACCATGTGGACCTGT | 178 bp |
| p21 R | p21 | 108 | GGATTAGGGCTTCCTCTTGG | |
| PDL1 F | Human | 109 | TGTGACCAGCACACTGAGAA | 130 bp |
| PDL1 R | PDL1 | 110 | TGGAGGATGTGCCAGAGGTA | |
| SOD1 F | Human | 122 | AAGCATTAAAGGACTGACTGAAGG | 154 bp |
| SOD1 R | SOD1 | 123 | CAAGTCTCCAACATGCCTCTC | |
| TBP F | Human | 111 | TGCTCACCCACCAACAATTTAG | 139 bp |
| TBP R | TBP | 112 | TCTGCTCTGACTTTAGCACCTG | |
| GAPDH F | Human | 113 | ATCACCATCTTCCAGGAGCGA | 101 bp |
| GAPDH R | GAPDH | 114 | TTCTCCATGGTGGTGAAGACG | |
| Tbp F | Mouse | 115 | GCTCTGGAATTGTACCGCAG | 126 bp |
| Tbp R | Tbp | 116 | CTGCAGCAAATCGCTTGGGA | |
| Gapdh F | Mouse | 117 | TGAAGGTCGGTGTGAACGG | 112 bp |
| Gapdh R | Gapdh | 118 | TTGAGGTCAATGAAGGGGTCG | |

TABLE 20
RT reaction

| | Volume (μl) |
|---|---|
| RT reaction-1 (Takara, RR047A) | |
| 5 × gDNA Eraser Buffer | 2 |
| gDNA Eraser | 1 |
| Total RNA (1 μg) + RNase Free dH₂O | 7 |
| Total | 10 |
| 42° C. 5 min, store at 4° C. | |
| RT Reagent-2 (Takara, RR047A) | |
| 5 × PrimeScript Buffer2 | 4 |
| PrimeScript RT Enzyme Mix I | 1 |
| RT Prime Mix | 1 |
| RNase free dH₂O | 4 |
| RT reaction-1 | 10 |
| Total | 20 |

37° C. 15 min, 85° C. 5 sec, store at 4° C.

TABLE 21
RT-qPCR reaction

| Reagent (Takara, RR820A) | Volume (μl) |
|---|---|
| SYBR Premix Ex Taq II (2×) | 5 |
| PCR Primer (forward + reverse) 5 μM | 1 |
| cDNA (RT product) | 4 |
| Total | 10 |

Semi-quantitative RT-PCR/DdeI digestion assay

To amplify both full length SMN2 (SMN2FL) and SMN2 lacking exon 7 (SMN2A7) in one reaction, cDNA was amplified by semi-quantitative RT-PCR using primers that spans exon 7 of SMN2 (Table 22). The PCR reaction conditions were: 94° C. for 2 minutes (1 cycle), 98° C. for 10 seconds, 60° C. for 15 seconds, 72° C. for 32 seconds, cycled for 30 times with a final 5 minute extension at 72° C. The PCR reaction is listed in Table 23. To further differentiate SMN1 mRNA from SMN2, the resultant PCR products of SMN were digested by DdeI restriction enzyme (R0175L, New England Biolabs, Ipswich, MA, USA) and then separated by 2% agarose gel. Due to a nucleotide variant on exon 8 of SMN2 (a DdeI recognition site exists in PCR products amplified from SMN2 gene but not from SMN1 gene), DdeI digestion releases a 115 bp fragment from both SMN2FL and SMN2Δ7, and resulting 3 fragments: 507 (SMN1FL), 338 (SMN2A7), 392 (SMN2FL) and the 115 bp fragments. TBP gene was also amplified as a RNA loading control. The DdeI digestion reaction conditions were: 37° C. for 60 minutes and 65° C. for 20 minutes, 1 cycle. The DdeI digestion reactions are listed in Table 24.

TABLE 22
Primer sequences for Semi-quantitative RT-PCR assay

| Primer name | SEQ ID NO. | Sequence (5′-3′) | Product size |
|---|---|---|---|
| SMN-exon6 F | 105 | CCCCCACCACCTCCCATATG | 507 bp |
| SMN-exon8 R | 106 | CCCTTCTCACAGCTCATAAAATTAC | 453 bp |

TABLE 23
Semi-quantitative RT-PCR reaction

| Reagents (Takara, R010A) | Volume (μl) |
|---|---|
| 5 × prime STAR Buffer | 5 |
| dNTP Mixture | 2 |
| Prime F + R (5 μM) | 1 |
| Template | 2 |
| PrimeSTAR HS DNA Polymerase | 0.25 |
| DD-Water | 14.75 |
| Total | 25 |

TABLE 24
Ddel digestion reaction

| Reagents (NEB, R0175L) | Volume (μl) |
|---|---|
| Restriction Enzyme | 1 |
| 10 × NEB Buffer | 1 |
| cDNA | 6 |
| DD-Water | 2 |
| Total | 10 |

Western blotting

Proteins were harvested from transfected cells using 1× RIPA Buffer including protease inhibitors and the protein concentration was detected by BCA protein assay kits (Beyotime, P0010, Shanghai, China). Protein electrophoresis was performed (10 μg protein/well) with the use of a sodium dodecyl sulfate polyacrylamide gel electrophoresis (PAGE) gel, which was then transferred to a polyvinylidene difluoride (0.45 μm PVDF) membrane. The membranes were blotted with primary anti-SMN (CST, 19276, USA) or anti-α/β-Tubulin (CST, 2148s, USA) antibodies at 4° C. overnight. After three washes with TBST buffer, the membranes were incubated with anti-IgG, horseradish peroxidase-conjugated secondary antibodies (CST, 7074s and 7076s, USA) for 1 h at room temperature (RT). The membranes were then washed with TBST buffer three times for 10 minutes each and analyzed by Image Lab (BIO-RAD, Chemistry Doctm MP Imaging System). Band densities of SMN protein and α/β-Tubulin were quantified using ImageJ software.

ELISA assay

To quantifying human IFN-α protein expression levels, supernatant of cultivated PBMC cells were collected and detected by OD value using ELISA kit (70-EK199-96, MULTI SCIENCES, China). Tests were conducted according to the following ELISA kit instructions with detailed procedures.
a) Prepare all required reagents and working concentration standards.
b) Remove the unneeded slats, put them back in the aluminum foil bag with desiccant, and re-seal.
c) Soak the ELISA plate: Add 300 μl 1× lotion and let it stand and soak for 30 seconds. Soaking is necessary in order to obtain the desired experimental results. After discarding the lotion, pat the microplate dry on absorbent paper. After washing the plate, please use the microplate immediately and do not let the microplate dry.
d) Add standard product: add 100 μl standard product diluted 2 times to the standard product well. Add 100 μl of standard diluent or medium to the blank wells
e) Add sample: serum/plasma: add 50 μl 1× detection buffer and 50 μl sample to the sample hole. Cell culture supernatant: Add 100 μl of cell culture supernatant to the sample well.
f) Add detection antibody: Add 50 μl diluted detection antibody (1:100 dilution) to each well. Ensure that steps d), e), and f) continue to add samples without interruption. The sample addition process is completed within 15 minutes.
g) Incubation: Use sealing film to seal the plate. Shake at 300 rpm and incubate at room temperature for 3 hours.
h) Washing: Discard the liquid, add 300 μl washing solution to each well to wash the plate, and wash 6 times. Each time you wash the plates, pat them dry on absorbent paper. In order to obtain the ideal experimental performance, the residual liquid must be completely removed.
i) Add substrate for color development: Add 100 μl of color development substrate TMB to each well, protect from light, and incubate at room temperature for 5-30 minutes.
j) Add stop solution: add 100 μl stop solution to each well. The color changed from blue to yellow. If the color is green or the color change is obviously uneven, please tap the frame lightly to mix well.
k) Detection reading: Within 30 minutes, use a microplate reader to perform dual-wavelength detection, and determine the OD value at 450 nm maximum absorption wavelength.
   Animal procedures

All animal procedures were conducted by certified laboratory personnel using protocols consistent with local and state regulations and approved by the Institutional Animal Care and Use Committee. SMA-like mice created by homozygous knock-out of mouse Smn1 (also known as Smn) exon 7 with transgene of human SMN2 (Smn1SMN2^{+/-}) as previously described Hsieh-Li et al. (Hsieh-Li et al. 2000) were obtained from Jackson Laboratories (Bar Harbor, ME, USA). Tail snips were gathered at postnatal day 0 (P0), and each pup was identified by paw tattooing and genotyped by PCR analysis using a set of 3 specific primers: S1, 5′-ATAACACCACCACTCTTACTC-3′ (SEQ ID NO: 119); S2, 5′-GTAGCCGTGATGCCATTGTCA-3′ (SEQ ID NO: 120) (1,150 bp band for wild type alleles); and S1 and H1, 5′-AGCCTGAAGAACGAGATCAGC-3′ (SEQ ID NO: 121) (950 bp band for mutant alleles). The PCR products were detected by 1% agarose gel. Severe SMA mice (Smn1″, SMN2^{+/-}) were generated. Littermates heterozygous (Het) for mouse Smn (Smn1^{+/-}, SMN2^{+/-}) were used as controls.

Intracerebral ventricle (ICV) injection

ICV injection was conducted in postnatal day 1 (P1) pups. Two microliters of oligonucleotides dissolved in 0.9% saline was injected using a 5-μl micro-syringe with a 33-gauge removable needle into either of the two cerebral lateral ventricles. An opaque tracer (Fast Green, 0.1%, W/V) was added to the reagent to visualize the borders of the lateral ventricle after injection.

TABLE 25
DNA sequences of saRNA hotspot regions on SMN2 promoter

| | | |
|---|---|---|
| H1 1481-1639 | agtcgcactctgtcactcaggctggagtgcagtggcgtgatcttggct | (SEQ ID |
| | cactgcaacctccgcctcccgagttcaagtgattctcctggctcagcc | NO: 124) |
| | tcccaagcagctgtcattacaggcctgcaccaccacacccggctgatt | |
| | tttgtatttttagga | |
| H2 1008-1090 | aatactggaggcccggtgtggtggctcacacctgtaatcccagcactt | (SEQ ID |
| | tgggaggccgaggcggtcggattacgaggtcagg | NO: 125) |
| H3 180-944 | ctggccaacatggtgaaaccccatctttactaaaaatacaaaaattag | (SEQ ID |
| | ccgggtgtggtggtgggcgcctgtaatcccagctactcggggggctga | NO: 126) |
| | ggcagaattgcttgaacctgggaggcagaggttgcagtgagctgagat | |
| | cacgccactgcattccagcctgggtgacagagcaatactctgtcgcaa | |
| | aaaaaaaaaagaatactggaggctgggcgaggtggctcacacctgtaa | |
| | tcccagcattttgggatgccagaggcgggcggaatatcttgagctcag | |
| | gagttcgagaccagcctacacaatatgctccaaacgccgcctctacaa | |
| | aacatacagaaactagccgggtgtggtggcgtgcccctgtggtcctag | |
| | ctacttgggaggttgaggcgggaggatcgcttgagctcgggaggtcga | |
| | ggctgcaatgagccgagatggtgccactgcactctgacgacagagcga | |
| | gactccgtctcaaaacaaacaacaaataaggttgggggatcaaatatc | |
| | ttctagtgtttaaggatctgccttccttcctgcccccatgtttgtctt | |
| | tccttgtttgtctttatatagatcaagcaggttttaaattcctagtag | |
| | gagcttacatttacttttccaagggggagggggaataaatatctacac | |
| | acacacacacacacacacacacacacacacactggagttcgagacgag | |
| | gcctaagcaacatgccgaaaccccgtctctactaaatacaaaaaatag | |
| | ctgagcttggtggcgcacgcctatagtcctagctactggggaggctg | |
| H4 37-144 | ctgcagtgagccgagatcgcgccgctgcactccagcctgagcgacagg | (SEQ ID |
| | gcgaggctctgtctcaaaacaaacaaacaaaaaaaaaaggaaaggaaa | NO: 127) |
| | tataacacagtg | |

### 6. EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

### 7. References

Coovert, D. D., T. T. Le, et al. (1997). “The survival motor neuron protein in spinal muscular atrophy.” Human molecular genetics 6(8): 1205-1214.
Crooke, S. T. (1991). “Antisense technology.” Current opinion in biotechnology 2(2): 282-287.
Crooke, S. T., J. L. Witztum, et al. (2018). “RNA-Targeted Therapeutics.” Cell metabolism 27(4): 714-739.
Fire, A., S. Xu, et al. (1998). “Potent and specific genetic interference by double-stranded RNA in Caenorhabditis elegans.” Nature 391(6669): 806-811.
Horrobin, D. F. (2001). “Realism in drug discovery-could Cassandra be right?” Nature biotechnology 19(12): 1099-1100.
Hsieh-Li, H. M., J. G. Chang, et al. (2000). “A mouse model for spinal muscular atrophy.” Nature genetics 24(1): 66-70.
Hua, Y., T. A. Vickers, et al. (2007). “Enhancement of SMN2 exon 7 inclusion by antisense oligonucleotides targeting the exon.” PLOS biology 5(4): e73.
Hua, Y., T. A. Vickers, et al. (2008). “Antisense masking of an hnRNP A1/A2 intronic splicing silencer corrects SMN2 splicing in transgenic mice.” American journal of human genetics 82(4): 834-848.
Janowski, B. A., S. T. Younger, et al. (2007). “Activating gene expression in mammalian cells with promoter-targeted duplex RNAs.” Nature chemical biology 3(3): 166-173.
Kang, M. R., G. Li, et al. (2017). “Development of Therapeutic dsP21-322 for Cancer Treatment.” Advances in experimental medicine and biology 983: 217-229.
Le, T. T., L. T. Pham, et al. (2005). “SMNDelta7, the major product of the centromeric survival motor neuron (SMN2) gene, extends survival in mice with spinal muscular atrophy and associates with full-length SMN.” Human molecular genetics 14(6): 845-857.
Levin, A. A. (2017). “Targeting Therapeutic Oligonucleotides.” The New England journal of medicine 376(1): 86-88.
Li, L. C. (2017). “Small RNA-Guided Transcriptional Gene Activation (RNAa) in Mammalian Cells.” Advances in experimental medicine and biology 983: 1-20.
Li, L. C., S. T. Okino, et al. (2006). “Small dsRNAs induce transcriptional activation in human cells.” Proceedings of the National Academy of Sciences of the United States of America 103(46): 17337-17342.
Liang, X. H., H. Sun, et al. (2017). “RNase H1-Dependent Antisense Oligonucleotides Are Robustly Active in Directing RNA Cleavage in Both the Cytoplasm and the Nucleus.” Molecular therapy: the journal of the American Society of Gene Therapy 25(9): 2075-2092.
Setten, R. L., J. J. Rossi, et al. (2019). “The current state and future directions of RNAi-based therapeutics.” Nature reviews. Drug discovery 18(6): 421-446.
Zamore, P. D., T. Tuschl, et al. (2000). “RNAi: double-stranded RNA directs the ATP-dependent cleavage of mRNA at 21 to 23 nucleotide intervals.” Cell 101(1): 25-33.
Pagliarini, V., M. Guerra, V. Di Rosa, C. Compagnucci, and C. Sette. 2020. ‘Combined treatment with the histone deacetylase inhibitor LBH589 and a splice-switch antisense oligonucleotide enhances SMN2 splicing and SMN expression in Spinal Muscular Atrophy cells’, Journal of neurochemistry, 153: 264-75.

1. A multi-valent oligonucleotide agent comprising two or more functional oligonucleotides that are covalently linked, wherein the two or more functional oligonucleotides are independently selected from:
a) a double stranded RNA (dsRNA); and
b) an antisense oligonucleotide (ASO).

2. The multi-valent oligonucleotide agent of claim 1, wherein the number of the functional oligonucleotides comprised in the multi-valent oligonucleotide agent is ranged from 2 to X, wherein X is an integer ranged from 3 to 10.

3. The multi-valent oligonucleotide agent of claim 2, wherein the number of dsRNA comprised in the agent is from 0 to X, with the rest functional oligonucleotides being ASO(s).

4. The multi-valent oligonucleotide agent of claim 1, wherein
the dsRNA(s) are independently selected from a small interfering RNA (siRNA) and a small activating RNA (saRNA); and/or
the ASO(s) are independently selected from a gapmer and a mixmer.

5. (canceled)

6. (canceled)

7. The multi-valent oligonucleotide agent of claim 1, wherein each dsRNA comprises a sense strand that is at least 10 contiguous nucleotides and an antisense strand that is at least 10 contiguous nucleotides.

8. (canceled)

9. The multi-valent oligonucleotide agent of claim 1, wherein each ASO has a nucleotide sequence that is at least 5 contiguous nucleotides in length.

10. (canceled)

11. The multi-valent oligonucleotide agent of claim 1, wherein the two or more functional oligonucleotides have a total length ranging from 12 to 200 nucleotides.

12. The multi-valent oligonucleotide agent of claim 1, wherein any two adjacent functional oligonucleotides of the two or more functional oligonucleotides are covalently linked by a linking component or with no linking component.

13. The multi-valent oligonucleotide agent of claim 12, wherein the linking component is selected from the following linkers or derivatives thereof:

| Name | Formula | Structure |
|---|---|---|
| Spacer-9 | C₃₆H₄₉N₂O₇P | 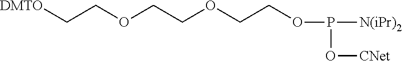 |
| Spacer-18 | C₄₂H₆₁N₂O₁₀P | 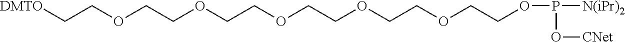 |
| Spacer-C3 | C₃₃H₄₃N₂O₅P | 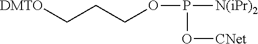 |
| Spacer-C6 | C₃₆H₄₉N₂O₅P | 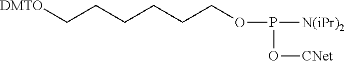 |

14. The multi-valent oligonucleotide agent of claim 12, wherein the two adjacent functional oligonucleotides are covalently linked by: 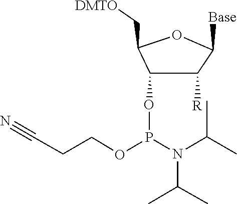
wherein R represents —H or —OH or —OMe, or -MOE, or —F, or other 2′ chemical modifications; and/or by a phosphodiester bond or a phosphorothioate bond; and/or by one or more nucleotides.

15. The multi-valent oligonucleotide agent of claim 1, wherein one or more of the functional oligonucleotides comprise at least one chemically modified nucleotide.

16. (canceled)

17. The multi-valent oligonucleotide agent of claim 1, wherein each of the ASO in the agent is covalently linked to the adjacent targeting oligonucleotide in a 3′ to 5′ orientation or in a 5′ to 3′ orientation.

18. The multi-valent oligonucleotide agent of claim 1, wherein each of the dsRNA in the agent is covalently linked to an adjacent ASO at its 3′ end of the sense or antisense strand; or at its 5′ end of the sense or antisense strand.

19. (canceled)

20. The multi-valent oligonucleotide agent of claim 1,
wherein the multi-valent oligonucleotide agent comprises:
a) a first double stranded RNA (dsRNA) and a first antisense oligonucleotide (ASO);
b) a first double stranded RNA (dsRNA) and a second dsRNA;
c) a first antisense oligonucleotide (ASO) and a second ASO;
d) a first double stranded RNA (dsRNA), a second dsRNA, and a third dsRNA;
e) a first double stranded RNA (dsRNA), a second dsRNA, and a first antisense oligonucleotide (ASO);
f) a first double stranded RNA (dsRNA), a first antisense oligonucleotide (ASO) and a second ASO; or
g) a first antisense oligonucleotide (ASO), a second ASO, and a third ASO,
wherein in any one of a)˜ g), the functional oligonucleotides are arranged in any order and covalently linked with or without linking component(s).

21. The multi-valent oligonucleotide agent of claim 20, wherein if presented, the first dsRNA, the second dsRNA and the third dsRNAs are independently selected from a siRNA and a saRNA; and/or
wherein if presented, the first ASO, the second ASO and the third ASO are independently selected from a gapmer and a mixmer.

22. The multi-valent oligonucleotide agent of claim 21, wherein the multi-valent oligonucleotide agent comprises functional oligonucleotides selected from:
a) siRNA-siRNA; b) siRNA-saRNA; c) saRNA-saRNA; d) siRNA-gapmer;
e) siRNA-mixmer; f) saRNA-gapmer; g) saRNA-mixmer; h) gapmer-gapmer;
i) gapmer-mixmer; j) mixmer-mixmer;
k) siRNA-siRNA-siRNA; 1) siRNA-siRNA-saRNA; m) siRNA-saRNA-saRNA;
n) saRNA-saRNA-saRNA; o) siRNA-siRNA-gapmer; p) siRNA-siRNA-mixmer;
q) siRNA-saRNA-gapmer; r) siRNA-saRNA-mixmer; s) saRNA-saRNA-gapmer;
t) saRNA-saRNA-mixmer; u) siRNA-gapmer-gapmer; v) saRNA-gapmer-gapmer;
w) siRNA-gapmer-mixmer; x) saRNA-gapmer-mixmer; y) siRNA-mixmer-mixmer;
z) saRNA-mixmer-mixmer; aa) gapmer-gapmer-gapmer; ab) gapmer-gapmer-mixmer;
ac) gapmer-mixmer-mixmer; and, ad) mixmer-mixmer-mixmer,
wherein in any one of a)˜ ad), the functional oligonucleotides are arranged in any order and covalently linked with or without linking component(s).

23. (canceled)

24. (canceled)

25. The multi-valent oligonucleotide agent of claim 1, comprising one or more additional targeting oligonucleotide(s).

26. The multi-valent oligonucleotide agent of claim 25, wherein the additionally targeting oligonucleotide(s) are independently selected from: a double stranded RNA (dsRNA) and an antisense oligonucleotide (ASO).

27. (canceled)

28. (canceled)

29. The multi-valent oligonucleotide agent of claim 1, wherein one or more of functional oligonucleotides increase the expression of a SMN2 gene or protein.

30. The multi-valent oligonucleotide agent of claim 29, wherein the dsRNA(s) increases the expression of the SMN2 gene or protein; and/or the ASO(s) increases the production of functional SMN protein by modulating SMN2 mRNA splicing or stability (SMN2 mRNA modulators).

31. The multi-valent oligonucleotide agent of claim 29, wherein the dsRNA(s) comprises a saRNA having a nucleotide sequence of a sense strand that is at least 90% identical to the nucleotide sequence selected from:
a) DS06-0004 (SEQ ID NO: 5);
b) DS06-0031 (SEQ ID NO: 7);
c) DS06-0067 (SEQ ID NO: 9);
d) DS06-4A3 (SEQ ID NO: 146);
e) R6-04-S1 (SEQ ID NO: 59); and
f) R6-04(20)-S1V1v(CM-4) (SEQ ID NO: 60); and/or
wherein the dsRNA(s) comprises a saRNA having a nucleotide sequence of an antisense strand that is at least 90% identical to the nucleotide sequence selected from:
a′) DS06-0004 (SEQ ID NO: 6);
b′) DS06-0031 (SEQ ID NO: 8);
c′) DS06-0067 (SEQ ID NO: 10);
d′) DS06-4A3 (SEQ ID NO: 147);
e′) R6-04-S1 (SEQ ID NO: 53); and
f′) R6-04M1-27A-S1L1V3(CM-26) (SEQ ID NO: 17).

32. The multi-valent oligonucleotide agent of claim 29, wherein the dsRNA(s) comprises a saRNA having a pair of nucleotide sequences of a sense strand and an antisense strand that is at least 90% identical to the nucleotide sequence pairs selected from:
a) DS06-0004: SEQ ID NO: 5 and SEQ ID NO: 6;
b) DS06-0031: SEQ ID NO: 7 and SEQ ID NO: 8;
c) DS06-0067: SEQ ID NO: 9 and SEQ ID NO: 10;
d) DS06-4A3: SEQ ID NO: 146 and SEQ ID NO: 147;
e) R6-04-S1: SEQ ID NO: 59 and SEQ ID NO: 53; and
f) R6-04(20)-S1V1v(CM-4): SEQ ID NO: 60 and SEQ ID NO: 17.

33. The multi-valent oligonucleotide agent of claim 29, wherein the dsRNA comprises a siRNA having a nucleotide sequence of a sense strand that is at least 90% identical to the nucleotide sequence of DS06-332i (SEQ ID NO: 3) or siSOD1-388-ESC (SEQ ID NO:138); and/or
wherein the dsRNA comprises a siRNA having a nucleotide sequence of an antisense strand that is at least 90% identical to the nucleotide sequence of DS06-332i (SEQ ID NO: 4) or siSOD1-388-ESC (SEQ ID NO: 139).

34. (canceled)

35. The multi-valent oligonucleotide agent of claim 29, wherein the ASO has a nucleotide sequence that is at least 90% identical to the nucleotide sequence of ASO10-27 (SEQ ID NO: 11) or 5′UTR ASO (SEQ ID NO: 142).

36. The multi-valent oligonucleotide agent of claim 29, wherein the multi-valent oligonucleotide agent has a nucleotide sequence that is at least 90% identical to the nucleotide sequences selected from:
a) DA06-4A-27A (SEQ ID NO: 14) and an antisense saRNA strand having a nucleotide sequence of SEQ ID NO: 13 that has partial complementarity with the sense saRNA strand of SEQ ID NO: 14;
b) DA06-4A-27B (SEQ ID NO: 15) and an antisense saRNA strand having a nucleotide sequence of SEQ ID NO: 13 that has partial complementarity with the sense saRNA strand of SEQ ID NO: 15;
c) R6-04M1-27A-S1L1V3 (SEQ ID NO: 18) and an antisense saRNA strand having a nucleotide sequence of SEQ ID NO: 13 that has partial complementarity with the sense saRNA strand of SEQ ID NO: 18;
d) DA06-31A-27A (SEQ ID NO: 19) and an antisense saRNA strand having a nucleotide sequence of SEQ ID NO: 8 that has partial complementarity with the sense saRNA strand of SEQ ID NO: 19;
e) DA06-31B-27A (SEQ ID NO: 20) and a sense saRNA strand having a nucleotide sequence of SEQ ID NO: 7 that has partial complementarity with the antisense saRNA strand of SEQ ID NO: 20;
f) DA06-67A-27A (SEQ ID NO: 21) and an antisense saRNA strand having a nucleotide sequence of SEQ ID NO: 10 that has partial complementarity with the sense saRNA strand of SEQ ID NO: 21;
g) DA06-67B-27A (SEQ ID NO: 22) and a sense saRNA strand having a nucleotide sequence of SEQ ID NO: 9 that has partial complementarity with the antisense saRNA strand of SEQ ID NO: 22;
h) DA6-67A3′L0-27A (SEQ ID NO: 23) and an antisense saRNA strand having a nucleotide sequence of SEQ ID NO: 10 that has partial complementarity with the sense saRNA strand of SEQ ID NO: 23;
j) DA6-67A3′L9-27A (SEQ ID NO: 24) and an antisense saRNA strand having a nucleotide sequence of SEQ ID NO: 10 that has partial complementarity with the sense saRNA strand of SEQ ID NO: 24;
k) DA6-67A3′L4-27A (SEQ ID NO: 25) and an antisense saRNA strand having a nucleotide sequence of SEQ ID NO: 10 that has partial complementarity with the sense saRNA strand of SEQ ID NO: 25;
l) DA6-67B3′L0-27A (SEQ ID NO: 26) and a sense saRNA strand having a nucleotide sequence of SEQ ID NO: 9 that has partial complementarity with the antisense saRNA strand of SEQ ID NO: 26;
m) DA6-67B5′L1-27A (SEQ ID NO: 27) and a sense saRNA strand having a nucleotide sequence of SEQ ID NO: 9 that has partial complementarity with the antisense saRNA strand of SEQ ID NO: 27;
o) DA6-67B5′L9-27A (SEQ ID NO: 29) and a sense saRNA strand having a nucleotide sequence of SEQ ID NO: 9 that has partial complementarity with the antisense saRNA strand of SEQ ID NO: 29;
p) DA6-67B5′L4-27A (SEQ ID NO: 30) and a sense saRNA strand having a nucleotide sequence of SEQ ID NO: 9 that has partial complementarity with the antisense saRNA strand of SEQ ID NO: 30;
q) DA6-67B3′L9-27A (SEQ ID NO: 31) and a sense saRNA strand having a nucleotide sequence of SEQ ID NO: 9 that has partial complementarity with the antisense saRNA strand of SEQ ID NO: 31;
r) DA6-67B3′L4-27A (SEQ ID NO: 32) and a sense saRNA strand having a nucleotide sequence of SEQ ID NO: 9 that has partial complementarity with the antisense saRNA strand of SEQ ID NO: 32;
S) DA06-67A21L1-27A (SEQ ID NO: 33) and an antisense saRNA strand having a nucleotide sequence of SEQ ID NO: 34 that has partial complementarity with the sense saRNA strand of SEQ ID NO: 33;
t) DA06-67B21L1-27A (SEQ ID NO: 36) and sense saRNA strand of SEQ ID NO: 35 that has partial complementarity with the antisense saRNA strand of SEQ ID NO: 36;
u) DA6-04A3′L0-27A (SEQ ID NO: 37) and an antisense saRNA strand having a nucleotide sequence of SEQ ID NO: 6 that has partial complementarity with the sense saRNA strand of SEQ ID NO: 37;
v) DA6-04A5′L1-27A (SEQ ID NO: 38) and an antisense saRNA strand having a nucleotide sequence of SEQ ID NO: 6 that has partial complementarity with the sense saRNA strand of SEQ ID NO: 38;
w) DA6-04A5′L9-27A (SEQ ID NO: 39) and an antisense saRNA strand having a nucleotide sequence of SEQ ID NO: 6 that has partial complementarity with the sense saRNA strand of SEQ ID NO: 39;
x) DA6-04A5′L4-27A (SEQ ID NO: 40) and an antisense saRNA strand having a nucleotide sequence of SEQ ID NO: 6 that has partial complementarity with the sense saRNA strand of SEQ ID NO: 40;
y) DA6-04A3′L1-27A (SEQ ID NO: 41) and an antisense saRNA strand having a nucleotide sequence of SEQ ID NO: 6 that has partial complementarity with the sense saRNA strand of SEQ ID NO: 41;
z) DA6-04A3′L9-27A (SEQ ID NO: 42) and an antisense saRNA strand having a nucleotide sequence of SEQ ID NO: 6 that has partial complementarity with the sense saRNA strand of SEQ ID NO: 42;
aa) DA6-04A3′L4-27A (SEQ ID NO: 43) and an antisense saRNA strand having a nucleotide sequence of SEQ ID NO: 6 that has partial complementarity with the sense saRNA strand of SEQ ID NO: 43;
bb) DA6-04B3′L0-27A (SEQ ID NO: 44) and a sense saRNA strand having a nucleotide sequence of SEQ ID NO: 5 that has partial complementarity with the antisense saRNA strand of SEQ ID NO: 44;
cc) DA6-04B3′L1-27A (SEQ ID NO: 45) and a sense saRNA strand having a nucleotide sequence of SEQ ID NO: 5 that has partial complementarity with the antisense saRNA strand of SEQ ID NO: 45;
dd) DA6-04B3′L9-27A (SEQ ID NO: 46) and a sense saRNA strand having a nucleotide sequence of SEQ ID NO: 5 that has partial complementarity with the antisense saRNA strand of SEQ ID NO: 46;
ee) DA6-04B3′L4-27A (SEQ ID NO: 47) and a sense saRNA strand having a nucleotide sequence of SEQ ID NO: 5 that has partial complementarity with the antisense saRNA strand of SEQ ID NO: 47;
ff) DA06-04A21L1-27A (SEQ ID NO: 48) and an antisense saRNA strand having a nucleotide sequence of SEQ ID NO: 49 that has partial complementarity with the sense saRNA strand of SEQ ID NO: 48;
gg) DA06-04B21L1-27A (SEQ ID NO: 51) and a sense saRNA strand of SEQ ID NO: 50 that has partial complementarity with the antisense saRNA strand of SEQ ID NO: 51;
hh) R6-04M1-16nt-S1L1V3v (SEQ ID NO: 79) and an antisense saRNA strand of SEQ ID NO: 17 that has partial complementarity with the sense saRNA strand of SEQ ID NO: 79;
ii) R6-04M1-15nt-S1L1V3v (SEQ ID NO: 80) and an antisense saRNA strand of SEQ ID NO: 17 that has partial complementarity with the sense saRNA strand of SEQ ID NO: 80;
jj) R6-04M1-14nt-S1L1V3v (SEQ ID NO: 81) and an antisense saRNA strand of SEQ ID NO: 17 that has partial complementarity with the sense saRNA strand of SEQ ID NO: 81;
kk) R6-04M1-13nt-S1L1V3v (SEQ ID NO: 82) and an antisense saRNA strand of SEQ ID NO: 17 that has partial complementarity with the sense saRNA strand of SEQ ID NO: 82;
ll) R6-04M1-(12nt-B)-S1L1V3v (SEQ ID NO: 83) and an antisense saRNA strand of SEQ ID NO: 17 that has partial complementarity with the sense saRNA strand of SEQ ID NO: 83;
mm) R6-04M1-11nt-S1L1V3v (SEQ ID NO: 84) and an antisense saRNA strand of SEQ ID NO: 17 that has partial complementarity with the antisense saRNA strand of SEQ ID NO: 84;
nn) R6-04M1-10nt-S1L1V3v (SEQ ID NO: 85) and an antisense saRNA strand of SEQ ID NO: 17 that has partial complementarity with the antisense saRNA strand of SEQ ID NO: 85;
oo) R6-04M1-9nt-S1L1V3v (SEQ ID NO: 86) and an antisense saRNA strand of SEQ ID NO: 17 that has partial complementarity with the sense saRNA strand of SEQ ID NO: 86;
pp) R6-04M1-8nt-S1L1V3v (SEQ ID NO: 87) and an antisense saRNA strand of SEQ ID NO: 17 that has partial complementarity with the sense saRNA strand of SEQ ID NO: 87;
qq) R6-04M1-7nt-S1L1V3v (SEQ ID NO: 88) and an antisense saRNA strand of SEQ ID NO: 17 that has partial complementarity with the sense saRNA strand of SEQ ID NO: 88;
rr) R6-04M1-6nt-S1L1V3v (SEQ ID NO: 89) and an antisense saRNA strand of SEQ ID NO: 17 that has partial complementarity with the sense saRNA strand of SEQ ID NO: 89;
ss) DS06-4A-S2L5V (SEQ ID NO: 128) and an antisense saRNA strand having a nucleotide sequence of SEQ ID NO: 17 that has partial complementarity with the sense saRNA strand of SEQ ID NO: 128;
ss′) DS06-4A-S2L1v (SEQ ID NO: 16) and an antisense saRNA strand having a nucleotide sequence of SEQ ID NO: 17 that has partial complementarity with the sense saRNA strand of SEQ ID NO: 16;
tt) DA6-27A-5′UTR (SEQ ID NO: 143);
uu) DA6-5′UTR-27A (SEQ ID NO: 144);
vv) R6-67M3-27A-S1L1V3 (SEQ ID NO: 129) and an antisense saRNA strand having a nucleotide sequence of SEQ ID NO: 130 that has partial complementarity with the sense saRNA strand of SEQ ID NO: 129;
ww) R6-67M3-16nt-S1L1V3 (SEQ ID NO: 129) and an antisense saRNA strand having a nucleotide sequence of SEQ ID NO: 131 that has partial complementarity with the sense saRNA strand of SEQ ID NO: 129;
xx) R6-67M3-15nt-S1L1V3 (SEQ ID NO: 129) and an antisense saRNA strand having a nucleotide sequence of SEQ ID NO: 132 that has partial complementarity with the sense saRNA strand of SEQ ID NO: 129;
yy) R6-67M3-14nt-S1L1V3 (SEQ ID NO: 129) and an antisense saRNA strand having a nucleotide sequence of SEQ ID NO: 133 that has partial complementarity with the sense saRNA strand of SEQ ID NO: 129;
zz) R6-67M3-13nt-S1L1V3 (SEQ ID NO: 129) and an antisense saRNA strand having a nucleotide sequence of SEQ ID NO: 134 that has partial complementarity with the sense saRNA strand of SEQ ID NO: 129;
aaa) R6-67M3-12nt-S1L1V3 (SEQ ID NO: 129) and an antisense saRNA strand having a nucleotide sequence of SEQ ID NO: 135 that has partial complementarity with the sense saRNA strand of SEQ ID NO: 129;
bbb) R6-67M3-9nt-S1L1V3 (SEQ ID NO: 129) and an antisense saRNA strand having a nucleotide sequence of SEQ ID NO: 136 that has partial complementarity with the sense saRNA strand of SEQ ID NO: 129;
ccc) R6-67M3-8nt-S1L1V3 (SEQ ID NO: 129) and an antisense saRNA strand having a nucleotide sequence of SEQ ID NO: 137 that has partial complementarity with the sense saRNA strand of SEQ ID NO: 129;
wherein linkers selected from the group consisting of L1, L4 and L9 is present or absent, wherein L1 represents spacer-18; L4 represents spacer-C6; and L9 represents spacer-9.

37. The multi-valent oligonucleotide agent of claim 29, wherein the multi-valent oligonucleotide agent has a nucleotide sequence that is at least 90% identical to the nucleotide sequences selected from:
a) R6-04S1&67S1R-L1V2 (SEQ ID NO: 52) and an antisense saRNA strand having a nucleotide sequence of SEQ ID NO: 53 that has partial complementarity with the strand of SEQ ID NO: 52 and an antisense saRNA strand having a nucleotide sequence of SEQ ID NO: 78 that has partial complementarity with the strand of SEQ ID NO: 52;
b) R6-04S1&67S5-L1V2 (SEQ ID NO: 56) and an antisense saRNA strand having a nucleotide sequence of SEQ ID NO: 53 that has partial complementarity with the strand of SEQ ID NO: 56 and an antisense saRNA strand having a nucleotide sequence of SEQ ID NO: 78 that has partial complementarity with the strand of SEQ ID NO: 56; and
c) R6-04M1&R17-388E-L1V2 (SEQ ID NO: 140) and an antisense saRNA strand of SEQ ID NO: 17 that has partial complementarity with the strand of SEQ ID NO: 140 and an antisense siRNA strand of SEQ ID NO: 141 that has partial complementarity with the strand of SEQ ID NO: 140; and/or
wherein the multi-valent oligonucleotide agent has a nucleotide sequence that is at least 90% identical to the nucleotide sequences selected from:
a′) R6-04S1&27A&67S1R-L1V2 (SEQ ID NO: 54) and an antisense saRNA strand having a nucleotide sequence of SEQ ID NO: 53 that has partial complementarity with the strand of SEQ ID NO: 54 and an antisense saRNA strand having a nucleotide sequence of SEQ ID NO: 78 that has partial complementarity with the strand of SEQ ID NO: 54;
b′) R6-04S1&67S1R&27A-L1V2 (SEQ ID NO: 55) and an antisense saRNA strand having a nucleotide sequence of SEQ ID NO: 53 that has partial complementarity with the strand of SEQ ID NO: 55 and an antisense saRNA strand having a nucleotide sequence of SEQ ID NO: 78 that has partial complementarity with the strand of SEQ ID NO: 55;
c′) R6-04S1&27A&67S5-L1V2 (SEQ ID NO: 57) and an antisense saRNA strand having a nucleotide sequence of SEQ ID NO: 53 that has partial complementarity with the strand of SEQ ID NO: 57 and an antisense saRNA strand having a nucleotide sequence of SEQ ID NO: 78 that has partial complementarity with the strand of SEQ ID NO: 57; and
d′) R6-04S1&67S5&27A-L1V2 (SEQ ID NO: 58) and an antisense saRNA strand having a nucleotide sequence of SEQ ID NO: 53 that has partial complementarity with the strand of SEQ ID NO: 58 and an antisense saRNA strand having a nucleotide sequence of SEQ ID NO: 78 that has partial complementarity with the strand of SEQ ID NO: 58.

38. The multi-valent oligonucleotide agent of claim 1, wherein the multi-valent oligonucleotide agent is selected from or has at least 90% sequence identity to those shown in any one of Tables 7-20, and wherein the linking components and/or linkage bonds and/or orientation of those multi-valent oligonucleotide agents are changeable.

39. The multi-valent oligonucleotide agent of claim 1, wherein one or more of the functional oligonucleotides increase the expression of CDKN1A/p21 gene or protein and/or decreases the expression CD274/PDL-1.

40. The multi-valent oligonucleotide agent of claim 39, wherein the dsRNA(s) are independently selected from: a saRNA that increases the expression of the CDKN1A/p21 gene or protein; and a siRNA that decreases the expression CD274/PDL-1; and/or
wherein the ASO(s) are independently selected from an ASO that increases the expression of CDKN1A/p21 gene or protein and/or decreases the expression CD274/PDL-1.

41. The multi-valent oligonucleotide agent of claim 39, wherein the dsRNA is a saRNA having a nucleotide sequence of a sense strand that is at least 90% identical to the nucleotide sequence of RAG1-40 (SEQ ID NO: 62); and/or
wherein the dsRNA is a saRNA having a nucleotide sequence of an antisense strand that is at least 90% identical to the nucleotide sequence of RAG1-40 (SEQ ID NO: 63).

42. The multi-valent oligonucleotide agent of claim 39, wherein the dsRNA is a saRNA having a nucleotide sequence of a sense strand that is at least 90% identical to the nucleotide sequence of RAG1-40 (SEQ ID NO: 62) and a nucleotide sequence of an antisense strand that is at least 90% identical to the nucleotide sequence of RAG1-40 (SEQ ID NO: 63.

43. The multi-valent oligonucleotide agent of claim 39, wherein the dsRNA is a siRNA having a nucleotide sequence of a sense strand that is at least 90% identical to the nucleotide sequence selected from:
a) siPDL1-2 (SEQ ID NO: 64); and
b) siPDL1-3 (SEQ ID NO: 66); and/or
wherein the dsRNA is a siRNA having a nucleotide sequence of an antisense strand that is at least 90% identical to the nucleotide sequence selected from:
a) siPDL1-2 (SEQ ID NO: 65); and
b) siPDL1-3 (SEQ ID NO: 67).

44. The multi-valent oligonucleotide agent of claim 39, wherein the dsRNA is a siRNA selected from:
a) a siRNA having a nucleotide sequence of a sense strand that is at least 90% identical to the nucleotide sequence siPDL1-2 (SEQ ID NO: 64) and a nucleotide sequence of an antisense strand that is at least 90% identical to the nucleotide sequence of siPDL1-2 (SEQ ID NO: 65); and
b) a siRNA having a nucleotide sequence of a sense strand that is at least 90% identical to the nucleotide sequence siPDL1-2 (SEQ ID NO: 66) and a nucleotide sequence of an antisense strand that is at least 90% identical to the nucleotide sequence of siPDL1-2 (SEQ ID NO: 67); and/or
wherein the ASO has a nucleotide sequence that is at least 90% identical to the nucleotide sequence selected from:
a″) aPDL1-1 (SEQ ID NO: 68);
b″) aPDL1-2 (SEQ ID NO: 69); and
c″) aPDL1-3 (SEQ ID NO: 70); and/or
wherein the multi-valent oligonucleotide agent has a nucleotide sequence that is at least 90% identical to the nucleotide sequences selected from:
a′) saP21-40/siPDL1-2 (SEQ ID NO: 71) and an antisense saRNA strand having a nucleotide sequence of SEQ ID NO: 63 that has partial complementarity with the strand of SEQ ID NO: 71 and an antisense siRNA strand having a nucleotide sequence of SEQ ID NO: 65 that has partial complementarity with the strand of SEQ ID NO: 71;
b′) saP21-40/siPDL1-3 (SEQ ID NO: 100) and an antisense saRNA strand having a nucleotide sequence of SEQ ID NO: 63 that has partial complementarity with the sense saRNA strand of SEQ ID NO: 100 and an antisense saRNA strand having a nucleotide sequence of SEQ ID NO: 65 that has partial complementarity with the strand of SEQ ID NO: 100;
c′) saP21-40/aPDL1-1 (SEQ ID NO: 72) and an antisense saRNA strand having a nucleotide sequence of SEQ ID NO: 63 that has partial complementarity with the sense saRNA strand of SEQ ID NO: 72;
d′) saP21-40/aPDL1-2 (SEQ ID NO: 73) and an antisense saRNA strand having a nucleotide sequence of SEQ ID NO: 63 that has partial complementarity with the sense saRNA strand of SEQ ID NO: 73;
e′) saP21-40/aPDL1-3 (SEQ ID NO: 74) and an antisense saRNA strand having a nucleotide sequence of SEQ ID NO: 63 that has partial complementarity with the sense saRNA strand of SEQ ID NO: 74;
f′) saP21-40/aPDL1-1R (SEQ ID NO: 75) and an antisense saRNA strand having a nucleotide sequence of SEQ ID NO: 63 that has partial complementarity with the sense saRNA strand of SEQ ID NO: 75;
g′) saP21-40/aPDL1-2R (SEQ ID NO: 76) and an antisense saRNA strand having a nucleotide sequence of SEQ ID NO: 63 that has partial complementarity with the sense saRNA strand of SEQ ID NO: 76; and
h′) saP21-40/aPDL1-3R (SEQ ID NO: 77) and an antisense saRNA strand having a nucleotide sequence of SEQ ID NO: 63 that has partial complementarity with the sense saRNA strand of SEQ ID NO: 77.

45. (canceled)

46. A product comprising the multi-valent oligonucleotide agent of claim 1.

47. The product of claim 46, wherein the product is selected from a pharmaceutical composition further comprising at least one pharmaceutically acceptable carrier or a kit.

48. (canceled)

49. (canceled)

50. A method for disease treatment comprising administering sufficient amount of one or more of the multi-valent oligonucleotide agent of claim 1 to a subject in need of such treatment.

51.-55. (canceled)

56. The method of claim 50, wherein the patient is suffering from cancer or in high risk of having cancer; and/or
wherein the cancer is a solid tumor or a non-solid tumor.

57.-61. (canceled)

62. A method for the preparation of the multi-valent oligonucleotide agent of claim 1 comprising:
providing said two or more functional oligonucleotides and covalently linking the same; or
synthesizing the full length oligonucleotide agent.

63.-69. (canceled)

70. An isolated or synthesized oligonucleotide comprising: a nucleotide sequence of a saRNA sense strand that is at least 90% identical to the nucleotide sequence of SEQ ID NO: 62.

71.-73. (canceled)

74. A pharmaceutical composition or kit comprising the isolated or synthesized oligonucleotide of claim 70.

75. A method for disease treatment comprising administering sufficient amount of one or more of the isolated or synthesized oligonucleotide of claim 70 to a subject in need of such treatment.
